(19) United States
(12) Patent Application Publication
(10) Pub. No.: US 2023/0021583 A1
(43) Pub. Date: Jan. 26, 2023

(54) MEASLES-VECTORED COVID-19 IMMUNOGENIC COMPOSITIONS AND VACCINES

(71) Applicant: INSTITUT PASTEUR, PARIS (FR)

(72) Inventors: NICOLAS Robert, Xavier ESCRIOU, Paris (FR); FREDERIC TANGY, Paris (FR); CHRISTIANE GERKE, Paris (FR)

(21) Appl. No.: 17/797,570

(22) PCT Filed: Feb. 12, 2021
(86) PCT No.: PCT/EP2021/053540

§ 371 Date: Aug. 4, 2022

Related U.S. Application Data
(60) Provisional application No. 62/976,083, filed on Feb. 13, 2020.

(30) Foreign Application Priority Data
Feb. 13, 2020 (EP) 20305141.2
Jul. 8, 2020 (EP) 20305790.6

(51) Int. Cl. A61K 39/215; A61P 31/14; A61K 39/165; C12N 15/86

(52) U.S. Cl. CPC A61K 39/215; A61P 31/14; A61K 39/165; C12N 15/86; A61K 2039/5254

(57) ABSTRACT

The invention relates to the field of immunity against Coronaviruses. In this respect, the invention provides vectorized antigens derived from Coronaviruses that trigger an immune response against Coronaviruses. The invention accordingly relates to an active ingredient which is a live attenuated recombinant measles virus expressing Coronavirus antigen(s) and to its use in eliciting immunity, in particular protective immunity against SARS-CoV-2 strain and advantageously broad-spectrum protective immunity against various strains of Coronaviruses.

### FIELD OF THE INVENTION

The invention relates to the field of immunity against Coronaviruses.

### BACKGROUND

Coronaviruses are enveloped, positive-sense single-stranded RNA viruses with large genome (from 26 to 32 kb). Four genera (alpha, beta, gamma and delta) have been described and among them betacoronavirus has been subdivided in four lineages (A, B, C and D). Among the host identified for coronaviruses avian and mammalian specified, including humans have been especially shown to be infected either by strains circulating annually or by strains capable of giving rise to pandemic outbreaks. Human coronaviruses include annual strains HCoV-OC43, HCoV-229E, HCoV-HKU1, HCoV-NL63 and pandemic strains such as SARS-CoV (Severe Acute Respiratory Syndrome coronavirus) isolated in 2003 or MERS-CoV (Middle East Respiratory Syndrome coronavirus) isolated in 2012 and still circulating. SARS-CoV and MERS-CoV belong to the betacoronavirus lineage B and lineage C respectively. These coronaviruses are airborne transmitted and have been shown to have human-to-human transmission.

Beside these known strains, a new Coronavirus strain designated 2019-nCoV (or more recently designated severe acute respiratory syndrome coronavirus 2 (SARS-CoV-2) has been identified in 2019 as currently infecting people in China and that has spread around the world resulting in severe illness or death for a large number of the infected human hosts. This highly pathogenic stain emerged into the human population from animal reservoirs and has already proved to be responsible of high case-fatality rates including by human-to-human transmission causing great concerns for a coronavirus pandemic. On Jan. 30, 2020, the outbreak was declared a Public Health Emergency of International Concern by the World Health Organization (WHO) and then characterized as a pandemic on Mar. 11, 2020. The WHO also announced a name for this new coronavirus disease: COVID-19.

Almost 100 million people worldwide have been affected by the SARS-CoV-2 pandemic to date and more than 2 million have died of COVID-19. The pandemic has resulted in unprecedented global social and economic disruption, with a projected “optimistic loss” of $3.3 trillion and a worst-case scenario loss of $82 trillion worldwide (The GDP Risk; 2020). While the virus is now explosively expanding in a second wave in Europe and the northern hemisphere, no specific treatment has been shown to prevent or cure the disease. Together with enforcing public health measures, effective vaccines are needed for a return to pre-COVID-19 normalcy. There is therefore a need to propose vaccine candidates for inducing an immune response against SARS-CoV-2 and potentially providing protection against SARS-CoV-2 or possibly against a broader range of coronavirus strains, e.g., epidemic or pandemic strains.

This invention meets these and other needs.

### SUMMARY

The invention provides vectorized antigens derived from Coronaviruses that trigger an immune response against Coronaviruses. The invention accordingly relates to an active ingredient which is a live attenuated recombinant measles virus expressing Coronavirus antigen(s) and to its use in eliciting immunity, in particular protective immunity against 2019-nCoV (SARS-CoV-2) strain and advantageously broad-spectrum protective immunity against various strains of Coronaviruses. The invention also relates to polypeptides derived from the native antigens of SARS-CoV-2 wherein the polypeptides have useful properties to design efficient immunogens, in particular to design a vaccine candidate against coronavirus infection. The invention also relates to polynucleotides encoding the native antigens of SARS-CoV-2 or encoding polypeptides derived from the native antigens of SARS-CoV-2, in particular polynucleotides adapted for expression by a recombinant measles virus or for improved recovery from producing cells.

The invention is also directed to means for the preparation of recombinant measles virus expressing the polypeptides obtained from antigens of SARS-CoV-2 and to recombinant measles virus thus obtained.

The invention also concerns an immunogenic composition comprising recombinant measles virus expressing the polypeptides obtained from antigens of SARS-CoV-2. The invention also relates to the use of such immunogenic composition for eliciting a protective immune response in an animal host, in particular a mammalian host, especially a human host, against SARS-CoV-2 and optionally against other coronaviruses or against disease caused by the infection. The invention also relates to a method for the treatment of a host in need thereof, in particular for prophylactic treatment, against the infection by SARS-CoV-2 and optionally against other coronaviruses or against disease caused by the infection.

In particular, in a first aspect the invention provides a nucleic acid construct comprising: a cDNA molecule encoding a full length, antigenomic (+) RNA strand of an attenuated strain of measles virus (MV); and a first heterologous polynucleotide encoding: (a) a spike (S) protein of SARS-CoV-2 of SEQ ID NO: 3, or (b) an immunogenic fragment of the full-length S protein in (a) selected from the group consisting of the S1 polypeptide of SEQ ID NO: 11, the S2 polypeptide of SEQ ID NO: 13, the Secto polypeptide of SEQ ID NO: 7 and the tri-Secto polypeptide of SEQ ID NO: 16, or (c) a variant of (a) or (b) in which from 1 to 10 amino acids are modified by insertion, substitution, or deletion. In some embodiments the variant in (c) encodes a polypeptide comprising: (i) a mutation that maintains the expressed full length S protein in its prefusion conformation, and/or (ii) a mutation that inactivates the furin cleavage site of the S protein, and/or (iii) a mutation that inactivates the Endoplasmic Reticulum Retrieval Signal (EERS), and/or (iv) a mutation that maintains the receptor-binding domain (RBD) localized in the S1 domain of the S protein in the closed conformation, and wherein the first heterologous polynucleotide is positioned in an additional transcription unit (ATU) located between the P gene and the M gene of the MV (ATU2) or in an ATU located downstream of the H gene of the MV (ATU3). In some embodiments the mutation that maintains the expressed full length S protein in its prefusion conformation is a mutation by substitution of two proline residues at positions 986 and 987 (K986P and V987P) of the amino acid sequence of the S protein of SARS-CoV-2 of SEQ ID NO: 3, or a mutation by substitution of six proline residues at positions 817, 892, 899, 942, 986 and 987 (F817P, A892P, A899P, A942P, K986P and V987P) of the amino acid sequence of the S protein of SARS-CoV-2 of SEQ ID NO: 3, and/or the mutation that inactivates the furin cleavage site of the S protein is a mutation by substitution of three amino acid residues occurring in the S1/S2 furin cleavage site at positions 682, 683 and 685 (R682G, R683S and R685G) of the amino acid sequence of the S protein of SARS-CoV-2 of SEQ ID NO: 3, or a mutation by deletion of the loop encompassing the S1/S2 furin cleavage site between amino acid at position 675 and amino acid at position 685 of the S protein of SARS-CoV-2 of SEQ ID NO: 3, the loop consisting of the amino acid sequence QTQTNSPRRAR of SEQ ID NO: 50, and/or the mutation that inactivates the EERS is a mutation by substitution of two alanine residues at positions 1269 and 1271 of the amino acid sequence of SEQ ID NO: 3, and/or the mutation that maintains the RBD localized in the S1 domain of the S protein in the closed conformation is a mutation by substitution of two cysteine residues at positions 383 and 985 (S383C and D985C) of the amino acid sequence of the S protein of SARS-CoV-2 of SEQ ID NO: 3, or a mutation by substitution of two cysteine residues at positions 413 and 987 (G413C and P987C) of the amino acid sequence of the S protein of SARS-CoV-2 of SEQ ID NO: 3; and/or the variant in (c) encodes a polypeptide comprising a mutation selected from the group consisting of a deletion of the amino acid residues at positions 69 and 70 of the amino acid sequence of SEQ ID NO: 3, a deletion of the amino acid residues at positions 144 and 145 of the amino acid sequence of SEQ ID NO: 3, a mutation by substitution of the tyrosine residue at position 501 of the amino acid sequence of SEQ ID NO: 3 (N501Y), a mutation by substitution of the aspartic acid residue at position 570 of the amino acid sequence of SEQ ID NO: 3 (A570D), a mutation by substitution of the histidine residue at position 681 of the amino acid sequence of SEQ ID NO: 3 (P681H), a mutation by substitution of the isoleucine residue at position 716 of the amino acid sequence of SEQ ID NO: 3 (T7161), a mutation by substitution of the alanine residue at position 982 of the amino acid sequence of SEQ ID NO: 3 (S982A), a mutation by substitution of the histidine residue at position 1118 of the amino acid sequence of SEQ ID NO: 3 (D1118H), a mutation by substitution of the lysine residue at position 484 of the amino acid sequence of SEQ ID NO: 3 (E484K), a mutation by substitution of the asparagine residue at position 417 of the amino acid sequence of SEQ ID NO: 3 (K417N), a mutation by substitution of the threonine residue at position 417 of the amino acid sequence of SEQ ID NO: 3 (K417T) and a mutation by substitution of the glycine residue at position 614 of the amino acid sequence of SEQ ID NO: 3 (D614G).

In some embodiments of the first aspect the nucleic acid construct further comprises a second heterologous polynucleotide encoding at least one polypeptide of SARS-CoV-2 selected from the group consisting of: nucleocapsid (N) polypeptide or a variant thereof having at least 90% identity with the N polypeptide, matrix (M) polypeptide or a variant thereof having at least 90% identity with M polypeptide, E polypeptide or a variant thereof having at least 90% identity with E polypeptide, 8a polypeptide or a variant thereof having at least 90% identity with 8a polypeptide, 7a polypeptide or a variant thereof having at least 90% identity with 7a polypeptide, 3A polypeptide or a variant thereof having at least 90% identity with 3a polypeptide, and immunogenic fragments thereof; the second heterologous polynucleotide positioned within an additional transcription unit (ATU) at a location different from the ATU of the first heterologous polynucleotide.

In some embodiments of the first aspect the first heterologous polynucleotide encodes a polypeptide having an amino acid sequence selected from the group consisting of SEQ ID NOs: 5, 7, 9, 15, 17, 19, 43, 45, 47, 49, 52, 54, 56, 58, 60, 62 and 65.

In some embodiments of the first aspect the second heterologous polynucleotide encodes at least one of the N polypeptide of SEQ ID NO: 22, the M polypeptide of sequence SEQ ID NO: 24 or its endodomain, the E polypeptide of sequence SEQ ID NO: 23, the ORF8 polypeptide of SEQ ID NO: 25, the ORF7a polypeptide of SEQ ID NO: 27, and the ORF3a polypeptide of SEQ ID NO: 26.

In some embodiments of the first aspect the first heterologous polynucleotide has the open reading frame selected from the group consisting of:
i. SEQ ID NO: 1 or 2 or 36 which encodes the S polypeptide,
ii. SEQ ID NO: 10 which encodes the S1 polypeptide,
iii. SEQ ID NO: 12 which encodes the S2 polypeptide,
iv. SEQ ID NO: 4 which encodes the stab-S polypeptide (S2P),
v. SEQ ID NO: 6 which encodes the Secto polypeptide,
vi. SEQ ID NO: 8 which encodes the stab-Secto polypeptide,
vii. SEQ ID NO:14 which encodes the stab-S2 polypeptide,
viii. SEQ ID NO: 16 which encodes the tri-Secto polypeptide,
ix. SEQ ID NO: 18 which encodes the tristab-Secto polypeptide,
x. SEQ ID NO: 42 which encodes the S3F polypeptide,
xi. SEQ ID NO: 44 which encodes the S2P3F polypeptide,
xii. SEQ ID NO: 46 which encodes the S2PΔF polypeptide,
xiii. SEQ ID NO: 48 which encodes the S2PΔF2A polypeptide,
xiv. SEQ ID NO: 51 which encodes the T4-S2P3F polypeptide (tristab-Secto-3F),
xv. SEQ ID NO: 53 which encodes the S6P polypeptide,
xvi. SEQ ID NO: 55 which encodes the S6P3F polypeptide,
xvii. SEQ ID NO: 57 which encodes the S6PΔF polypeptide,
xviii. SEQ ID NO: 59 which encodes the SCCPP polypeptide,
xix. SEQ ID NO: 61 which encodes the SCC6P polypeptide,
xx. SEQ ID NO: 63 which encodes the S_{MVopt}2P polypeptide,
xxi. SEQ ID NO: 64 which encodes the S_{MVopt}ΔF polypeptide, and
xxii. SEQ ID NO: 66 which encodes the S_{MVopt}2PΔF polypeptide.

In some embodiments of the first aspect the nucleic acid construct is a cDNA construct comprising from 5′ to 3′ end the following polynucleotides coding for open reading frames:
(a) a polynucleotide encoding the N protein of the MV;
(b) a polynucleotide encoding the P protein of the MV;
(c) the first heterologous polynucleotide as defined in any one of claims 1-3, 4 and 6;
(d) a polynucleotide encoding the M protein of the MV;
(e) a polynucleotide encoding the F protein of the MV;
(f) a polynucleotide encoding the H protein of the MV;
(g) a polynucleotide encoding the L protein of the MV; and

wherein the polynucleotides are operatively linked within the nucleic acid construct and are under the control of a viral replication and transcriptional regulatory elements such as MV leader and trailer sequences and are framed by a T7 promoter and a T7 terminator and are framed by restriction sites suitable for cloning in a vector to provide a recombinant MV-CoV expression cassette.

In some embodiments of the first aspect the nucleic acid construct further comprises (a) a GGG motif followed by a hammerhead ribozyme sequence at the 5′-end of the nucleic acid construct, adjacent to a first nucleotide of the nucleotide sequence encoding a full-length antigenomic (+)RNA strand of an attenuated MV strain, in particular of a Schwarz strain or of a Moraten strain, and (b) a nucleotide sequence of a ribozyme, in particular the sequence of the Hepatitis delta virus ribozyme (6), at the 3′-end of the recombinant MV-CoV nucleic acid molecule, adjacent to the last nucleotide of the nucleotide sequence encoding the full length anti-genomic (+)RNA strand.

In some embodiments of the first aspect having the second heterologous polynucleotide, the second heterologous polynucleotide encodes the N polypeptide of SARS-CoV-2, and the second heterologous polynucleotide being cloned in an ATU at a different location with respect to the ATU used for cloning the first heterologous polynucleotide.

In some embodiments of the first aspect (i) the first heterologous polynucleotide comprises a sequence selected from the group consisting of SEQ ID NO: 36, SEQ ID NO: 63, SEQ ID NO: 64 and SEQ ID NO: 66, and is positioned within ATU2, or (ii) the first heterologous polynucleotide comprises a sequence selected from the group consisting of SEQ ID NO: 2, SEQ ID NO: 4, SEQ ID NO: 44, SEQ ID NO: 46, SEQ ID NO: 48, SEQ ID NO: 51, SEQ ID NO: 53, SEQ ID NO: 55, SEQ ID NO: 57, SEQ ID NO: 59 and SEQ ID NO: 61, and is positioned within ATU3.

In some embodiments of the first aspect (i) the first heterologous polynucleotide is positioned within ATU3 and the second heterologous polynucleotide, is positioned within ATU2, or (ii) the first heterologous polynucleotide is positioned within ATU2 and the second heterologous polynucleotide, is positioned within ATU3.

In some embodiments of the first aspect the measles virus is an attenuated virus strain selected from the group consisting of the Schwarz strain, the Zagreb strain, the AIK-C strain, the Moraten strain, the Philips strain, the Beckenham 4A strain, the Beckenham 16 strain, the CAM-70 strain, the TD 97 strain, the Leningrad-16 strain, the Shanghai 191 strain and the Belgrade strain.

In a second aspect the invention provides nucleic acid constructs comprising: (1) a cDNA molecule encoding a full length antigenomic (+) RNA strand of an attenuated strain of measles virus (MV); and (2) a first heterologous polynucleotide encoding a S protein or immunogenic fragment thereof of SARS-CoV-2 comprising an insertion, substitution, or deletion in the 11 amino acid residue sequence of the S protein aligned with positions 1263 to 1273 of the amino acid sequence of SEQ ID NO: 3, and wherein the insertion, substitution, or deletion increases cell surface expression of the S protein or immunogenic fragment thereof, wherein the first heterologous polynucleotide is positioned in an additional transcription unit (ATU) located between the P gene and the M gene of the MV (ATU2) or in an ATU located 3′ of the H gene of the MV (ATU3). In some embodiments the S protein or immunogenic fragment thereof comprises a substitution in the 11 amino acid residue sequence of the S protein aligned with positions 1263 to 1273 of the amino acid sequence of SEQ ID NO: 3. In some embodiments the S protein or immunogenic fragment thereof comprises a deletion of all or part of the 11 amino acid residue sequence of the S protein aligned with positions 1263 to 1273 of the amino acid sequence of SEQ ID NO: 3. In some embodiments the encoded S protein or immunogenic fragment thereof further comprises one or more additional substitutions that maintain the expressed S protein in its prefusion conformation. In some embodiments the encoded S protein or immunogenic fragment thereof further comprises the amino acid substitutions K986P and V987P at the amino acid positions corresponding to positions K986 and V987 of the amino acid sequence of SEQ ID NO: 3. In some embodiments the encoded S protein or immunogenic fragment thereof is a dual domain S protein. In some embodiments the first heterologous polynucleotide is positioned in ATU2. In some embodiments the first heterologous polynucleotide encodes: (a) a prefusion-stabilized SF-2P-dER polypeptide of SEQ ID NO: 76, or a variant thereof having at least 90% identity with SEQ ID NO: 76, wherein the variant does not vary at positions 986 and 987; or (b) a prefusion-stabilized SF-2P-2a polypeptide of SEQ ID NO: 82, or a variant thereof having at least 90% identity with SEQ ID NO: 82, wherein the variant does not vary at positions 986, 987, 1269, and 1271. In some embodiments the first heterologous polynucleotide encodes: (a) a prefusion-stabilized SF-2P-dER polypeptide of SEQ ID NO: 76; or (b) a prefusion-stabilized SF-2P-2a polypeptide of SEQ ID NO: 82. In some embodiments the first heterologous polynucleotide comprises SEQ ID NO: 75 which encodes the SF-2P-dER polypeptide, or SEQ ID NO: 81 which encodes the SF-2P-2a polypeptide. In some embodiments the first heterologous polynucleotide comprises SEQ ID NO: 75 which encodes the SF-2P-dER polypeptide.

In some embodiments of the second aspect the nucleic acid construct further comprises a second heterologous polynucleotide encoding at least one polypeptide of SARS-CoV-2 selected from the group consisting of: nucleocapsid (N) polypeptide or a variant thereof having at least 90% identity with the N polypeptide; matrix (M) polypeptide or a variant thereof having at least 90% identity with M polypeptide; E polypeptide or a variant thereof having at least 90% identity with E polypeptide; 8a polypeptide or a variant thereof having at least 90% identity with 8a polypeptide; 7a polypeptide or a variant thereof having at least 90% identity with 7a polypeptide; 3A polypeptide or a variant thereof having at least 90% identity with 3 polypeptide; and immunogenic fragments thereof, the second heterologous polynucleotide being positioned within an additional transcription unit (ATU) at a location different from the ATU of the first heterologous polynucleotide.

In some embodiments of the second aspect the nucleic acid construct further comprises a second heterologous polynucleotide encoding at least one polypeptide of SARS-CoV-2 selected from the group consisting of: nucleocapsid (N) polypeptide; matrix (M) polypeptide; E polypeptide; 8a polypeptide; 7a polypeptide; 3A polypeptide; and immunogenic fragments thereof, the second heterologous polynucleotide being positioned within an additional transcription unit (ATU) at a location different from the ATU of the first heterologous polynucleotide. In some embodiments the second heterologous polynucleotide encodes N polypeptide, the second heterologous polynucleotide being positioned within an additional transcription unit (ATU) at a location different from the ATU of the first heterologous polynucleotide.

In some embodiments of the second aspect the second heterologous polynucleotide encodes at least one of the N polypeptide of SEQ ID NO: 22, the M polypeptide of sequence SEQ ID NO: 24 or its endodomain, the E polypeptide of sequence SEQ ID NO: 23, the ORF8 polypeptide of SEQ ID NO: 25, the ORF7a polypeptide of SEQ ID NO: 27 and/or the ORF3a polypeptide of SEQ ID NO: 26, the second heterologous polynucleotide being positioned within an additional transcription unit (ATU) at a location different from the ATU of the first heterologous polynucleotide.

In some embodiments of the second aspect the second heterologous protein is within an ATU that is upstream of the N gene of the MV (ATU1), between the P and M genes of the MV (ATU2), or between the H and L genes of the MV (ATU3).

In some embodiments of the second aspect the nucleic acid construct further comprises from 5′ to 3′ the following polynucleotides coding for open reading frames:
(a) a polynucleotide encoding the N protein of the MV;
(b) a polynucleotide encoding the P protein of the MV;
(c) the first heterologous polynucleotide;
(d) a polynucleotide encoding the M protein of the MV;
(e) a polynucleotide encoding the F protein of the MV;
(f) a polynucleotide encoding the H protein of the MV;
(g) a polynucleotide encoding the L protein of the MV; and

wherein the polynucleotides are operatively linked within the nucleic acid construct, are under the control of MV leader and trailer sequences, are framed by a T7 promoter and a T7 terminator, and are framed by restriction sites suitable for cloning in a vector to provide a recombinant MV-CoV expression cassette.

In some embodiments of the second aspect the nucleic acid construct further comprises:
(a) a GGG motif followed by a hammerhead ribozyme sequence at the 5′-end of the nucleic acid construct, adjacent to the first nucleotide of a nucleotide sequence encoding a full-length antigenomic (+)RNA strand of an attenuated MV strain; and
(b) a nucleotide sequence of the Hepatitis delta virus ribozyme (6) at the 3′-end of the nucleic acid construct, adjacent to a last nucleotide of the nucleotide sequence encoding the full length anti-genomic (+)RNA strand of the attenuated MV strain.

In some embodiments of the second aspect the measles virus is an attenuated virus strain selected from the group consisting of the Schwarz strain, the Zagreb strain, the AIK-C strain, the Moraten strain, the Philips strain, the Beckenham 4A strain, the Beckenham 16 strain, the CAM-70 strain, the TD 97 strain, the Leningrad-16 strain, the Shanghai 191 strain, and the Belgrade strain. In some embodiments the nucleic acid construct further comprises the measles virus is the Schwarz strain.

The nucleic acid constructions of the first and second aspects of the invention may be incorporated into further aspects of the invention.

In a third aspect the invention provides transfer vectors for the rescue of a recombinant Measles virus (MV), comprising the nucleic acid construct of the invention. In some embodiments the transfer vector comprises a sequence encoding a polypeptide of SARS-CoV-2 that is selected from the group consisting of:
i. SEQ ID NO: 1 or 2 or 36 (construct S),
ii. SEQ ID NO: 4 (construct stab-S),
iii. SEQ ID NO: 6 (construct Secto),
iv. SED ID NO: 8 (construct stab-Secto),
v. SEQ ID NO: 10 (construct S1),
vi. SEQ ID NO: 12 (construct S2),
vii. SEQ ID NO: 14 (construct stab-S2),
viii. SEQ ID NO: 16 (construct tri-Secto),
ix. SEQ ID NO: 18 (construct tristab-Secto),
x. SEQ ID NO: 42 (construct S3F),
xi. SEQ ID NO: 44 (construct S2P3F),
xii. SEQ ID NO: 46 (construct S2PΔF),
xiii. SEQ ID NO: 48 (construct S2PΔF2A),
xiv. SEQ ID NO: 21 or 37 (construct N),
xv. SEQ ID NO: 51 (construct T4-S2P3F (tristab-Secto-3F)),
xvi. SEQ ID NO: 53 (construct S6P),
xvii. SEQ ID NO: 55 (construct S6P3F),
xviii. SEQ ID NO: 57 (construct S6PΔF),
xix. SEQ ID NO: 59 (construct SCCPP),
xx. SEQ ID NO: 61 (construct SCC6P),
xxi. SEQ ID NO: 63 (construct S_{MVopt}2P),
xxii. SEQ ID NO: 64 (construct S_{MVopt}ΔF), and
xxiii. SEQ ID NO: 66 (construct S_{MVopt}2PΔF).

In a fourth aspect the invention provides a plasmid vector comprising a nucleic acid construct of the invention, wherein the plasmid vector is SEQ ID NO: 29 (pTM2-MVSchw-gfp, also named pTM-MVSchw2-GFPbis or pTM-MVSchwarz-ATU2) or SEQ ID NO: 38 (pTM3-MVSchw-gfp, also named pTM-MVSchw3-GFP or pTM-MVSchwarz-ATU3).

In a fifth aspect the invention provides a recombinant measles virus comprising a nucleic acid construct of the invention. In some embodiments the recombinant measles virus is of the Schwarz strain. In some embodiments the recombinant measles virus comprises in its genome an expression cassette operatively linked thereto, the expression cassette comprising the nucleic acid construct according to the invention. In some embodiments the recombinant measles virus further expresses at least one polypeptide selected from N, M, E, ORF7a, ORF8 and ORF3a of the SARS-CoV-2 strain, or an immunogenic fragment thereof.

In a sixth aspect the invention provides immunogenic compositions and vaccines comprising a recombinant measles virus of the invention. In some embodiments the immunogenic composition or the vaccine is for use in inducing an immune response against SARS-CoV-2 virus in a subject. In some embodiments the immunogenic compositions and vaccines comprise (i) an effective dose of a recombinant measles virus of the invention, and (ii) a pharmaceutically acceptable vehicle, wherein the composition or the vaccine elicits a neutralizing humoral response and/or a cellular response against a polypeptide(s) of SARS-CoV-2 in an animal host after a single immunization. In some embodiments the immunogenic composition or vaccine is for use in eliciting a protective humoral immune response and/or a cellular immune response against SARS-CoV-2 in a host in need thereof.

In a seventh aspect the invention provides a process for rescuing recombinant measles virus of the invention. The process may comprise:
(a) co-transfecting helper cells stably expressing T7 RNA polymerase and measles virus N and P proteins with (i) a nucleic acid construct according to the invention or with a plasmid vector comprising the nucleic acid construct according to the invention, and (ii) a vector encoding the MV L polymerase;
(b) maintaining the transfected helper cells in conditions suitable for the production of recombinant measles virus;
(c) infecting cells enabling propagation of the recombinant measles virus by co-cultivating them with the transfected helper cells of step (b);
(d) harvesting recombinant measles virus.

In an eighth aspect the invention provides nucleic acid molecules comprising a polynucleotide selected from the group consisting of:
i. SEQ ID NO: 1 or 2 or 36 (construct S);
ii. SEQ ID NO: 4 (construct stab-S);
iii. SEQ ID NO: 6 (construct Secto);
iv. SED ID NO: 8 (construct stab-Secto);
v. SEQ ID NO: 10 (construct S1),
vi. SEQ ID NO: 12 (construct S2),
vii. SEQ ID NO: 14 (construct stab-S2),
viii. SEQ ID NO: 16 (construct tri-Secto),
ix. SEQ ID NO: 18 (construct tristab-Secto),
x. SEQ ID NO: 42 (construct S3F),
xi. SEQ ID NO: 44 (construct S2P3F),
xii. SEQ ID NO: 46 (construct S2PΔF),
xiii. SEQ ID NO: 48 (construct S2PΔF2A),
xiv. SEQ ID NO: 21 or 37 (construct N),
xv. SEQ ID NO: 51 (construct T4-S2P3F (tristab-Secto-3F)),
xvi. SEQ ID NO: 53 (construct S6P),
xvii. SEQ ID NO: 55 (construct S6P3F),
xviii. SEQ ID NO: 57 (construct S6PΔF),
xix. SEQ ID NO: 59 (construct SCCPP),
xx. SEQ ID NO: 61 (construct SCC6P),
xxi. SEQ ID NO: 63 (construct S_{MVopt}2P),
xxii. SEQ ID NO: 64 (construct S_{MVopt}ΔF),
xxiii. SEQ ID NO: 66 (construct S_{MVopt}2PΔF),
xxiv. SEQ ID NO: 75 (construct SF-2P-dER), and
xxv. SEQ ID NO: 81 (construct SF-2P-2a).

In a ninth aspect the invention provides polypeptides comprising an amino acid sequence selected from the group consisting of:
i. SEQ ID NO: 3 (construct S);
ii. SEQ ID NO: 5 (construct stab-S);
iii. SEQ ID NO: 7 (construct Secto);
iv. SED ID NO: 9 (construct stab-Secto);
v. SEQ ID NO: 11 (construct S1),
vi. SEQ ID NO: 13 (construct S2),
vii. SEQ ID NO: 15 (construct stab-S2),
viii. SEQ ID NO: 17 (construct tri-Secto),
ix. SEQ ID NO: 19 (construct tristab-Secto),
x. SEQ ID NO: 43 (construct S3F),
xi. SEQ ID NO: 45 (construct S2P3F),
xii. SEQ ID NO: 47 (construct S2PΔF),
xiii. SEQ ID NO: 49 (construct S2PΔF2A),
xiv. SEQ ID NO: 22 (construct N),
xv. SEQ ID NO: 52 (construct T4-S2P3F (tristab-Secto-3F)),
xvi. SEQ ID NO: 54 (construct S6P),
xvii. SEQ ID NO: 56 (construct S6P3F),
xviii. SEQ ID NO: 58 (construct S6PΔF),
xix. SEQ ID NO: 60 (construct SCCPP),
xx. SEQ ID NO: 62 (construct SCC6P),
xxi. SEQ ID NO: 65 (construct S_{MVopt}ΔF),
xxii. SEQ ID NO: 76 (construct SF-2P-dER), and
xxiii. SEQ ID NO: 82 (construct SF-2P-2a).

In a tenth aspect the invention provides recombinant proteins expressed by a transfer vector of the invention. The recombinant proteins may be expressed in vitro or in vivo. In some embodiments the recombinant proteins further comprise an amino acid tag for purification.

In an eleventh aspect the invention provides the in vitro use of an antigen having the sequence of any one of SEQ ID NOs: 3, 5, 7, 9, 11, 13, 15, 17, 19, 22, 23, 24, 25, 26, 27, 43, 45, 47, 49, 52, 54, 56, 58, 60, 62, 65, 76 and 82 for the detection of the presence of antibodies against the antigen in a biological sample previously obtained from an individual suspected of being infected by SARS-CoV-2, wherein the polypeptide is contacted with the biological sample to determine the presence of antibodies against the antigen.

In a twelfth aspect the invention provides a method comprising contacting a biological sample with a polypeptide comprising the amino acid sequence of any one of SEQ ID NOs: 3, 5, 7, 9, 11, 13, 15, 17, 19, 22, 23, 24, 25, 26, 27, 43, 45, 47, 49, 52, 54, 56, 58, 60, 62, 65, 76, and 82 or an immunogenic fragment thereof and detecting the formation of antibody-antigen complexes between antibodies present in the biological sample and the polypeptide. In some embodiments the biological sample is obtained from an individual suspected of being infected by SARS-CoV-2.

In a thirteenth aspect the invention provides methods for treating or preventing an infection by SARS-CoV-2 in a subject (for example a human host), comprising administering an immunogenic composition or vaccine according to the invention to the subject. Also provided are methods for inducing a protective immune response against SARS-CoV-2 in a subject (for example a human host), comprising administering an immunogenic composition or vaccine according to the invention to the subject. In some embodiments of the methods of treating or preventing an infection or inducing an immune response, the method comprises a first administration of the immunogenic composition or vaccine and a second administration of the immunogenic composition or vaccine. In some embodiments, the second administration is performed at from one to two months after the first administration.

### BRIEF DESCRIPTION OF THE FIGURES

FIG. 1: Restriction map of plasmid pKP-MVSchw (17858 bp).

FIG. 2: Schematic of the primary structure of the SARS-CoV-2 Spike protein and position of mutations. The spike protein consists of 2 subdomains, S1 and S2, separated by a furin cleavage site. In the S2 domain, the heptad repeat 1 (HR1), central helix (CH), connector domain (CD), heptad repeat 2 (HR2), transmembrane domain (TM), and cytoplasmic tail (CT) are shown. Positions of described mutations are indicated using arrows. Mutation (3F:R682G+R683S+R685G) or deletion (DetaF: 6756QTQTNSPRRAR-685) of the furin cleavage site to stabilize the full-length protein; Mutation (2P: K986P+V987P) locks the protein in the pre-fusion form; Mutation (K1269A+H1271A) of the endoplasmic reticulum retrieval signal to potentially enhance cell surface expression.

FIGS. 3A to 3C: Schematic representation of SARS-CoV-2 Spike Constructs of recombinant MV Vector. 3A. Simplified schematic of the S protein and positions of modifications. 3B/3C. Synthetic sequences of SARS-CoV-2 spike were cloned into the ATU3 (3B) or ATU2 (3C) position of the MV vector. All constructs in ATU3 are based on a fully human codon-optimized sequence of the full-length, membrane-bound S protein (SEQ ID NO: 2). All constructs in ATU2 are based on a measles-optimized sequence (MVopt, SEQ ID NO: 36). Modifications of the S protein in the different constructs are indicated and the names of the corresponding rescued viruses are shown. MV proteins are depicted as follows: N (nucleoprotein), P (phosphoprotein), M (matrix), F (fusion protein), H (hemagglutinin), L (large protein), T7 RNA polymerase promoter (T7), T7 RNA polymerase terminator (T7t), hammerhead ribozyme (hh), hepatitis delta virus ribozyme (hδh).

FIGS. 4A to 4B: Detection of SARS-CoV-2 S by Western Blot in cell lysates. Vero cells were infected at a MOI of 0.05 with A) MV-ATU3-S, or B) MV-AUT3-S2P, MV-ATU3-S2P3F, MV-ATU3-S2PΔF or MV-ATU3-S2PΔF2A, or the parental MV Schwarz strain (MVSchw) or were not infected (NI). Total cell extracts were prepared at 39 h post-infection, separated by electrophoresis on NuPAGE 4-12% Bis-Tris gel, transferred onto a PVDF membrane and detected with anti-SARS-CoV-1 spike polyclonal rabbit antibodies (Escriou et al., Virology, 2014), AlexaFluor 680-conjugated anti-rabbit antibodies and nearIR imaging. As loading control, the N protein of measles was detected using anti-MV nucleoprotein polyclonal rabbit antibodies (Covalab). The position of the SARS-CoV-2 spike protein, the S1 and S2 subdomains, and the measles N protein as well as molecular weight markers (in kDa) are shown.

FIGS. 5A and 5B: Comparative fusogenic properties of the various recombinant MVs. Vero cells were infected at a MOI of 0.05 with A) MV-ATU3-S, or B) MV-AUT3-S2P, MV-ATU3-S2P3F, MV-ATU3-S2PΔF or MV-ATU3-S2PΔF2A, or the parental MV Schwarz strain (MVSchw). Cell monolayers were observed at 39 h post-infection and areas of fused cells were marked.

FIGS. 6A and 6B: Antibody response to measles (A) and SARS-CoV-2 S (B) in IFNAR-KO mice after prime and boost immunization with recombinant MV expressing SARS-CoV-2 spike. Mice were immunized with the parental MV Schwarz strain (Schw), MV-ATU3-S(S), MV-AUT3-S2P (S2P), MV-ATU3-S2P3F (S2P3F), MV-ATU3-S2PΔF (S2PΔF) or MV-ATU3-S2PΔF2A (S2PΔF2A). Antibody responses were measured by measles-specific ELISA (A) and SARS-CoV-2 spike-specific ELISA (B) in sera collected following after prime (left parts of the graphs) or boost (right part of the graphs). Bars show medians. Detection limit (dotted line) in the anti-MV ELISA was 50 ELISA units. Detection limit for the anti-S ELISA was 200 ELISA units for sera collected after the boost and was 50 ELISA units in all other analyses. Representative results of two or more independent experiments are shown.

FIGS. 7A and 7B: SARS-CoV-2 microneutralization titers in IFNAR-KO mice after prime and boost immunization with recombinant MV expressing SARS-CoV-2 spike. A. Mice were immunized with the parental MV Schwarz strain (Schw), MV-ATU3-S(S), MV-AUT3-S2P (S2P), MV-ATU3-S2P3F (S2P3F), MV-ATU3-S2PΔF (S2PΔF) or MV-ATU3-S2PΔF2A (S2PΔF2A). Neutralization titers were measured by microneutralization assay following prime (left part of the graph) or prime/boost (right part of the graph) immunization(s) and were expressed as reciprocals of serum dilutions that resulted in the neutralization of 50% SARS-CoV-2 infectivity scored by cytopathic effect. Bars show medians. Detection limit was a titer of 20. Samples with undetectable neutralization activity were assigned a value of 10, equal to half the detection limit. Representative results of two or more independent experiments are shown. B. Assessment of the research reagents 20/118 (a panel comprising 4 convalescent human sera, 20/120, 122, 124, 126 and a control human serum, 128) and 20/130 provided by the National Institute of Biological Standards and Controls (NIBSC) in the microneutralization assay enabled comparison of results with other assays. The research reagents were measured four times on different days in the neutralization assay together with a pool of sera (S2PΔF-IS2 pool) obtained after the second immunization with MV-ATU3-S2PΔF. Two different stocks (C2 and C3.4) of the same SARS-CoV-2 strain were used. Back-titrated SARS-CoV-2 TCID50s used in the assay are shown. The results provided by NIBSC are listed for comparison, expressed as reciprocals of serum dilutions that resulted in inhibition of 50% of virus infectivity as scored by cytopathic effect (CPE) or plaque assay (PRNT50).

FIG. 8: MV and S-specific IFN-γ T cell in immunized mice after boost immunization. Mice were immunized twice at a 3-week interval with MV-ATU3-S2PΔF2A (S2PΔF2A) or parental MV Schwarz (MVSchw). Summed IFN-γ ELISpot responses (spot forming units, SPU) in splenocytes stimulated with peptide pools spanning the S protein (S1, S2), or a pool of two specific measles peptides are shown. Bars show medians.

FIGS. 9A to 9C: MV and S-specific CD4⁺ and CD8⁺ T cell responses in mice immunized with recombinant MV expressing SARS-CoV-2 spike. Mice were immunized with MV-ATU3-S(S), MV-AUT3-S2P (S2P), MV-ATU3-S2P3F (S2P3F), MV-ATU3-S2PΔF (S2PΔF), MV-ATU3-S2PΔF2A (S2PΔF2A) or parental MV Schwarz (Schw). The frequencies of splenic CD4⁺ T cells (A) or CD8⁺ T cells (B) producing Th1-characteristic cytokines IFN-γ and TNF-α or Th2-characteristic cytokines IL-5 and IL-13 in response to peptide pools spanning the S1 or S2 domain of the SARS-CoV-2 spike protein are shown cumulatively (summed responses to S1 and S2 pools). INF-γ/TNF-α or IL-5/IL-13 CD8⁺ T cells responses to MV (pool of two H-2b class I—restricted measles peptides) are shown in panel C.

FIGS. 10A to 10D: MV and S-specific double and single cytokine-positive CD4⁺ and CD8⁺ T cell responses in mice immunized with MV-ATU3-S2PΔF2A. Mice were immunized with MV-ATU3-S2PΔF2A (S2PΔF2A) or parental MV Schwarz (MVSchw). The frequencies of splenic CD4⁺ T cells (A) or CD8⁺ T cells (C) producing Th1-characteristic cytokines IFN-γ and TNF-α or Th2-characteristic cytokines IL-5 and IL-13 (double positive cells, respectively) in response to peptide pools spanning the S1 or S2 domain of the SARS-CoV-2 spike protein are shown cumulatively (summed responses to S1 and S2 pools). In addition, a pool of two H-2b class I—restricted measles peptides was used to assess T cell responses to the MV backbone. The frequency of single cytokine producing CD4⁺ T cells (B) or CD8⁺ T cells (D) in response to S peptide pools was not significantly different (ns) in mice immunized with MV-ATU3-S2PΔF2A or MVSchw control, except for TNF-α producing CD4⁺ T cells. Statistical analysis was performed using Mann-Whitney test (** p≤0.005, * p≤0.05).

FIGS. 11A to 11D: IgG1 and IgG2a response in IFNAR-KO mice after prime and boost immunization with recombinant MV expressing SARS-CoV-2. Mice were immunized with the parental MV Schwarz strain (Schw), MV-ATU3-S(S), MV-AUT3-S2P (S2P), MV-ATU3-S2P3F (S2P3F), MV-ATU3-S2PΔF (S2PΔF) or MV-ATU3-S2PΔF2A (S2PΔF2A). A. Isotype-specific (IgG1 and IgG2a) antibody responses against SARS-CoV-2 spike measured by ELISA. Bars show medians. Detection limit was 50 ELISA units. B/D. Ratios of IgG2a to IgG1 calculated for each construct/immunogen. C. Control experiments were performed by immunizing wt 129/Sv mice with alum-adjuvanted trimerized spike ectodomain expressed in HEK293 cells (T4S2P3F-8H).

FIGS. 12A to 12B: Protection of mice against challenge with SARS-CoV-2 after prime and boost (A) or after single (B) immunization. A. Mice were immunized twice at a 4-week interval with the parental MV Schwarz strain (Schw), MV-ATU3-S(S), MV-AUT3-S2P (S2P), MV-ATU3-S2PΔF (S2PΔF) or MV-ATU3-S2PΔF2A (S2PΔF2A). Blood samples were taken 20 days after the second immunization and respective neutralization titers determined (μNT). The mice were instilled Ad5:hACE2 25 days after boost immunization and were challenged with SARS-CoV-2 4 days later. B. Mice were immunized once with the parental MV Schwarz strain (Schw) or MV-ATU3-S2PΔF2A (S2PΔF2A). Blood samples were taken 165 days post-immunization and μNT titers determined. The mice were instilled with Ad5:hACE2 on day 173 and challenged 4 days later. In both experiments, lungs were harvested 4 days after challenge. Lung viral loads were determined for RNA levels in genome equivalents (GEQ) or infectious titers in plaque forming units (PFU) per lung. Statistical significance of the differences in microneutralization titers, GEQ, and infectious virus was assessed using the non-parametric Kruskal-Wallis test with Dunn's uncorrected post-hoc analysis (A) or Mann-Whitney test (B). * p<0.05, ** p<0.005, *** p<0.0005, **** p<0.0001. Analyses were performed using GraphPad Prism 8.

FIGS. 13A to 13B: Detection of SARS-CoV-2 S by Western Blot in cell lysates in ATU2 and ATU3 constructs. Vero cells were infected at MOI=1 with (A) the parental MV Schwarz strain (Schw), 4 different clones (1-4) of MV-ATU3-S(S), or 6 different clones (1-6) of MV-ATU2-S_{MVopt} (S_{MVopt}) and (B) the parental MV Schwarz strain (Schw), one representative clone of MV-ATU2-S_{MVopt} (S_{MVopt}), MV-ATU3-S2P (S2P), MV-ATU3-S2P3F (S2P3F), MV-ATU3-S2PΔF (S2PΔF) or 2 different clones (1, 2) of MV-ATU2-S_{MVopt}2P (S_{MVopt}2P), MV-ATU2-S_{MVopt}ΔF (S_{MVopt}ΔF), MV-ATU2-S_{MVopt}2PΔF (S_{MVopt}2PΔF). Protein cell extracts were prepared at 24 h post-infection, separated by electrophoresis on NuPAGE 4-12% Bis-Tris gel, transferred onto a PVDF membrane and probed with anti-SARS-CoV-2 spike polyclonal rabbit antibodies, AlexaFluor 680-conjugated anti-rabbit antibodies and nearIR imaging. As loading control, the N protein of measles was detected using anti-MV nucleoprotein polyclonal rabbit antibodies (Covalab). The position of the SARS-CoV-2 spike protein, the S1 and S2 subdomains, and the measles N protein as well as molecular weight markers (in kDa) are shown.

FIGS. 14A to 14C: Antibody response to measles (A) and SARS-CoV-2 S (B) and microneutralization titers (C) in IFNAR-KO mice after immunization with recombinant MV expressing SARS-CoV-2 spike in ATU2 or ATU3. Mice were immunized with the parental MV Schwarz strain (Schw), MV-ATU3-S(S), or MV-ATU2-S_{MVopt} (S_{MVopt}). Antibody responses in sera collected after prime or boost were measured by measles-specific ELISA (A) and SARS-CoV-2 spike-specific ELISA (B), neutralizing antibodies were measured by microneutralization assay (C). Bars show medians. Lower limits of quantification nare indicated by dotted lines.

FIG. 15: In vitro and in vivo evaluation of recombinant MV Schwarz expressing 6P-stabilized SARS-CoV-2 spike.

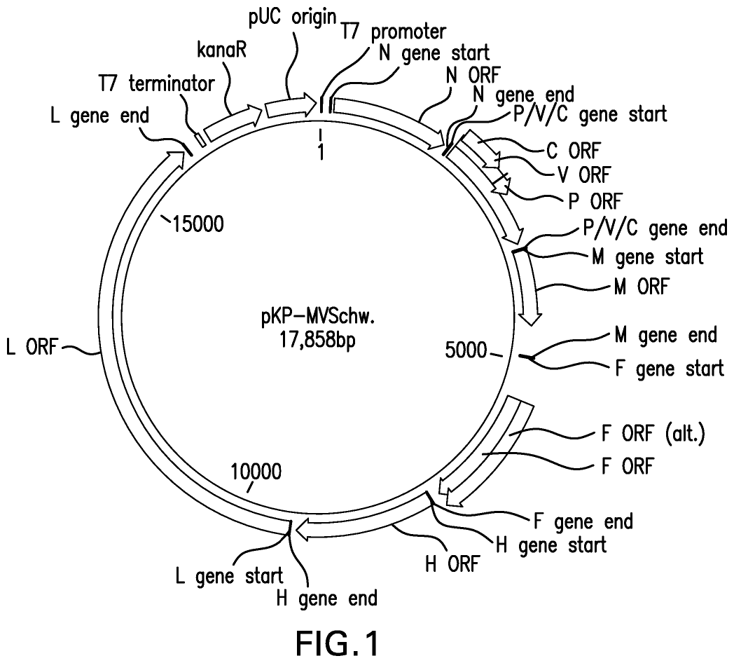

Vero cells were infected at a MOI of 1 with MV-ATU3-S, MV-ATU3-S2P, MV-ATU3-S2PΔF or MV-AUT3-S6P (2 viral clones), or the parental MV Schwarz strain (MVSchw) or were not infected (NI). Total cell extracts were prepared at 24 h post-infection, separated by electrophoresis on NuPAGE 4-12% Bis-Tris gel, transferred onto a PVDF membrane and detected with anti-SARS-CoV-2 spike polyclonal rabbit antibodies, AlexaFluor 680-conjugated anti-rabbit antibodies and nearIR imaging (A, upper panel). As loading control, the MV N protein was probed using anti-MV nucleoprotein polyclonal rabbit antibodies (Covalab) (A, lower panel). The position of the SARS-CoV-2 spike protein, the S1 and S2 subdomains, and the measles N protein as well as molecular weight markers (in kDa) are shown.
IFNAR-KO mice were immunized twice at a 4-week interval with the parental MV Schwarz strain (Schw), MV-ATU3-S2P (S2P), MV-AUT3-S2PΔF (S2PΔF) or MV-ATU3-S6P (S6P). Antibody responses were measured in sera collected 3 weeks after prime or boost by measles-specific ELISA (B), SARS-CoV-2 spike-specific ELISA (C) and SARS-CoV-2 microneutralization assay (μNT, D). The mice were instilled with Ad5::hACE2 24 days after boost immunization and challenged 4 days later. Lungs were harvested 4 days after challenge. Lung viral loads were determined as RNA levels (GEQ) per lung (E). Bars show medians. Lower limits of quantification are indicated by dotted lines. Statistical significance of the differences in GEQ titers was assessed using the Kruskal-Wallis test with Dunn's uncorrected post-hoc analysis. ** p<0.005, *** p<0.0005. Analyses were performed using GraphPad Prism 8.

FIG. 16: Comparative analysis of the fusion properties of S and the various mutated S proteins encoded by recombinant MVs. HEK-293T-GFP10 cells were transfected with plasmids allowing transient expression of S (wt-S), S2P (S-2P), S3F (S-3F), S2P3F (S-2P&3F), or S2PΔF (S-2P&ΔF) and co-cultured with HEK-293T-GFP11 cells transfected with hACE2 expression plasmid, allowing reconstitution of GFP activity if fusion occurs between the two cell subpopulations, according to the assay described for S in Buchrieser et al (2020). Negative (neg, mock-transfected) and positive (pos, transfected with a plasmid expressing S at high levels) controls were included. Images of the cell sheets were recorded at 18 h post-transfection. Percentages of fusion were scored as GFP areas per cell area and plotted in the graph below images.

FIG. 17: Protection of mice against challenge with SARS-CoV-2 after prime only immunization. Mice were immunized once with the parental MV Schwarz strain (Schw), MV-ATU3-S2P (S2P) or MV-ATU3-S2PΔF2A (S2PΔF2A). Blood samples were taken 3 weeks post-immunization and antibody responses were measured by measles-specific ELISA (A), SARS-CoV-2 spike-specific ELISA (B) and SARS-CoV-2 microneutralization assay (μNT, C). The mice were instilled with Ad5:hACE24 weeks post-immunization and challenged 4 days later. Lungs were harvested 4 days after challenge. Lung viral loads were determined as RNA levels (GEQ) or infectious titers (PFU) per lung. Bars show medians. Lower limits of quantification are indicated by dotted lines. Statistical significance of the differences in microneutralization titers, GEQ, and infectious virus was assessed using the Kruskal-Wallis test with Dunn's uncorrected post-hoc analysis. * p<0.05, ** p<0.005, *** p<0.0005. Analyses were performed using GraphPad Prism 8.

FIG. 18: Optimization of SARS-CoV-2 spike ectodomain constructs for efficient secretion and assembly into homotrimers. (A). Schematic of a secreted and trimerized form of the spike (tri-Secto) corresponding to the full-length ectodomain of S fused at its C-terminus to a foldon (T4 or GCN4) through a Ser-Gly-Gly connecting linker followed by the Twin-strep-tag (Strep Tag). The positions of the signal peptide, subdomains S1 and S2, furin cleavage site, fusion peptide, heptad repeats (HR) 1 and 2, and connector domain (CD) are indicated. Positions of mutations described in the text are indicated using arrows underneath the schematic. The constructs were named according to the combination of mutations and foldon: as an example, T4-S2P3F combined the 2P and 3F mutations with the T4 fibritin foldon. Mutation (3F:R682G+R683S+R685G) or deletion (DetaF: 6756QTQTNSPRRAR-685) of the furin cleavage site to stabilize the full-length protein; Mutation (2P: K986P+V987P) locks the protein in the pre-fusion form; Trimerisation foldon:T4 or GCN4 (B). HEK 293T cells were transiently transfected with the indicated pCI-Spike_ectomain plasmid DNAs (right part of the panel, foldon T4 or GCN4 for secreted ectodomains is indicated), or, as controls, with pCI-S2P, pCI-S2PΔF and pCI-S3F plasmid DNAs, which encode full-length variants of spike (left part of the panel, full-length membrane-anchored (mb) spike). Supernatants were collected at 48 h post-transfection, separated by electrophoresis on NuPAGE 4-12% Bis-Tris gel, transferred onto a PVDF membrane and detected with anti-SARS-CoV-2 spike polyclonal rabbit antibodies, AlexaFluor 680-conjugated anti-rabbit antibodies and nearIR imaging. The position of molecular weight markers (in kDa) are shown. (C). The T4-S2P3F, GCN4-S2P3F and T4-S2P polypeptides were separated by size exclusion chromatography on a Superdex200 column. The elution profiles were recorded by absorbance at 280 nm (mAU).

FIG. 19: Detection of SARS-CoV-2 spike ectodomain in supernatants of MV-ATU3-T4-S2P3F infected cells. Vero cells were infected at a MOI of 0.05 with MV-ATU3-S2P3F, MV-ATU3-Secto, MV-ATU3-T4-S2P3F (4 viral clones), or the parental MV Schwarz strain (MVSchw) or were not infected (NI). Supernatants (upper panel) were collected and total cell extracts (middle panel) were prepared at 39 h post-infection, separated by electrophoresis on NuPAGE 4-12% Bis-Tris gel, transferred onto a PVDF membrane and detected with anti-SARS-CoV-2 spike polyclonal rabbit antibodies, AlexaFluor 680-conjugated anti-rabbit antibodies and nearIR imaging. As loading control for total cell extracts, the MV N protein was probed using anti-MV nucleoprotein polyclonal rabbit antibodies (Covalab) (lower panel). The position of the SARS-CoV-2 spike protein/ectodomain, the S1 and S2 subdomains, and the measles N protein as well as molecular weight markers (in kDa) are shown.

FIG. 20: Expression levels of SARS-CoV-2 N in lysates from cells infected with ATU2-N and ATU2-N_{MVopt} viruses. Vero cells were infected at a MOI of 1 with MV-ATU2-N (4 viral clones), MV-ATU2-N_{MVopt} (4 viral clones), or the parental MV Schwarz strain (MVSchw) or were not infected (NI). Total cell extracts were prepared at 24 h post-infection, separated by electrophoresis on NuPAGE 4-12% Bis-Tris gel, transferred onto a PVDF membrane and detected with anti-SARS-CoV-2 nucleoprotein polyclonal rabbit antibodies, AlexaFluor 680-conjugated anti-rabbit antibodies and nearIR imaging (upper panel). As loading control, the MV N protein was probed using anti-MV nucleoprotein polyclonal rabbit antibodies (Covalab) (lower panel). The position of the SARS-CoV-2 nucleoprotein, and the measles N protein as well as molecular weight markers (in kDa) are shown.

FIG. 21. Schematic of the native S protein of SARS-CoV-2. The native S protein is 1273 amino acids (aa) in length. The protein contains 2 subunits, S1 and S2, generated by cleavage at the furin cleavage site (F). S1 contains the signal peptide (SP), N-terminal domain (NTD) and receptor-binding domain (RBD). S2 contains the fusion peptide (FP), heptad repeats 1 (HR1) and 2 (HR2), transmembrane domain (TM), and cytoplasmic tail (CT). The 2P indicates the two mutated prolines, K986P and V987P. The letters KLHYT indicate the endoplasmic reticulum retrieval signal (ERRS) motif KxHxx of SEQ ID NO: 149, in the CT. dER indicates constructs carrying a deletion of the 11 C-terminal amino acids from the CT.

FIGS. 22A to 22D. Schematic of S gene constructs and characterization of S-expressing rMVs. a The native S gene of SARS-CoV-2 with notable domains is indicated relative to the S gene constructs cloned into the MV vector. 2P and dER modifications are also indicated. All S constructs were cloned into either the second (ATU2) or third (ATU3) additional transcription units of pTM-MVSchwarz (MV Schwarz), the MV vector plasmid. The MV genome comprises the nucleoprotein (N), phosphoprotein (P), V and C accessory proteins, matrix (M), fusion (F), hemagglutinin (H) and polymerase (L) genes. Plasmid elements include the T7 RNA polymerase promoter (T7), hammerhead ribozyme (hh), hepatitis delta virus ribozyme (8), and T7 RNA polymerase terminator (T7t). b Growth kinetics of rMV constructs used to infect Vero cells at an MOI of 0.1. Cell-associated virus titers are indicated in TCID₅₀/ml. c Western blot analysis of SARS-CoV-2 S protein in cell lysates of Vero cells infected with the rMVs expressing Sf-dER or S2-dER from either ATU2 or ATU3, with or without the 2P mutation. d Immunofluorescence staining of Vero cells infected with the indicated rMVs 24 h after infection. Permeabilized or non-permeabilized cells were stained for S, MV N and nuclei.

FIGS. 23A to 23F. Induction of humoral responses by prime-boost vaccination. a Homologous prime-boost of IFNAR −/− mice (n=6 or n=4 for the empty MV control) immunized Intraperitoneally with 1×10⁵ TCID₅₀ of the indicated rMV at days 0 and 28. Sera were collected 28 and 42 days after immunization and assessed for specific antibody responses to b MV antigens or c S-SARS-CoV-2 S. The data show the reciprocal endpoint dilution titers with each data point representing an individual animal. d Neutralizing antibody responses to SARS-CoV-2 virus expressed as 50% plaque reduction neutralization test (PRNT₅₀) titers. e IgG subclass of S-specific antibody responses in mice 4 weeks after the first immunization. f Ratio of IgG2a/IgG1 or Th1/Th2 responses. Data are represented as geometric mean with line and error bars indicating geometric SD. Statistical significance was determined by a two-way ANOVA adjusted for multiple comparisons. Asterisks (*) indicate significant mean differences (** p<0.01, and **** p<0.001) as determined by the Mann-Whitney U-test.

FIGS. 24A to 24D. Induction of S-specific cellular responses by rMV vaccination. a Immunization of IFNAR −/− mice (n=12 or n=3 for the empty MV control) immunized intraperitoneally with 1×10⁵ TCID₅₀ of the indicated rMVs. Seven days after immunization, ELISPOT for IFNγ was performed on freshly extracted splenocytes. The data are shown as IFNγ-secreting cells or spot-forming cells (SFC) per 1×10⁶ splenocytes detected after stimulating with b MV Schwarz or c SARS-CoV-2 S peptide pools specific to CD8⁺ or CD4⁺ T cells. d Ratio of IFNγ-secreting cells stimulated by CD4⁺ or CD8⁺ peptides to those stimulated by MV Schwarz. Each data point represents an individual mouse. Asterisks (*) indicate significant mean differences (* p<0.05; ** p<0.01, and **** p<0.001) as determined by the Mann-Whitney U-test.

FIGS. 25A and 25B. Cytokine expression profile of T cells. IFNAR −/− mice (n=12 or n=3 for the empty MV control) immunized intraperitoneally (i.p.) with 1×10⁵ TCID₅₀ of the indicated rMVs and splenocytes were stimulated with S-specific peptide pools. S-specific a CD8⁺ and b CD4⁺ T-cells were stained for intracellular IFNγ, TNFα and IL-5. Asterisks (*) indicate significant mean differences (* p<0.05; ** p<0.01, and **** p<0.001) as determined by the Mann-Whitney U-test.

FIGS. 26A to 26F. Persistence of neutralizing antibodies and immune protection. a Immunization and challenge schedule for IFNAR −/− mice (n=6). Animals were immunized interperitoneally by homologous prime-boost at days 0 and 28. Sera were collected at days 52, 72, and 110. Animals were challenged on day 110 by intranasal inoculation of mouse-adapted SARS-CoV-2 virus (MACo3) at 1.5×10⁵ PFU. Sera were assessed for levels of specific antibodies against b MV and c SARS-CoV-2 S. d Neutralizing antibody responses against SARS-CoV-2 virus, expressed as 50% plaque reduction neutralization test (PRNT₅₀) titers. e SARS-CoV-2 viral RNA copies detected by RT-qPCR in homogenized lungs of challenged animals, calculated as copies/lung. f Titer of infectious viral particles recovered from the homogenized lung of the immunized animals expressed as PFU/lung. Data are represented as geometric means with line and error bars indicating geometric SD. Statistical significance was determined by a two-way ANOVA adjusted for multiple comparisons. Asterisks (*) indicate significant mean differences (* p<0.05; ** p<0.01, and **** p<0.001).

FIGS. 27A to 27G. Immune responses and protection after a single immunization. a Immunization and challenge schedule for IFNAR^{-/-} mice (n=6). Animals were immunized interperitoneally on day 0. Sera were collected at days 24 and 48. Animals were challenged on day 48 by intranasal inoculation of mouse-adapted SARS-CoV-2 virus (MACo3) at 1.5×10⁵ PFU. Sera were assessed For levels of specific antibodies to b MV and c S-SARS-CoV-2 protein. d Neutralizing antibody responses against SARS-CoV-2 virus, expressed as 50% plaque reduction neutralization test (PRNT₅₀) titers. e SARS-CoV-2 viral RNA copies detected by RT-qPCR in homogenized lungs of challenged animals, calculated as copies/lung. f Titer of infectious viral particles recovered from the homogenized lung of the immunized animals expressed as PFU/lung. Data are represented as geometric means with line and error bars indicating geometric SD. Statistical significance for antibody responses (top panels) was determined by two-way ANOVA adjusted for multiple comparisons. The rest of the data (bottom panels) was analyzed by the Mann-Whitney U-test. Asterisks (*) indicate significant mean differences (* p<0.05; ** p<0.01, and **** p<0.001).

FIG. 28. Expression of SARS-CoV-2 S antigens on the surface of transfected HEK293T cells. Cells transfected with pcDNA expression vectors encoding full-length S or S2 subunit antigens were stained for indirect immunofluorescence with an anti-S antibody followed by Alexa Fluor 488-conjugated goat anti-rabbit IgG. Propidium iodide was used to exclude dead cells by gating (upper dot plots). Histograms show surface expression of full-length S (left histograms) or S2 subunit proteins (right histograms). Native-conformation S antigens (light grey), prefusion-stabilized S (dark grey), mock-transfected control cells (black histograms) and corresponding mean fluorescence intensities (MFI) are shown.

FIG. 29. S protein-mediated syncytium formation in transfected Vero cells. Images of Vero cells transfected with pcDNA expression vectors encoding SARS-CoV-2 S proteins were acquired 24 hours post-transfection. Upper images show Vero cells transfected with plasmids encoding native-conformation S antigens, while lower images depict cells transfected with prefusion-stabilized S antigens and non-transfected control Vero cells. Grey lines delineate the borders of syncytia. Native SF indicates native-conformation full-length S protein with an intact CT.

FIG. 30. Immunofluorescence analysis of intracellular S protein expression in Vero cells infected with recombinant MV vaccines. Vero cells were infected with rMVs expressing SARS-CoV-2 S proteins or empty MV Schwarz. Twenty-four hours after infection, S protein was detected in saponin-permeabilized cells using a rabbit anti-S antibody followed by Cy3-conjugated goat anti-rabbit IgG. MV N protein was visualized using mouse monoclonal anti-N antibody followed by Alexa Fluor 488-conjugated goat anti-mouse IgG. Nuclei were stained with DAPI. Images were acquired using a fluorescence microscope.

FIG. 31. Immunofluorescence analysis of S protein surface expression in Vero cells infected with recombinant MV vaccines. Vero cells were infected with rMVs expressing SARS-CoV-2 S proteins or empty MV Schwarz. Twenty-four hours after infection, S protein was detected on the surface of the non-permeabilized cells using a rabbit anti-S antibody followed by Cy3-conjugated goat anti-rabbit IgG. MV N protein was visualized using a mouse monoclonal anti-N antibody followed by Alexa Fluor 488-conjugated goat anti-mouse IgG. Nuclei were stained with DAPI. Images were acquired using a fluorescence microscope.

FIG. 32. Western blot analysis of S protein expression in Vero cells infected with recombinant MV vaccines from serial passages. MV ATU2 vaccines expressing SF-dER or SF-2P-dER antigens were serially passaged on Vero cells from P1 up to P10 and S protein expression was determined by immunoblotting of P1, P5, and P10 cell lysates. Vero cells infected with empty MV were examined in parallel and served as negative controls.

FIGS. 33A to 33C. Cytokine expression profile of T cells assessed in IFNAR^{-/-} mice (n=5 or n=3 for a control Empty MV group) immunized intraperitoneally (i.p.) with 1×10⁵ TCID₅₀ of MV-ATU2-SF-2P-dER or Empty MV. Splenocytes were stimulated with either S-specific CD4 or CD8 peptide (Table 6A and 6B). S-specific a CD8⁺ and b CD4⁺ T-cells were stained for intracellular IFNγ, TNFα, IL-5 and IL13. c S-specific CD4⁺ memory T cells were stained for intracellular IL-5 and IL13. Asterisks (*) indicate significant mean differences (* p<0.05) as determined by Kruskal-Wallis ANOVA with multiple comparisons tests.

FIGS. 34A to 34C. Dose-dependent homologous prime-boost immunization. IFNAR^{-/-} mice (n=6 or n=4 for the empty MV control) were immunized intraperitoneally with the indicated rmV vaccine candidates at 1×10⁵ TCID₅₀ or 1×10⁴ TCID₅₀ at days 0 and 28. Sera were collected 28 and 50 days after immunization and assessed for specific antibody responses to a MV antigen or b SARS-CoV-2 S protein. The data show the reciprocal endpoint dilution titers with each data point represents an individual animal. c Neutralizing antibody response to SARS-CoV-2 virus expressed as 50% plaque reduction neutralization test (PRNT₅₀) titers. Data are represented as geometric means with lines and error bars indicating geometric SD. Statistical significance was determined by a two-way ANOVA adjusted for multiple comparisons. Asterisks (*) indicate significant mean differences (*p<0.05, **p<0.01, and **** p<0.001).

FIG. 35. FACS analysis of transfected HEK293T cells with pCDNA expressing S protein of SARS-CoV-2. Cells transfected with pcDNA expression vectors encoding full-length S or S2 subunit antigens were stained for indirect immunofluorescence with an anti-S antibody followed by Alexa Fluor 488-conjugated goat anti-rabbit IgG. Propidium iodide was used to exclude dead cells by gating (upper dot plots). Histograms show surface expression of full-length S (left histograms) or S2 subunit proteins (right histograms). Native-conformation S antigens (light grey), prefusion-stabilized S (dark grey), mock-transfected control cells (black histograms) and corresponding mean fluorescence intensities (MFI) are shown.

### DETAILED DESCRIPTION

### Definitions

As used herein, the articles “a” and “an” refer to one or to more than one (i.e., to at least one) of the grammatical object of the article. By way of example, “an element” means one element or more than one element. Furthermore, use of the term “including” as well as other forms, such as “include,” “includes,” and “included,” is not limiting.

As used herein, the term “about” in quantitative terms refers to plus or minus 10% of the value it modifies (rounded up to the nearest whole number if the value is not sub-dividable, such as a number of molecules or nucleotides).

All ranges disclosed herein are inclusive of the recited endpoint and independently combinable (for example, the range of “from 50 mg to 500 mg” is inclusive of the endpoints, 50 mg and 500 mg, and all the intermediate values).

As used herein, the term “comprising” may include the embodiments “consisting of” and “consisting essentially of.” The terms “comprise(s),” “include(s),” “having,” “has,” “contain(s),” and variants thereof, as used herein, are intended to be open-ended transitional phrases, terms, or words that require the presence of the named ingredients/steps and permit the presence of other ingredients/steps. However, such description should be construed as encompassing within its scope compositions or processes as “consisting of” and “consisting essentially of” the enumerated components, which allows the presence of only the named components or compounds, along with any acceptable carriers or fluids, and excludes other components or compounds.

The terms “upstream” and “downstream” are used herein to refer to the relative position of a nucleic acid sequence within a longer nucleic acid sequence that is relative to the direction of RNA transcription (5′ to 3′) of the longer nucleic acid sequence. The term “upstream” refers to a nucleic acid sequence as being nearer to the 5′ end of a longer nucleic acid sequence (earlier in RNA transcription). The term “downstream” refers to a nucleic acid sequence as being nearer to the 3′ end of a longer nucleic acid sequence (later in RNA transcription).

As used herein, the term “antigenic polypeptide” refers to a polypeptide which is capable of inducing an immune response to the virus from which the polypeptide is derived.

As used herein, the term “immunogenic fragment” of a polypeptide refers to a polypeptide fragment which is capable of inducing an immune response to the virus from which the polypeptide is derived. Non-limiting examples of immunogenic fragments include: Secto polypeptide of SARS-CoV-2, stab-Secto polypeptide of SARS-CoV-2, S1 polypeptide of SARS-CoV-2, S2 polypeptide of SARS-CoV-2, tri-Secto polypeptide of SARS-CoV-2, tristab-Secto polypeptide of SARS-CoV-2, and S mutated in the domain involved in endoplasmic reticulum retention.

For the purpose of the present invention virus strain SARS-CoV-2 will be described in particular by reference to its nucleotide sequence (wild type sequence) disclosed in Genbank as MN908947 sequence and publicly available from NBCBI since 20 Jan. 2020 and that has been updated since that date as MN908947.3.

Throughout the text, figures and sequence listing, the expressions “coronavirus 2019-nCoV”, “2019-nCoV”, “nCoV” or “SARS-CoV-2” are interchangeable.

The expression “polypeptide” or “polypeptide of a coronavirus in particular of SARS-CoV-2” defines a molecule resulting from a concatenation of amino acid residues.

As used herein, “increased cell surface expression” of an S-protein or dual domain S-protein having a mutation by insertion, substitution, or deletion in the cytoplasmic tail is measured by transfecting human embryonic kidney cells (HEK) 293T (ATCC CRL-3216) with an expression construct to express the mutated protein in parallel to control HEK 293T cells transfected with a corresponding non-mutated protein and measuring cell-surface expression using an immune-assay. The cell surface expression can be further increased by an additional mutation by insertion, substitution, or deletion, for example an additional mutation that maintains the S protein in the pre-fusion form such as the 2P mutation. An exemplary assay is described in the Examples and certain results from the assay are presented in FIG. 28 and FIG. 35.

As defined herein, the expression “dER” refers to a mutation by deletion of the 11 C-terminal amino acid residues (aa 1263-1273) from the cytoplasmic tail of the S protein, especially of the S protein of SARS-CoV-2 of SEQ ID NO: 3. The deletion of the domain from the cytoplasmic tail increases surface expression of the polypeptide fragment of S in the cells infected with the recombinant MV expressing this polypeptide fragment.

As defined herein, the term “2P” refers to a mutation of 2 amino acid residues, i.e. mutation by substitution of two proline residues at positions 986 and 987 (K986P+V987P) of the amino acid sequence of the S protein of SARS-CoV-2 of SEQ ID NO: 3, that maintains the S protein in the pre-fusion form, the mutation occurring in the S2 domain, e.g. between the heptad repeat 1 (HR1) and the central helix (CH).

As defined herein, the term “2A” or “2a” refers to a mutation of two amino acid residues (K1269A+H1271A) of the endoplasmic reticulum retrieval signal in the amino acid sequence of the S protein of SARS-CoV-2 of SEQ ID NO: 3 to potentially increase cell surface expression.

As used herein, the phrase “dual domain S protein” refers to a coronavirus spike (S) protein that includes both the S1 and S2 domains. A dual domain S protein may include mutations (substitutions, deletions, and/or additions), but is not missing an entire S1 or S2 domain.

As used herein, the expression “encoding” defines the ability of the nucleic acid molecules to be transcribed and where appropriate translated for product expression into selected cells or cell lines. Accordingly, the nucleic acid construct may comprise regulatory elements controlling the transcription of the coding sequences, in particular promoters and termination sequences for the transcription and possibly enhancer and other cis-acting elements. These regulatory elements may be heterologous with respect to the CoV, in particular the SARS-CoV-2 polynucleotide sequences.

The expression “operatively linked” or “operably linked” refers to the functional link existing between the different polynucleotides of the nucleic acid construct of the invention such that the different polynucleotides and nucleic acid construct are efficiently transcribed and if appropriate translated, in particular in cells or cell lines, especially in cells or cell lines used as part of a rescue system for the production or amplification of recombinant infectious MV particles of the invention or in host cells, especially in mammalian or in human cells.

As used herein the term “replicon” refers to any genetic element (e.g., plasmid, chromosome, viral RNA) that functions as an autonomous unit of DNA or RNA replication (i.e. self-replicating). A replicon may originate from a viral genome, and may contain viral nonstructural genes for viral genome replication with one or more structural proteins deleted or replaced by genes foreign to the wild type viral genome.

As used herein, the term “recombining” means introducing at least one polynucleotide into a cell, for example under the form of a vector, the polynucleotide integrating (entirely or partially) or not integrating into the cell genome (such as defined above).

The term “transfer” as used herein refers to the plating of the recombinant cells onto a different type of cells, and particularly onto monolayers of a different type of cells. These latter cells are competent to sustain both the replication and the production of infectious MV-CoV particles, i.e., respectively the formation of infectious viruses inside the cell and possibly the release of these infectious viruses outside of the cells. This transfer results in the co-culture of the recombinant cells of the invention with competent cells as defined in the previous sentence. The above transfer may be an additional, i.e., optional, step when the recombinant cells are not sufficiently efficient virus-producing culture, i.e., when infectious MV-CoV particles cannot be efficiently recovered from these recombinant cells.

As used herein, the phrase “effective dose” in reference to a dose or amount of a vaccine composition disclosed herein refers to a dose required to elicit antibodies and/or a cellular immune response that significantly reduce the likelihood or severity of infectivity of an infectious agent, e.g., coronavirus, during a subsequent challenge. In some embodiments, the effective dose is a dose listed in a package insert for the vaccine composition.

As used herein, when referring to a prophylactic composition, such as a vaccine, the term “booster” refers to an extra administration of the immunogenic composition of the present disclosure, or of another prophylactic or therapeutic compound.

As used herein, the term “virus-like particle” (VLP) refers to a structure that comprises the measles virus structural proteins and at least one SARS-CoV-2 S polypeptide or immunogenic fragment thereof, as encoded by a nucleic acid construct of this disclosure, but does not comprise the nucleic acid construct. The VLPs of the invention are non-infectious and non-replicative.

As used herein, the terms “associated” or “in association” refer to the presence, in a unique composition, of two or more listed elements, such as a recombinant infectious replicating MV-CoV particle and a CoV protein and/or CoV containing VLP. The associated elements may be physically separate entities.

The term “identity,” as known in the art, refers to a relationship between the sequences of two or more polypeptides or polynucleotides, as determined by comparing the sequences. In the art, identity also means the degree of sequence relatedness between two sequences as determined by the number of matches between strings of two or more amino acid residues or nucleic acid residues. Identity measures the percent of identical matches between the smaller of two or more sequences with gap alignments (if any) addressed by a particular mathematical model or computer program (e.g., “algorithms”). Identity of related peptides can be readily calculated by known methods. The term “% identity” as it applies to polypeptide or polynucleotide sequences is defined as the percentage of residues (amino acid residues or nucleic acid residues) in the candidate amino acid or nucleic acid sequence that are identical with the residues in the amino acid sequence or nucleic acid sequence of a second sequence after aligning the sequences and introducing gaps, if necessary, to achieve the maximum percent identity. Methods and computer programs for the alignment are well known in the art. Identity depends on a calculation of percent identity but may differ in value due to gaps and penalties introduced in the calculation. Generally, variants of a particular polynucleotide or polypeptide have at least 40%, 45%, 50%, 55%, 60%, 65%, 70%, 75%, 80%, 85%, 90%, 91%, 92%, 93%, 94%, 95%, 96%, 97%, 98%, 99% but less than 100% sequence identity to that particular reference polynucleotide or polypeptide as determined by sequence alignment programs and parameters described herein and known to those skilled in the art. Such tools for alignment include those of the BLAST suite (Stephen F. Altschul, et al. (1997). “Gapped BLAST and PSI-BLAST: a new generation of protein database search programs,” Nucleic Acids Res. 25:3389-3402). Another popular local alignment technique is based on the Smith-Waterman algorithm (Smith, T. F. & Waterman, M. S. (1981) “Identification of common molecular subsequences.” J. Mol. Biol. 147:195-197). A general global alignment technique based on dynamic programming is the Needleman-Wunsch algorithm (Needleman, S. B. & Wunsch, C. D. (1970) “A general method applicable to the search for similarities in the amino acid sequences of two proteins.” J. Mol. Biol. 48:443-453). More recently, a Fast Optimal Global Sequence Alignment Algorithm (FOGSAA) was developed that purportedly produces global alignment of nucleotide and protein sequences faster than other optimal global alignment methods, including the Needleman-Wunsch algorithm. Other tools are described herein, specifically in the definition of “identity” below.

As used herein, the term “homology” refers to the overall relatedness between polymeric molecules, e.g. between nucleic acid molecules (e.g. DNA molecules) and/or between polypeptide molecules. Polymeric molecules (e.g. nucleic acid molecules (e.g. DNA molecules) and/or polypeptide molecules) that share a threshold level of similarity or identity determined by alignment of matching residues are termed homologous. Homology is a qualitative term that describes a relationship between molecules and can be based upon the quantitative similarity or identity. Similarity or identity is a quantitative term that defines the degree of sequence match between two compared sequences. In some embodiments, polymeric molecules are “homologous” to one another if their sequences are at least 25%, 30%, 35%, 40%, 45%, 50%, 55%, 60%, 65%, 70%, 75%, 80%, 85%, 90%, 95%, 96%, 97%, 98% or 99% identical or similar. The term “homologous” necessarily refers to a comparison between at least two sequences (polynucleotide or polypeptide sequences). Two polynucleotide sequences are considered homologous if the polypeptides they encode are at least 50%, 60%, 70%, 80%, 90%, 95%, or even 99% for at least one stretch of at least 20 amino acids. In some embodiments, homologous polynucleotide sequences are characterized by the ability to encode a stretch of at least 4-5 uniquely specified amino acids. For polynucleotide sequences less than 60 nucleotides in length, homology is determined by the ability to encode a stretch of at least 4-5 uniquely specified amino acids. Two protein sequences are considered homologous if the proteins are at least 50%, 60%, 70%, 80%, or 90% identical for at least one stretch of at least 20 amino acids.

Homology implies that the compared sequences diverged in evolution from a common origin. The term “homolog” refers to a first amino acid sequence or nucleic acid sequence (e.g., gene (DNA or RNA) or protein sequence) that is related to a second amino acid sequence or nucleic acid sequence by descent from a common ancestral sequence. The term “homolog” may apply to the relationship between genes and/or proteins separated by the event of speciation or to the relationship between genes and/or proteins separated by the event of genetic duplication. “Orthologs” are genes (or proteins) in different species that evolved from a common ancestral gene (or protein) by speciation. Typically, orthologs retain the same function during evolution. “Paralogs” are genes (or proteins) related by duplication within a genome. Orthologs retain the same function during evolution, whereas paralogs evolve new functions, even if the new functions are related to the original function.

As used herein, the term “variant” is a molecule that differs in its amino acid sequence or nucleic acid sequence relative to a native sequence or a reference sequence. Sequence variants may possess substitutions, deletions, insertions, or a combination of any two or three of the foregoing, at certain positions within the sequence, as compared to a native sequence or a reference sequence. Ordinarily, variants possess at least 50% identity to a native sequence or a reference sequence. In some embodiments, variants share at least 80% identity or at least 90%, 91%, 92%, 93%, 94%, 95%, 96%, 97%, 98% or 99% identity with a native sequence or a reference sequence.

As defined herein, the term “6P” refers to a mutation of 6 amino acid residues, i.e. mutation by substitution of six proline residues at positions 817, 892, 899, 942, 986 and 987 (F817P+A892P+A899P+A942P+K986P+V987P) of the amino acid sequence of the S protein of SARS-CoV-2 of SEQ ID NO: 3, that maintains the S protein in the pre-fusion form, said mutation occurring in the S2 domain (Hsieh et al., 2020). The K986P and V987P mutations occur between the heptad repeat 1 (HR1) and the central helix (CH), the F817P, A892P and A899P occur in the connecting region between the fusion peptide (FP) and HR1, and the A942P mutation occurs in HR1.

As defined herein, the term “CC” refers to a mutation by substitution of two cysteine residues at positions 383 and 985 (S383C and D985C) of the amino acid sequence of the S protein of SARS-CoV-2 of SEQ ID NO: 3 or a mutation by substitution of two cysteine residues at positions 413 and 987 (G413C and P987C) of the amino acid sequence of the S protein of SARS-CoV-2 of SEQ ID NO: 3 that keeps the receptor-binding domain (RBD) in the closed conformation (McCallum et al., 2020).

As defined herein, the term “foldon” refers to an artificial trimerization domain, in particular the T4 foldon (i.e. the trimerization domain of the fibritin of the bacteriophage T4) that promotes trimerization of the ectodomain of the S protein and allows its expression in soluble trimeric form, i.e. soluble trimerized form of the S protein. For example, the T4 foldon has been used in the sequences of SEQ ID NO: 16, SEQ ID NO: 17, SEQ ID NO: 18, SEQ ID NO: 19, SEQ ID NO: 51 and SEQ ID NO: 52. Another example of a foldon is the GCN4 foldon, which is derived from the trimerization domain of yeast GCN4 transactivator.

As defined herein, the term “ΔF” refers to a mutation by substitution of three amino acid residues occurring in the S1/S2 furin cleavage site at positions 682, 683 and 685 of the amino acid sequence of the S protein of SARS-CoV-2 of SEQ ID NO: 3: R682G+R683S+R685G.

As defined herein, the term “ΔF” refers to the deletion of the loop encompassing the S1/S2 furin cleavage site between amino acid at position 675 and amino acid at position 685 of the S protein of SARS-CoV-2 of SEQ ID NO: 3, i.e. deletion of the amino acid sequence QTQTNSPRRAR of SEQ ID NO: 50.

As defined herein, the “S3F polypeptide of SARS-CoV-2” refers to a polypeptide comprising a stabilized S protein, wherein the S1/S2 furin cleavage site has been inactivated, e.g. by mutation of 3 amino acid residues at positions 682, 683 and 685 (R682G+R683S+R685G) of the amino acid sequence of the S protein of SARS-CoV-2 of SEQ ID NO: 3.

As defined herein, the “S2P3F polypeptide of SARS-CoV-2” refers to a polypeptide comprising a stabilized S protein with a 2P mutation at positions 986 and 987 (K986P+V987P) of the amino acid sequence of the S protein of SARS-CoV-2 of SEQ ID NO: 3 and inactivation of the S1/S2 furin cleavage site, e.g. by mutation of 3 amino acid residues at positions 682, 683 and 685 (R682G+R683S+R685G) of the amino acid sequence of the S protein of SARS-CoV-2 of SEQ ID NO: 3.

As defined herein, the “S2PΔF polypeptide of SARS-CoV-2” is directed to a polypeptide comprising a stabilized S protein with a 2P mutation at positions 986 and 987 (K986P+V987P) of the amino acid sequence of the S protein of SARS-CoV-2 of SEQ ID NO: 3 and deletion of the loop encompassing the S1/S2 furin cleavage site, i.e. deletion of the amino acid sequence QTQTNSPRRAR of SEQ ID NO: 50 between position 675 and 685 of the amino acid sequence of the S protein of SARS-CoV-2 of SEQ ID NO: 3. In the S2PΔF polypeptide of SARS-CoV-2, the 2P mutation occurs in positions 975 and 976 (K975P+V976P) of SEQ ID NO: 47.

As defined herein, the “S2PΔF2A polypeptide of SARS-CoV-2” is directed to a polypeptide comprising a stabilized S protein with a 2P mutation at positions 986 and 987 (K986P+V987P) of the amino acid sequence of the S protein of SARS-CoV-2 of SEQ ID NO: 3 and deletion of the loop encompassing the S1/S2 furin cleavage site, i.e. deletion of the amino acid sequence QTQTNSPRRAR of SEQ ID NO: 50 between position 675 and 685 of the amino acid sequence of the S protein of SARS-CoV-2 of SEQ ID NO: 3, with inactivation of the ERR signal, e.g. by mutation of 2 amino acid residues at positions 1269 and 1271 (K1269A+H1271A) of the amino acid sequence of the S protein of SARS-CoV-2 of SEQ ID NO: 3. In the S2PΔF2A polypeptide of SARS-CoV-2, the 2P mutation occurs in positions 975 and 976 (K975P+V976P) of SEQ ID NO: 49 and inactivation of the ERR signal occurs e.g. by mutation of 2 amino acid residues at positions 1258 and 1260 (K1258A+H1260A) of the amino acid sequence of SEQ ID NO: 49.

As defined herein, the “T4-S2P3F polypeptide of SARS-CoV-2” (also named tristab-Secto-3F) refers to a polypeptide comprising a soluble trimerized form of the S protein with 2P and 3F mutations, in particular a polypeptide comprising a T4 foldon trimerization domain, a stabilized S protein with a 2P mutation at positions 986 and 987 (K986P+V987P) of the amino acid sequence of the S protein of SARS-CoV-2 of SEQ ID NO: 3 and inactivation of the S1/S2 furin cleavage site, e.g. by mutation of 3 amino acid residues at positions 682, 683 and 685 (R682G+R683S+R685G) of the amino acid sequence of the S protein of SARS-CoV-2 of SEQ ID NO: 3.

As defined herein, the “S6P polypeptide” of SARS-CoV-2 or the “S_{MVopt}6P polypeptide” of SARS-CoV-2 refers to a polypeptide comprising a stabilized S protein with a 6P mutation at positions 817, 892, 899, 942, 986 and 987 (F817P+A892P+A899P+A942P+K986P+V987P) of the amino acid sequence of the S protein of SARS-CoV-2 of SEQ ID NO: 3.

As defined herein, the “S6P3F polypeptide” of SARS-CoV-2 or the “S_{MVopt}6P3F polypeptide” of SARS-CoV-2 refers to a polypeptide comprising a stabilized S protein with a 6P mutation at positions 817, 892, 899, 942, 986 and 987 (F817P+A892P+A899P+A942P+K986P+V987P) of the amino acid sequence of the S protein of SARS-CoV-2 of SEQ ID NO: 3 and inactivation of the S1/S2 furin cleavage site, e.g. by mutation of 3 amino acid residues at positions 682, 683 and 685 (R682G+R683S+R685G) of the amino acid sequence of the S protein of SARS-CoV-2 of SEQ ID NO: 3.

As defined herein, the “S6PΔF polypeptide” of SARS-CoV-2 or the “S_{MVopt}6PΔF polypeptide” of SARS-CoV-2 refers to a polypeptide comprising a stabilized S protein with a 6P mutation at positions 817, 892, 899, 942, 986 and 987 (F817P+A892P+A899P+A942P+K986P+V987P) of the amino acid sequence of the S protein of SARS-CoV-2 of SEQ ID NO: 3 and deletion of the loop encompassing the S1/S2 furin cleavage site, i.e. deletion of the amino acid sequence QTQTNSPRRAR of SEQ ID NO: 50 between position 675 and 685 of the amino acid sequence of the S protein of SARS-CoV-2 of SEQ ID NO: 3. In the S6PΔF polypeptide of SARS-CoV-2, the 6P mutation occurs at positions 806, 881, 888, 931, 975 and 976 of SEQ ID NO: 58.

As defined herein, the “SCCPP polypeptide” of SARS-CoV-2 refers to a polypeptide comprising a stabilized S protein with a 2P mutation at positions 986 and 987 (K986P+V987P) of the amino acid sequence of the S protein of SARS-CoV-2 of SEQ ID NO: 3 and a CC mutation at positions 383 and 985 (S383C and D985C) of the amino acid sequence of the S protein of SARS-CoV-2 of SEQ ID NO: 3.

As defined herein, the “SCC6P polypeptide” of SARS-CoV-2 refers to a polypeptide comprising a stabilized S protein with a 6P mutation at positions 817, 892, 899, 942, 986 and 987 (F817P+A892P+A899P+A942P+K986P+V987P) of the amino acid sequence of the S protein of SARS-CoV-2 of SEQ ID NO: 3 and a CC mutation at positions 383 and 985 (S383C and D985C) of the amino acid sequence of the S protein of SARS-CoV-2 of SEQ ID NO: 3.

As used herein, the phrase “full length S protein” refers to a coronavirus spike (S) protein that includes both the S1 and S2 domains. A full length S protein may include mutations (substitutions, deletions, and/or additions), but is not missing an entire S1 or S2 domain. A full length S protein may include a furin cleavage site, or a mutated furin cleavage site between the S1 and S2 domains.

Biology of the Coronavirus

SARS-CoV-2 is an enveloped single-stranded positive-sense RNA virus belonging to the Coronavidae family and the β-coronavirus genus (Zhou, 2020). Whole genome sequencing of SARS-CoV-2 revealed 79.6% nucleotide sequence similarity with SARS-CoV-1 (Wu, 2020). The genome of SARS-CoV-2 encodes 4 structural proteins: the spike protein (S), the envelope protein (E), the membrane protein (M), and the nucleocapsid (N). The S protein, a trimeric class I fusion protein localized on the surface of the virion, plays a central role in viral attachment and entry into host cells. Cleavage of the S protein into S1 and S2 subunits by host proteases (Jaimes, 2020) is essential for viral infection. The S1 subunit contains the receptor-binding-domain (RBD), which enables the virus to bind to its entry receptor, the angiotensin-converting enzyme 2 (ACE2) (Zhou, 2020; Hoffmann, 2020). After docking with the receptor, the S1 subunit is released and the S2 subunit reveals its fusion peptide to mediate membrane fusion and viral entry (Du, 2020).

The coronavirus replicates in the cytoplasm of the host cells. The 5′ end of the RNA genome has a capped structure and the 3′ end contains a polyA tail. The envelope of the virus comprises, at its surface, peplomeric structures called spicules (or spike protein).

The genome comprises the following open reading frames or ORFs, from its 5′ end to its 3′ end: ORF1a and ORF1b corresponding to the proteins of the transcription-replication complex, and ORF-S, ORF-E, ORF-M and ORF-N corresponding to the structural proteins S, E, M and N. It also comprises ORFs corresponding to proteins of unknown function encoded by the region situated between ORF-S and ORF-E and overlapping the latter, the region situated between ORF-M and ORF-N, and the region included in ORF-N.

The S protein is a membrane glycoprotein (200-220 kDa) existing in the form of spicules or spikes emerging from the surface of the viral envelope. It is responsible for the attachment of the virus to the receptors of the host cell and for inducing the fusion of the viral envelope with the cell membrane. The S protein may be functionally divided into two sub-regions S1 and S2 wherein S1 forms the head of the S protein involved in the binding to the virus receptor on host cells and S2 forms a stalk structure. The S protein contains the major epitopes targeted by neutralizing antibodies and is thus considered as a main antigen for developing vaccines against human coronaviruses (Du, 2020; Escriou, 2014; Liniger, 2008; Bodmer, 2018; Zhu, 2020). Antibodies targeting the RBD may neutralize virus by blocking viral binding to receptors on host cells and preventing entry. Additionally, it has been observed that synthetic peptides mimicking and antibodies targeting the second heptad region (HR2) in the S2 subunit of SARS-CoV have strong neutralizing activity (Bosh, 2004; Keng, 2005; Lip, 2006; Zhang, 2004; Zhong, 2005), likely by preventing the conformational changes required for membrane fusion. Efforts to develop a SARS-CoV-2 vaccine have thus focused on eliciting responses against the S protein.

The small envelope protein (E), also called sM (small membrane), which is a nonglycosylated transmembrane protein of about 10 kDa, is the protein present in the smallest quantity in the virion. It is involved in the coronavirus budding process which occurs at the level of the intermediate compartment in the endoplasmic reticulum (ER) and the Golgi apparatus.

The M protein or matrix protein (25-30 kDa) is a more abundant membrane glycoprotein which is integrated into the viral particle by an M/E interaction, whereas the incorporation of S into the particles is directed by an S/M interaction. It appears to be important for the viral maturation of coronaviruses and for the determination of the site where the viral particles are assembled.

The N protein or nucleocapsid protein (45-50 kDa) which is the most conserved among the coronavirus structural proteins is necessary for encapsidating the genomic RNA and then for directing its incorporation into the virion. This protein is probably also involved in the replication of the RNA.

When the host cell is infected, the reading frame (ORF) situated in the 5′ region of the viral genome is translated into a polyprotein which is cleaved by the viral proteases and then releases several nonstructural proteins such as the RNA-dependent RNA polymerase (Rep) and the ATPase helicase (Hel). These two proteins are involved in the replication of the viral genome and in the generation of transcripts which are used in the synthesis of the viral proteins. The mechanisms by which these subgenomic mRNAs are produced are not completely understood; however, recent facts indicate that the sequences for regulation of transcription at the 5′ end of each gene represent signals which regulate the discontinuous transcription of the subgenomic mRNAs.

The proteins of the viral membrane (S, E and M proteins) are inserted into the intermediate compartment, whereas the replicated RNA (+ strand) is assembled with the N (nucleocapsid) protein. This protein-RNA complex then combines with the M protein contained in the membranes of the endoplasmic reticulum and the viral particles form when the nucleocapsid complex buds into the endoplasmic reticulum. The virus then migrates across the Golgi complex and eventually leaves the cell, for example by exocytosis. The site of attachment of the virus to the host cell is at the level of the S protein.

Recombinant Measles Viruses

With the aim of developing a vaccine against existing or emerging, possibly pandemic coronaviruses, in particular a vaccine that could be used in children (in particular in young children or babies) or in adult population or both, the inventors designed a strategy based on the expression of polypeptides derived from selected antigens (or suitable portion(s) thereof) by a measles virus vector, wherein in particular the measles virus (MV or MeV) is selected from live attenuated measles viruses such as vaccine measles viruses. In certain embodiments the live attenuated measles virus is the Schwarz strain.

The invention proposes a new approach to provide coronavirus antigens or polypeptides derived therefrom including spike derived antigens to the immune system of the host and especially provides use of measles virus vector to express such polypeptides or antigens, in particular for eliciting an immune response in a mammalian host, especially a human host, to confer protection, especially preventive protection, against the disease caused by coronavirus in particular SARS-CoV-2 strain. This approach using measles virus as a vector of the immunogenic polypeptides of coronavirus also takes benefits from the vector properties in particular of the immune properties of the vector to improve the quality of the immune response in the host. The inventors hence provide a recombinant infectious live attenuated measles virus, such as recombinant measles virus obtained using the Schwarz strain, capable of eliciting an immune response in mammalian, in particular in human individuals that would be effective and long lasting against illness resulting from coronavirus infection, especially from SARS-CoV-2 infection.

The invention thus relates to the use of measles virus as a vector to express coronavirus immunogens or epitopes of coronavirus antigens. In some embodiments said immunogens or epitopes encompass or derive from polypeptides derived from the wild type antigens of the SARS-CoV-2 as generally described hereabove such as the S, E, N, ORF3a, ORF8, ORF7a and M proteins of a coronavirus or specifically described for the SARS-CoV-2 strain and illustrated in the present description.

The recombinant measles virus particles may express a wild type SARS-CoV-2 antigen, fragments thereof that comprise epitopes sufficient for eliciting an immune response in a mammalian host, or mutated or truncated antigens, wherein the mutations or truncations or the fragments resulting from deletions of amino acid residues or of regions of the native antigen preserve the immunogenic properties of the antigen and enable their production or their use in immunogenic compositions. Accordingly mutated antigens or fragments of antigens may have improved stability in cells and/or enable recovery of solubilized forms of the antigens and/or multimeric forms of the antigens, in particular trimers thereof (such as for the spike derived antigens). The polypeptides disclosed herein especially originate from the CoV, in particular from SARS-CoV-2, and are structural proteins that may be identical to native proteins or alternatively that may be derived thereof by mutation, especially targeted point mutations, including by substitution (in particular by conservative amino acid residues) or by addition of amino acid residues or by secondary modification after translation (including glycosylation) or by deletion of portions of the native proteins(s) resulting in fragments having a shortened size with respect to the native protein of reference. Fragments are encompassed within the present invention to the extent that they bear epitopes of the native protein suitable for eliciting an immune response in a host in particular in a mammalian host, especially a human host, preferably a response that enables the protection against CoV, in particular SARS-CoV-2. Epitopes are in particular of the type of B cell epitopes involved in eliciting a humoral immune response through the activation of the production of antibodies in a host to whom the protein has been administered or in whom it is expressed following administration of the infectious replicative particles of the invention. Epitopes may alternatively be of the type of T cell epitopes involved in elicitation of Cell Mediated Immune response (CMI response). Fragments may have a size representing more than 50% of the amino-acid sequence size of the native protein of CoV, in particular of SARS-CoV-2, preferably at least 90% or 95%. Alternatively, fragments may be short polypeptides with at least 10 amino acid residues, which harbor epitope(s) of the native protein. Fragments in this respect also include polyepitopes as defined herein. In a particular embodiment the polypeptide is a fragment of the native antigen that contains or consists in the soluble portion of the antigen and/or is point mutated (such as with 1, 2 or by less than 5% substitutions in the amino acid residues of the native antigen). Mutations may be designed to improve its stability in the cells. The polypeptides (such as the S polypeptide) may in particular be expressed as trimeric or trimerized forms of the coronavirus native or modified antigens. The words “polypeptide” and “antigens” are used interchangeably to define a “polypeptide of the coronavirus in particular of SARS-CoV-2” according to the invention in accordance with the definition provided herein. The amino acid sequence of a polypeptide is hence either identical to a counterpart in an antigen of a strain of CoV, in particular SARS-CoV-2, including for a polypeptide which is a native mature or precursor protein of CoV, or is modified by insertion, substitution, or deletion to define an immunogenic fragment thereof or a variant thereof.

In particular a fragment or a variant having at least 50%, at least 80%, at least 90% or at least 95% amino acid sequence identity to a naturally occurring CoV polypeptide. Amino acid sequence identity can be determined as defined herein. Fragments or mutants of CoV proteins of the invention may be defined with respect to the particular amino acid sequences illustrated herein.

In a first aspect of the invention, heterologous polypeptides for expression by the recombinant measles virus are derived from glycoprotein S of a coronavirus, in particular of SARS-CoV-2: they may be the S polypeptide as such in its glycosylated or non-glycosylated form, or they may be fragments thereof such as immunogenic fragments S1 and/or S2 or shorter fragments thereof, including shorter fragments of the full-length S polypeptides that are devoid of or modified in functional domain(s), i.e, domain(s) that impact the life cycle of the virus. According to the invention, fragments of the S polypeptide of the coronavirus, especially of SARS-CoV-2 comprise epitopes suitable to elicit an immune response in the context of the recombinant virus particles. Particular fragments of S or mutated fragments of S or mutated antigens of S according to the invention are the polypeptides listed below, especially encoded by the nucleotide sequence disclosed hereafter or having the amino acid sequence described herein: S polypeptide of SARS-CoV-2, stab-S polypeptide of SARS-CoV-2 (also named S2P polypeptide of SARS-CoV-2), Secto polypeptide of SARS-CoV-2, stab-Secto polypeptide of SARS-CoV-2, S1 polypeptide of SARS-CoV-2, S2 polypeptide of SARS-CoV-2, tri-Secto polypeptide of SARS-CoV-2, tristab-Secto polypeptide of SARS-CoV-2, S3F polypeptide of SARS-CoV-2, S2P3F polypeptide of SARS-CoV-2, S2PΔF polypeptide of SARS-CoV-2, S2PΔF2A polypeptide of SARS-CoV-2, T4-S2P3F (tristab-Secto-3F) polypeptide of SARS-CoV-2, S6P polypeptide of SARS-CoV-2, S6P3F polypeptide of SARS-CoV-2, S6PΔF polypeptide of SARS-CoV-2, SCCPP polypeptide of SARS-CoV-2, SCC6P polypeptide of SARS-CoV-2, S_{MVopt}2P polypeptide of SARS-CoV-2, S_{MVopt}ΔF polypeptide of SARS-CoV-2, S_{MVopt}2PΔF polypeptide of SARS-CoV-2, preferably is selected from the group consisting of S, stab-S (also named S2P), S3F, S2P3F, S2PΔF and S2PΔF2A polypeptides of SARS-CoV-2. The fragments may be obtained from the wild type sequence or may be mutated and/or deleted with respect to the wild type sequence. Preferred fragments of S or mutated fragments of S according to the invention are selected from the group consisting of S polypeptide of SARS-CoV-2, stab-S polypeptide of SARS-CoV-2 (also named S2P polypeptide of SARS-CoV-2), S3F polypeptide of SARS-CoV-2, S2P3F polypeptide of SARS-CoV-2, S2PΔF polypeptide of SARS-CoV-2, S2PΔF2A polypeptide of SARS-CoV-2, T4-S2P3F (tristab-Secto-3F) polypeptide of SARS-CoV-2, S6P polypeptide of SARS-CoV-2, S6P3F polypeptide of SARS-CoV-2, S6PΔF polypeptide of SARS-CoV-2, SCCPP polypeptide of SARS-CoV-2, SCC6P polypeptide of SARS-CoV-2, S_{MVopt}2P polypeptide of SARS-CoV-2, S_{MVopt}ΔF polypeptide of SARS-CoV-2 and S_{MVopt}2PΔF polypeptide of SARS-CoV-2. More preferred fragments of S or mutated fragments of S or mutated antigens of S according to the invention are selected from the group consisting of S2P3F polypeptide of SARS-CoV-2, S2PΔF polypeptide of SARS-CoV-2, S2PΔF2A polypeptide of SARS-CoV-2, preferably S2PΔF polypeptide of SARS-CoV-2, more preferably S2PΔF2A polypeptide of SARS-CoV-2.

Preferably, 1, 2, 3 or more amino acid mutation(s), i.e. amino acid substitution(s), insertion(s) and/or deletion(s), is(are) introduced into the amino acid sequence of the S protein of CoV, in particular coronavirus SARS-CoV-2:
to maintain the expressed protein in its prefusion state (2P mutation), and/or
to prevent S1/S2 cleavage (furin cleavage site inactivation, either through 3F mutation or through ΔF deletion of the encompassing loop), and/or
to inactivate the Endoplasmic Reticulum retrieval signal (ERRS) (2A mutation as defined below), and/or
to maintain the receptor-binding domain (RBD) localized in the S1 domain of the S protein in the closed conformation (i.e. in down position or in a closed down state).

In another aspect of the invention heterologous polypeptides for expression by the recombinant measles virus are derived from one of the following antigens of a coronavirus, in particular of SARS-CoV-2, E, N, ORF3a, ORF8, ORF7a or M proteins, in particular N protein.

The invention thus relates to a nucleic acid construct comprising:
(1) a cDNA molecule encoding a full length, infectious antigenomic (+) RNA strand of a measles virus (MV); and
(2) a first heterologous polynucleotide encoding at least one polypeptide of a coronavirus (CoV), in particular of SARS-CoV-2, in particular said first polynucleotide encoding at least the spike (S) polypeptide of a coronavirus (CoV), in particular of coronavirus SARS-CoV-2, or an immunogenic fragment thereof, or a variant of the S polypeptide or fragment thereof that has 1, 2, 3 or more amino acid substitution(s), insertion(s) and/or deletion(s), including those especially disclosed above, and wherein the first heterologous polynucleotide is positioned (operatively cloned) within an additional transcription unit (ATU) inserted within the cDNA of the antigenomic (+) RNA to provide a recombinant MV-CoV, in particular MV-CoVS nucleic acid molecule.

In a preferred embodiment of the invention, the nucleic acid construct comprises:
(1) a cDNA molecule encoding a full length, infectious antigenomic (+) RNA strand of a measles virus (MV); and
(2) a first heterologous polynucleotide encoding
   (a) a full length spike (S) protein of SARS-CoV-2 of SEQ ID NO: 3, or
   (b) an immunogenic fragment of the full length S protein in (a) selected from the group consisting of the S1 polypeptide of SEQ ID NO: 11, the S2 polypeptide of SEQ ID NO: 13, the Secto polypeptide of SEQ ID NO: 7 and the tri-Secto polypeptide of SEQ ID NO: 16, or
   (c) a variant of (a) or (b) that has 1, 2, 3 or more amino acid residue substitution(s), insertion(s) and/or deletion(s), in particular less than 10, or less than 5 amino acid residue substitutions, insertions, and/or deletions, preferably a mutated antigen comprising
      (i) a mutation that maintains the expressed full length S protein in its prefusion conformation, in particular a mutation by substitution of amino acid residue(s) occurring in the S2 domain, preferably a mutation by substitution of at least two proline residues occurring in the S2 domain, and/or
      (ii) a mutation that inactivates the furin cleavage site of the S protein, in particular a mutation by insertion, substitution or deletion of amino acid residue(s) occurring in the S1/S2 furin cleavage site, and/or
      (iii) a mutation that inactivates the Endoplasmic Reticulum Retrieval Signal (EERS), and/or
      (iv) a mutation that maintains the receptor-binding domain (RBD) localized in the S1 domain of the S protein in the closed conformation, and wherein the first heterologous polynucleotide is positioned in an additional transcription unit (ATU) located between the P gene and the M gene of the MV (ATU2) or in an ATU located downstream of the H gene of the MV (ATU3).

In a particular embodiment of the invention, in the nucleic acid construct:
(i) the mutation that maintains the expressed full length S protein in its prefusion conformation is a mutation by substitution of two proline residues at positions 986 and 987 (K986P and V987P) of the amino acid sequence of the S protein of SARS-CoV-2 of SEQ ID NO: 3, or a mutation by substitution of six proline residues at positions 817, 892, 899, 942, 986 and 987 (F817P, A892P, A899P, A942P, K986P and V987P) of the amino acid sequence of the S protein of SARS-CoV-2 of SEQ ID NO: 3, and/or
(ii) the mutation that inactivates the furin cleavage site of the S protein is a mutation by substitution of three amino acid residues occurring in the S1/S2 furin cleavage site at positions 682, 683 and 685 (R682G, R683S and R685G) of the amino acid sequence of the S protein of SARS-CoV-2 of SEQ ID NO: 3, or a mutation by deletion of the loop encompassing the S1/S2 furin cleavage site between amino acid at position 675 and amino acid at position 685 of the S protein of SARS-CoV-2 of SEQ ID NO: 3, the loop consisting of the amino acid sequence QTQTNSPRRAR of SEQ ID NO: 50, and/or
(iii) the mutation that inactivates the EERS is a mutation by substitution of two alanine residues at positions 1269 and 1271 of the amino acid sequence of SEQ ID NO: 3, and/or
(iv) the mutation that maintains the RBD localized in the S1 domain of the S protein in the closed conformation is a mutation by substitution of two cysteine residues at positions 383 and 985 (S383C and D985C) of the amino acid sequence of the S protein of SARS-CoV-2 of SEQ ID NO: 3 or a mutation by substitution of two cysteine residues at positions 413 and 987 (G413C and P987C) of the amino acid sequence of the S protein of SARS-CoV-2 of SEQ ID NO: 3; and/or
(v) the variant in (c) encodes a polypeptide comprising a mutation selected from the group consisting of a deletion of the amino acid residues at positions 69 and 70 of the amino acid sequence of SEQ ID NO: 3, a deletion of the amino acid residues at positions 144 and 145 of the amino acid sequence of SEQ ID NO: 3, a mutation by substitution of the tyrosine residue at position 501 of the amino acid sequence of SEQ ID NO: 3 (N501Y), a mutation by substitution of the aspartic acid residue at position 570 of the amino acid sequence of SEQ ID NO: 3 (A570D), a mutation by substitution of the histidine residue at position 681 of the amino acid sequence of SEQ ID NO: 3 (P681H), a mutation by substitution of the isoleucine residue at position 716 of the amino acid sequence of SEQ ID NO: 3 (T7161), a mutation by substitution of the alanine residue at position 982 of the amino acid sequence of SEQ ID NO: 3 (S982A), a mutation by substitution of the histidine residue at position 1118 of the amino acid sequence of SEQ ID NO: 3 (D1118H), a mutation by substitution of the lysine residue at position 484 of the amino acid sequence of SEQ ID NO: 3 (E484K), a mutation by substitution of the asparagine residue at position 417 of the amino acid sequence of SEQ ID NO: 3 (K417N), a mutation by substitution of the threonine residue at position 417 of the amino acid sequence of SEQ ID NO: 3 (K417T) and a mutation by substitution of the glycine residue at position 614 of the amino acid sequence of SEQ ID NO: 3 (D614G), in particular a mutation selected from the group consisting of a mutation by substitution of the tyrosine residue at position 501 of the amino acid sequence of SEQ ID NO: 3 (N501Y), a mutation by substitution of the lysine residue at position 484 of the amino acid sequence of SEQ ID NO: 3 (E484K), a mutation by substitution of the asparagine residue at position 417 of the amino acid sequence of SEQ ID NO: 3 (K417N) and a mutation by substitution of the threonine residue at position 417 of the amino acid sequence of SEQ ID NO: 3 (K417T).

In some embodiments, the SARS-CoV-2 antigenic polypeptide is a full length S protein of SARS-CoV-2 of SEQ ID NO: 3.

In some embodiments, the immunogenic fragment or the antigenic fragment of the full length S protein is selected from the group consisting of the S1 polypeptide of SEQ ID NO: 11, the S2 polypeptide of SEQ ID NO: 13, the Secto polypeptide of SEQ ID NO: 7 and the tri-Secto polypeptide of SEQ ID NO: 16.

In some embodiments, the full length S protein of SARS-CoV-2 antigenic polypeptide further comprises one or more additional substitutions that maintain(s) the expressed full length S protein in its prefusion conformation. In some embodiments, the full length S protein further comprises the amino acid mutations K986P and V987P of SEQ ID NO: 3, or the amino acid mutations F817P, A892P, A899P, A942P, K986P and V987P of SEQ ID NO: 3.

In some embodiments, the full length S protein of SARS-CoV-2 antigenic polypeptide further comprises one or more additional substitutions that inactivate(s) the furin cleavage site of the S protein. In some embodiments, the full length S protein further comprises the amino acid mutations R682G, R683S and R685G of SEQ ID NO: 3, or the deletion of the loop encompassing the S1/S2 furin cleavage site between amino acid at position 675 and amino acid at position 685 of the S protein of SARS-CoV-2 of SEQ ID NO: 3, the loop consisting of the amino acid sequence QTQTNSPRRAR of SEQ ID NO: 50.

In some embodiments, the full length S protein of SARS-CoV-2 antigenic polypeptide further comprises one or more additional substitutions that inactivate(s) the EERS. In some embodiments, the full length S protein further comprises a substitution of two alanine residues at positions 1269 and 1271 of the amino acid sequence of SEQ ID NO: 3.

In some embodiments, the full length S protein of SARS-CoV-2 antigenic polypeptide further comprises one or more additional substitutions that maintains the RBD localized in the S1 domain of the S protein in the closed conformation. In some embodiments, the full length S protein further comprises the amino acid mutations S383C and D985C of SEQ ID NO: 3. In some embodiments, the full length S protein further comprises the amino acid mutations G413C and P987C of SEQ ID NO: 3.

In some embodiments, the full length S protein of SARS-CoV-2 antigenic polypeptide further comprises a deletion of the amino acid residues at positions 69 and 70 of the amino acid sequence of SEQ ID NO: 3. In some embodiments, the full length S protein of SARS-CoV-2 antigenic polypeptide further comprises a deletion of the amino acid residues at positions 144 and 145 of the amino acid sequence of SEQ ID NO: 3. In some embodiments, the full length S protein of SARS-CoV-2 antigenic polypeptide further comprises the amino acid mutation N501Y of SEQ ID NO: 3. In some embodiments, the full length S protein of SARS-CoV-2 antigenic polypeptide further comprises the amino acid mutation A570D of SEQ ID NO: 3. In some embodiments, the full length S protein of SARS-CoV-2 antigenic polypeptide further comprises the amino acid mutation P681H of SEQ ID NO: 3. In some embodiments, the full length S protein of SARS-CoV-2 antigenic polypeptide further comprises the amino acid mutation T7161 of SEQ ID NO: 3. In some embodiments, the full length S protein of SARS-CoV-2 antigenic polypeptide further comprises the amino acid mutation S982A of SEQ ID NO: 3. In some embodiments, the full length S protein of SARS-CoV-2 antigenic polypeptide further comprises the amino acid mutation D1118H of SEQ ID NO: 3. In some embodiments, the full length S protein of SARS-CoV-2 antigenic polypeptide further comprises the amino acid mutation E484K of SEQ ID NO: 3. In some embodiments, the full length S protein of SARS-CoV-2 antigenic polypeptide further comprises the amino acid mutation K417N of SEQ ID NO: 3. In some embodiments, the full length S protein of SARS-CoV-2 antigenic polypeptide further comprises the amino acid mutation K417T of SEQ ID NO: 3. In some embodiments, the full length S protein of SARS-CoV-2 antigenic polypeptide further comprises the amino acid mutation D614G of SEQ ID NO: 3.

In some embodiments, the mutated antigen of the full length S protein or of the immunogenic fragment or the antigenic fragment is (a) the TA-S2P3F polypeptide of SEQ ID NO: 52, or a variant thereof having at least 90% identity with SEQ ID NO: 52, wherein the variant does not vary at positions 682, 683, 685, 986 and 987; or (b) the S6P polypeptide of SEQ ID NO: 54, or a variant thereof having at least 90% identity with SEQ ID NO: 54, wherein the variant does not vary at positions 817, 892, 899, 942, 986 and 987; or (c) the S6P3F polypeptide of SEQ ID NO: 56, or a variant thereof having at least 90% identity with SEQ ID NO: 56, wherein the variant does not vary at positions 682, 683, 685, 817, 892, 899, 942, 986 and 987; or (d) the S6PΔF polypeptide of SEQ ID NO: 58, or a variant thereof having at least 90% identity with SEQ ID NO: 58, wherein the variant does not vary at positions 806, 881, 888, 931, 975 and 976; or (e) the SCCPP polypeptide of SEQ ID NO: 60, or a variant thereof having at least 90% identity with SEQ ID NO: 60, wherein the variant does not vary at positions 383, 985, 986 and 987; or (f) the SCC6P polypeptide of SEQ ID NO: 62, or a variant thereof having at least 90% identity with SEQ ID NO: 62, wherein the variant does not vary at positions 383, 817, 892, 899, 942, 985, 986 and 987; or (g) the S_{MVopt}2P polypeptide of SEQ ID NO: 5, or a variant thereof having at least 90% identity with SEQ ID NO: 5, wherein the variant does not vary at positions 986 and 987; or (h) the S_{MVopt}ΔF polypeptide of SEQ ID NO: 65, or a variant thereof having at least 90% identity with SEQ ID NO: 65; or (i) the S_{MVopt}2PΔF polypeptide of SEQ ID NO: 47, or a variant thereof having at least 90% identity with SEQ ID NO: 47, wherein the variant does not vary at positions 975 and 976; or (j) the S_{MVopt}6P polypeptide, or a variant thereof having at least 90% identity with the S_{MVopt}6P polypeptide, wherein the variant does not vary at positions 817, 892, 899, 942, 986 and 987; or (k) the S_{MVopt}6PΔF polypeptide, or a variant thereof having at least 90% identity with the S_{MVopt}6PΔF polypeptide, wherein the variant does not vary at positions 806, 881, 888, 931, 975 and 976; or (l) the S_{MVopt}6P3F polypeptide, or a variant thereof having at least 90% identity with the S_{MVopt}6P3F polypeptide, wherein the variant does not vary at positions 682, 683, 685, 817, 892, 899, 942, 986 and 987. In some embodiments, the mutated antigen is (a) the TA-S2P3F polypeptide of SEQ ID NO: 52; or (b) the S6P polypeptide of SEQ ID NO: 54, or (c) the S6P3F polypeptide of SEQ ID NO: 56, or (d) the S6PΔF polypeptide of SEQ ID NO: 58, or (e) the SCCPP polypeptide of SEQ ID NO: 60, or (f) the SCC6P polypeptide of SEQ ID NO: 62, or (g) the S_{MVopt}2P polypeptide of SEQ ID NO: 5, or (h) the S_{MVopt}ΔF polypeptide of SEQ ID NO: 65 or (i) the S_{MVopt}2PΔF polypeptide of SEQ ID NO: 47.

In some embodiments, the nucleic acid construct can be designed using the measles optimized-gene S_{MVopt} of SEQ ID NO: 36 instead of the fully optimized gene S of SEQ ID NO: 2.

In a particular embodiment, the first heterologous polynucleotide is positioned in an ATU2 located between the P gene and the M gene of the MV or in an ATU3 located downstream of the H gene of the MV. Preferably, the first heterologous polynucleotide is positioned in an ATU3 located downstream of the H gene of the MV.

A nucleic acid construct according to the invention is in particular a purified DNA molecule, obtained or obtainable by recombination of various polynucleotides of different origins, operably linked together. It is also and interchangeably designated as a cDNA as a result of the designation as a cDNA, of the molecule encoding a full length, infectious antigenomic (+) RNA strand of a measles virus (MV).

The expression “operatively linked” or “operably linked” refers to the functional link existing between the different polynucleotides of the nucleic acid construct of the invention such that the different polynucleotides and nucleic acid construct are efficiently transcribed and if appropriate translated, in particular in cells or cell lines, especially in cells or cell lines used as part of a rescue system for the production or amplification of recombinant infectious MV particles of the invention or in host cells, especially in mammalian or in human cells.

In another aspect of the invention, additional heterologous polypeptides for expression by the recombinant measles virus are derived from glycoprotein S of a coronavirus, in particular of SARS-CoV-2: they may be the S polypeptide as such in its glycosylated or non-glycosylated form, or they may be fragments thereof such as immunogenic fragments S1 and/or S2 or shorter fragments thereof, including shorter fragments of the full-length S polypeptides that are devoid of or modified in functional domain(s), i.e, domain(s) that impact the life cycle of the virus. According to the invention, fragments of the S polypeptide of the coronavirus, especially of SARS-CoV-2 comprise epitopes suitable to elicit an immune response in the context of the recombinant virus particles. Particular fragments of S or mutated fragments of S or mutated antigens of S according to the invention are the polypeptides listed below, especially encoded by the nucleotide sequence disclosed hereafter or having the amino acid sequence described herein: S polypeptide of SARS-CoV-2 (SEQ ID NO: 3), stab-S polypeptide of SARS-CoV-2 (also named S2P polypeptide of SARS-CoV-2) (SEQ ID NO: 5), Secto polypeptide of SARS-CoV-2 (SEQ ID NO: 7), stab-Secto polypeptide of SARS-CoV-2 (SEQ ID NO: 9), S1 polypeptide of SARS-CoV-2 (SEQ ID NO: 11), S2 polypeptide of SARS-CoV-2 (SEQ ID NO: 13), tri-Secto polypeptide of SARS-CoV-2 (SEQ ID NO: 17), tristab-Secto polypeptide of SARS-CoV-2 (SEQ ID NO: 19), or S mutated in the domain involved in endoplasmic reticulum retention. In some embodiments, S mutated in the domain involved in endoplasmic reticulum retention preferably is, or is derived from, S (SEQ ID NO: 3) or stab-S (also named S2P) (SEQ ID NO: 5) polypeptides of SARS-CoV-2. The fragments may be obtained from the wild type sequence or may be mutated and/or deleted with respect to the wild type sequence.

Preferred fragments of S or mutated fragments of S according to the invention are selected from the group consisting of S polypeptide of SARS-CoV-2 (SEQ ID NO: 3), stab-S polypeptide of SARS-CoV-2 (also named S2P polypeptide of SARS-CoV-2) (SEQ ID NO: 5).

Preferably, 1, 2, 3 or more amino acid mutation(s), i.e. amino acid substitution(s), insertion(s) and/or deletion(s), is(are) introduced into the amino acid sequence of the S protein of CoV, in particular SARS-CoV-2:
to maintain the expressed protein in its prefusion state (P2 mutation), and/or
to inactivate the Endoplasmic Reticulum retrieval signal (ERRS) (2A mutation or deletion of a KXHXX motif of SEQ ID NO: 149), and/or
to prevent the activity of intracellular retention, in particular retention involving cycling between Golgi and Endoplasmic Reticulum (ER) compartments.

In a particular embodiment, a mutation by insertion, substitution, or deletion in the cytoplasmic tail of the S protein at least impairs the retrieval of the polypeptide in the ER, wherein the mutation by insertion, substitution, or deletion is carried out in the 11 amino acid residue sequence of the S protein that aligns with positions 1263 to 1273 of the amino acid sequence of SEQ ID NO: 3 and encompasses a mutation by insertion, substitution, or deletion of all or part of the amino acid residues of the ERRS signal encompassing the KXHXX motif of SEQ ID NO: 149. The mutation in this particular domain allows transport of the resulting polypeptide to the plasma membrane of the cells.

In a particular embodiment, a mutation by insertion, substitution, or deletion in the cytoplasmic tail of the S protein at least increases cell surface expression of the dual domain S protein, wherein the mutation by insertion, substitution, or deletion is carried out in the 11 amino acid residues sequence of the S protein that may be aligned with positions 1263 to 1273 of the amino acid sequence of SEQ ID NO: 3 and encompasses a mutation by insertion, substitution, or deletion of all or part of the amino acid residues of the ERRS signal encompassing the KXHXX motif of SEQ ID NO: 149. The mutation in this particular domain allows transport of the resulting polypeptide to the plasma membrane of the cells. In a particular embodiment, the cell surface expression of the dual domain S protein having a mutation by insertion, substitution, or deletion in the cytoplasmic tail is increased by at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, compared to cell surface expression of wild type full length S protein. In a particular embodiment, the cell surface expression of the dual domain S protein having a mutation by insertion, substitution, or deletion in the cytoplasmic tail is increased by at least 5%, 10%, 15%, 20%, 25%, 30%, 35%, 40%, 45%, 50%, 55%, 60%, 65%, 70%, 75%, 80%, 85%, 90%, 95%, or 100%, compared to cell surface expression of wild type full length S protein. In a particular embodiment, the cell surface expression of the dual domain S protein having a mutation by insertion, substitution, or deletion in the cytoplasmic tail is increased from between about 1% and about 100%, between about 5% and about 100%, between about 10% and about 100%, between about 15% and about 100%, between about 20% and about 100%, between about 25% and about 100%, between about 30% and about 100%, between about 35% and about 100%, between about 40% and about 100%, between about 45% and about 100%, between about 50% and about 100%, between about 55% and about 100%, between about 60% and about 100%, between about 65% and about 100%, between about 70% and about 100%, between about 75% and about 100%, between about 80% and about 100%, between about 85% and about 100%, between about 90% and about 100%, or between about 95% and about 100%. In a particular embodiment, the cell surface expression of the dual domain S protein having a mutation by insertion, substitution, or deletion in the cytoplasmic tail is increased from between 1% and 100%, between 5% and 100%, between 10% and 100%, between 15% and 100%, between 20% and 100%, between 25% and 100%, between 30% and 100%, between 35% and 100%, between 40% and 100%, between 45% and 100%, between 50% and 100%, between 55% and 100%, between 60% and 100%, between 65% and 100%, between 70% and 100%, between 75% and 100%, between 80% and 100%, between 85% and 100%, between 90% and 100%, or between 95% and 100%.

In another aspect of the invention, the recombinant measles virus may express a second heterologous polypeptide, which is an antigenic polypeptide derived from one of the following antigens of a coronavirus, in particular of SARS-CoV-2, E (SEQ ID NO: 23), N (SEQ ID NO: 22), ORF3a (SEQ ID NO: 26), ORF8 (SEQ ID NO: 25), ORF7a (SEQ ID NO: 27) or M (SEQ ID NO: 24) proteins, in particular N (SEQ ID NO: 22) protein.

The invention thus relates to a nucleic acid construct comprising:
(1) a cDNA molecule encoding a full length antigenomic (+) RNA strand of an attenuated strain of measles virus (MV); and
(2) a first heterologous polynucleotide encoding at least one polypeptide of a coronavirus (CoV), in particular of coronavirus SARS-CoV-2, in particular said first polynucleotide encoding at least the spike (S) polypeptide of a coronavirus (CoV), in particular of coronavirus SARS-CoV-2, or an immunogenic fragment thereof that has 1, 2, 3 or more amino acid substitution(s), insertion(s) and/or deletion(s), including those especially disclosed above, and wherein the first heterologous polynucleotide is positioned within an additional transcription unit (ATU) inserted within the cDNA of the antigenomic (+) RNA to provide a recombinant MV-CoV, in particular MV-CoVS nucleic acid molecule.

In a preferred embodiment of the invention, the nucleic acid construct comprises:
(1) a cDNA molecule encoding a full length antigenomic (+) RNA strand of an attenuated strain of measles virus (MV); and
(2) a first heterologous polynucleotide encoding
   (a) a dual domain S protein polypeptide of SARS-CoV-2 comprising:
      a mutation by insertion, substitution, or deletion in the cytoplasmic tail of the dual domain S protein, wherein the mutation by insertion, substitution, or deletion is in the 11 amino acid residue sequence of the S protein aligned with positions 1263 to 1273 of the amino acid sequence of SEQ ID NO: 3 and encompasses a mutation by insertion, substitution, or deletion of all or part of the amino acid residues of the ERRS signal encompassing the KXHXX motif of SEQ ID NO: 149, and wherein said mutation by insertion, substitution, or deletion at least impairs the retrieval of the polypeptide in the Endoplasmic Reticulum (ER), in particular a dual domain S protein of SARS-CoV-2 comprising a mutation by insertion, substitution, or deletion of all or part of the amino acid residues from position 1263 to position 1273 of the amino acid sequence of SEQ ID NO: 3, with the proviso that at least two amino acid residues of a KLHYT motif of SEQ ID NO: 150 from position 1269 to position 1273 of the amino acid sequence of SEQ ID NO: 3 are mutated by substitution or that a KLHYT motif of SEQ ID NO: 150 from position 1269 to position 1273 of the amino acid sequence of SEQ ID NO: 3 is deleted, and
      optionally an additional mutation by substitution that maintains the expressed dual domain S protein in its prefusion conformation, or
   (b) an immunogenic fragment of the dual domain S protein in (a) or a mutated antigen thereof that has 1, 2, 3 or more amino acid substitution(s), insertion(s) and/or deletion(s), and wherein the first heterologous polynucleotide is positioned in an additional transcription unit located between the P gene and the M gene of the MV (ATU2) or in an additional transcription unit located downstream of the H gene of the MV (ATU3), preferably in ATU2.

In a particular embodiment of the invention, the dual domain S protein of SARS-CoV-2 antigenic polypeptide further comprises a mutation selected from the group consisting of a deletion of the amino acid residues at positions 69 and 70 of the amino acid sequence of SEQ ID NO: 3, a deletion of the amino acid residues at positions 144 and 145 of the amino acid sequence of SEQ ID NO: 3, a mutation by substitution of the tyrosine residue at position 501 of the amino acid sequence of SEQ ID NO: 3 (N501Y), a mutation by substitution of the aspartic acid residue at position 570 of the amino acid sequence of SEQ ID NO: 3 (A570D), a mutation by substitution of the histidine residue at position 681 of the amino acid sequence of SEQ ID NO: 3 (P681H), a mutation by substitution of the isoleucine residue at position 716 of the amino acid sequence of SEQ ID NO: 3 (T7161), a mutation by substitution of the alanine residue at position 982 of the amino acid sequence of SEQ ID NO: 3 (S982A), a mutation by substitution of the histidine residue at position 1118 of the amino acid sequence of SEQ ID NO: 3 (D1118H), a mutation by substitution of the lysine residue at position 484 of the amino acid sequence of SEQ ID NO: 3 (E484K), a mutation by substitution of the asparagine residue at position 417 of the amino acid sequence of SEQ ID NO: 3 (K417N), a mutation by substitution of the threonine residue at position 417 of the amino acid sequence of SEQ ID NO: 3 (K417T) and a mutation by substitution of the glycine residue at position 614 of the amino acid sequence of SEQ ID NO: 3 (D614G), in particular a mutation selected from the group consisting of a mutation by substitution of the tyrosine residue at position 501 of the amino acid sequence of SEQ ID NO: 3 (N501Y), a mutation by substitution of the lysine residue at position 484 of the amino acid sequence of SEQ ID NO: 3 (E484K), a mutation by substitution of the asparagine residue at position 417 of the amino acid sequence of SEQ ID NO: 3 (K417N) and a mutation by substitution of the threonine residue at position 417 of the amino acid sequence of SEQ ID NO: 3 (K417T).

In some embodiments, the dual domain S protein of SARS-CoV-2 antigenic polypeptide further comprises a deletion of the amino acid residues at positions 69 and 70 of the amino acid sequence of SEQ ID NO: 3. In some embodiments, the dual domain S protein of SARS-CoV-2 antigenic polypeptide further comprises a deletion of the amino acid residues at positions 144 and 145 of the amino acid sequence of SEQ ID NO: 3. In some embodiments, the dual domain S protein of SARS-CoV-2 antigenic polypeptide further comprises the amino acid mutation N501Y of SEQ ID NO: 3. In some embodiments, the dual domain S protein of SARS-CoV-2 antigenic polypeptide further comprises the amino acid mutation A570D of SEQ ID NO: 3. In some embodiments, the dual domain S protein of SARS-CoV-2 antigenic polypeptide further comprises the amino acid mutation P681H of SEQ ID NO: 3. In some embodiments, the dual domain S protein of SARS-CoV-2 antigenic polypeptide further comprises the amino acid mutation T7161 of SEQ ID NO: 3. In some embodiments, the dual domain S protein of SARS-CoV-2 antigenic polypeptide further comprises the amino acid mutation S982A of SEQ ID NO: 3. In some embodiments, the dual domain S protein of SARS-CoV-2 antigenic polypeptide further comprises the amino acid mutation D1118H of SEQ ID NO: 3. In some embodiments, the dual domain S protein of SARS-CoV-2 antigenic polypeptide further comprises the amino acid mutation E484K of SEQ ID NO: 3. In some embodiments, the dual domain S protein of SARS-CoV-2 antigenic polypeptide further comprises the amino acid mutation K417N of SEQ ID NO: 3. In some embodiments, the dual domain S protein of SARS-CoV-2 antigenic polypeptide further comprises the amino acid mutation K417T of SEQ ID NO: 3. In some embodiments, the dual domain S protein of SARS-CoV-2 antigenic polypeptide further comprises the amino acid mutation D614G of SEQ ID NO: 3.

In a particular embodiment of the mutation by insertion, substitution, or deletion in the cytoplasmic tail of the S protein, all the amino acid residues of the ERRS signal encompassing the KXHXX motif of SEQ ID NO: 149 are substituted or deleted.

In another particular embodiment of the mutation by insertion, substitution, or deletion in the cytoplasmic tail of the S protein, part of the amino acid residues of the ERRS signal encompassing the KXHXX motif of SEQ ID NO: 149 are substituted or deleted.

In a particular embodiment of the invention, the mutation by insertion, substitution, or deletion in the cytoplasmic tail of the S protein encompasses a mutation by insertion, substitution, or deletion of all or part of the amino acid residues of the ERRS signal encompassing the KXHXX motif of SEQ ID NO: 149 and a mutation by insertion, substitution, or deletion of 1, 2, 3, 4, 5 or 6 amino acid residue(s), the mutation occurring in the 11 amino acid residues sequence of the S protein that may be aligned with positions 1263 to 1273 of the amino acid sequence of SEQ ID NO: 3.

Preferably, the first heterologous polynucleotide encodes a dual domain S protein of SARS-CoV-2 comprising a mutation by substitution of two alanine residues at positions 1269 and 1271 of the amino acid sequence of SEQ ID NO: 3, or a deletion of the amino acid residues from position 1269 to position 1273 of the amino acid sequence of SEQ ID NO: 3, or a deletion of the amino acid residues from position 1263 to position 1273 of the amino acid sequence of SEQ ID NO: 3.

Even more preferably, the first heterologous polynucleotide encodes (a) a prefusion-stabilized SF-2P-dER polypeptide of SARS-CoV-2 comprising a mutation by substitution of two proline residues at positions 986 and 987 of the amino acid sequence of SEQ ID NO: 3 and a deletion of its 11 C-terminal amino acid residues from position 1263 to position 1273 of the amino acid sequence of SEQ ID NO: 3, or (b) a prefusion-stabilized SF-2P-2a polypeptide of SARS-CoV-2 comprising a mutation by substitution of two proline residues at positions 986 and 987 of the amino acid sequence of SEQ ID NO: 3 and a mutation by substitution of two alanine residues at positions 1269 and 1271 of the amino acid sequence of SEQ ID NO: 3. In a particular embodiment, the first heterologous polynucleotide is positioned in an ATU2 located between the P gene and the M gene of the MV or in an ATU3 located downstream of the H gene of the MV. Preferably, the first heterologous polynucleotide is positioned in an ATU2 located between the P gene and the M gene of the MV. A nucleic acid construct according to the invention is a purified DNA molecule, obtained or obtainable by recombination of various polynucleotides of different origins, operably linked together. A nucleic acid construct may include a cDNA molecule encoding a full length antigenomic (+) RNA strand of a measles virus (MV).

In a particular embodiment of the invention, the nucleic acid construct further comprises a second heterologous polynucleotide encoding at least one polypeptide, an immunogenic fragment thereof (including a wild type or a mutated fragment) or a mutated antigen thereof that has 1, 2, 3 or more amino acid substitution(s), insertion(s) and/or deletion(s), of a coronavirus, in particular of coronavirus SARS-CoV-2, wherein the polypeptide is different from at least one polypeptide encoded by the first heterologous polynucleotide or the polypeptide encoded by the first heterologous polynucleotide and in particular is selected from the group consisting of the nucleocapsid (N) polypeptide, the matrix (M), the E polypeptide, the ORF8 polypeptide, the ORF7a polypeptide and the ORF3a polypeptide, or immunogenic fragments thereof or mutated fragments thereof or a mutated antigen thereof that has 1, 2, 3 or more amino acid substitution(s), insertion(s) and/or deletion(s), the second heterologous polynucleotide being positioned within an ATU at a location distinct from the location of the first heterologous polynucleotide.

In a particular embodiment of the invention, the nucleic acid construct further comprises a second heterologous polynucleotide encoding at least one polypeptide, an immunogenic fragment thereof or an antigenic fragment thereof (including a wild type or a mutated fragment) or a mutated antigen thereof that has 1, 2, 3 or more amino acid substitution(s), insertion(s) and/or deletion(s), of a coronavirus, in particular of SARS-CoV-2, wherein the polypeptide is different from at least one polypeptide encoded by the first heterologous polynucleotide and in particular is selected from the group consisting of the nucleocapsid (N) polypeptide, the matrix (M), the E polypeptide, the ORF8 polypeptide, the ORF7a polypeptide and the ORF3a polypeptide, or immunogenic fragments thereof or mutated fragments thereof or a mutated antigen thereof that has 1, 2, 3 or more amino acid substitution(s), insertion(s) and/or deletion(s), the second heterologous polynucleotide being positioned within an additional transcription unit (ATU) at a location distinct from the ATU of the first heterologous polynucleotide, in particular within an additional transcriptional unit upstream of the N gene of the MV (ATU1), or in particular within ATU2 or in particular within ATU3.

The ATU for cloning of the second heterologous polynucleotide is located at a different location with respect to the ATU used for cloning the first heterologous polynucleotide, in particular is located upstream of the N gene of the MV in the ATU1, or in particular within an ATU at a location between the P gene and the M gene of the MV in the ATU2 or in particular within an ATU at a location downstream of the H gene of the MV in the ATU3.

In a further aspect the invention concerns a nucleic acid construct comprising:
(1) a cDNA molecule encoding a full length, infectious antigenomic (+) RNA strand of a measles virus (MV); and
(2) a heterologous polynucleotide encoding at least one polypeptide of a coronavirus (CoV), in particular of SARS-CoV-2, selected from the group consisting of the nucleocapsid (N) polypeptide, the matrix (M), the E polypeptide, the ORF8 polypeptide, the ORF7a polypeptide and the ORF3a polypeptide, or immunogenic or antigenic fragments thereof or mutated fragments thereof or mutated antigens thereof that have 1, 2, 3 or more amino acid substitution(s), insertion(s) and/or deletion(s), the second heterologous polynucleotide being positioned within an ATU.

Additional transcriptional unit (ATU) sequences, especially ATU1, ATU2, ATU3 used for the invention, are sequences in the cDNA of the MV that are used for cloning heterologous polynucleotides into the cDNA of MV. ATU sequences comprise cis-acting sequences necessary for MV-dependent expression of a transgene, such as a promoter of the gene preceding, the insert represented by the polynucleotide, e.g., the first or the second polynucleotide encoding at least one polypeptide of a coronavirus, in particular encoding the spike (S) polypeptide of a coronavirus (CoV), in particular of SARS-CoV-2, or an immunogenic fragment t thereof that has 1, 2, 3 or more amino acid substitution(s), insertion(s) and/or deletion(s), or in particular encoding the nucleocapsid (N) polypeptide, the matrix (M), the E polypeptide, the ORF8 polypeptide, the ORF7a polypeptide or the ORF3a polypeptide, or immunogenic fragments thereof or mutated fragments thereof or a mutated antigen thereof that have 1, 2, 3 or more amino acid substitution(s), insertion(s) and/or deletion(s) and a multiple cloning sites cassette for insertion of said polynucleotide. In addition to the intergenic sequence of the genes (including Gene Start (GS) promoting the transcription and Gene End (GE) of the P gene of MV terminating the transcription of the insert (heterologous polynucleotide)), an ATU comprises a polylinker sequence for the insertion of the heterologous polynucleotide. ATU sequences are illustrated in the constructs of the invention.

When used to carry out the invention, the ATU is advantageously located within the N-terminal sequence of the cDNA molecule encoding the full-length (+)RNA strand of the antigenome of the MV and is especially located upstream from the N gene (ATU1) or between the P and M genes of this virus (ATU2) or between the H and L genes (ATU3). It has been observed that the transcription of the viral RNA of MV follows a gradient from the 5′ to the 3′ end. Thus, an ATU inserted in the 5′ end of the coding sequence of the cDNA will enable a greater expression of the heterologous DNA sequence within the ATU than an ATU inserted closer to the 3′ end of the coding sequence of the cDNA. An exemplary ATU may comprise the polynucleotide encoding at least one polypeptide such as a spike (S) polypeptide of a coronavirus (CoV), in particular of SARS-CoV-2, or an immunogenic fragment thereof that has 1, 2, 3 or more amino acid substitution(s), insertion(s) and/or deletion(s)) that it contains.

The polynucleotide encoding at least the spike (S) polypeptide of a coronavirus (CoV), in particular of SARS-CoV-2, or an immunogenic fragment thereof that has 1, 2, 3 or more amino acid substitution(s), insertion(s) and/or deletion(s), may thus be inserted in any intergenic region of the cDNA molecule of the MV, in particular in an ATU. Particular constructs of the invention are those illustrated in the examples.

In a preferred embodiment of the invention, the polynucleotide encoding at least the spike (S) polypeptide of a CoV, in particular of SARS-CoV-2, or an immunogenic fragment thereof that has 1, 2, 3 or more amino acid substitution(s), insertion(s) and/or deletion(s), is inserted in the intergenic region between the P and M genes of the MV cDNA molecule (ATU2), or between the H and L genes of the MV cDNA molecule (ATU3), preferably in an ATU3.

In a particular embodiment of the invention, the construct is prepared by cloning a polynucleotide encoding at least one polypeptide in particular the spike (S) E, N, ORF3a, ORF8, ORF7a or M polypeptide of a coronavirus (CoV), in particular of SARS-CoV-2 (such as a S polypeptide having the sequence disclosed in Genbank MN908947.3 or any of the polypeptides derived from the native S antigens and illustrated herein, especially as fragments of S or modified fragments of S), or an immunogenic fragment thereof (including a mutated fragment) as disclosed herein or a mutated antigen thereof that has 1, 2, 3 or more amino acid substitution(s), insertion(s) and/or deletion(s), in the cDNA encoding a full-length, antigenomic (+) RNA strand of a MV.

Alternatively, a nucleic acid construct of the invention may be prepared using steps of synthesis of nucleic acid fragments or polymerization from a template, including by PCR.

The nucleic acid construct of the invention and the MV-CoV of the invention encodes or expresses at least one polypeptide selected from the group consisting of S, E, N, ORF3a, ORF8, ORF7a or M proteins of a coronavirus or specifically described for the SARS-CoV-2 strain, in particular the spike (S) polypeptide of a coronavirus (CoV), in particular of SARS-CoV-2, or an immunogenic fragment thereof that has 1, 2, 3 or more amino acid substitution(s), insertion(s) or deletion(s).

According to a preferred embodiment, the invention also concerns modifications and optimization of the polynucleotide to allow an efficient expression of the at least one polypeptide selected from the group consisting of S, E, N, ORF3a, ORF8, ORF7a or M proteins of a coronavirus or specifically described for the SARS-CoV-2 strain, in particular a spike (S) polypeptide of a coronavirus (CoV), in particular of SARS-CoV-2, or an immunogenic fragment thereof that has 1, 2, 3 or more amino acid substitution(s), insertion(s) and/or deletion(s), at the surface of chimeric infectious particles of MV-CoV in the host, in particular the human host.

According to this embodiment, optimization of the polynucleotide sequence can be operated avoiding cis-active domains of nucleic acid molecules, including: internal TATA-boxes, chi-sites and ribosomal entry sites; AT-rich or GC-rich sequence stretches; AU-rich sequence elements (ARE), inhibitory sequence elements (INS), and cis-acting repressor (CRS) sequence elements; repeat sequences and RNA secondary structures; cryptic splice donor and acceptor sites, and branch points.

The optimized polynucleotides may also be codon optimized for expression in a specific cell type. This optimization allows increasing the efficiency of chimeric infectious particles production in cells without impacting the expressed protein(s).

In a particular embodiment of the invention, the polynucleotide encoding at least a spike (S) polypeptide of a coronavirus (CoV), in particular of SARS-CoV-2, or an immunogenic fragment thereof that has 1, 2, 3 or more amino acid substitution(s), insertion(s) and/or deletion(s), has been codon optimized for use in humans.

The optimization of the polynucleotide encoding at least a spike (S) polypeptide of a coronavirus (CoV), in particular of SARS-CoV-2, or an immunogenic fragment thereof, or a variant of the S polypeptide or fragment thereof that has 1, 2, 3 or more amino acid substitution(s), insertion(s) and/or deletion(s) may be performed by modification of the wobble position in codons without impacting the identity of the amino acid residue translated from the codon with respect to the original one.

Optimization is also performed to remove certain sequences from Measles virus that may result in transcript editing. The editing of Measles virus transcript occurs in particular in the transcript encoded by the P gene of Measles virus. This editing, by the insertion of extra G residues at a specific site within the P transcript, gives rise to a new protein truncated compared to the P protein. Addition of only a single G residue results in the expression of the V protein, which contains a unique carboxyl terminus (Cattaneo R et al., Cell. 1989 Mar. 10; 56(5):759-64).

In a particular embodiment of the invention, measles transcript editing sequences have been changed from the polynucleotide encoding a spike (S) polypeptide of a coronavirus (CoV), in particular of SARS-CoV-2, or an immunogenic fragment thereof that has 1, 2, 3 or more amino acid substitution(s), insertion(s) and/or deletion(s). The following measles transcript editing sequences can be mutated: AAAGGG, AAAAGG, GGGAAA, GGGGAA, TTAAA, AAAA, as well as their complementary sequence: TTCCCC, TTTCCC, CCTTTT, CCCCTT, TTTAA, TTTT. For example, AAAGGG can be mutated in AAAGGC, AAAAGG can be mutated in AGAAGG or in TAAAGG or in GAAAGG, and GGGAAA in GCGAAA.

In a particular embodiment of the invention, the native and codon-optimized nucleotide sequences of the polynucleotide encoding particular peptides/proteins/antigen as well as the amino acid sequences of these peptides/proteins/antigen of the invention are selected from the sequences disclosed as SEQ ID NOs: 1-49, 51-66 and 73-82. See Tables 1 and 3 below for additional information regarding many of these sequences.

Codon-optimized genes are useful both to promote high-level expression of poorly transcribed/translated genes and to recover Measles-based vaccine candidates with high and stable antigen expression. However, they suffer from major drawbacks, which are intrinsically linked to their design and final codon (most used codons in the final host genome) and nucleotide (high GC/AT ratio) compositions. Thus codon-optimized genes most often promote high level translation that, if highly transcribed, leads to saturation of the translational and post-translational cellular machineries and associated consequences on the quality of the expressed protein and on the cells itself (ER and Golgi stress). Their naturally high GC/composition (usually more than 55-60%) is also certainly not optimal for engineering of stable recombinant MV vectors since the GC composition of the MV genome is much lower (47.4%). The inventors thus decided to engineer optimized genes for the MV platform with the following features in the polynucleotides encoding the polypeptide(s) of a coronavirus, in particular of SARS-CoV-2, specifically:
absence of MV editing (AnGn, n≥3)- and core gene end (A4CKT)-like sequences on both strands,
removal, where applicable, of internal TATA-boxes, chi-sites and ribosomal entry sites (for translation efficiency),
removal, where applicable, of AT-rich or GC-rich sequence stretches, RNA instability motifs, repeat sequences and RNA secondary structures (for transcription efficiency, mRNA stability and also translation efficiency), and/or
balanced codon composition, avoiding, where applicable, rare codons and high usage of the most frequent codons (for efficiency, accuracy and speed of translation without saturation of the translation machinery),
target GC composition of 44-50% (to adjust to that of MV genome).

In addition, a criteria was added for the removal of cryptic splice donor and acceptor sites in higher eukaryotes, which is of no importance for the MV platform but allows monitoring for the synthetic gene expression by transient transfection in mammalian cells.

BsiWI and BssHII restriction sites were added at the 5′ and 3′ ends, respectively, of the designed nucleotide sequences and appropriate spacer sequence were inserted so that the resulting cDNAs comply with the “rule of six”, which stipulates that the number of nucleotides of the MV genome must be a multiple of 6.

The resulting cDNAs were named S_MV optimized synthetic gene and N_MV optimized synthetic gene is herein disclosed as SEQ ID NO: 36 and SEQ ID NO: 37 respectively.

In a particular embodiment of the invention, the transfer vector plasmid has the optimized sequence of SEQ ID NO: 34 (pKM-ATU2-S_2019-nCoV (i.e. SARS-CoV-2)) or SEQ ID NO: 35 (pKM-ATU3-S_2019-nCoV (i.e. SARS-CoV-2)), as mentioned in Table 1 below.

In a particular embodiment of the invention, the transfer vector plasmid has the optimized sequence selected from the group consisting of SEQ ID NO: 144 (pTM2-SF-dER_SARS-CoV-2), SEQ ID NO: 145 (pTM2-S2-dER_SARS-CoV-2), SEQ ID NO: 146 (pTM2-SF-2P-dER_SARS-CoV-2), SEQ ID NO: 147 (pTM2-S2-2P-dER_SARS-CoV-2) and SEQ ID NO: 148 (pTM2-SF-2P-2a_SARS-CoV-2), preferably has the sequence of SEQ ID NO: 146 (pTM2-SF-2P-dER_SARS-CoV-2) or SEQ ID NO: 148 (pTM2-SF-2P-2a_SARS-CoV-2), even more preferably has the sequence of SEQ ID NO: 146 (pTM2-SF-2P-dER_SARS-CoV-2).

In a particular embodiment of the invention, insertion of the nucleic acid construct as defined herein within the transfer vector plasmid can lead to mutations, in particular silent mutation(s).

In a particular embodiment of the invention, the first heterologous polynucleotide encodes the wild type S polypeptide of SEQ ID NO: 3, or a fragment thereof. The fragment thereof may include the S1 domain of SEQ ID NO: 11 or the S2 domain of SEQ ID NO: 13 of the S polypeptide, preferably the wild type S polypeptide of SEQ ID NO: 3, or a mutated antigen thereof that has 1, 2, 3 or more amino acid residue substitution(s) or insertion(s) and/or deletion(s), in particular less than 10, or less than 5 amino acid residue substitutions. The substitutions may be designed to improve stability.

In a particular embodiment of the invention, the first heterologous polynucleotide encodes the wild type S polypeptide of SEQ ID NO: 3, or an immunogenic fragment thereof. The immunogenic fragment thereof may include the S1 domain of SEQ ID NO: 11 or the S2 domain of SEQ ID NO: 13 of the S polypeptide, preferably the wild type S polypeptide of SEQ ID NO: 3, or a mutated antigen thereof that has 1, 2, 3 or more amino acid residue substitution(s) or insertion(s) and/or deletion(s), in particular less than 10, or less than 5 amino acid residue substitutions especially a mutated antigen that has 1, 2, 3 or more amino acid residue substitution(s), in particular less than 10, or less than 5 amino acid residue substitutions and that has up to 11 amino acid residue deletion in the cytoplasmic tail as disclosed herein. The substitutions may in particular be designed to improve stability. The deletion may be designed to improve surface expression of the polypeptide in cells. According to a particular embodiment, the first heterologous polynucleotide encodes a polypeptide of the amino acid sequences selected from the group consisting of SEQ ID NOs: 5, 7, 9, 15, 17 and 19, in particular SEQ ID NO: 5. According to a preferred embodiment, the first heterologous polynucleotide encodes the SF-2P-dER polypeptide of SEQ ID NO: 76, or the SF-2P-2a polypeptide of SEQ ID NO: 82, preferably the SF-2P-dER polypeptide of SEQ ID NO: 76, or a mutated antigen thereof that has 1, 2, 3 or more amino acid residue substitution(s) or insertion(s) and/or deletion(s), in particular less than 10, or less than 5 amino acid residue substitutions or additions and/or deletions.

According to a particular embodiment, the first heterologous polynucleotide encodes a mutated antigen having an amino acid sequence selected from the group consisting of SEQ ID NOs: 5, 7, 9, 15, 17, 19, 43, 45, 47, 49, 52, 54, 56, 58, 60, 62 and 65, in particular SEQ ID NOs: 5, 43, 45, 47 and 49, preferably SEQ ID NOs: 43, 45, 47 and 49, more preferably SEQ ID NOs: 45, 47 and 49, even more preferably SEQ ID NO: 47 or SEQ ID NO: 49, and even more preferably SEQ ID NO: 49.

In a particular embodiment of the invention, a single polypeptide of a coronavirus, in particular of SARS-CoV-2, is encoded by the nucleic acid construct and the polypeptide is the S polypeptide or a portion or fragment thereof as described herein.

In a particular embodiment of the nucleic acid construct of the invention, the second heterologous polynucleotide encodes (i) the N polypeptide of SEQ ID NO: 22, an immunogenic fragment thereof or a mutated antigen of the N polypeptide that has 1, 2, 3 or more amino acid residue substitution(s) or insertion(s) and/or deletion(s), in particular less than 10, or less than amino acid residue substitutions or additions or deletions, and/or (ii) the M polypeptide of SEQ ID NO: 24 or its endodomain, (iii) the E polypeptide of SEQ ID NO: 23, (iv) the ORF8 polypeptide of SEQ ID NO: 25, (v) the ORF7a polypeptide of SEQ ID NO: 27 and/or (vi) the ORF3a polypeptide of SEQ ID NO: 26 of a coronavirus, in particular of SARS-CoV-2, an immunogenic fragment thereof or an antigenic fragment thereof, or a mutated antigen thereof that has 1, 2, 3 or more amino acid residue substitution(s) or insertion(s) and/or deletion(s), in particular less than 10, or less than 5 amino acid residue substitutions or additions and/or deletions. In a preferred embodiment of the invention, the heterologous polynucleotide encoding the N polypeptide has the sequence of SEQ ID NO: 20, 21 or 37, preferably the sequence of SEQ ID NO: 21 or SEQ ID NO: 37.

In a preferred embodiment of the invention, the heterologous polynucleotide encoding the S polypeptide, S1 polypeptide or S2 polypeptide, an immunogenic fragment thereof that has 1, 2, 3 or more amino acid substitution(s), insertion(s) and/or deletion(s) comprises or consists in the open reading frame of the wild type gene or has a codon-optimized open reading frame(s) (coORF) for expression in mammalian cells and/or in Drosophila cells, in particular, the heterologous polynucleotide comprises one of the following sequences:
SEQ ID NO: 1 or 2 or 36 which encodes the S polypeptide, preferably SEQ ID NO: 2 or,
SEQ ID NO: 10 which encodes the S1 polypeptide or,
SEQ ID NO: 12 which encodes the S2 polypeptide or,
SEQ ID NO: 4 which encodes the stab-S polypeptide (also named S2P polypeptide) or,
SEQ ID NO: 6 which encodes the Secto polypeptide or,
SEQ ID NO: 8 which encodes the stab-Secto polypeptide or,
SEQ ID NO:14 which encodes the stab-S2 polypeptide or,
SEQ ID NO: 16 which encodes the tri-Secto polypeptide or,
SEQ ID NO: 18 which encodes the tristab-Secto polypeptide or
SEQ ID NO: 42 which encodes the S3F polypeptide or,
SEQ ID NO: 44 which encodes the S2P3F polypeptide or,
SEQ ID NO: 46 which encodes the S2PΔF polypeptide or,
SEQ ID NO: 48 which encodes the S2PΔF2A polypeptide or,
SEQ ID NO: 51 which encodes the T4-S2P3F polypeptide (also named tristab-Secto-3F) or,
SEQ ID NO: 53 which encodes the S6P polypeptide or,
SEQ ID NO: 55 which encodes the S6P3F polypeptide or,
SEQ ID NO: 57 which encodes the S6PΔF polypeptide or,
SEQ ID NO: 59 which encodes the SCCPP polypeptide or,
SEQ ID NO: 61 which encodes the SCC6P polypeptide or,
SEQ ID NO: 63 which encodes the SMVopt2P polypeptide or,
SEQ ID NO: 64 which encodes the SMVoptΔF polypeptide or,
SEQ ID NO: 66 which encodes the SMVopt2PΔF polypeptide, preferably the heterologous polynucleotide comprises the sequence selected from the group consisting of SEQ ID NO: 2, SEQ ID NO: 4, SEQ ID NO: 42, SEQ ID NO: 44, SEQ ID NO: 46 and SEQ ID NO: 48, more preferably the heterologous polynucleotide comprises the sequence of SEQ ID NO: 48 which encodes the S2PΔF2A polypeptide.

In a particular embodiment of the invention, the nucleic acid construct is a cDNA construct comprising from 5′ to 3′ end the following polynucleotides coding for ORFs:
(a) a polynucleotide encoding the N protein of the MV;
(b) a polynucleotide encoding the P protein of the MV;
(c) the first heterologous polynucleotide encoding an S polypeptide, an immunogenic fragment thereof that has 1, 2, 3 or more amino acid substitution(s), insertion(s) and/or deletion(s), of a coronavirus, in particular of SARS-CoV-2, and wherein the first heterologous polynucleotide is positioned within an additional transcription unit (ATU) inserted within the cDNA of the antigenomic (+) RNA, in particular within ATU2 or ATU3, preferably ATU3;
(d) a polynucleotide encoding the M protein of the MV;
(e) a polynucleotide encoding the F protein of the MV;
(f) a polynucleotide encoding the H protein of the MV;
(g) a polynucleotide encoding the L protein of the MV; and

wherein the polynucleotides are operatively linked within the nucleic acid construct and are under the control of a viral replication and transcriptional regulatory elements such as MV leader and trailer sequences and are framed by a T7 promoter and a T7 terminator and additionally are framed by restrictions sites suitable for cloning in a vector to provide a recombinant MV-CoV expression cassette.

In a preferred embodiment of the invention, the nucleic acid construct is a cDNA construct comprising from 5′ to 3′ end the following polynucleotides coding for open reading frames:
(a) a polynucleotide encoding the N protein of the MV;
(b) a polynucleotide encoding the P protein of the MV;
(c) the first heterologous polynucleotide as defined above;
(d) a polynucleotide encoding the M protein of the MV;
(e) a polynucleotide encoding the F protein of the MV;
(f) a polynucleotide encoding the H protein of the MV;
(g) a polynucleotide encoding the L protein of the MV; and

wherein the polynucleotides are operatively linked within the nucleic acid construct and are under the control of a viral replication and transcriptional regulatory elements such as MV leader and trailer sequences and are framed by a T7 promoter and a T7 terminator and are framed by restriction sites suitable for cloning in a vector to provide a recombinant MV-CoV expression cassette. In a more preferred embodiment of the nucleic acid construct of the invention, (i) the first heterologous polynucleotide comprises a measles virus-optimized nucleotide sequence, in particular a sequence selected from the group consisting of SEQ ID NO: 36, SEQ ID NO: 63, SEQ ID NO: 64 and SEQ ID NO: 66, and is positioned within ATU2, or (ii) the first heterologous polynucleotide comprises a codon-optimized nucleotide sequence, in particular a sequence selected from the group consisting of SEQ ID NO: 2, SEQ ID NO: 4, SEQ ID NO: 44, SEQ ID NO: 46, SEQ ID NO: 48, SEQ ID NO: 51, SEQ ID NO: 53, SEQ ID NO: 55, SEQ ID NO: 57, SEQ ID NO: 59 and SEQ ID NO: 61, and is positioned within ATU3.

In a more preferred embodiment of the nucleic acid construct of the invention, (i) the first heterologous polynucleotide is positioned within ATU3 and the second heterologous polynucleotide, in particular the second heterologous polynucleotide encoding the N polypeptide, is positioned within ATU2, or (ii) the first heterologous polynucleotide is positioned within ATU2 and the second heterologous polynucleotide, in particular the second heterologous polynucleotide encoding the N polypeptide, is positioned within ATU3.

In another embodiment the first heterologous polynucleotide is replaced by the second heterologous polynucleotide.

In another aspect of the invention, the nucleic acid construct comprises only one heterologous polynucleotide such as the so-called second heterologous polynucleotide as defined herein, positioned within ATU2 or ATU3. In some embodiments, this second heterologous polynucleotide encodes the N polypeptide. In some embodiments, this second heterologous polynucleotide encoding the N polypeptide has the sequence of SEQ ID NO: 20, 21 or 37, preferably the sequence of SEQ ID NO: 21 or SEQ ID NO: 37. This nucleic acid construct may further comprise another heterologous polynucleotide, for example the so-called first heterologous polynucleotide as defined herein. All the definitions and embodiments disclosed herein apply to this other aspect of the invention and all paragraphs can be combined together.

In a preferred embodiment of the invention, the heterologous polynucleotide encoding the S polypeptide, S1 polypeptide or S2 polypeptide, an immunogenic fragment thereof that has 1, 2, 3 or more amino acid substitution(s), insertion(s) and/or deletion(s) comprises or consists in the open reading frame of the wild type gene or has a codon-optimized open reading frame(s) (coORF) for expression in mammalian cells and/or in Drosophila cells, in particular, the heterologous polynucleotide comprises one of the following sequences:
SEQ ID NO: 1 or 2 or 36 which encodes the S polypeptide, preferably SEQ ID NO: 2 or,
SEQ ID NO: 10 which encodes the S1 polypeptide or,
SEQ ID NO: 12 which encodes the S2 polypeptide or,
SEQ ID NO: 4 which encodes the stab-S polypeptide (also named S2P polypeptide) or,
SEQ ID NO: 6 which encodes the Secto polypeptide or,
SEQ ID NO: 8 which encodes the stab-Secto polypeptide or,
SEQ ID NO:14 which encodes the stab-S2 polypeptide or,
SEQ ID NO: 16 which encodes the tri-Secto polypeptide or,
SEQ ID NO: 18 which encodes the tristab-Secto polypeptide, preferably the heterologous polynucleotide comprises the sequence of SEQ ID NO: 2 or SEQ ID NO: 4.

In an even more preferred embodiment of the invention, the heterologous polynucleotide encoding the SF-2P-dER polypeptide or SF-2P-2a polypeptide, an immunogenic fragment thereof that has 1, 2, 3 or more amino acid substitution(s), insertion(s) and/or deletion(s) has the open reading frame of a codon-optimized open reading frame(s) (coORF) for expression in mammalian cells and/or in drosophila cells, in particular, the heterologous polynucleotide comprises one of the following sequences:
i. SEQ ID NO: 75 which encodes the SF-2P-dER polypeptide or,
ii. SEQ ID NO: 81 which encodes the SF-2P-2a polypeptide, preferably the heterologous polynucleotide comprises the sequence of SEQ ID NO: 75 which encodes the SF-2P-dER polypeptide.

In a particular embodiment of the invention, the nucleic acid construct is a cDNA construct comprising from 5′ to 3′ end the following polynucleotides coding for ORFs:
(a) a polynucleotide encoding the N protein of the MV;
(b) a polynucleotide encoding the P protein of the MV;
(c) the first heterologous polynucleotide encoding at least an S polypeptide, an immunogenic fragment thereof that has 1, 2, 3 or more amino acid substitution(s), insertion(s) and/or deletion(s), of a coronavirus, in particular of SARS-CoV-2, and wherein the first heterologous polynucleotide is positioned within an additional transcription unit (ATU) inserted within the cDNA of the antigenomic (+) RNA, in particular within ATU2 or ATU3, preferably ATU2;
(d) a polynucleotide encoding the M protein of the MV;
(e) a polynucleotide encoding the F protein of the MV;
(f) a polynucleotide encoding the H protein of the MV;
(g) a polynucleotide encoding the L protein of the MV; and

wherein the polynucleotides are operatively linked within the nucleic acid construct and are under the control of a viral replication and transcriptional regulatory elements such as MV leader and trailer sequences and are framed by a T7 promoter and a T7 terminator and additionally are framed by restriction sites suitable for cloning in a vector to provide a recombinant MV-CoV expression cassette.

In a preferred embodiment of the invention, the nucleic acid construct is a cDNA construct comprising from 5′- to 3′-end the following polynucleotides coding for open reading frames:
(a) a polynucleotide encoding the N protein of the MV;
(b) a polynucleotide encoding the P protein of the MV;
(c) the first heterologous polynucleotide according to the invention, in particular the first heterologous polynucleotide encoding the SF-2P-dER or SF-2P-2a polypeptide, an immunogenic fragment thereof that has 1, 2, 3 or more amino acid substitution(s), insertion(s) and/or deletion(s), of SARS-CoV-2, and wherein the first heterologous polynucleotide is positioned within ATU2 or ATU3, preferably ATU2;
(d) a polynucleotide encoding the M protein of the MV;
(e) a polynucleotide encoding the F protein of the MV;
(f) a polynucleotide encoding the H protein of the MV;
(g) a polynucleotide encoding the L protein of the MV; and

wherein the polynucleotides are operatively linked within the nucleic acid construct and are under the control of a viral replication and transcriptional regulatory elements such as MV leader and trailer sequences and are framed by a T7 promoter and a T7 terminator and additionally are framed by restrictions sites suitable for cloning in a vector to provide a recombinant MV-CoV expression cassette.

In another embodiment the first nucleic acid construct is replaced by the second nucleic acid construct.

The expressions “N protein”, “P protein”, “M protein”, “F protein”, “H protein” and “L protein” refer respectively to the nucleoprotein (N), the phosphoprotein (P), the matrix protein (M), the fusion protein (F), the hemagglutinin protein (H) and the RNA polymerase large protein (L) of a MV Fields, Virology (Knipe & Howley, 2001).

In the construct of the invention the polynucleotide sequences disclosed herein in respect of MV sequences taken together with the added polynucleotide sequences that will remain in the replicon of the recombinant genome comply with the “rule of six” featuring the requirement that the MV genome be an exact multiple of six nucleotides in length for reverse genetics for correctly take place in order to enable efficient rescue.

In a particular embodiment of the invention, the sequence of the recombinant MV-CoV nucleic acid molecule between the first nucleotide of the cDNA encoding the MV antigenome and the last nucleotide of the cDNA encoding the MV antigenome is a multiple of 6 nucleotides.

The “rule of six” accordingly also applies to this construct prepared according to the invention that comprises the sequences encoding the coronavirus antigen(s).

The “rule of six” is expressed in the fact that the total number of nucleotides present in a nucleic acid representing the MV(+) strand RNA genome or in nucleic acid constructs comprising same is a multiple of six. The “rule of six” has been acknowledged in the state of the art as a requirement regarding the total number of nucleotides in the genome of the MV, which enables efficient or optimized replication of the MV genomic RNA. In the embodiments of the present invention defining a nucleic acid construct that meets the rule of six, the rule applies to the nucleic acid construct specifying the cDNA encoding the full-length MV (+) strand RNA genome and all inserted sequences, when taken individually or collectively.

In particular, the nucleic acid construct of the invention complies with the rule of six (6) of the MV genome when recombined with the polynucleotide encoding at least a spike (S) polypeptide of a coronavirus (CoV), in particular of SARS-CoV-2, or an immunogenic fragment thereof that has 1, 2, 3 or more amino acid substitution(s), insertion(s) and/or deletion(s), taken together with the cDNA molecule encoding the full-length, infectious antigenomic (+) RNA strand of the MV consist of a number of nucleotides that is a multiple of six. In a particular embodiment, the rule of six applies to the cDNA encoding the full-length infectious antigenomic (+) RNA strand of the MV and to the polynucleotide cloned into the cDNA and encoding at least a spike (S) polypeptide of a CoV, in particular of SARS-CoV-2, or an immunogenic fragment thereof that has 1, 2, 3 or more amino acid substitution(s), insertion(s) and/or deletion(s). Alternatively, compliance with the rule of six may be determined taking into account the whole construct or the transcript obtained from the construct in cells used for the rescue of recombinant measles virus.

The organization of the genome of MVs and their replication and transcription process have been fully identified in the prior art and are especially disclosed in Horikami S. M. and Moyer S. A. (Curr. Top. Microbiol. Immunol. (1995) 191, 35-50) or in Combredet C. et al (Journal of Virology, November 2003, p 11546-11554) for the Schwarz vaccination strain of the virus or for broadly considered negative-sense RNA viruses, in Neumann G. et al (Journal of General Virology (2002) 83, 2635-2662).

In a preferred embodiment of the invention, the measles virus is an attenuated virus strain.

An “attenuated strain” of measles virus is defined as a strain that is avirulent or less virulent than the parent strain in the same host, while maintaining immunogenicity and possibly adjuvanticity when administered in a host i.e., preserving immunodominant T and B cell epitopes and possibly the adjuvanticity such as the induction of T cell costimulatory proteins or the cytokine IL-12.

An attenuated strain of a MV accordingly refers to a strain which has been serially passaged on selected cells and, possibly, adapted to other cells to produce seed strains suitable for the preparation of vaccine strains, harboring a stable genome which would not allow reversion to pathogenicity nor integration in host chromosomes. As a particular “attenuated strain”, an approved strain for a vaccine is an attenuated strain suitable for the invention when it meets the criteria defined by the FDA (US Food and Drug Administration) i.e., it meets safety, efficacy, quality and reproducibility criteria, after rigorous reviews of laboratory and clinical data (www.fda.gov/cber/vaccine/vacappr.htm).

Particular attenuated strains that can be used to implement the present invention and especially to derive the MV cDNA of the nucleic acid construct are the Schwarz strain, the Zagreb strain, the AIK-C strain and the Moraten strain, more preferably the Schwarz strain. All these strains have been described in the prior art and access to them is provided in particular as commercial vaccines. In a particular embodiment of the invention, the recombinant DNA or cDNA of the MV-CoV molecule is placed under the control of heterologous expression control sequences. The insertion of such a control for the expression of the DNA/cDNA, is favorable when the expression of this DNA/cDNA is sought in cell types which do not enable full transcription of the DNA/cDNA with its native control sequences.

In a particular embodiment of the invention, the heterologous expression control sequence comprises the T7 promoter and T7 terminator sequences. These sequences are respectively located 5′ and 3′ of the coding sequence for the full length antigenomic (+)RNA strand of MV and from the adjacent sequences around this coding sequence. Accordingly in a particular embodiment the nucleic acid construct of the invention comprises these additional control sequences.

In a particular embodiment of the invention, the recombinant nucleic acid molecule or the nucleic acid construct encoding the antigenomic RNA of the measles virus recombined with the heterologous polynucleotide, which is defined herein is further modified i.e., comprises additional nucleotide sequences or motifs.

In a preferred embodiment, the nucleic acid construct or the recombinant nucleic acid molecule encoding the antigenomic RNA of the measles virus recombined with the heterologous polynucleotide according to the invention further comprises, (a) a GGG motif followed by a hammerhead ribozyme sequence at the 5′-end of the nucleic acid construct, adjacent to a first nucleotide of the nucleotide sequence encoding a full-length antigenomic (+)RNA strand of an attenuated MV vaccine strain, in particular of a Schwarz strain or of a Moraten strain, and also comprises, (b) a nucleotide sequence of a ribozyme in particular the sequence of the Hepatitis delta virus ribozyme (6), at the 3′-end of the recombinant MV-CoV nucleic acid molecule, adjacent to the last nucleotide of the nucleotide sequence encoding the full length anti-genomic (+)RNA strand. The Hepatitis delta virus ribozyme (6) is advantageously hence provided at the 3′-end, adjacent to the last nucleotide of the nucleotide sequence encoding the full length anti-genomic (+)RNA strand.

The GGG motif placed at the 5′ end, adjacent to the first nucleotide of the above coding sequence improves the efficiency of the transcription of the cDNA coding sequence. The proper assembly of measles virus particles requires that the cDNA encoding the antigenomic (+)RNA of the nucleic acid construct of the invention complies with the rule of six, such that when the GGG motif is added, a ribozyme is also added at the 5′ end of the coding sequence of the cDNA, 3′ from the GGG motif, thereby enabling cleavage of the transcript at the first coding nucleotide of the full-length antigenomic (+)RNA strand of MV.

In a particular embodiment of the invention, in order to prepare the nucleic acid construct of the invention, the preparation of a cDNA molecule encoding the full-length antigenomic (+) RNA of a MV disclosed in the prior art is achieved by known methods. The cDNA provides especially the genome vector when it is inserted in a vector such as a plasmid.

A particular cDNA molecule suitable for the preparation of the nucleic acid construct of the invention is the one obtained using the Schwarz strain of MV. Accordingly, the cDNA coding for the antigenome of the measles virus used within the present invention may be obtained as disclosed in WO2004/000876 or may be obtained from plasmid pTM-MVSchw deposited by Institut Pasteur at the Collection Nationale de Culture de Microorganismes (CNCM), 28 rue du Dr Roux, 75724 Paris Cedex 15, France, under No I-2889 on Jun. 12, 2002, the sequence of which is disclosed in WO2004/000876 incorporated herein by reference. The plasmid pTM-MVSchw was obtained from a Bluescript plasmid and comprises the polynucleotide coding for the full-length measles virus (+) RNA strand of the Schwarz strain placed under the control of the promoter of the T7 RNA polymerase. Plasmid pTM-MVSchw has 18967 nucleotides and the sequence of SEQ ID NO: 28. cDNA molecules (also designated cDNA of the measles virus or MV cDNA for convenience) from other MV strains may be similarly obtained starting from the nucleic acid purified from viral particles of attenuated MV such as those described herein.

The cDNA coding for the antigenome of the measles virus used within the present invention may also be obtained from plasmid pTM2-MVSchw-gfp deposited by Institut Pasteur at the Collection Nationale de Culture de Microorganismes (CNCM), 28 rue du Dr Roux, 75724 Paris Cedex 15, France, under No I-2890 on Jun. 12, 2002. It has 19795 nucleotides and a sequence represented as SEQ ID NO: 29. This plasmid contains the sequence encoding the eGFP marker that may be deleted.

The above cited pTM-MVSchw plasmids may also be used for the preparation of the nucleic acid constructs of the invention, by cloning the heterologous polynucleotide encoding a polypeptide derived from an antigen of a coronavirus, in particular of the SARS-CoV-2 strain, (in particular a polypeptide derived from the S antigen as disclosed herein) in the cDNA encoding the antigenome of the measles virus, using one or more ATU inserted at position known for insertion of ATU1 or ATU2 or ATU3, preferably ATU3.

In a particular embodiment, the nucleic acid construct of the invention comprises or consists of the recombinant MV-CoV nucleic acid molecule located from position 1 to position 20152 in the sequence of SEQ ID NO: 34 or SEQ ID NO: 35. This construct encodes the S polypeptide of SARS-CoV-2, respectively located in either the ATU2 or in the ATU3 inserted in the cDNA encoding the measles virus antigenome.

In a particular embodiment, the invention relates to a nucleic acid construct derived from the above by replacement of the sequence encoding the S protein by a polynucleotide encoding another polypeptide of a coronavirus, in particular of the SARS-CoV-2, such as the sequence of a fragment of the S antigen as disclosed herein, in particular a nucleotide sequence encoding one of the stab-S (also named S2P), Secto, stab-Secto, S1, S2, stab-S2, tri-Secto, tristab-Secto, S3F, S2P3F, S2PΔF, S2PΔF2A polypeptide, in particular a polynucleotide of sequence disclosed as SEQ ID NO: 4, 6, 8, 10, 12, 14, 16, 18, 42, 44, 46 or 48 respectively, preferably SEQ ID NO: 4, 42, 44, 46 or 48, more preferably SEQ ID NO: 44, 46 or 48, even more preferably SEQ ID NO: 46 or SEQ ID NO: 48, even more preferably SEQ ID NO: 48, or a polynucleotide encoding the amino acid sequence of SEQ ID NO: 5, 7, 9, 11, 13, 15, 17, 19, 43, 45, 47 or 49 respectively, preferably SEQ ID NO: 5, 43, 45, 47 or 49, more preferably SEQ ID NO: 45, 47 or 49, even more preferably SEQ ID NO: 47 or SEQ ID NO: 49, even more preferably SEQ ID NO: 49. In such embodiment, the polynucleotide region to be replaced in the sequence of SEQ ID NO: 34 is from position 3538 to position 7362 and the polynucleotide region to be replaced in the sequence of SEQ ID NO: 35 is from position 9340 to position 13164.

In a particular embodiment, the invention relates to a nucleic acid construct derived from the above by replacement of the sequence encoding the S protein by a polynucleotide encoding another polypeptide of a coronavirus, in particular of SARS-CoV-2, such as the sequence of a fragment of the S antigen as disclosed herein, in particular a nucleotide sequence encoding one of the stab-S (also named S2P), Secto, stab-Secto, S1, S2, stab-S2, tri-Secto, tristab-Secto, SF-2P-dER or SF-2P-2a polypeptide, in particular a polynucleotide of sequence disclosed as SEQ ID NO: 4, 6, 8, 10, 12, 14, 16, 18, 75 or 81 respectively, preferably SEQ ID NO: 4, 75 or 81, more preferably SEQ ID NO: 75 or 81, even more preferably SEQ ID NO: 75, or a polynucleotide encoding the amino acid sequence of SEQ ID NO: 5, 7, 9, 11, 13, 15, 17, 19, 76 or 82 respectively, preferably SEQ ID NO: 5, 76 or 82, more preferably SEQ ID NO: 76 or 82, even more preferably SEQ ID NO: 76. In such embodiment, the polynucleotide region to be replaced in the sequence of SEQ ID NO: 34 is from position 3538 to position 7362 and the polynucleotide region to be replaced in the sequence of SEQ ID NO: 35 is from position 9340 to position 13164.

In another embodiment the invention relates to a nucleic acid construct derived from the above by replacement of the sequence encoding the S protein by a polynucleotide encoding another polypeptide of a coronavirus, in particular of the SARS-CoV-2, such as the sequence encoding the N, E, M, ORF3a, ORF7a, ORF8 polypeptide of a coronavirus, in particular of SARS-CoV-2, or an antigenic or immunogenic fragment thereof that may be obtained using the a sequence disclosed in Genbank MN908947.3 or that may have the nucleotide sequences disclosed herein.

In a preferred embodiment of the invention, the nucleic acid construct comprises or consists of a recombinant MV-CoV nucleic acid molecule that comprises a second heterologous polynucleotide that encodes the N polypeptide of a coronavirus, in particular of SARS-CoV-2, or an immunogenic fragment thereof that has 1, 2, 3 or more amino acid substitution(s), insertion(s) and/or deletion(s), the second heterologous polynucleotide being cloned in an ATU at a different location with respect to the ATU used for cloning the first heterologous polynucleotide.

In a particular embodiment such nucleic acid construct is inserted, in particular cloned in an expression vector or a transfer vector, for example in a plasmid. Examples of suitable plasmids are the pTM plasmid know from Combredet et al (2003) or from WO 04/00876, or the pKM plasmid disclosed herein.

Any nucleic acid construct described herein is suitable and intended for the preparation of recombinant infectious replicative measles—coronavirus virus (MV-CoV) and accordingly the nucleic acid construct: (i) is used for insertion in a transfer genome vector that as a result comprises the cDNA molecule of the measles virus, especially of the Schwarz strain, for the production of the MV-CoV and yield of at least one polypeptide of a coronavirus (CoV), in particular of SARS-CoV-2, or an immunogenic fragment thereof that has 1, 2, 3 or more amino acid substitution(s), insertion(s) and/or deletion(s), in particular the spike (S) polypeptide or an immunogenic fragment thereof as disclosed herein or (ii) is such transfer vector, especially plasmid vector.

The nucleic acid construct may also be used for the production of viral-like particles (VLPs), in particular CoV VLPs.

As an example, the pTM-MVSchw plasmid or the pTM2-MVSchw plasmid is suitable to prepare the transfer vector, by insertion of the CoV polynucleotide(s) necessary for the expression of at least a spike (S) polypeptide or another antigen such as N, M, E, ORF7a, ORF3a or ORF8 of a coronavirus (CoV), in particular of SARS-CoV-2, or an immunogenic fragment thereof that has 1, 2, 3 or more amino acid substitution(s), insertion(s) and/or deletion(s). Alternatively such transfer vector may be the pKM vectors described in detail in the examples, including pKP-MVSchw-ATU1(eGFP), pKP-MVSchw-ATU2(eGFP), pKP-MVSchw-ATU3(eGFP) wherein the nucleotide sequence of the eGFP is replaced by the polynucleotide encoding the spike (S) polypeptide or another antigen such as N, M, E, ORF7a, ORF3a or ORF8 of a coronavirus (CoV), in particular of SARS-CoV-2, or an immunogenic fragment thereof that has 1, 2, 3 or more amino acid substitution(s), insertion(s) and/or deletion(s). The herein disclosed sequences enable the person skilled in the art to have access to the position of the inserts contained in the plasmids to design and prepare insert substitution especially using the disclosure in the examples.

All the plasmids cited herein by reference to their deposit at the CNCM have been deposited at the Collection Nationale de Cultures de Microorganismes, 25 rue du Docteur Roux, 75724 Paris Cedex 15 (France).

According to a particular aspect of a nucleic acid construct, the invention relates to a transfer vector, in particular a plasmid vector, suitable for the rescue of a recombinant Measles virus (MV) comprising the nucleic acid construct according to the invention, in particular a transfer vector selected from the group consisting of plasmid of SEQ ID NO: 28 (pTM-MVSchwarz), plasmid of SEQ ID NO: 29 (pTM2-MVSchw-gfp, also named pTM-MVSchw2-GFPbis or pTM-MVSchwarz-ATU2-CNCM I-3034 deposited on May 26, 2003 with the insertion of the GFPbis coding sequence), plasmid of SEQ ID NO: 38 (pTM3-MVSchw-gfp, also named pTM-MVSchw3-GFP or pTM-MVSchwarz-ATU3-CNCM I-3037 deposited on May 26, 2003 with the insertion of the GFP coding sequence), plasmid of SEQ ID NO: 30 (pKP-MVSchwarz), plasmid of SEQ ID NO: 31 (pKP-MVSchwarz-ATU1), plasmid of SEQ ID NO: 32 (pKP-MVSchwarz-ATU2) and plasmid of SEQ ID NO: 33 (pKP-MVSchwarz-ATU3) wherein the transfer vector is recombined with (i) a first heterologous DNA polynucleotide encoding at least a spike polypeptide of a coronavirus, in particular of SARS-CoV-2, or an immunogenic fragment thereof that has 1, 2, 3 or more amino acid substitution(s), insertion(s) and/or deletion(s) has been positioned within an additional transcription unit (ATU) inserted within the cDNA of the antigenomic (+) RNA or with (ii) a second heterologous polynucleotide encoding another polypeptide of a coronavirus, in particular of SARS-CoV-2, such as the sequence of the N, E, M, ORF3a, ORF7a, ORF8 polypeptide of a coronavirus, in particular of SARS-CoV-2 such as a sequence derived from the sequence disclosed in Genbank MN908947.3 or corresponding to a sequence disclosed herein.

According to a preferred aspect of the nucleic acid construct, the invention relates to a transfer vector, in particular a plasmid vector, suitable for the rescue of a recombinant Measles virus (MV) comprising the nucleic acid construct according to the invention, in particular a transfer vector selected from the group consisting of plasmid of SEQ ID NO: 32 (pKP-MVSchwarz-ATU2) and plasmid of SEQ ID NO: 33 (pKP-MVSchwarz-ATU3) wherein the transfer vector is recombined with a first heterologous DNA polynucleotide encoding the polypeptide of SARS-CoV-2 as defined in any one of claims 1, 2, 4 and 6 that has been positioned within ATU2 or ATU3.

In a particular embodiment, the transfer vector is a plasmid, especially one of the above plasmids recombined with a recombinant DNA MV-CoV sequence wherein the sequence encoding a polypeptide of SARS-CoV-2 is selected from the group consisting of:
SEQ ID NO: 1 or 2 or 36 (construct S);
SEQ ID NO: 4 (construct stab-S, also named construct S2P);
SEQ ID NO: 6 (construct Secto);
SED ID NO: 8 (construct stab-Secto);
SEQ ID NO: 10 (construct S1),
SEQ ID NO: 12 (construct S2),
SEQ ID NO: 14 (construct stab-S2),
SEQ ID NO: 16 (construct tri-Secto),
SEQ ID NO: 18 (construct tristab-Secto),
SEQ ID NO: 42 (construct S3F),
SEQ ID NO: 44 (construct S2P3F),
SEQ ID NO: 46 (construct S2PΔF),
SEQ ID NO: 48 (construct S2PΔF2A),
SEQ ID NO: 21 or 37 (construct N),
SEQ ID NO: 51 (construct T4-S2P3F (tristab-Secto-3F)),
SEQ ID NO: 53 (construct S6P),
SEQ ID NO: 55 (construct S6P3F),
SEQ ID NO: 57 (construct S6PΔF),
SEQ ID NO: 59 (construct SCCPP),
SEQ ID NO: 61 (construct SCC6P),
SEQ ID NO: 63 (construct S_{MVopt}2P),
SEQ ID NO: 64 (construct S_{MVopt}ΔF), and
SEQ ID NO: 66 (construct S_{MVopt}2PΔF).

In a preferred embodiment, the transfer vector is a plasmid, especially one of the above plasmids recombined with a recombinant DNA MV-CoV sequence wherein the sequence encoding a polypeptide of SARS-CoV-2 is selected from the group consisting of:
SEQ ID NO: 2 (construct S);
SEQ ID NO: 4 (construct stab-S, also named construct S2P);
SEQ ID NO: 42 (construct S3F),
SEQ ID NO: 44 (construct S2P3F),
SEQ ID NO: 46 (construct S2PΔF), and
SEQ ID NO: 48 (construct S2PΔF2A).

In a more preferred embodiment, the transfer vector is a plasmid, especially one of the above plasmids recombined with a recombinant DNA MV-CoV sequence wherein the sequence encoding a polypeptide of SARS-CoV-2 is of SEQ ID NO: 48 (construct S2PΔF2A). When the sequence encoding the eGFP is present in the plasmid it is advantageously substituted by a sequence selected in the group defined above that is inserted in an ATU.

In a particular embodiment, the transfer vector is derived from the plasmids selected among:
pKP-MVSchwarz (or pKM-Schwarz) deposited under No. CNCM I-5493 on Feb. 12, 2020,
pKM-ATU2(eGFP) deposited under No. CNCM I-5494 on Feb. 12, 2020,
pKM-ATU3(eGFP) deposited under No. CNCM I-5495 on Feb. 12, 2020, in particular is one of these plasmids comprising a polynucleotide selected among the polynucleotides having the sequence of SEQ ID NO: 1, 2 or 36, preferably of SEQ ID NO: 2, or of SEQ ID NO: 4, 6, 8, 10, 12, 14, 16, 18, 42, 44, 46, 48, 21 or 37, in particular of SEQ ID NO: 4, 42, 44, 46, 48, preferably of SEQ ID NO: 44, 46 or 48, preferably of SEQ ID NO: 46 or SEQ ID NO: 48, even more preferably of SEQ ID NO: 48 inserted in an ATU in particular to replace the eGFP coding sequence, preferably in an ATU3.
   or is one of the plasmids selected from the group consisting of:
pKM-ATU2-S_2019-nCoV (i.e. SARS-CoV-2) deposited under No. CNCM I-5496 on Feb. 12, 2020,
pKM-ATU3-S_2019-nCoV (i.e. SARS-CoV-2) deposited under No. CNCM I-5497 on Feb. 12, 2020,
pKM-ATU3-S2PΔF_2019-nCoV (i.e. SARS-CoV-2) deposited under No. CNCM I-5532 on Jul. 1, 2020,
pKM-ATU3-S2PΔF2A_2019-nCoV (i.e. SARS-CoV-2) deposited under No. CNCM I-5533 on Jul. 1, 2020,
pKM-ATU3-S2P3F_2019-nCoV (i.e. SARS-CoV-2) deposited under No. CNCM I-5534 on Jul. 1, 2020,
pKM-ATU3-S3F_2019-nCoV (i.e. SARS-CoV-2) deposited under No. CNCM I-5535 on Jul. 1, 2020, and
pKM-ATU3-stab-S_2019-nCoV (i.e. SARS-CoV-2) (also named pKM-ATU3-S2P_2019-nCoV (i.e. SARS-CoV-2)) deposited under No. CNCM I-5536 on Jul. 7, 2020,
   preferably is the plasmid selected from the group consisting of pKM-ATU3-S2P3F_2019-nCoV (i.e. SARS-CoV-2), pKM-ATU3-S2PΔF_2019-nCoV (i.e. SARS-CoV-2) and pKM-ATU3-S2PΔF2A_2019-nCoV (i.e. SARS-CoV-2), more preferably is the plasmid pKM-ATU3-S2PΔF2A_2019-nCoV (i.e. SARS-CoV-2) deposited under No. CNCM I-5533 on Jul. 1, 2020.

According to a preferred aspect of the nucleic acid construct, the invention relates to a transfer vector, in particular a plasmid vector, suitable for the rescue of a recombinant Measles virus (MV) comprising the nucleic acid construct according to the invention, in particular a transfer vector consisting of a plasmid of SEQ ID NO: 29 (pTM2-MVSchw-gfp, also named pTM-MVSchw2-GFPbis or pTM-MVSchwarz-ATU2) or plasmid of SEQ ID NO: 38 (pTM3-MVSchw-gfp, also named pTM-MVSchw3-GFP or pTM-MVSchwarz-ATU3), wherein the transfer vector is recombined with a first heterologous DNA polynucleotide encoding the SF-2P-dER polypeptide or the SF-2P-2a polypeptide of SARS-CoV-2, or consisting of an immunogenic fragment thereof that has 1, 2, 3 or more amino acid substitution(s), insertion(s) and/or deletion(s) that has been positioned within ATU2 or ATU3, preferably ATU2.

In a particular embodiment, the transfer vector is a plasmid, especially one of the above plasmids recombined with a recombinant DNA MV-CoV sequence wherein the sequence encoding a polypeptide of SARS-CoV-2 is selected from the group consisting of:
SEQ ID NO: 1 or 2 or 36 (construct S);
SEQ ID NO: 4 (construct stab-S, also named construct S2P);
SEQ ID NO: 6 (construct Secto);
SED ID NO: 8 (construct stab-Secto);
SEQ ID NO: 10 (construct S1),
SEQ ID NO: 12 (construct S2),
SEQ ID NO: 14 (construct stab-S2),
SEQ ID NO: 16 (construct tri-Secto),
SEQ ID NO: 18 (construct tristab-Secto), and
SEQ ID NO: 21 or 37 (construct N).

In a preferred embodiment, the transfer vector is a plasmid, especially one of the above plasmids recombined with a recombinant DNA MV-CoV sequence wherein the sequence encoding a polypeptide of SARS-CoV-2 is selected from the group consisting of:
SEQ ID NO: 2 (construct S); and
SEQ ID NO: 4 (construct stab-S, also named construct S2P).

In another preferred embodiment, the transfer vector is a plasmid recombined with a recombinant DNA of MV-CoV, wherein the sequence encoding the SF-2P-dER polypeptide of SARS-CoV-2 is SEQ ID NO: 75 and the sequence encoding the SF-2P-2a polypeptide of SARS-CoV-2 is SEQ ID NO: 81, preferably the sequence encoding the SF-2P-dER polypeptide of SARS-CoV-2 is SEQ ID NO: 75. When the sequence encoding the eGFP is present in the plasmid it is advantageously substituted by a sequence selected in the group defined above that is inserted in an ATU.

In a particular embodiment, the transfer vector is derived from the plasmids selected among:
pKP-MVSchwarz (or pKM-Schwarz) deposited under No. CNCM I-5493 on Feb. 12, 2020,
pKM-ATU2(eGFP) deposited under No. CNCM I-5494 on Feb. 12, 2020,
pKM-ATU3(eGFP) deposited under No. CNCM I-5495 on Feb. 12, 2020, in particular is one of these plasmids comprising a polynucleotide selected among the polynucleotides having the sequence of SEQ ID NO: 1, 2 or 36, preferably of SEQ ID NO: 2, or of SEQ ID NO: 4, 6, 8, 10, 12, 14, 16, 18, 21 or 37, in particular of SEQ ID NO: 4 inserted in an ATU in particular to replace the eGFP coding sequence, preferably in an ATU3.
   or is one of the plasmids selected from the group consisting of:
pKM-ATU2-S_2019-nCoV (i.e. SARS-CoV-2) deposited under No. CNCM I-5496 on Feb. 12, 2020,
pKM-ATU3-S_2019-nCoV (i.e. SARS-CoV-2) deposited under No. CNCM I-5497 on Feb. 12, 2020, and
pKM-ATU3-stab-S_2019-nCoV (i.e. SARS-CoV-2) (also named pKM-ATU3-S2P_2019-nCoV (i.e. SARS-CoV-2)) deposited under No. CNCM I-5536 on Jul. 7, 2020.

In another particular embodiment, the transfer vector is one of the plasmids selected from the group consisting of pTM2-SF-dER_SARS-CoV-2 of SEQ ID NO: 144, pTM2-S2-dER_SARS-CoV-2 of SEQ ID NO: 145, pTM2-SF-2P-dER_SARS-CoV-2 of SEQ ID NO: 146, pTM2-S2-2P-dER_SARS-CoV-2 of SEQ ID NO: 147 and pTM2-SF-2P-2a_SARS-CoV-2 of SEQ ID NO: 148, preferably is pTM2-SF-2P-dER_SARS-CoV-2 of SEQ ID NO: 146 or pTM2-SF-2P-2a_SARS-CoV-2 of SEQ ID NO: 148, even more preferably is pTM2-SF-2P-dER_SARS-CoV-2 of SEQ ID NO: 146.

The invention also concerns the use of said transfer vector to transform cells suitable for rescue of viral MV-CoV particles, in particular to transfect or to transduce such cells respectively with plasmids or with viral vectors harboring the nucleic acid construct of the invention, the cells being selected for their capacity to express required MV proteins for appropriate replication, transcription and encapsidation of the recombinant genome of the virus corresponding to the nucleic acid construct of the invention in recombinant infectious replicating MV-CoV particles.

In a preferred embodiment, the invention relates to a host cell which is a helper cell, an amplification cell or a production cell, transfected with the nucleic acid construct according to the invention or with the transfer plasmid vector according to the invention, or infected with the recombinant measles virus according to the invention, in particular a mammalian cell, VERO NK cells, CEF cells, human embryonic kidney cell line 293 or lines derived therefrom (293T or 293T-T7 cells deposited at the CNCM (Paris France) under number I-3618 deposited on 14 Jun. 2006) or MRC5 cells.

Polynucleotides are thus present in the cells, which encode proteins that include in particular the N, P and L proteins of a MV (i.e., native MV proteins or functional variants thereof capable of forming ribonucleoprotein (RNP) complexes as a replicon), as stably expressed proteins at least for the N and P proteins or as or transitorily expressed proteins, functional in the transcription and replication of the recombinant viral MV-CoV particles. The N and P proteins may be expressed in the cells from a plasmid comprising their coding sequences or may be expressed from a DNA molecule inserted in the genome of the cell. The L protein may be expressed from a different plasmid. It may be expressed transitory. The helper cell is also capable of expressing a RNA polymerase suitable to enable the synthesis of the recombinant RNA derived from the nucleic acid construct of the invention, possibly as a stably expressed RNA polymerase. The RNA polymerase may be the T7 phage polymerase or its nuclear form (nlsT7).

In an embodiment of the invention, the cDNA clone of MV is from the same MV strain as the N protein and/or the P protein and/or the L protein. In another embodiment of the invention, the cDNA clone of a MV is from a different strain of virus than the N protein and/or the P protein and/or the L protein.

The invention also relates to a process for the preparation of recombinant infectious measles virus (MV) particles comprising:
1) transferring, in particular transfecting, the nucleic acid construct of the invention or the transfer vector containing such nucleic acid construct in a helper cell line which also expresses proteins necessary for transcription, replication and encapsidation of the antigenomic (+)RNA sequence of MV from its cDNA and under conditions enabling viral particles assembly; and
2) recovering the recombinant infectious MV-CoV particles expressing at least one polypeptide consisting of the spike (S) polypeptide of a coronavirus (CoV), in particular of SARS-CoV-2, or consisting of an immunogenic fragment thereof that has 1, 2, 3 or more amino acid substitution(s), insertion(s) and/or deletion(s).

In a particular embodiment of the invention, this process comprises:
1) transfecting helper cells with a nucleic acid construct according to the invention and with a transfer vector, wherein the helper cells are capable of expressing helper functions to express an RNA polymerase, and to express the N, P and L proteins of a MV virus;
2) co-cultivating the transfected helper cells of step 1) with passaged cells suitable for the passage of the MV attenuated strain from which the cDNA originates;
3) recovering the recombinant infectious MV-CoV particles expressing at least one polypeptide consisting of the spike (S) polypeptide of a coronavirus (CoV), in particular of SARS-CoV-2, or consisting of an immunogenic fragment thereof that has 1, 2, 3 or more amino acid substitution(s), insertion(s) and/or deletion(s).

In another particular embodiment of the invention, the method for the production of recombinant infectious MV-CoV particles comprises:
1) recombining a cell or a culture of cells stably producing a RNA polymerase, the N protein of a MV and the P protein of a MV, with a nucleic acid construct of the invention and with a vector comprising a nucleic acid encoding the L protein of a MV, and
2) recovering the recombinant infectious MV-CoV particles from the recombinant cell or culture of recombinant cells.

In a particular embodiment of the process, recombinant MV are produced, which express at least one polypeptide consisting of the spike (S) polypeptide of a coronavirus (CoV), in particular of SARS-CoV-2, or consisting of an immunogenic fragment thereof that has 1, 2, 3 or more amino acid substitution(s), insertion(s) and/or deletion(s), in particular CoV VLPs expressing the same CoV protein(s).

The invention thus relates to recombinant infectious replicating MV-CoV particles that may be recovered from rescue helper cells or in production cells. Optionally, VLP expressing the CoV antigens disclosed in accordance with the invention may additionally be recovered.

In a particular embodiment, the recombinant MV are produced, which express at least one polypeptide consisting of the spike (S) polypeptide of a coronavirus (CoV), in particular of SARS-CoV-2, or consisting of an immunogenic fragment thereof that has 1, 2, 3 or more amino acid substitution(s), insertion(s) and/or deletion(s) according to the various embodiments disclosed herein.

In a particular embodiment, the recombinant MV particles express at least one polypeptide consisting of the N, E, M, ORF7a, ORF3a, ORF8 polypeptide of a coronavirus (CoV), in particular of SARS-CoV-2, or consisting of an immunogenic fragment thereof that has 1, 2, 3 or more amino acid substitution(s), insertion(s) and/or deletion(s) according to the various embodiments disclosed herein.

In a particular embodiment, the recombinant MV particles express at least one polypeptide consisting of the spike (S) polypeptide of a coronavirus (CoV), in particular of SARS-CoV-2, or consisting of an immunogenic fragment thereof that has 1, 2, 3 or more amino acid substitution(s), insertion(s) and/or deletion(s) according to the various embodiments disclosed herein and additionally express at least one polypeptide consisting of the N, E, M, ORF7a, ORF3a, ORF8 polypeptide of a coronavirus (CoV), in particular of SARS-CoV-2, or consisting of an immunogenic fragment thereof that has 1, 2, 3 or more amino acid substitution(s), insertion(s) and/or deletion(s) according to the various embodiments disclosed herein.

In a particular embodiment of the invention, the particles are obtained from a measles virus which is an attenuated virus strain selected from the group consisting of the Schwarz strain according to all embodiments disclosed herein, the Zagreb strain, the AIK-C strain, the Moraten strain, the Philips strain, the Beckenham 4A strain, the Beckenham 16 strain, the CAM-70 strain, the TD 97 strain, the Leningrad-16 strain, the Shanghai 191 strain and the Belgrade strain, in particular the Schwarz strain.

In a particular embodiment the recombinant measles virus, in particular the recombinant measles virus of the Schwarz strain, comprises in its genome a nucleic acid construct which encodes at least one polypeptide consisting of the spike polypeptide of a coronavirus, in particular of SARS-CoV-2, or an immunogenic fragment thereof that has 1, 2, 3 or more amino acid substitution(s), insertion(s) and/or deletion(s) or one polypeptide consisting of the N, E, M, ORF7a, ORF3a, ORF8 polypeptide of a coronavirus (CoV), in particular of SARS-CoV-2, or consisting of an immunogenic fragment thereof that has 1, 2, 3 or more amino acid substitution(s), insertion(s) and/or deletion(s), in particular comprises in its genome a transcript of a nucleotide construct of the invention, in particular a nucleic acid construct as defined above which is a replicon of a transfer vector of the invention, the nucleic acid construct being operatively linked with the genome in an expression cassette. In a preferred embodiment, the recombinant measles virus, in particular a recombinant measles virus of the Schwarz strain, comprises in its genome the nucleic acid construct according to the invention, in particular a nucleic acid construct which encodes the SF-2P-dER polypeptide or the SF-2P-2a polypeptide of SARS-CoV-2, or an immunogenic fragment thereof that has 1, 2, 3 or more amino acid substitution(s), insertion(s) and/or deletion(s), in particular a polypeptide encoded by a nucleotide sequence of SEQ ID NO: 75 or SEQ ID NO: 81, preferably of SEQ ID NO: 75, in particular a nucleic acid construct which is a replicon of a transfer vector of the invention, the nucleic acid construct being operatively linked with the genome in an expression cassette. In a more preferred embodiment, the recombinant measles virus, in particular a recombinant measles virus of the Schwarz strain, expresses the SF-2P-dER polypeptide or the SF-2P-2a polypeptide of the SARS-CoV-2 strain, or an immunogenic fragment thereof that has 1, 2, 3 or more amino acid substitution(s), insertion(s) and/or deletion(s), and optionally further expresses at least one of a N polypeptide, M polypeptide, E polypeptide, ORF7a, ORF8 or ORF3a polypeptide of the SARS-CoV-2 strain or an immunogenic fragment thereof that has 1, 2, 3 or more amino acid substitution(s), insertion(s) and/or deletion(s).

In a more preferred embodiment, the recombinant measles virus further expresses at least one of a N polypeptide, M polypeptide, E polypeptide, ORF7a, ORF8 or ORF3a polypeptide of the SARS-CoV-2 strain, in particular further expressing the N polypeptide of SEQ ID NO: 22, an immunogenic fragment thereof or an antigenic fragment thereof, or a mutated antigen of the N polypeptide by substitution of 1, 2 or less than 10 amino acid residue(s), in particular less than 5 amino acid residues and/or the M polypeptide of sequence SEQ ID NO: 24 or its endodomain, the E polypeptide of sequence SEQ ID NO: 23, the ORF8 polypeptide of SEQ ID NO: 25, the ORF7a polypeptide of SEQ ID NO: 27 and/or the ORF3a polypeptide of SEQ ID NO: 26 of SARS-CoV-2, or an immunogenic fragment thereof that has 1, 2, 3 or more amino acid substitution(s), insertion(s) and/or deletion(s).

In a particular embodiment, the nucleotide sequence of the nucleic acid molecule encoding the polypeptide of a coronavirus, in particular of SARS-CoV-2 is selected from the group consisting of SEQ ID NOs: 1, 2, 4, 6, 8, 10, 12, 14, 16, 18, 20, 21, 36, 37, 42, 44, 46 and 48.

In a particular embodiment, the nucleotide sequence of the nucleic acid molecule encoding the polypeptide of a coronavirus, in particular of SARS-CoV-2 is selected from the group consisting of SEQ ID NOs: 1, 2, 4, 6, 8, 10, 12, 14, 16, 18, 20, 21, 36 and 37.

In a preferred embodiment of the invention, the nucleic acid molecule comprises a polynucleotide of SEQ ID NO: 75 (construct SF-2P-dER) or SEQ ID NO: 81 (construct SF-2P-2a), preferably a polynucleotide of SEQ ID NO: 75 (construct SF-2P-dER).

In a preferred embodiment, the nucleotide sequence of the nucleic acid molecule encoding the polypeptide of a coronavirus, in particular of SARS-CoV-2 is selected from the group consisting of SEQ ID NOs: 2, 4, 42, 44, 46 and 48, preferably is selected from the group consisting of SEQ ID NOs: 2, 4, 42 or from the group consisting of SEQ ID NOs: 44, 46 and 48, even more preferably is selected from the group consisting of SEQ ID NOs: 44, 46 and 48.

In a preferred embodiment, the nucleotide sequence of the nucleic acid molecule encoding the polypeptide of a coronavirus, in particular of SARS-CoV-2 is of SEQ ID NO: 2 or SEQ ID: 4.

In an even more preferred embodiment, the nucleotide sequence of the nucleic acid molecule encoding the polypeptide of a coronavirus, in particular of SARS-CoV-2 is of SEQ ID NO: 46 or SEQ ID NO: 48, preferably is of SEQ ID NO: 48.

The invention also relates to a process for rescuing recombinant measles virus expressing at least one polypeptide consisting of at least one of the (i) N, E, M, ORF7a, ORF3a, ORF8 polypeptide of a coronavirus (CoV), in particular of SARS-CoV-2, or consisting of an immunogenic fragment thereof that has 1, 2, 3 or more amino acid substitution(s), insertion(s) and/or deletion(s) according to the various embodiments described herein or (ii) a polypeptide consisting of the spike (S) polypeptide of a coronavirus, in particular of SARS-CoV-2 or an immunogenic fragment thereof that has 1, 2, 3 or more amino acid substitution(s), insertion(s) and/or deletion(s) according to the various embodiments described herein comprising:
(a) co-transfecting cells, in particular helper cells, in particular HEK293 helper cells, stably expressing T7 RNA polymerase and measles virus N and P proteins with (i) the nucleic acid construct according to the invention or with the transfer plasmid vector according to the invention that encodes the at least one polypeptide, and with (ii) a vector, especially a plasmid, encoding the MV L polymerase,
(b) maintaining the transfected cells in conditions suitable for the production of recombinant measles virus;
(c) infecting cells enabling propagation of the recombinant measles virus by co-cultivating them with the transfected cells of step (b), in particular VERO cells;
(d) harvesting the recombinant measles virus expressing at least the polypeptide of the coronavirus or an immunogenic fragment thereof that has 1, 2, 3 or more amino acid substitution(s), insertion(s) and/or deletion(s) of a coronavirus, in particular of SARS-CoV-2.

According to a preferred embodiment of the invention, the process for rescuing recombinant measles virus expresses the polypeptide of SARS-CoV-2 encoded by the first heterologous polynucleotide of SARS-CoV-2 as defined above comprising:
(a) co-transfecting cells, in particular helper cells, in particular HEK293 helper cells, stably expressing T7 RNA polymerase and measles virus N and P proteins with (i) the nucleic acid construct of the invention or with the transfer plasmid vector of the invention, and with (ii) a vector, especially a plasmid, encoding the MV L polymerase;
(b) maintaining the transfected cells in conditions suitable for the production of recombinant measles virus;
(c) infecting cells enabling propagation of the recombinant measles virus by co-cultivating them with the transfected cells of step (b), in particular VERO cells;
(d) harvesting the recombinant measles virus expressing the polypeptide of SARS-CoV-2 encoded by the first heterologous polynucleotide of SARS-CoV-2 as defined above and optionally at least one of the N, M or 3A polypeptide or an immunogenic fragment thereof or a mutated antigen thereof that has 1, 2, 3 or more amino acid substitution(s), insertion(s) and/or deletion(s) of SARS-CoV-2.

According to a preferred embodiment of the process, the recombinant measles virus expresses a mutated polypeptide as defined above, wherein the mutation at least impairs the retrieval of the polypeptide in the Endoplasmic Reticulum (ER) and optionally maintains the expressed protein in its prefusion state, in particular the SF-2P-dER polypeptide, in particular of SEQ ID NO: 76, or the SF-2P-2a polypeptide, in particular of SEQ ID NO: 82.

According to a particular embodiment of the process, the transfer vector plasmid has the sequence of SEQ ID NO: 34, SEQ ID NO: 35, or is one of the vectors deposited at the CNCM and disclosed herein under numbers I-5496, I-5497 and I-5536.

According to a particular embodiment of the process, the transfer vector plasmid has the sequence of SEQ ID NO: 34, SEQ ID NO: 35, or is one of the vectors deposited at the CNCM and disclosed herein under numbers I-5496, I-5497, I-5532, I-5533, I-5534, I-5535 and I-5536.

According to another particular embodiment of said process, the transfer vector plasmid has the sequence of SEQ ID NO: 146 or SEQ ID NO: 148, preferably of SEQ ID NO: 146.

According to a particular embodiment, recombination can be obtained with a first polynucleotide, which is the nucleic acid construct of the invention. Recombination can, also or alternatively, encompass introducing a polynucleotide, which is a vector encoding a RNA polymerase large protein (L) of a MV, whose definition, nature and stability of expression has been described herein.

In accordance with the invention, the cell or cell lines or a culture of cells stably producing a RNA polymerase, a nucleoprotein (N) of a measles virus and a polymerase cofactor phosphoprotein (P) of a measles virus is a cell or cell line as defined in the present specification or a culture of cells as defined in the present specification, i.e., are also recombinant cells to the extent that they have been modified by the introduction of one or more polynucleotides as defined above. In a particular embodiment of the invention, the cell or cell line or culture of cells, stably producing the RNA polymerase, the N and P proteins, does not produce the L protein of a measles virus or does not stably produce the L protein of a measles virus, e.g., enabling its transitory expression or production.

The production of recombinant infectious replicating MV-CoV particles of the invention may involve a transfer of cells transformed as described herein. This step is introduced after further recombination of the recombinant cells of the invention with nucleic acid construct of the invention, and optionally a vector comprising a nucleic acid encoding a RNA polymerase large protein (L) of a measles virus.

In a particular embodiment of the invention, a transfer step is required since the recombinant cells, usually chosen for their capacity to be easily recombined are not efficient enough in the sustaining and production of recombinant infectious MV-CoV particles. In the embodiment, the cell or cell line or culture of cells of step 1) of the above-defined methods is a recombinant cell or cell line or culture of recombinant cells according to the invention.

Cells suitable for the preparation of the recombinant cells of the invention are prokaryotic or eukaryotic cells, particularly animal or plant cells, and more particularly mammalian cells such as human cells or non-human mammalian cells or avian cells or yeast cells. In a particular embodiment, cells, before recombination of its genome, are isolated from either a primary culture or a cell line. Cells of the invention may be dividing or non-dividing cells.

According to a preferred embodiment, helper cells are derived from human embryonic kidney cell line 293, which cell line 293 is deposited with the ATCC under No. CRL-1573. Particular cell line 293 is the cell line disclosed in the international application WO2008/078198 (i.e. the HEK-293-T7-NP or HEK-293T-NP MV cell line deposited with the CNCM (Paris, France) on Jun. 14, 2006, under number I-3618) and referred to in the following examples.

According to another aspect of this process, the cells suitable for passage are CEF cells. CEF cells can be prepared from fertilized chicken eggs as obtained from EARL Morizeau, 8 rue Moulin, 28190 Dangers, France, or from any other producer of fertilized chicken eggs.

The process which is disclosed according to the present invention is used advantageously for the production of infectious replicative MV-CoV particles that may be used in an immunogenic composition. Optionally VLPs expressing CoV antigens may also be expressed that are appropriate for use in immunization compositions. Accordingly the invention concerns the recombinant MV-CoV particles of the invention for use in eliciting a humoral, especially a protective, in particular a neutralizing humoral response and/or a cellular response in an animal host, in particular a mammalian host, especially in a human being. The recombinant MV-CoV particles are in particular for use in eliciting a prophylactic response against infection by a coronavirus, in particular SARS-CoV-2.

The invention thus relates to an immunogenic composition, advantageously a vaccine composition comprising (i) an effective dose of the recombinant measles virus according to the invention, and/or of the recombinant VLPs according to the invention and (ii) a pharmaceutically acceptable vehicle, wherein the composition or the vaccine elicits a humoral, especially a protective, in particular a neutralizing humoral response and/or a cellular response in an animal host, especially in a human being, in particular after a single immunization, against the polypeptide(s) of the coronavirus, in particular of SARS-CoV-2 or their fragments, that it expresses.

In a particular embodiment of the invention, the composition is used in the elicitation of a protective, and preferentially prophylactic, immune response against SARS-CoV-2 or against SARS-CoV-2 and against further distinct coronavirus(es), by the elicitation of antibodies recognizing coronavirus protein(s) or antigenic fragment(s) thereof or mutated antigen(s) thereof that has(have) 1, 2, 3 or more amino acid substitution(s), insertion(s) and/or deletion(s), and/or by the elicitation of a cellular and/or humoral and cellular response against the Coronavirus, in a host in need thereof, in particular a human host, in particular a child. Preferably, the composition is devoid of added adjuvant.

The invention also relates to an immunogenic or vaccine composition comprising (i) an effective dose of the recombinant measles virus according to the invention, and/or of the recombinant VLPs according to the invention and (ii) a pharmaceutically acceptable vehicle for use in the prevention or treatment of an infection by CoV, in particular SARS-CoV-2 or in the prevention of clinical outcomes of infection by CoV in a host in need thereof, in particular a human host, in particular a child. In a particular embodiment, the composition is for administration to children, adolescents or travelers.

Methods of Treatment

Provided herein are compositions (e.g., pharmaceutical compositions), methods, kits and reagents for prevention and/or treatment of a coronavirus infection, particularly SARS-CoV-2 virus infection in humans and/or other mammals. The measles viruses of this disclosure may be used to induce an immune response or as therapeutic or prophylactic agents, including as vaccines. They may be used in medicine to prevent and/or treat infectious disease. In exemplary aspects, the recombinant measles virus vaccines of the present disclosure are used to provide prophylactic protection from coronavirus, particularly SARS-CoV-2 virus. Prophylactic protection from SARS-CoV-2 virus can be achieved following administration of a recombinant measles virus and/or immunogenic composition of the present disclosure. Vaccines can be administered once, twice, three times, four times or more. It is possible, although less desirable, to administer the vaccine to an infected individual to achieve a therapeutic response. Dosing may be adjusted accordingly in certain embodiments.

In some embodiments, the recombinant measles virus and immunogenic compositions of the present disclosure can be used as a method of preventing a coronavirus infection, particularly SARS-CoV-2 infection, in a subject, the method comprising administering to said subject at least one recombinant measles virus or immunogenic composition as provided herein. In some embodiments, the recombinant measles viruses or immunogenic compositions of the present disclosure can be used as a method of treating a coronavirus infection, particularly SARS-CoV-2 infection, in a subject, the method comprising administering to the subject at least one recombinant measles virus or immunogenic composition as provided herein. In some embodiments, the recombinant measles virus or immunogenic composition of the present disclosure can be used as a method of reducing an incidence of coronavirus infection, particularly SARS-CoV-2 infection, in a subject, the method comprising administering to the subject at least recombinant measles virus or immunogenic composition as provided herein. In some embodiments, the recombinant measles virus or immunogenic composition of the present disclosure can be used as a method of inhibiting spread of coronavirus, particularly SARS-CoV-2, from a first subject infected with coronavirus to a second subject not infected with coronavirus, particularly SARS-CoV-2, the method comprising administering to at least one of the first subject and said second subject at least one recombinant measles virus or immunogenic composition as provided herein.

A method of inducing an immune response in a subject against coronavirus, particularly SARS-CoV-2 is provided in aspects of the invention. The method involves administering to the subject a recombinant measles virus or immunogenic composition described herein, thereby inducing in the subject an immune response specific to coronavirus antigenic polypeptide or an immunogenic fragment thereof, particularly a full length SARS-CoV-2 antigenic polypeptide.

In some embodiments, the mutated antigen of the full length S protein or of the immunogenic fragment or the antigenic fragment is (a) the TA-S2P3F polypeptide of SEQ ID NO: 52, or a variant thereof having at least 90% identity with SEQ ID NO: 52, wherein the variant does not vary at positions 682, 683, 685, 986 and 987; or (b) the S6P polypeptide of SEQ ID NO: 54, or a variant thereof having at least 90% identity with SEQ ID NO: 54, wherein the variant does not vary at positions 817, 892, 899, 942, 986 and 987; or (c) the S6P3F polypeptide of SEQ ID NO: 56, or a variant thereof having at least 90% identity with SEQ ID NO: 56, wherein the variant does not vary at positions 682, 683, 685, 817, 892, 899, 942, 986 and 987; or (d) the S6PΔF polypeptide of SEQ ID NO: 58, or a variant thereof having at least 90% identity with SEQ ID NO: 58, wherein the variant does not vary at positions 806, 881, 888, 931, 975 and 976; or (e) the SCCPP polypeptide of SEQ ID NO: 60, or a variant thereof having at least 90% identity with SEQ ID NO: 60, wherein the variant does not vary at positions 383, 985, 986 and 987; or (f) the SCC6P polypeptide of SEQ ID NO: 62, or a variant thereof having at least 90% identity with SEQ ID NO: 62, wherein the variant does not vary at positions 383, 817, 892, 899, 942, 985, 986 and 987; or (g) the S_{MVopt}2P polypeptide of SEQ ID NO: 5, or a variant thereof having at least 90% identity with SEQ ID NO: 5, wherein the variant does not vary at positions 986 and 987; or (h) the S_{MVopt}ΔF polypeptide of SEQ ID NO: 65, or a variant thereof having at least 90% identity with SEQ ID NO: 65; or (i) the S_{MVopt}2PΔF polypeptide of SEQ ID NO: 47, or a variant thereof having at least 90% identity with SEQ ID NO: 47, wherein the variant does not vary at positions 975 and 976; or (j) the S_{MVopt}6P polypeptide, or a variant thereof having at least 90% identity with the S_{MVopt}6P polypeptide, wherein the variant does not vary at positions 817, 892, 899, 942, 986 and 987; or (k) the S_{MVopt}6PΔF polypeptide, or a variant thereof having at least 90% identity with the S_{MVopt}6PΔF polypeptide, wherein the variant does not vary at positions 806, 881, 888, 931, 975 and 976; or (l) the S_{MVopt}6P3F polypeptide, or a variant thereof having at least 90% identity with the S_{MVopt}6P3F polypeptide, wherein the variant does not vary at positions 682, 683, 685, 817, 892, 899, 942, 986 and 987. In some embodiments, the mutated antigen is (a) the TA-S2P3F polypeptide of SEQ ID NO: 52; or (b) the S6P polypeptide of SEQ ID NO: 54, or (c) the S6P3F polypeptide of SEQ ID NO: 56, or (d) the S6PΔF polypeptide of SEQ ID NO: 58, or (e) the SCCPP polypeptide of SEQ ID NO: 60, or (f) the SCC6P polypeptide of SEQ ID NO: 62, or (g) the S_{MVopt}2P polypeptide of SEQ ID NO: 5, or (h) the S_{MVopt}ΔF polypeptide of SEQ ID NO: 65 or (i) the S_{MVopt}2PΔF polypeptide of SEQ ID NO: 47.

In some embodiments, the SARS-CoV-2 antigenic polypeptide is a dual domain S protein of SARS-CoV-2. In some embodiments, the dual domain S protein of SARS-CoV-2 antigenic polypeptide comprises an insertion, substitution, or deletion in the 11 amino acid residue sequence of the S protein aligned with positions 1263 to 1273 of the amino acid sequence of SEQ ID NO: 3, wherein the insertion, substitution, or deletion increases cell surface expression of the dual domain S protein. In some embodiments, the dual domain S protein further comprises one or more additional substitutions that maintain the expressed dual domain S protein in its prefusion conformation. In some embodiments, the dual domain S protein further comprises the amino acid mutations K986P and V987P of SEQ ID NO: 3. In some embodiments, the dual domain protein is (a) a prefusion-stabilized SF-2P-dER polypeptide of SEQ ID NO: 76, or a variant thereof having at least 90% identity with SEQ ID NO: 76, wherein the variant does not vary at positions 986 and 987; or (b) a prefusion-stabilized SF-2P-2a polypeptide of SEQ ID NO: 82, or a variant thereof having at least having at least 90% identity with SEQ ID NO: 82, wherein the variant does not vary at positions 986, 987, 1269, and 1271. In some embodiments, the dual domain S protein is (a) a prefusion-stabilized SF-2P-dER polypeptide of SEQ ID NO: 76; or (b) a prefusion-stabilized SF-2P-2a polypeptide of SEQ ID NO: 82.

In some embodiments, the dual domain S protein of SARS-CoV-2 antigenic polypeptide further comprises a deletion of the amino acid residues at positions 69 and 70 of the amino acid sequence of SEQ ID NO: 3. In some embodiments, the dual domain S protein of SARS-CoV-2 antigenic polypeptide further comprises a deletion of the amino acid residues at positions 144 and 145 of the amino acid sequence of SEQ ID NO: 3. In some embodiments, the dual domain S protein of SARS-CoV-2 antigenic polypeptide further comprises the amino acid mutation N501Y of SEQ ID NO: 3. In some embodiments, the dual domain S protein of SARS-CoV-2 antigenic polypeptide further comprises the amino acid mutation A570D of SEQ ID NO: 3. In some embodiments, the dual domain S protein of SARS-CoV-2 antigenic polypeptide further comprises the amino acid mutation P681H of SEQ ID NO: 3. In some embodiments, the dual domain S protein of SARS-CoV-2 antigenic polypeptide further comprises the amino acid mutation T7161 of SEQ ID NO: 3. In some embodiments, the dual domain S protein of SARS-CoV-2 antigenic polypeptide further comprises the amino acid mutation S982A of SEQ ID NO: 3. In some embodiments, the dual domain S protein of SARS-CoV-2 antigenic polypeptide further comprises the amino acid mutation D1118H of SEQ ID NO: 3. In some embodiments, the dual domain S protein of SARS-CoV-2 antigenic polypeptide further comprises the amino acid mutation E484K of SEQ ID NO: 3. In some embodiments, the dual domain S protein of SARS-CoV-2 antigenic polypeptide further comprises the amino acid mutation K417N of SEQ ID NO: 3. In some embodiments, the dual domain S protein of SARS-CoV-2 antigenic polypeptide further comprises the amino acid mutation K417T of SEQ ID NO: 3. In some embodiments, the dual domain S protein of SARS-CoV-2 antigenic polypeptide further comprises the amino acid mutation D614G of SEQ ID NO: 3.

In some embodiments of the foregoing method, the coronavirus antigenic polypeptide or an immunogenic fragment thereof comprises or consists of at least one polypeptide of SARS-CoV-2 selected from the group consisting of: nucleocapsid (N) polypeptide or a variant thereof having at least 90% identity with the N polypeptide; matrix (M) polypeptide or a variant thereof having at least 90% identity with M polypeptide; E polypeptide or a variant thereof having at least 90% identity with E polypeptide; 8a polypeptide or a variant thereof having at least 90% identity with 8a polypeptide; 7a polypeptide or a variant thereof having at least 90% identity with 7a polypeptide; 3A polypeptide or a variant thereof having at least 90% identity with 3 polypeptide.

Prophylactic and Therapeutic Compositions

A prophylactically effective dose is a therapeutically effective dose that prevents infection with the virus at a clinically acceptable level. In some embodiments the therapeutically effective dose is a dose listed in a package insert for the vaccine.

Provided herein are compositions (e.g., pharmaceutical compositions), methods, kits and reagents for prevention, treatment or diagnosis of coronavirus infection, particularly SARS-CoV-2 infection, in humans and other mammals, for example. Coronavirus compositions can be used as prophylactic or therapeutic agents. They may be used in medicine to prevent and/or treat infectious disease. In some embodiments, the compositions of the present disclosure are used for the priming of immune effector cells, for example, to activate peripheral blood mononuclear cells (PBMCs) ex vivo, which are then infused (re-infused) into a subject. In some embodiments, compositions in accordance with the present disclosure may be used for treatment of coronavirus infection, particularly SARS-CoV-2.

Immunogenic compositions may be administered prophylactically or therapeutically as part of an active immunization scheme to healthy individuals or early in infection during the incubation phase or during active infection after onset of symptoms. In some embodiments, the amount of immunogenic composition of the present disclosure provided to a cell, a tissue or a subject may be an amount effective for immune prophylaxis.

Immunogenic compositions of the present disclosure may be administered with other prophylactic or therapeutic compounds. As a non-limiting example, a prophylactic or therapeutic compound may be an adjuvant or a booster. A booster (or booster vaccine) may be given after an earlier administration of the prophylactic composition. The time of administration between the initial administration of the prophylactic composition and the booster may be, but is not limited to, 1 minute, 2 minutes, 3 minutes, 4 minutes, 5 minutes, 6 minutes, 7 minutes, 8 minutes, 9 minutes, 10 minutes, 15 minutes, 20 minutes 35 minutes, 40 minutes, 45 minutes, 50 minutes, 55 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 36 hours, 2 days, 3 days, 4 days, 5 days, 6 days, 1 week, 10 days, 2 weeks, 3 weeks, 1 month, 2 months, 3 months, 4 months, 5 months, 6 months, 7 months, 8 months, 9 months, 10 months, 11 months, 1 year, 18 months, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, 25 years, 30 years, 35 years, 40 years, 45 years, 50 years, 55 years, 60 years, 65 years, 70 years, 75 years, 80 years, 85 years, 90 years, 95 years or more than 99 years. In some embodiments, the time of administration between the initial administration of the prophylactic composition and the booster may be, but is not limited to, 1 week, 2 weeks, 3 weeks, 1 month, 2 months, 3 months, 6 months, or 1 year.

In some embodiments, immunogenic compositions of this disclosure may be administered intramuscularly or intradermally. In some embodiments, immunogenic compositions are administered intramuscularly.

Immunogenic compositions of this disclosure may be utilized in various settings depending on the prevalence of the infection or the degree or level of unmet medical need.

Vaccines have superior properties in that they produce much larger antibody titers and/or cellular immune responses, and produce responses earlier than commercially available anti-viral agents/compositions.

Provided herein are pharmaceutical compositions comprising a recombinant measles virus of this disclosure and/or a recombinant VLP of this disclosure, optionally in combination with one or more pharmaceutically acceptable excipients. The immunogenic composition may comprise a suitable vehicle for administration e.g. a pharmaceutically acceptable vehicle to a host, especially a human host and may further comprise but not necessarily adjuvant to enhance immune response in a host. Pharmaceutically acceptable vehicles useful in the compositions of the invention include any compatible agent that is nontoxic to patients at the dosages and concentrations employed, such as water, saline, dextrose, glycerol, ethanol, buffers, and the like, and combinations thereof. The vehicle may also contain additional components such as a stabilizer, a solubilizer, a tonicity modifier, such as NaCl, MgCl₂, or CaCl₂ etc., a surfactant, and mixtures thereof. The inventors have indeed shown that the administration of the active ingredients of the invention may elicit an immune response without the need for an external adjuvant. In some embodiments, immunogenic compositions disclosed herein do not include an adjuvant (they are adjuvant free).

Such a vaccine composition comprises advantageously active principles (active ingredients) which comprise recombinant infectious replicating MV-CoV particles rescued from the vector and constructs as defined herein optionally associated with VLPs comprising the same CoV proteins.

The administration scheme and dosage regime may require a unique administration of a selected dose of the recombinant infectious replicating MV-CoV particles according to the invention in association with the above-mentioned CoV proteins, in particular in association with CoV-VLPs expressing the same CoV proteins.

Alternatively it may require administration of multiple doses.

In a particular embodiment the administration is performed in accordance with a prime-boost regimen. Priming and boosting may be achieved with identical active ingredients consisting of the recombinant infectious replicating MV-CoV particles in association with the above-mentioned CoV proteins, in particular in association with CoV-VLPs expressing the same CoV proteins.

Alternatively priming and boosting administration may be achieved with different active ingredients, involving the recombinant infectious replicating MV-CoV particles in association with the above-mentioned CoV proteins, in particular in association with CoV-VLPs expressing the same CoV proteins, in at least one of the administration steps and other active immunogens of CoV, such as the above-mentioned CoV polypeptides or CoV-VLPs expressing the same CoV proteins, in other administration steps.

Administration of recombinant infectious replicating MV-CoV particles according to the invention in association with CoV-VLPs expressing the same CoV proteins elicits an immune response and may elicit antibodies that are cross-reactive for various CoV strains. Accordingly, administration of the active ingredients according to the invention, when prepared with the coding sequences of a particular strain of CoV, may elicit an immune response against a group of strains of CoV.

Considering that the currently known doses for human MV vaccines are in the range of 10³ to 10⁵ TCID50, a suitable dose of recombinant MV-CoV to be administered may be in the range of 0.1 to 10 ng, in particular 0.2 to 6 ng, and in some embodiments as low as 0.2 to 2 ng.

According to a particular embodiment of the invention, the immunogenic or vaccine composition defined herein may also be used for protection against an infection by the measles virus.

The invention also relates to a method for preventing a coronavirus virus related disease, in particular a disease related to infection by SARS-CoV-2, i.e. COVID-19, the method comprising the immunization of a mammalian, especially a human, in particular a child, by the injection, in particular by subcutaneous injection, of recombinant measles virus according to the invention.

The invention also relates to a method for treating a coronavirus virus related disease, in particular a disease related to infection by SARS-CoV-2, i.e. COVID-19, the method comprising the immunization of a mammalian, especially a human, in particular a child, by the injection, in particular subcutaneous injection, of recombinant measles virus according to the invention.

Modes of Vaccine Administration

Immunogenic compositions may be administered by any route which results in a therapeutically effective outcome. These include, but are not limited, to intradermal, intramuscular, intranasal and/or subcutaneous administration. The present disclosure provides methods comprising administering immunogenic compositions to a subject in need thereof. The exact amount required will vary from subject to subject, depending on the species, age, and general condition of the subject, the severity of the disease, the particular composition, its mode of administration, its mode of activity, and the like. Immunogenic compositions are typically formulated in dosage unit form for ease of administration and uniformity of dosage. It will be understood, however, that the total daily usage of vaccine compositions may be decided by the attending physician within the scope of sound medical judgment. The specific therapeutically effective, prophylactically effective, or appropriate imaging dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

An immunogenic composition described herein can be formulated into a dosage form described herein, such as an intranasal, intratracheal, or injectable (e.g., intravenous, intraocular, intravitreal, intramuscular, intradermal, intracardiac, intraperitoneal, intranasal and subcutaneous).

Immunogenic Formulations and Methods of Use

Some aspects of the present disclosure provide formulations of the immunogenic composition, wherein the vaccine is formulated in an effective amount to produce an antigen specific immune response in a subject (e.g., production of antibodies specific to a coronavirus antigenic polypeptide). An “effective amount” is a dose of an immunogenic composition effective to produce an antigen-specific immune response. Also provided herein are methods of inducing an antigen-specific immune response in a subject.

In some embodiments, the antigen-specific immune response is characterized by measuring an anti-antigenic polypeptide antibody titer produced in a subject administered an immunogenic composition as provided herein. An antibody titer is a measurement of the amount of antibodies within a subject, for example, antibodies that are specific to a particular antigen (e.g., a mutated full length S protein or a mutated dual domain S protein) or epitope of an antigen. Antibody titer is typically expressed as the inverse of the greatest dilution that provides a positive result. Enzyme-linked immunosorbent assay (ELISA) is a common assay for determining antibody titers, for example.

In some embodiments, an antibody titer is used to assess whether a subject has had an infection or to determine whether immunizations are required. In some embodiments, an antibody titer is used to determine the strength of an autoimmune response, to determine whether a booster immunization is needed, to determine whether a previous vaccine was effective, and to identify any recent or prior infections. In accordance with the present disclosure, an antibody titer may be used to determine the strength of an immune response induced in a subject by the immunogenic composition.

The invention also relates to a nucleic acid molecule that encodes a polypeptide of SARS-CoV-2 and which has been modified with respect to the native sequence. In particular the invention relates to the nucleic acid molecule comprising or consisting of a polynucleotide of sequence as disclosed in Table 1.

TABLE 1
Spike Polypeptides of SARS-CoV-2

| Construct | SEQ ID NO |
|---|---|
| S polypeptide of nCoV (i.e. | SEQ ID NO: 1 or SEQ ID NO: 2 |
| SARS-CoV-2) | |
| stab-S polypeptide of nCoV (i.e. | SEQ ID NO: 4 |
| SARS-CoV-2) | |
| (also named S2P polypeptide of | |
| nCoV (i.e. SARS-CoV-2)) | |
| Secto polypeptide of nCoV (i.e. | SEQ ID NO: 6 |
| SARS-CoV-2) | |
| stab-Secto polypeptide of nCoV | SEQ ID NO: 8 |
| (i.e. SARS-CoV-2) | |
| S1 polypeptide of nCoV (i.e. | SEQ ID NO: 10 |
| SARS-CoV-2) | |
| S2 polypeptide of nCoV (i.e. | SEQ ID NO: 12 |
| SARS-CoV-2) | |
| stab-S2 polypeptide of nCoV | SEQ ID NO: 14 |
| (i.e. SARS-CoV-2) | |
| tri-Secto polypeptide of nCoV | SEQ ID NO: 16 |
| (i.e. SARS-CoV-2) | |
| tristab-Secto polypeptide of | SEQ ID NO: 18 |
| nCoV (i.e. SARS-CoV-2) | |
| S3F polypeptide of nCoV (i.e. | SEQ ID NO: 42 |
| SARS-CoV-2) | |
| S2P3F polypeptide of nCoV (i.e. | SEQ ID NO: 44 |
| SARS-CoV-2) | |
| S2PΔF polypeptide of nCoV (i.e. | SEQ ID NO: 46 |
| SARS-CoV-2) | |
| S2PΔF2A polypeptide of nCoV | SEQ ID NO: 48 |
| (i.e. SARS-CoV-2) | |
| T4-S2P3F polypeptide of SARS- | SEQ ID NO: 51 |
| CoV-2 (also named tristab- | |
| Secto-3F) | |
| S6P polypeptide of SARS-CoV-2 | SEQ ID NO: 53 |
| S6P3F polypeptide of SARS- | SEQ ID NO: 55 |
| CoV-2 | |
| S6PΔF polypeptide of SARS- | SEQ ID NO: 57 |
| CoV-2 | |
| SCCPP polypeptide of SARS- | SEQ ID NO: 59 |
| CoV-2 | |
| SCC6P polypeptide of SARS- | SEQ ID NO: 61 |
| CoV-2 | |
| S_{MVopt}2P polypeptide of SARS- | SEQ ID NO: 63 |
| CoV-2 | |
| S_{MVopt}ΔF polypeptide of SARS- | SEQ ID NO: 64 |
| CoV-2 | |
| S_{MVopt}2PΔF polypeptide of | SEQ ID NO: 66 |
| SARS-CoV-2 | |
| SF-dER of nCoV (i.e. SARS- | SEQ ID NO: 73 |
| CoV-2) | |
| SF-2P-dER of nCoV (i.e. | SEQ ID NO: 75 |
| SARS-CoV-2) | |
| S2-dER of nCoV (i.e. SARS- | SEQ ID NO: 77 |
| CoV-2) | |
| S2-2P-dER of nCoV (i.e. | SEQ ID NO: 79 |
| SARS-CoV-2) | |
| SF-2P-2a of nCoV (i.e. SARS- | SEQ ID NO: 81 |
| CoV-2) | |

The invention also concerns the plasmids disclosed in Table 2.

TABLE 2
Measles Virus Plasmids encoding SARS-CoV-2 Spike protein

| Plasmid | SEQ ID NO |
|---|---|
| pKP-MVSchw | SEQ ID NO: 30 |
| pKP-MVSchw-ATU1(eGFP) | SEQ ID NO: 31 |
| pKP-MVSchw-ATU2(eGFP) | SEQ ID NO: 32 |
| pKP-MVSchw-ATU3(eGFP) | SEQ ID NO: 33 |
| pKM-ATU2-S_2019-nCoV (i.e. SARS-CoV-2) | SEQ ID NO: 34 |
| (optimized sequence) | |
| pKM-ATU3-S_2019-nCoV (i.e. SARS-CoV-2) | SEQ ID NO: 35 |
| (optimized sequence) | |
| pTM2-SF-dER_SARS-CoV-2 (optimized | SEQ ID NO: 144 |
| sequence) | |
| pTM2-S2-dER_SARS-CoV-2 (optimized | SEQ ID NO: 145 |
| sequence) | |
| pTM2-SF-2P-dER_SARS-CoV-2 (optimized | SEQ ID NO: 146 |
| sequence) | |
| pTM2-S2-2P-dER_SARS-CoV-2 (optimized | SEQ ID NO: 147 |
| sequence) | |
| pTM2-SF-2P-2a_SARS-CoV-2 (optimized | SEQ ID NO: 148 |
| sequence) | |

In a particular aspect the invention relates to the plasmid pKP-MVSchwarz deposited under No. CNCM I-5493 on Feb. 12, 2020 or the plasmid pKP-MVSchw having the sequence of SEQ ID NO:30. This plasmid may be used as plasmid for cloning any polynucleotide.

In another particular aspect the invention relates to the plasmid pTM-MVSchw deposited under No. CNCM I-2889 on Jun. 12, 2002 (or having the sequence of SEQ ID NO: 28), or the plasmid pTM2-MVSchw-gfp deposited under No. CNCM I-2890 on Jun. 12, 2002 (or having the sequence of SEQ ID NO: 29), or the plasmid pTM3-MVSchw-gfp having the sequence of SEQ ID NO: 38, preferably the plasmid pTM2-MVSchw-gfp deposited under No. CNCM I-2890 on Jun. 12, 2002 (or having the sequence of SEQ ID NO: 29). This plasmid may be used as plasmid for cloning any polynucleotide.

TABLE 3
Native and codon/MV-optimized nucleotide sequences of the polynucleotide
encoding particular peptides/proteins as well as amino acid sequences
of these peptides/proteins used in the invention.

| | | SEQ ID NO | SEQ ID NO | |
|---|---|---|---|---|
| | SEQ ID NO | of the codon- | of the MV- | |
| | of the native | optimized | optimized | |
| | nucleotide | nucleotide | nucleotide | SEQ ID NO |
| | sequence of the | sequence of the | sequence of the | of the amino |
| Name of the compound, i.e. | polynucleotide | polynucleotide | polynucleotide | acid sequence |
| peptide/protein/antigen | encoding the | encoding the | encoding the | of the |
| (abbreviation) | compound | compound | compound | compound |
| S polypeptide of nCoV (i.e. SARS- | 1 | 2 | 36 | 3 |
| CoV-2) | | | | |
| stab-S polypeptide of nCoV (i.e. | | 4 | | 5 |
| SARS-CoV-2) | | | | |
| (also named S2P polypeptide of | | | | |
| nCoV (i.e. SARS-CoV-2)) | | | | |
| Secto polypeptide of nCoV (i.e. | | 6 | | 7 |
| SARS-CoV-2) | | | | |
| stab-Secto polypeptide of nCoV | | 8 | | 9 |
| {i.e. SARS-CoV-2) | | | | |
| S1 polypeptide of nCoV (i.e. SARS- | | 10 | | 11 |
| CoV-2) | | | | |
| S2 polypeptide of nCoV (i.e. SARS- | | 12 | | 13 |
| CoV-2) | | | | |
| stab-S2 polypeptide of nCoV (i.e. | | 14 | | 15 |
| SARS-CoV-2) | | | | |
| tri-Secto polypeptide of nCoV {i.e. | | 16 | | 17 |
| SARS-CoV-2) | | | | |
| tristab-Secto polypeptide of nCoV | | 18 | | 19 |
| {i.e. SARS-CoV-2) | | | | |
| N polypeptide of nCoV (i.e. SARS- | 20 | 21 | 37 | 22 |
| CoV-2) | | | | |
| E polypeptide of nCoV (i.e. SARS- | | | | 23 |
| CoV-2) | | | | |
| M polypeptide of nCoV (i.e. SARS- | | | | 24 |
| CoV-2) | | | | |
| ORF8 polypeptide of nCoV (i.e. | | | | 25 |
| SARS-CoV-2) | | | | |
| ORF3a polypeptide of nCoV (i.e. | | | | 26 |
| SARS-CoV-2) | | | | |
| ORF7a polypeptide of nCoV (i.e. | | | | 27 |
| SARS-CoV-2) | | | | |
| S3F polypeptide of nCoV (i.e. SARS- | | 42 | | 43 |
| CoV-2) | | | | |
| S2P3F polypeptide of nCoV (i.e. | | 44 | | 45 |
| SARS-CoV-2) | | | | |
| S2PΔF polypeptide of nCoV (i.e. | | 46 | | 47 |
| SARS-CoV-2) | | | | |
| S2PΔF2A polypeptide of nCoV (i.e. | | 48 | | 49 |
| SARS-CoV-2) | | | | |
| T4-S2P3F polypeptide of SARS-CoV- | | 51 | | 52 |
| 2 (also named tristab-Secto-3F | | | | |
| polypeptide of SARS-CoV-2) | | | | |
| S6P polypeptide of SARS-CoV-2 | | 53 | | 54 |
| S6P3F polypeptide of SARS-CoV-2 | | 55 | | 56 |
| S6PΔF polypeptide of SARS-CoV-2 | | 57 | | 58 |
| SCCPP polypeptide of SARS-CoV-2 | | 59 | | 60 |
| SCC6P polypeptide of SARS-CoV-2 | | 61 | | 62 |
| S_{MVopt}2P polypeptide of SARS-CoV-2 | | | 63 | 5 |
| S_{MVopt}ΔF polypeptide of SARS-CoV-2 | | | 64 | 65 |
| S_{MVopt}2PΔF polypeptide of SARS- | | | 66 | 47 |
| CoV-2 | | | | |
| SF-dER of nCoV (i.e. SARS-CoV-2) | | 73 | | 74 |
| SF-2P-dER of nCoV (i.e. SARS-CoV-2) | | 75 | | 76 |
| S2-dER of nCoV (i.e. SARS-CoV-2) | | 77 | | 78 |
| S2-2P-dER of nCoV (i.e. SARS-CoV-2) | | 79 | | 80 |
| SF-2P-2a of nCoV (i.e. SARS-CoV-2) | | 81 | | 82 |

TABLE 4
Transfer Vector Plasmid and elements.

| Name of the transfer vector plasmid and elements | SEQ ID NO |
|---|---|
| pTM-MVSchw | 28 |
| pTM2-MVSchw-gfp | 29 |
| pKP-MVSchw | 30 |
| pKP-MVSchw-ATU1(eGFP) | 31 |
| pKP-MVSchw-ATU2(eGFP) | 32 |
| pKP-MVSchw-ATU3(eGFP) | 33 |
| pKM-ATU2-S_2019-nCoV (i.e. SARS-CoV-2) | 34 |
| (optimized sequence) | |
| pKM-ATU3-S_2019-nCoV (i.e. SARS-CoV-2) | 35 |
| (optimized sequence) | |
| pTM3-MVSchw-gfp | 38 |
| ATU1(eGFP) | 39 |
| ATU2(eGFP) | 40 |
| ATU3(eGFP) | 41 |

The disclosed sequences are further characterized by the following annotations relating to the nucleotide or amino acid residues:

The invention also concerns recombinant virus like particles (VLPs) comprising a S polypeptide or an immunogenic fragment thereof that has 1, 2, 3 or more amino acid substitution(s), insertion(s) or deletion(s), which is(are) encoded by the first and optionally the second heterologous polynucleotide(s) of the nucleic acid construct according to the invention, or of the transfer plasmid vector according to the invention, or the recombinant measles virus according to the invention or is produced within the host cell according to the invention.

In accordance with the present invention, VLPs can be produced in large quantities and are expressed together with recombinant infectious MV-CoV particles. The VLPs are VLPs of CoV.

According to a preferred embodiment of the invention, the recombinant MV vector is designed in such a way and the production process involves cells such that the virus particles produced in helper cells transfected or transformed with the vector, originated from a MV strain adapted for vaccination, enable the production of recombinant infectious replicating MV and the production of CoV-VLPs for use in immunogenic compositions, preferably protective or vaccine compositions.

Advantageously, the genome of the recombinant infectious MV-CoV particles of the invention is replication competent. By “replication competent’, it is meant a nucleic acid, which when transduced into a helper cell line expressing the N, P and L proteins of a MV, is able to be transcribed and expressed in order to produce new viral particles.

Replication of the recombinant virus of the invention obtained using MV cDNA for the preparation of the recombinant genome of MV-CoV can also be achieved in vivo in the host, in particular the human host to which recombinant MV-CoV is administered.

The invention also relates to a polypeptide of a coronavirus (CoV), in particular of SARS-CoV-2, encoded by the nucleic acid molecule according to the invention.

In a particular embodiment of the invention, the polypeptide has an amino acid sequence selected from the group consisting of:
i. SEQ ID NO: 3 (construct S);
ii. SEQ ID NO: 5 (construct stab-S);
iii. SEQ ID NO: 7 (construct Secto);
iv. SED ID NO: 9 (construct stab-Secto);
v. SEQ ID NO: 11 (construct S1),
vi. SEQ ID NO: 13 (construct S2),
vii. SEQ ID NO: 15 (construct stab-S2),
viii. SEQ ID NO: 17 (construct tri-Secto),
ix. SEQ ID NO: 19 (construct tristab-Secto),
x. SEQ ID NO: 43 (construct S3F),
xi. SEQ ID NO: 45 (construct S2P3F),
xii. SEQ ID NO: 47 (construct S2PΔF),
xiii. SEQ ID NO: 49 (construct S2PΔF2A),
xiv. SEQ ID NO: 22 (construct N),
xv. SEQ ID NO: 52 (construct T4-S2P3F (tristab-Secto-3F)),
xvi. SEQ ID NO: 54 (construct S6P),
xvii. SEQ ID NO: 56 (construct S6P3F),
xviii. SEQ ID NO: 58 (construct S6PΔF),
xix. SEQ ID NO: 60 (construct SCCPP), and
xx. SEQ ID NO: 62 (construct SCC6P),
   preferably the polypeptide has an amino acid sequence selected from the group consisting of SEQ ID NO: 5, SEQ ID NO: 43, SEQ ID NO: 45, SEQ ID NO: 47 and SEQ ID NO: 49, more preferably an amino acid sequence selected from the group consisting of SEQ ID NO: 45, SEQ ID NO: 47 and SEQ ID NO: 49, even more preferably the polypeptide of SEQ ID NO: 47 or SEQ ID NO: 49.

In a preferred embodiment of the invention, the polypeptide has an amino acid sequence of SEQ ID NO: 76 (construct SF-2P-dER) or SEQ ID NO: 82 (construct SF-2P-2a), preferably an amino acid sequence of SEQ ID NO: 76 (construct SF-2P-dER).

The present invention also relates to a recombinant protein expressed by the transfer vector according to the invention.

In a particular embodiment of the invention, the recombinant protein further comprises an amino acid tag for purification.

The present invention also relates to a recombinant protein expressed in vitro or in vivo by the transfer vector according to the invention.

The invention also relates to the in vitro use of an antigen of SARS-CoV-2 which is the spike antigen or an immunogenic fragment thereof that has 1, 2, 3 or more amino acid substitution(s), insertion(s) or deletion(s), in particular an antigen having the sequence of SEQ ID NO: 3, 5, 7, 9, 11, 13, 15, 17, 19, 22, 23, 24, 25, 26, 27, 43, 45, 47 or 49, preferably of SEQ ID NO: 3, 5, 43, 45, 47 or 49, more preferably of SEQ ID NO: 45, 47 or 49, even more preferably of SEQ ID NO: 47 or 49, even more preferably of SEQ ID NO: 49, for the detection in a biological sample, especially a blood or a serum sample previously obtained from an individual suspected of being infected by a coronavirus, in particular by SARS-CoV-2, wherein the antigen is contacted with the biological sample to determine the presence of antibodies against the antigen.

Preferably, the in vitro use of an antigen of SARS-CoV-2 is the spike antigen or the immunogenic fragment thereof or the mutated antigen thereof as defined above, in particular an antigen having the sequence of SEQ ID NOs: 3, 5, 7, 9, 11, 13, 15, 17, 19, 22, 23, 24, 25, 26, 27, 43, 45, 47, 49, 52, 54, 56, 58, 60, 62 or 65, preferably an antigen having the sequence of SEQ ID NOs: 3, 5, 43, 45, 47 or 49, more preferably an antigen of SEQ ID NO: 49, for the detection in a biological sample, especially a blood or a serum sample previously obtained from an individual suspected of being infected by SARS-CoV-2, wherein the antigen is contacted with the biological sample to determine the presence of antibodies against the antigen.

The invention also relates to the in vitro use of an antigen of SARS-CoV-2 which is the spike antigen or an immunogenic fragment thereof that has 1, 2, 3 or more amino acid substitution(s), insertion(s) or deletion(s), in particular an antigen having the sequence of SEQ ID NO: 3, 5, 7, 9, 11, 13, 15, 17, 19, 22, 23, 24, 25, 26 or 27, preferably of SEQ ID NO: 3 or 5, for the detection in a biological sample, especially a blood or a serum sample previously obtained from an individual suspected of being infected by a coronavirus, in particular by SARS-CoV-2, wherein the antigen is contacted with the biological sample to determine the presence of antibodies against the antigen.

Preferably, the In vitro use of the antigen of SARS-CoV-2 is the SF-2P-dER antigen or the SF-2P-2a antigen or an immunogenic fragment thereof that has 1, 2, 3 or more amino acid substitution(s), insertion(s) and/or deletion(s), in particular an antigen having the sequence of SEQ ID NO: 76 or SEQ ID NO: 82, preferably an antigen having the sequence of SEQ ID NO: 76, for the detection in a biological sample, especially a blood or a serum sample previously obtained from an individual suspected of being infected by SARS-CoV-2, wherein the antigen is contacted with the biological sample to determine the presence of antibodies against the antigen.

The invention also relates to the in vitro use of an antigen of SARS-CoV-2 which is the spike antigen or an immunogenic fragment thereof that has 1, 2, 3 or more amino acid substitution(s), insertion(s) or deletion(s), in particular an antigen having the sequence of SEQ ID NO: 3, 5, 7, 9, 11, 13, 15, 17, 19, 22, 23, 24, 25, 26, 27, 43, 45, 47 or 49, preferably of SEQ ID NO: 3, 5, 43, 45, 47 or 49, more preferably of SEQ ID NO: 45, 47 or 49, even more preferably of SEQ ID NO: 47 or 49, even more preferably of SEQ ID NO: 49, for diagnosis or vaccine purposes, or as a pre-fusion configuration.

A method for in vitro diagnosing a coronavirus, in particular coronavirus SARS-CoV-2, comprising the use of an antigen of SARS-CoV-2 which is the spike antigen or an immunogenic fragment thereof that has 1, 2, 3 or more amino acid substitution(s), insertion(s) or deletion(s), in particular an antigen having the sequence of SEQ ID NO: 3, 5, 7, 9, 11, 13, 15, 17, 19, 22, 23, 24, 25, 26, 27, 43, 45, 47 or 49, preferably of SEQ ID NO: 3, 5, 43, 45, 47 or 49, more preferably of SEQ ID NO: 45, 47 or 49, even more preferably of SEQ ID NO: 47 or 49, even more preferably of SEQ ID NO: 49.

The invention also relates to the in vitro use of an antigen of SARS-CoV-2 which is the spike antigen or an immunogenic fragment thereof that has 1, 2, 3 or more amino acid substitution(s), insertion(s) or deletion(s), in particular an antigen having the sequence of SEQ ID NO: 3, 5, 7, 9, 11, 13, 15, 17, 19, 22, 23, 24, 25, 26 or 27, preferably of SEQ ID NO: 3 or 5, for the detection in a biological sample, especially a blood or a serum sample previously obtained from an individual suspected of being infected by a coronavirus, in particular by SARS-CoV-2, wherein the antigen is contacted with the biological sample to determine the presence of antibodies against the antigen.

Preferably, the In vitro use of the antigen of SARS-CoV-2 is the SF-2P-dER antigen or the SF-2P-2a antigen or an immunogenic fragment thereof that has 1, 2, 3 or more amino acid substitution(s), insertion(s) and/or deletion(s), in particular an antigen having the sequence of SEQ ID NO: 76 or SEQ ID NO: 82, preferably an antigen having the sequence of SEQ ID NO: 76, for the detection in a biological sample, especially a blood or a serum sample previously obtained from an individual suspected of being infected by SARS-CoV-2, wherein the antigen is contacted with the biological sample to determine the presence of antibodies against the antigen.

In a particular embodiment of the invention, the antigen is placed in contact with a biological sample, especially a blood or a serum sample previously obtained from an individual suspected of being infected by a coronavirus, in particular by SARS-CoV-2, and the presence of antibodies against the antigen is determined.

The invention also relates to a method for treating or preventing an infection by SARS-CoV-2 in a host, in particular a human host, comprising administering the immunogenic or vaccine composition according to the invention to the host.

The invention also relates to a method for inducing a protective immune response against SARS-CoV-2 in a host, in particular a human host, comprising administering the immunogenic or vaccine composition according to the invention to the host.

In a particular embodiment of the methods, the administration comprises at least two successive administration steps. Preferably, the second administration is performed from two weeks up to 6 months after the first administration, in particular one or two months after the first administration.

### Antigens/Antigenic Polypeptides

In some embodiments, an antigenic polypeptide includes gene products, naturally occurring polypeptides, synthetic polypeptides, homologs, orthologs, paralogs, fragments and other equivalents, variants, and analogs of the foregoing. A polypeptide may be a single molecule or may be a multi-molecular complex such as a dimer, trimer or tetramer. Polypeptides may also comprise single chain polypeptides or multichain polypeptides, and may be associated or linked to each other. Most commonly, disulfide linkages are found in multichain polypeptides. The term “polypeptide” may also apply to amino acid polymers in which at least one amino acid residue is an artificial chemical analogue of a corresponding naturally-occurring amino acid.

As recognized by those skilled in the art, protein fragments, functional protein domains, and homologous proteins are also considered to be within the scope of polypeptides of interest. For example, provided herein is any protein fragment (meaning a polypeptide sequence at least one amino acid residue shorter than a reference polypeptide sequence but otherwise identical) of a reference protein having a length of 10, 20, 30, 40, 50, 60, 70, 80, 90, 100 or longer than 100 amino acids. In another example, any protein that comprises a stretch of 20, 30, 40, 50, or 100 (contiguous) amino acids that are 40%, 50%, 60%, 70%, 80%, 90%, 95%, 96%, 97%, 98%, 99% or 100% identical to any of the sequences described herein can be utilized in accordance with the disclosure. In some embodiments, a polypeptide comprises 2, 3, 4, 5, 6, 7, 8, 9, 10, or more mutations as shown in any of the sequences provided herein or referenced herein. In another example, any protein that comprises a stretch of 20, 30, 40, 50, or 100 amino acids that are greater than 80%, 90%, 95%, or 100% identical to any of the sequences described herein, wherein the protein has a stretch of 5, 10, 15, 20, 25, or 30 amino acids that are less than 80%, 75%, 70%, 65% to 60% identical to any of the sequences described herein can be utilized in accordance with the disclosure.

Polypeptide or polynucleotide molecules of the present disclosure may share a certain degree of sequence similarity or identity with the reference molecules (e.g., reference polypeptides or reference polynucleotides), for example, with art-described molecules (e.g., engineered or designed molecules or wild type molecules).

Other features and advantages of the invention will be apparent from the examples which follow and will also be illustrated in the figures, none of which are intended to be limiting.

### EXAMPLES

### A. Example 1

1. Materials and Methods

Design of Specific Antigenic Sequences of SARS-CoV-2

cDNAs encoding native spike and nucleoprotein antigens from SARS-CoV-2 were designed based on the Genbank MN908947 sequence publicly available from NBCBI on 20 Jan. 2020. These sequences were then processed through the Project Manager platform of GeneArt (ThermoFisher) to generate codon-optimized nucleotide sequences for high expression in mammalian and drosophila cells. Regions of very high (>80%) or low (<30%) GC content were avoided whenever possible, and cis-acting sequence motifs like internal TATA-boxes, chi-sites, ribosomal entry sites, ARE, INS, and CRS sequence elements, as well as repetitive sequences, RNA secondary structures and splice donor and acceptor sites, were avoided. Both sequences were further edited to remove MV editing (AnGn, n≥3)- and core gene end (A4CKT)-like sequences on both strands. BsiWI and BssHII restriction sites were then added at the 5′ and 3′ ends, respectively, of the nucleotide sequences. The resulting cDNAs respect the “rule of six”, which stipulates that the number of nucleotides of the MV genome must be a multiple of 6 and have the sequences 20AAP6IP-S-2019-nCoV (i.e. SARS-CoV-2)-opt(2×) (SEQ ID NO: 2) and 20AASUIP-N-2019-nCoV (i.e. SARS-CoV-2)-opt(2×) (SEQ ID NO: 21), respectively (see below).

The native spike and nucleoprotein sequences were also processed through the Project Manager platform of GeneArt (ThermoFisher) to generate optimized genes for the measles vaccine platform. The coding sequences were modified in order to generate sequences with a target GC composition of 44-50% and a balanced codon composition, avoiding, where applicable, rare codons and high usage of the most frequent codons. As for the generation of the fully codon-optimized sequences described above, regions of very high (>80%) or low (<30%) GC content were avoided whenever possible, and cis-acting sequence motifs like internal TATA-boxes, chi-sites, ribosomal entry sites, ARE, INS, and CRS sequence elements, as well as repetitive sequences, RNA secondary structures and splice donor and acceptor sites, were avoided. Both sequences were also further edited to remove MV editing (AnGn, n≥3)- and core gene end (A4CKT)-like sequences on both strands. BsiWI and BssHII restriction sites were then added at the 5′ and 3′ ends, respectively, of the nucleotide sequences. The resulting cDNAs respect the “rule of six”, which stipulates that the number of nucleotides of the MV genome must be a multiple of 6 and have the sequences 20AAS76C_S-2019-nCoV_mod (SEQ ID NO: 36) and 20AAS77C_N-2019-nCoV-HS_mod (SEQ ID NO: 37), respectively (see below).

The 4 resulting cDNAs were synthesized at Geneart (ThermoFisher) facilities.

Plasmid Vector Constructs and Vaccine Candidate Rescue

The MVSchw recombinant plasmid constructs have been derived from novel pKP-MVSchw-ATU1(eGFP), -ATU2 and -ATU3 plasmid vectors (abbreviated names: pKM1, pKM2, pKM3). These vectors were constructed from the original pTM-MVSchw-ATU1, -ATU2 and -ATU3 plasmid vectors, respectively (WO04/000876 and Combredet et al., J Virol, 2003). pKM and pTM plasmid vector series carry identical infectious cDNAs corresponding to the anti-genome of the Schwarz MV vaccine strain and an additional transcription unit containing unique BsiWI and BssHII restriction sites for the insertion of foreign open reading frames upstream from the N gene (ATU1), between the P and M genes (ATU2) and between the H and L genes (ATU3).

First, pKM2-eGFP was obtained by transferring the whole T7 rescue cassette of the original pTM2-eGFP plasmid (17038 bp located between the two Not1 sites) into a purposively-modified version of the commercial pENTR2 minimal plasmid (ThermoFisher). Next, pKM, pKM3-eGFP and pKM1-eGFP were sequentially derived from pKM2-eGFP. The novel pKM, pKM1-, pKM2- and pKM3-eGFP vectors were verified for their capacity to rescue the corresponding measles virus and vectors with similar efficiency to that observed for the pTM plasmid vector series. It is noteworthy to highlight that viruses rescued from the novel pKM1, pKM2 and pKM3 plasmid vectors have the same genomic sequence as viruses rescued from original pTM1, pTM2 and pTM3 vectors respectively.

pKM plasmid series are suitable for use to insert a variety of viral antigens in single, dual and triple recombinant vectors which much higher cloning efficiency, stability and DNA yield than the original pTM plasmid series, making them a most useful rescue tool for the measles vaccine platform.

cDNAs encoding the SARS-CoV-2 nucleoprotein and spike antigens described above have been inserted into BsiWI/BssHII-digested pKM2 and pKM3 vectors and the resulting pKM-nCoV plasmids are used to rescue single recombinant MV-nCoV vaccine candidates using a helper-cell-based system as previously described (Combredet et al., J Virol, 2003).

The plasmid pKM2-nCoV_NP and any of the pKM3-nCoV_Spike constructs (full length-S, stab-S, Secto, S1, tri-Secto, tristab-Secto) will be digested with SaII restriction enzyme and ligated to produce a series of double recombinant pKM-nCoV-N&S plasmids. Alternatively, double recombinant plasmids will be obtained by inserting the N and S ATU cassettes in tandem either between the P and M genes (position 3 of MV genome) or between the H and L gene (position 6 of MV genome). The resulting pKM-nCoV-N&S plasmids will be used to rescue dual recombinant MV-nCoV-N&S vaccine candidates as described above.

In Cellulo Characterization of the Vaccine Candidates

The single- and dual-recombinant vaccine candidates will be amplified on Vero-NK cells as disclosed in WO 04/000876. All viral stocks will be produced after infection at a MOI of 0.1, stored at −80° C. and titrated by an endpoint limiting dilution assay on Vero-NK cell monolayers. Infectious titers will be determined as 50% tissue culture infectious doses (TCID₅₀) according to the Reed and Munsch method (Reed et al., Am. J. Hyg., 1938).

Vaccine candidates will be characterized essentially as described for MV-SARS vaccine candidates (Escriou et al., Virology, 2014):
growth curves of vaccine candidates and parental MVSchw will be determined on Vero-NK cells infected at a MOI of 0.1,
expression level of SARS-CoV-2 antigens will be evaluated with available anti-SARS cross-reactive mouse and rabbit antibodies and anti-SARS-CoV-2 antibodies, by indirect immunofluorescence assays (IFA) performed on VeroNK-infected cells as well as by western blot on lysates prepared from infected cells,
genomic stability of the vaccine candidates will be assessed by serial passages in VeroNK cells and full genome NGS sequencing of the rescued and passaged viral stocks.

Generation of Recombinant MV Schwarz Viruses Expressing SARS-CoV-2 S Protein

Cloning of SARS-CoV-2 S Protein in Plasmids with Infectious MV cDNA

The plasmid pKM-MVSchw contains an infectious MV cDNA corresponding to the anti-genome of the Schwarz MV vaccine strain. It was derived from the previously described pTM-MVSchw (Combredet et al., J Virol, 2003). Both plasmids permit the rescue of the Schwarz MV vaccine strain. An optimized cDNA encoding the full-length, membrane-bound, native SARS-CoV2 spike glycoprotein from viruses circulating in early 2020 (publicly available) was chemically synthesized (GeneArt/ThermoFisher, Germany). The complete sequence respects the “rule of six”—which stipulates that the number of nucleotides added into the MV genome must be a multiple of six—and contains BsiWI and BssHII restriction sites at both ends. The spike nucleotide sequence was optimized both for transcription from MV vector (in particular but not limited to removal of cryptic signals and optimization of RNA primary sequence) and for translation in human cells (in particular but not limited to codon optimization and RNA secondary structure). This cDNA was inserted into BsiWI/BssHII-digested pKM-MVSchw-ATU3 which contains an additional transcription unit between the hemagglutinin and polymerase genes of the Schwarz MV genome. The resulting plasmid was named pKM-ATU3-S_2019-nCOV (i.e. SARS-CoV-2).

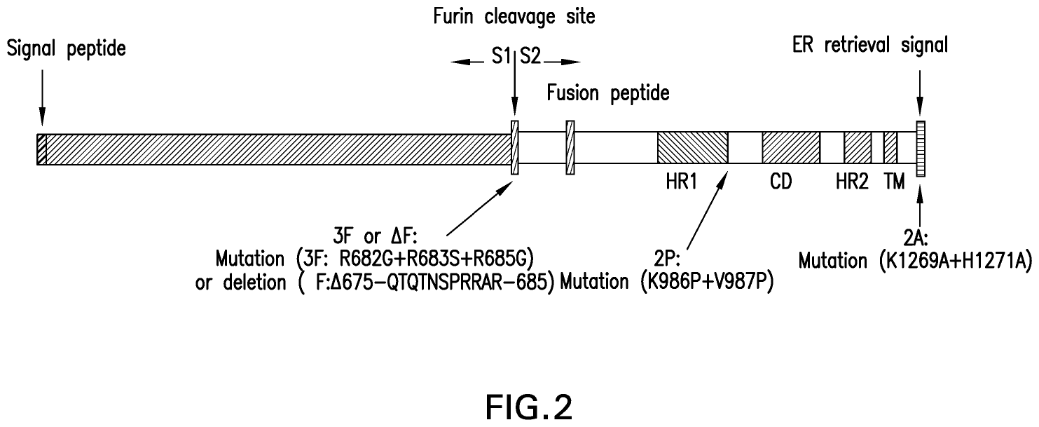

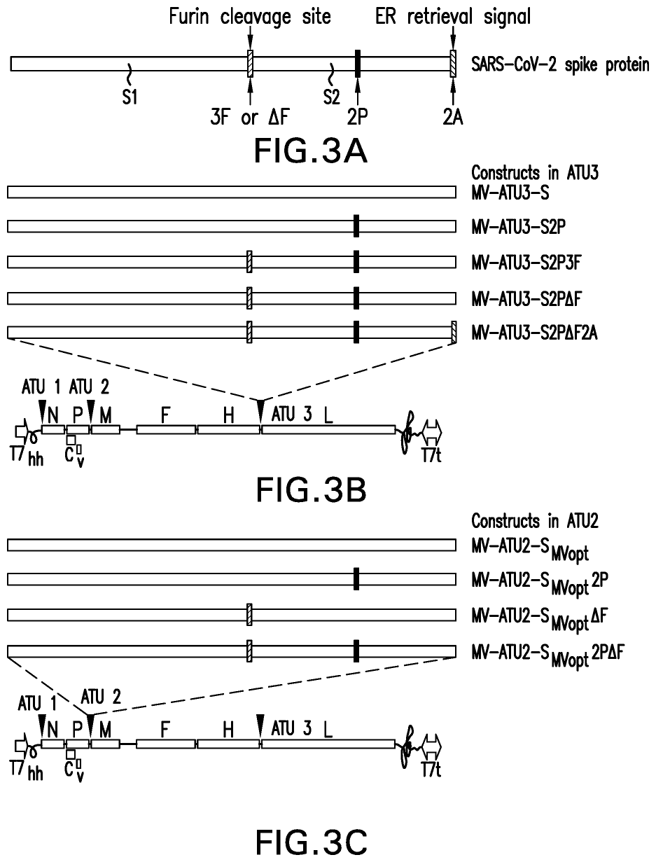

Starting from this plasmid, several mutations were introduced into the spike amino acid sequence (FIG. 2). The amino acid sequence was sequentially modified to lock the expressed protein in its prefusion state (2P mutation), prevent S1/S2 cleavage (furine cleavage site inactivation, either through 3F mutation or through ΔF deletion of the encompassing loop) and inactivate the Endoplasmic Reticulum retrieval signal (2A mutation). The resulting plasmids were named: pKM-ATU3-S_2019-nCOV (i.e. SARS-CoV-2), pKM-ATU3-stab S_2019-nCOV (i.e. SARS-CoV-2) (2P mutation), pKM-ATU3-S2P3F_2019-nCOV (i.e. SARS-CoV-2), pKM-ATU3-S2PΔF_2019-nCOV (i.e. SARS-CoV-2), pKM-ATU3-S2PΔF2A_2019-nCOV (i.e. SARS-CoV-2). In addition to the constructs shown in FIG. 3, pKM-ATU3-S3F_2019-nCOV was generated, lacking the 2P mutation.

Other constructs were designed to generate similar coding sequences at the ATU2 site. First, pKM-ATU2-S_2019-nCOV (i.e. SARS-CoV-2) of SEQ ID NO: 34 was generated by inserting the optimized SARS-CoV2 spike cDNA into BsiWI/BssHII-digested pKM-MVSchw-ATU2 which contains an additional transcription unit between the phosphoprotein and matrix genes of the Schwarz MV genome.

Furthermore, pKM-ATU2-S_{MVopt} was generated using a measles-optimized sequence (SEQ ID NO: 36) in an effort to fine-tune nucleotide composition and expression levels of the transgene and promote enhanced fitness and stability of the recombinant measles viruses. Starting from this plasmid, the 2P mutation, the deletion of the furin cleavage site (ΔF), and the combination of the two mutation/deletion (2PΔF) were introduced into the spike amino acid coding sequence (FIG. 3C) such as to generate similar insertions as in ATU3. The resulting plasmids were named: pKM-ATU2-S_{MVopt}, pKM-ATU2-S2P_{MVopt}, pKM-ATU2-SΔF_{MVopt}, and pKM-ATU2-S2PΔF_{MVopt}.

Seeding of HEK 293-T7-NP Cells

The helper cell line HEK 293-T7-NP (U.S. Pat. No. 8,586,364 patent) was freshly thawed and propagated. Upon reaching 90% confluency, the cells were harvested and resuspended in DMEM containing 10% FBS. Six-well plates were seeded with 2 mL cell suspension per well and incubated overnight at 37° C. and 5% CO₂.

Transfection with plasmids pKM-ATU3-S 2019-nCOV (i.e. SARS-CoV-2) of SEQ ID NO: 35, pKM-ATU2-S 2019-nCOV (i.e. SARS-CoV-2) of SEQ ID NO: 34, pKM-ATU2-Svopt or Derivatives and Heat Shock

At 50-70% confluency the medium was exchanged to fresh 2 mL DMEM+10% FCS and the plates were incubated for 4 h at 37° C. and 5% CO₂. Co-transfection of the pKM plasmid and the pEMC-La plasmid encoding Measles Schwarz polymerase was performed with calcium phosphate as previously described (Combredet et al., J Virol, 2003). The transfection mixture was added dropwise to the medium. The plates were shaken gently to distribute the transfection mix equally and incubated overnight at 37° C. and 5% CO₂.

The transfection medium was carefully replaced by 2 mL fresh DMEM+10% FCS. The transfected cells were incubated for 3 h at 37° C. followed by a 3-hour heat shock at 43.5° C. and 5% CO₂.

Incubation was subsequently continued at 37° C. for 2 days until confluence of the cell layer.

Co-Culture of HEK and VERO Cells

One day before co-cultivation, 48-well plaques were seeded with Vero cells at a concentration of 2×10⁴ cells/0.25 mL in DMEM+5% FCS. At day 3 post transfection i.e. day 0 of co-cultivation, the transfected HEK cells were gently resuspended in 2 mL of medium (contained in each well of the plates), diluted up to 24 mL and added to the Vero cells at 0.25 mL/well (co-culture). The co-culture plates were shaken gently for mixing and incubated at 37° C. and 5% CO₂. From day 3 of co-culture, the cells were observed daily for CPE and syncytium formation.

Harvest of Syncytia (Rescued Virus)

Wells of the co-culture plates showing single foci of CPE or syncytia were rinsed with PBS and harvested by trypsination (200 μl of trypsine/EDTA). Together with co-cultivation in 48-well plaques, this ensured clonality of the rescued viruses. After 2-5 minutes incubation at 37° C., the cells were transferred into a single well of a 6-well plates in a final volume of 2.5 mL of DMEM+5% FCS. Multiple wells containing single syncytia from cells transfected with the different plasmids were harvested and transferred to new 6-well plates. The 6-well plates were incubated at 37° C. and 5% CO₂ and observed daily for CPE or syncytia formation.

The rescued viruses were named:

| | | | Rescue recombinant |
|---|---|---|---|
| Plasmid | CNCM | SEQ ID NO | measles virus |
| pKM-ATU3-S_2019-nCOV | I-5497 | 35 | MV-ATU3-S |
| (i.e. SARS-CoV-2) | | | |
| pKM-ATU3-stab-S_2019- | I-5536 | | MV-ATU3-S2P |
| nCOV (i.e. SARS-CoV-2) | | | |
| pKM-ATU3-S2P3F_2019- | I-5534 | | MV-ATU3-S2P3F |
| nCOV (i.e. SARS-CoV-2) | | | |
| pKM-ATU3-S2PΔF_2019- | I-5532 | | MV-ATU3-S2PΔ |
| nCOV (i.e. SARS-CoV-2) | | | |
| pKM-ATU3-S2PΔF2A_2019- | I-5533 | | MV-AUT3-S2PΔF2A |
| nCOV (i.e. SARS-CoV-2) | | | |
| pKM-ATU3-S3F_2019- | I-5535 | | MV-ATU3-S3F |
| nCOV (i.e. SARS-CoV-2) | | | |
| pKM-ATU2-S_{MVopt}_SARS- | | | MV-ATU2-S_{MVopt} |
| CoV-2 | | | |

Expansion of Rescued Virus

When syncytia/CPE reached 30-50% of the cell monolayer, the rescued viral clones were further expanded. The cell monolayers of these wells were harvested by trypsination and transferred to a 75 cm² flask together with 2-3×10⁶ of trypsinated Vero cells originating from a T75 flask grown to confluence and incubated at 37° C. and 5% CO₂.

Harvest of the Expanded Rescued Virus

One to two days after virus expansion, syncytia/CPE reached 70-90% of the Vero cell monolayer infected with each of the rescued viral clones. The cell monolayers were lysed in 1.5 mL of supernatants by freeze/thaw and centrifuged for 5 min at 2000 g at 4° C. for clarification. The resulting supernatants were designated as passage 0 (P0), aliquoted and stored at −80° C.

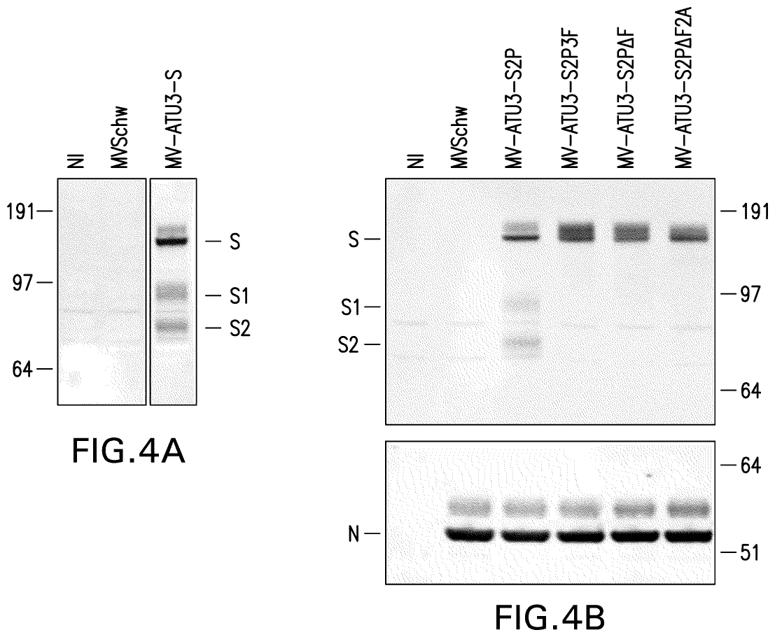

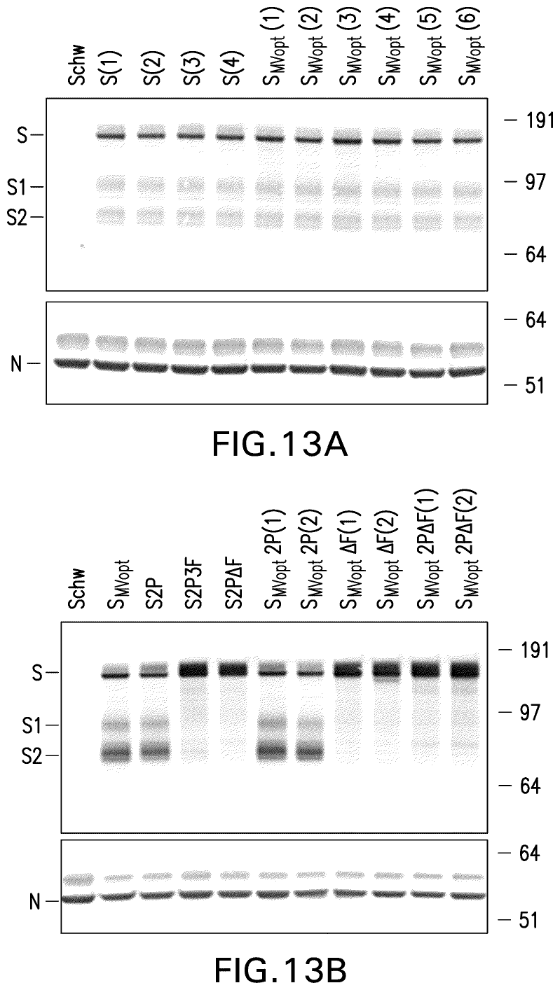

Antigen expression and sequence of the insert were checked by Western blot for P0 of each rescued clone (FIGS. 4 and 13) and Sanger sequencing respectively. Clones with good antigen expression and verified insert sequence were selected for further propagation.

Viral Clone Propagation in Cell Culture

The P0 seed of rescued recombinant MV clones were thawed. Virus propagation was carried out in Vero cells cultured in DMEM+5% FCS in T150 flasks, infected at a multiplicity (M.O.I) of 0.05 and incubated at 37° C.+5% CO₂. On day 2 to 3 post infection, when syncytia/CPE reached 80-95% of the Vero cell monolayer, virus was harvested by a freeze/thaw cycle of the cell monolayers scraped in 2.5 mL of supernatant. Cell debris were removed by centrifugation at 2000 g for 5 minutes at 4° C. (passage 1, P1). Antigen expression and sequence of the insert were checked for P1 of each selected clone. For further passaging and evaluation of genetic stability of the rescued viruses, these steps are repeated.

ELISA Assays Specific for MV and SARS-CoV-2 S

The induction of MV and SARS-CoV-2 specific antibodies in immunized mice was evaluated by indirect ELISA as described previously (Escriou et al., 2014). Microtiter plates were coated with purified measles antigen (Jena Bioscience) or recombinant trimerized SARS-CoV-2 spike ectodomain expressed in HEK 293T cells, respectively, and incubated with serial dilutions of the mouse sera. Bound antibodies were revealed with mouse-specific anti-IgG (gamma chain-specific) secondary antibody coupled to horseradish peroxidase (Southern Biotech) and TMB (KPL). ELISA IgG titers were calculated as the reciprocal of the highest dilution of individual serum giving an absorbance of 0.5.

SARS-CoV-2 Microneutralization Assay

Two or three-fold serial dilutions of heat-inactivated mouse serum samples in DMEM+1% BSA+10 mM Tricine were incubated at 37° C. for 2 hours with 20 TCID50 of SARS-CoV-2 and added to subconfluent monolayers of FRhK-4 cells plated in DMEM+5% FCS in a 96-well microtiter plates. Each serum dilution was tested in quadruplicate and cytopathic effect (CPE) endpoints were read up to 5 days after inoculation at 37° C.+5% CO₂. Neutralizing antibody titers were determined according to the Reed and Muench method (Reed and Muench, 1938) as the reciprocal of the highest dilution of serum, which prevented CPE in at least 2 out of 4 wells.

MV and SARS-CoV-2 ELISPOT Assay

Splenocytes from immunized or control mice were harvested, single cell suspensions were prepared and the frequency of MV and SARS-CoV-2-specific IFN-γ-producing T cells was quantified in a standard ELISPOT assay. Briefly, 96-wells Multi-screen PVDF plates (Millipore) were coated with 5 μg/ml rat anti-mouse IFN-γ antibodies (AN18, Becton-Dickinson) in PBS. Plates were washed and blocked with complete RPMI medium (RPMI 1640 supplemented with 10% FCS, 10 mM Hepes, 5×10-5 M β-mercaptoethanol, non-essential amino acids, Sodium Pyruvate, 100 U/ml penicillin and 100 μg/ml styreptomycin) for 2 h. Various numbers of splenocytes (typically 4×105, 2×105 and 1×105) from immunized and control mice were then plated in triplicate in the presence or absence of the appropriate peptide (1-10 μM) and IL2 (10 U/ml). The cells were incubated for 20 h at 37° C., and after extensive washes, the spots were revealed by successive incubations with biotinylated rat anti-mouse IFNγ antibodies (R46A2, Becton-Dickinson), alkaline phosphatase-conjugated streptavidin (Becton-Dickinson) and 5-bromo-4-chloro-3-indolylphosphate/nitroblue tetrazolium (NBT/BCIP, Sigma) as the substrate. The spots were counted using an automated ImmunoSpot® analyzer and associated Biospot 7.0 software (CTL). For each mouse, the number of peptide-specific IFNγ-producing cells was determined by calculating the difference between the number of spots generated in the presence of a negative control peptide and of the specific peptide. Results were expressed as the number of spot-forming cells (SFCs) per 10⁶ splenocytes.

In order to determine the responses against the SARS-CoV-2 spike protein, T cells were stimulated at a total concentration of 10 μM with peptide pools spanning the S1 and S2 domains, respectively, containing 15-mer peptides with 10 amino acid overlaps (Mimotopes).

These pools contained 135 and 118 peptides, respectively.

The measles H22-30 (RIVINREHL of SEQ ID NO: 67) and H446-454 (SNHNNVYWL of SEQ ID NO: 68) peptides and the NP366 (SCOT) peptide (ASNENMD™ of SEQ ID NO: 69) were synthesized by Eurogentec and used at a concentration of 1 μM each, as positive and negative control peptides, respectively.

Intracellular Cytokine Staining

Splenocytes from vaccinated or control mice were harvested and single cell suspensions were prepared. One million splenocytes were cultured in IMDM with Glutamax (ThermoFisher), 10% FBS, 1% penicillin-streptomycin, 10 IU/ml IL-2 (Miltenyi Biotec), 100 ng/ml IL-7 (Miltenyi Biotec). Cells were stimulated for 6 hours at 37° C. with peptide pools at a final concentration of 2 μg/ml per peptide (reconstituted in 5% DMSO). BD GolgiPlug and BD GolgiStop (BD Biosciences) were added for the final 4 hours. For positive controls cells were stimulated with 50 ng/ml PMA (Sigma Aldrich) and 1 μg/ml lonomycin (Sigma Aldrich), for negative controls medium with DMSO was used. Cells were incubated with mouse Fc receptor block (anti-mouse 2.4G2) and Fixable viability dye eFluor506 (eBioscience) to exclude dead cells. Cells were stained with α-CD45 BUV395 (BD Pharmingen #564279), α-CD19 AF700 (eBioscience #56-0193-80), α-CD11b AF700 (eBioscience #56-0112-82), α-CD11c AF700 (eBioscience #56-0114-82), α-CD3e BV650 (BD Pharmingen #564378), α-CD4 eFluor 450 (eBioscience #48-0041-80), α-CD8a PerCp Cy5.5 (BD Pharmingen #5511622), α-CD44 BV786 (BD Pharmingen #563736), α-CD62L APC-Cy7 (BioLegend #104427). Following fixation and permeabilization (BD Cytofix/Cytoperm, BD Biosciences) cells were stained intracellularly with α-TNFα FITC (eBioscience #11-7321-81), α-IFNγ PE-Cy7 (BD Pharmingen #55764), α-IL-5 PE (eBioscience #12-7052-82), α-IL-13 eFluor 660 (eBioscience #50-7133-82). Cells were acquired by flow cytometry using a Fortessa (BD Biosciences) and data was analyzed with FlowJo v10.7 software.

Challenge of IFNAR-KO Mice with SARS-CoV-2

Groups of six- to nine-week-old IFNα/βR−/− mice (IFNAR-KO) in a 129/Sv background permissive for measles vaccine were injected intraperitoneally (i.p.) with 10⁵ TCID50 of recombinant MVSchw/SARS-CoV-2-S or parental MVSchw. Booster injections were administered 4 weeks thereafter. Serum samples were collected at the indicated time points. To induce expression of the human ACE2 receptor, immunized mice were lightly anaesthetized with a Ketamine/Xylazine solution (50 mg/kg and 10 mg/kg, respectively) and administered intranasally with 6.0×10³ ICU of Ad5-hACE2 (adenovirus 5 expressing the human ACE2 receptor of SARS-CoV-2, Ku at al. 2021) in 30 μL PBS. Four days later, mice were anaesthetized as described above and inoculated intranasally with 2×10⁴ TCID50 (Tissue Culture Infectious Dose 50%) of the France/IDF0372/2020 SARS-CoV2 strain in 30 μL of PBS. Infected mice were euthanized at 4 days post-SARS-CoV-2 challenge by cervical dislocation. Halves of each lung lobes were removed aseptically, rinsed extensively in PBS and kept on ice until grinding in 750 μL of ice-cold PBS using a FastPrep®-24 homogenizer and Lysing Matrix M tubes containing a 6.35 mm diameter Zirconium Oxide ceramic grinding sphere (MP Biomedical) by two successive pulses at 4 m/s for 20s with incubation on ice between the two homogenization cycles. Homogenates were clarified by centrifugation for 10 min at 2000 g at 4° C. and kept at −80° C. in single-use aliquots.

SARS-CoV-2 genomic RNA loads in lungs were determined by extracting viral RNA from 70 μL of lung homogenate using QIAamp Viral RNA Mini Kit (Qiagen) according to the manufacturer's procedure. To remove putative non-particulate viral RNA in lung homogenates, 70 μL samples were treated with 100U RNaseI (Ambion) for 30 min at 37° C. prior to inactivation with the AVL buffer of the QIAamp Viral RNA Mini Kit. Viral loads (as expressed in genome equivalents (GEQ)/lung) were determined following reverse transcription and real-time TaqMan® PCR essentially as described by Corman et al. (Euro Surveill. 2020), using SuperScript™ Ill Platinum One-Step Quantitative RT-PCR System (Invitrogen) and primers and probe (Eurofins) targeting SARS-CoV-2 envelope (E) gene as listed in Table 7. In vitro transcribed RNA derived from plasmid pCI/SARS-CoV E was synthesized using T7 RiboMAX Express Large Scale RNA production system (Promega), then purified by phenol/chloroform extractions and two successive precipitations with ethanol. RNA concentration was determined by optical density measurement, then RNA was diluted to 10⁹ genome equivalents/μL in RNAse-free water containing 100 μg/mL tRNA carrier, and stored in single-use aliquots at −80° C. RNA quality was controlled on Agilent 2100 bioanalyzer_Eukaryote Total RNA Nano Series II. Serial dilutions of this in vitro transcribed RNA were prepared in RNAse-free water containing 10 μg/mL tRNA carrier and used to establish a standard curve in each assay. Thermal cycling conditions were as follows: (i) reverse transcription at 55° C. for 10 min, (ii) enzyme inactivation at 95° C. for 3 min, (iii) 45 cycles of denaturation/amplification at 95° C. for sec, 58° C. for 30 sec. Products were analyzed on an ABI 7500 Fast real-time PCR system (Applied Biosystems).

TABLE 7
Sequences of primers and probes for SARS-CoV-2
load determination.

| | | SEQ |
|---|---|---|
| Primer/Probe | | ID |
| Name | DNA Sequences | NO |
| E_Sarbeco_F1 | 5′- ACAGGTACGTTAATAGTTAATAGCGT -3′ | 70 |
| E_Sarbeco_R2 | 5′- ATATTGCAGCAGTACGCACACA -3′ | 71 |
| E_Sarbeco_P1 | 5′- FAM-ACACTAGCCATCCTTACTGCGCTTCG- | 72 |
| | BHQ-1 -3′ | |

Infectious SARS-CoV-2 titers in lung homogenates were determined in Vero-E6 cells seeded in 12-well plates. Cells in duplicate wells were infected with each 5-fold serial dilution of lung homogenates in DMEM medium containing 10 mM tricine and 1 mg/mL BSA. Following 1 h incubation at 37° C. under 5% CO₂ atmosphere, viral inoculum was withdrawn and cells were placed in medium containing 1 μg/mL trypsin and 1.2% Avicel RC581 (FMC Biopolymer), then further incubated at 37° C. for 72 h. Cell sheets were then fixed with formaldehyde and stained with 0.1% crystal violet and plaques were counted. Infectious viral titers were expressed as plaque forming units (PFU)/lung.

2. Results and Discussion

Rescue and Characterization of Recombinant MV in Cell Culture

Growth Characteristics

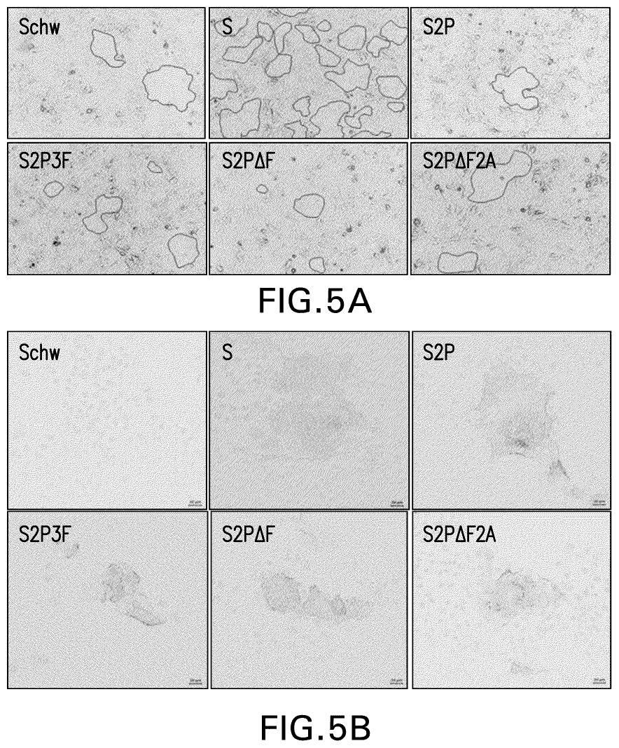

Recombinant MV expressing SARS-CoV-2 S from ATU3 was successfully rescued. MV-ATU3-S was propagated in Vero NK cells and grew to high titers of above 10⁷ tissue culture infectious dose 50 (TCID₅₀). Genetic stability was confirmed by Sanger sequencing of the ATU and NGS sequencing of the full-length genome of independent viral clones retrieved following transfection. Efficient S expression was demonstrated in cells infected with each clone of MV-ATU3-S as shown in FIG. 4A for a representative clone. In time-course experiments, the MV-ATU3-S virus reached the peak titer after 48 h and then declined by about 1 log titer, in contrast to MV-Schw which showed stable virus titers between 48 and 72 h. Upon infection of cells, measles virus typically induces the formation of multinucleated cells (syncytia, FIG. 5) as a result of the interaction between the virus fusion (F) and attachment (H) glycoproteins and the host cell plasma membrane. Ultimately, the first syncytia fuse progressively together, resulting in wide areas or fused cells. In cells infected with MV-ATU3-S an increased number of such large syncytia was observed at late times after infection (FIG. 5). This fusogenic phenotype of MV-ATU3-S was observed from early on after infection (not shown) and the greater extension of fused area coincided with the peak of viral titer at 48 h post-infection. At this timepoint, the large syncytia collapsed and the infected cell monolayer displayed extensive cytopathogenic effect (CPE), thus impacting viral replication of MV-ATU3-S compared to MV-Schw. The fusogenic phenotype suggested that in cells infected with MV-ATU3-S the native spike expressed on the cell surface was functional in the sense that it could interact with the ACE2 receptor on the neighboring cells and promote cell fusion. For generating a vaccine candidate, an enhanced fusogenic phenotype, such as that observed for MV-ATU3-S, was considered a potential safety risk as it might change the tropism of the measles vector virus. Thus, spike protein variants with reduced or abolished fusogenic properties were designed through two complementary approach, based on either stabilizing the spike in its prefusion state trough the “2P” mutations (K986P+V987P) or preventing cleavage at the S1/S2 junction through the “3F” mutations (R682G+R683S+R685G) of the multibasic cleavage site or “ΔF” deletion of the encompassing Q675-R685 loop (FIG. 2). Modified spike genes expressing the variant spike proteins with the aforementioned modifications and combinations thereof were generated and first evaluated for their ability to promote cell fusion after transient transfection in the presence of hACE2 expression in an assay based on cells harboring the split-GFP system (Buchrieser et al., 2020). Data showed that spike protein harboring either the “2P” mutation alone, the “3F” mutation of the multibasic S1/S2 furin cleavage site alone, or combinations of “2P” and “3F”, or “2P” and the deletion of the furin cleavage site “ΔF” (FIG. 2) did not induce fusion of hACE2-expressing HEK 293T cells in contrast to the native spike (FIG. 16).

As suggested by these results, locking the S protein in its pre-fusion state by introducing the “2P” mutation (FIG. 2) abolished the enhanced fusogenic phenotype of any recombinant measles vector viruses expressing such stabilized spike variants. Recombinant MV-ATU3-S2P, MV-ATU3-S2P3F, MV-ATU3-S2PΔF, and MV-ATU3-S2PΔF2A were indeed successfully rescued and all grew to high titers of above 10 TCID₅₀. Infection of Vero NK cells with MV-AUT3-S2P, MV-ATU3-S2P3F, MV-ATU3-S2PΔF, and MV-ATU3-S2PΔF2A resulted in similar syncytia formation as infection with the parental MV Schwarz (FIG. 5), confirming absence of enhanced fusion activity as compared to MV-ATU3-S expressing native S. Genetic stability of all 4 constructs harboring the 2P stabilizing mutation was confirmed after 5 passages in cell culture by Sanger sequencing of the ATU and NGS sequencing of the full-length viral genome.

Recombinant MV clones expressing native SARS-CoV-2 S from ATU2 grew to much lower titers than parental Measles Schwarz or developed nonsense compensating mutations in the spike transgene soon after rescue (P0) or after subsequent passage 1 (P1) that restored high titer growth. This indicates that the fully codon-optimized S gene generates unstable MV vector when inserted into position ATU2. A possible explanation for this may be linked to increased S expression levels driven from the upstream ATU2 position than from the downstream ATU3 position, in relation to the gradient of gene expression generated by MV replication (Plumet, 2005). Thus, measles vector constructs expressing native and variant spike proteins from the measles-optimized nucleotide sequence inserted in the ATU2 position (FIG. 3C) were generated. These constructs, MV-ATU2-S_{MVopt}, MV-ATU2-S_{MVopt}2P, MV-ATU2-S_{MVopt}ΔF, MV-ATU2-S_{MVopt}2PΔF grew to high titers above 10⁷ TCID₅₀. The correct sequence of the insert was confirmed by Sanger sequencing of the ATU, indicating that the measles-optimized S gene remarkably generates stable MV vectors when inserted into position ATU2.

Antigen Expression

Spike expression in recombinant MV-infected Vero cells was confirmed by Western Blot analysis using polyclonal rabbit antisera raised against recombinant S protein of SARS-CoV-1 (Escriou et al., Virology, 2014) or SARS-CoV-2. As shown in FIG. 4, a major band was detected for all samples with the expected apparent molecular mass of 180 kDa, indicating expression of full length S protein. Minor bands with apparent molecular masses of 100 and 80 kDa respectively were detected for lysates prepared from cells infected with MV-ATU3-S and MV-ATU3-S2P, demonstrating that the S was partially cleaved into its S1 and S2 domains. Inactivation of the furin cleavage site either by mutation (MV-ATU3-S2P3F) or deletion of the small encompassing loop (MV-ATU3-S2PΔF, MV-ATU3-S2PΔF2A) abolished the cleavage as expected (FIG. 4B). In addition, expression of spike polypeptide was readily detected by indirect immunofluorescence (IFA) of non-permeabilized syncytia, indicating that any of the native and mutated S was efficiently transported to the surface (FIG. 5B).

Insertion of the measles-optimized nucleotide sequence (SEQ ID NO: 36) in ATU2 resulted in similar expression levels as the insertion of the fully codon-optimized nucleotides sequence in ATU3 (FIG. 13A). Equivalently to the spike protein expressed from the fully codon-optimized nucleotide sequence in ATU3, S_{MVopt}2P protein expressed at ATU2 was partially cleaved into S1 and S2, whereas the spike protein with furin site deletion expressed by MV-ATU2-S_{MVopt}ΔF and MV-ATU2-S_{MVopt}2PΔF was not cleaved (FIG. 13B).

Immunogenicity

The immunogenicity of COVID-19 vaccine was evaluated in 129sv Type 1 interferon receptor (Interferon-α/β receptor, IFNAR)-deficient mice, a small animal model suitable for the assessment of measles-vectored vaccines. All experiments were approved and conducted in accordance with the guidelines of the Office of Laboratory Animal Care at the Institut Pasteur, Paris. IFNAR KO mice were housed under specific pathogen-free conditions at the Institut Pasteur animal facility. Groups of six 6-10 week-old IFNAR KO mice were immunized with two intraperitoneal injections at 3 to 4-week interval of 1×10⁵ TCID50 of the vaccine candidates or immunized only once. As a control, a group of mice injected with empty MV vector MV-Schwarz (1×10⁵ TCID50) was included in each study. Mouse sera were collected 18-21 days after the first and second injection.

Humoral Responses

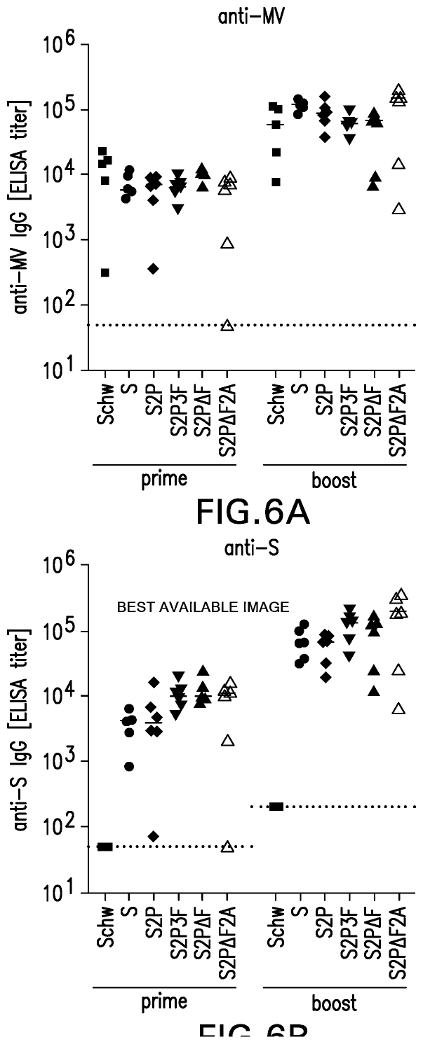

To assess the humoral responses, SARS-CoV-2 and MV-specific antibody responses were evaluated for each individual mouse by indirect ELISA. Mice immunized with the different recombinant measles viruses expressing SARS-CoV-2 spike seroconverted to measles virus after prime immunization as determined by measles-specific ELISA (FIG. 6A). Anti-measles responses were similar for all tested MV constructs and parental MV-Schwarz, suggesting that expression of the heterologous S protein by the recombinant viruses did not alter their replication in vivo nor modify their measles-specific immunogenicity. Some mice did not respond or responded poorly to the prime immunization but did respond after boost immunization. After boost, the anti-MV antibody levels increased by about 10-fold as expected from previous studies in mice.

All mice immunized with MV-ATU3-S, MV-ATU3-S2P, MV-ATU3-S2P3F, MV-ATU3-S2PΔF, and MV-ATU3-S2PΔF2A that responded with anti-measles antibodies also responded with high titers of spike-specific antibodies, in the 10⁴ range, after prime as measured by an ELISA specific for the ectodomain of the SARS-CoV-2 spike protein (FIG. 6B). After boost, the anti-S antibody levels increased by about 10-fold as seen for anti-MV antibodies. In contrast, control mice immunized with empty MV-Schwarz all tested negative. While not statistically significant, there was a slight trend of higher anti-S antibody levels elicited by constructs with inactivated furin cleavage site (MV-ATU3-S2P3F, MV-ATU3-S2PΔF, and MV-ATU3-S2PΔF2A) compared to those elicited by MV-AUT3-S and MV-ATU3-S2P.

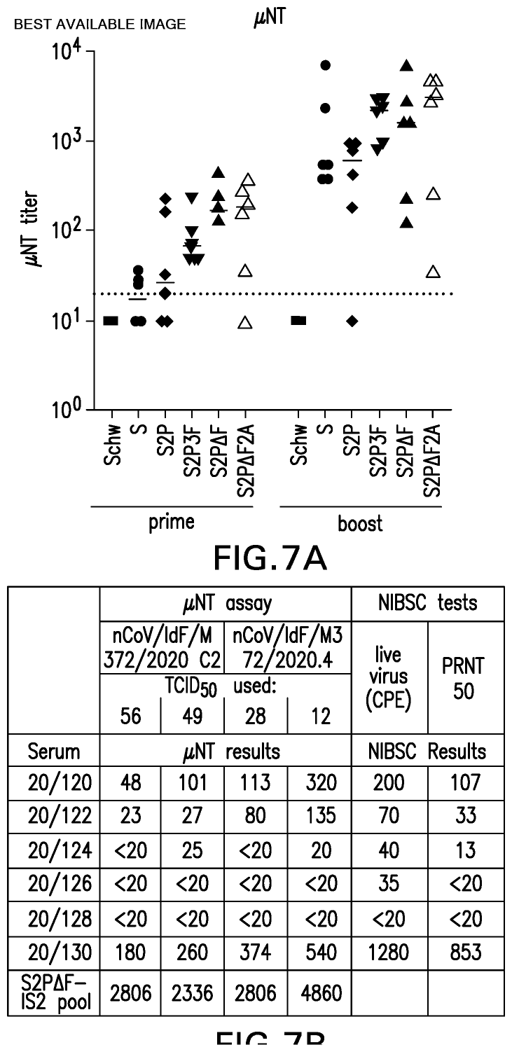

Anti-SARS-CoV-2 neutralizing antibodies were detected using a microneutralization assay. All recombinant MV expressing SARS-CoV-2 S elicited neutralizing antibodies after one immunization (FIG. 7). As shown in FIG. 7, and repeated in additional experiments (data not shown) there was a trend of increasing neutralizing titers after prime in mice immunized with the following constructs: MV-ATU3-S<MV-ATU3-S2P<MV-ATU3-S2P3F<MV-ATU3-S2PΔF/MV-ATU3-S2PΔF2A. After the boost, neutralizing titers increased by at least 10-fold. All recombinant MV expressing SARS-CoV-2 S with inactivated furin cleavage site elicit similar neutralizing antibody levels after boost and higher levels than MV-ATU3-S and MV-ATU3-S2P. The difference between the neutralizing titers after boost of MV-ATU3-S2P and MV-ATU3-S2P3F is statistically significant (p=0.0216 Mann-Whitney test). This was confirmed by plaque reduction neutralization test 90 (PRNT90).

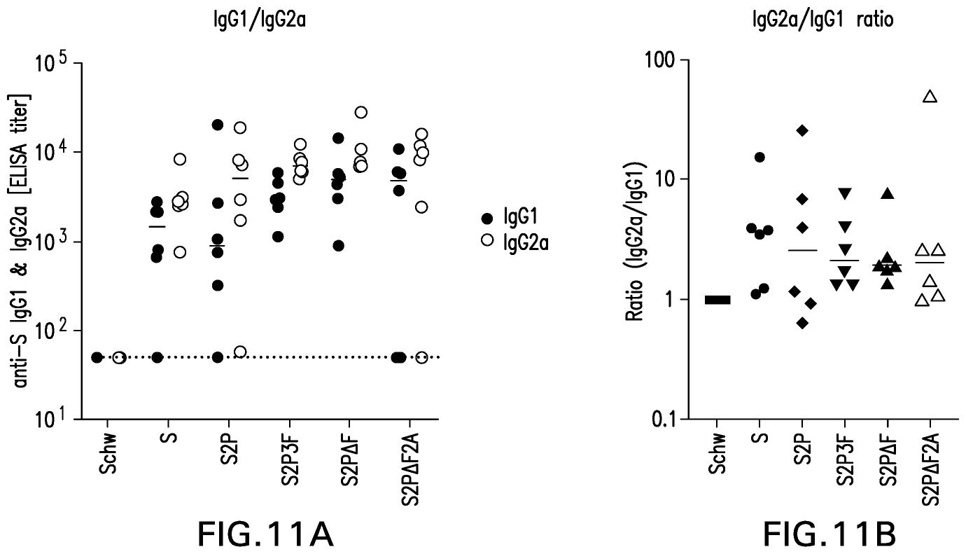

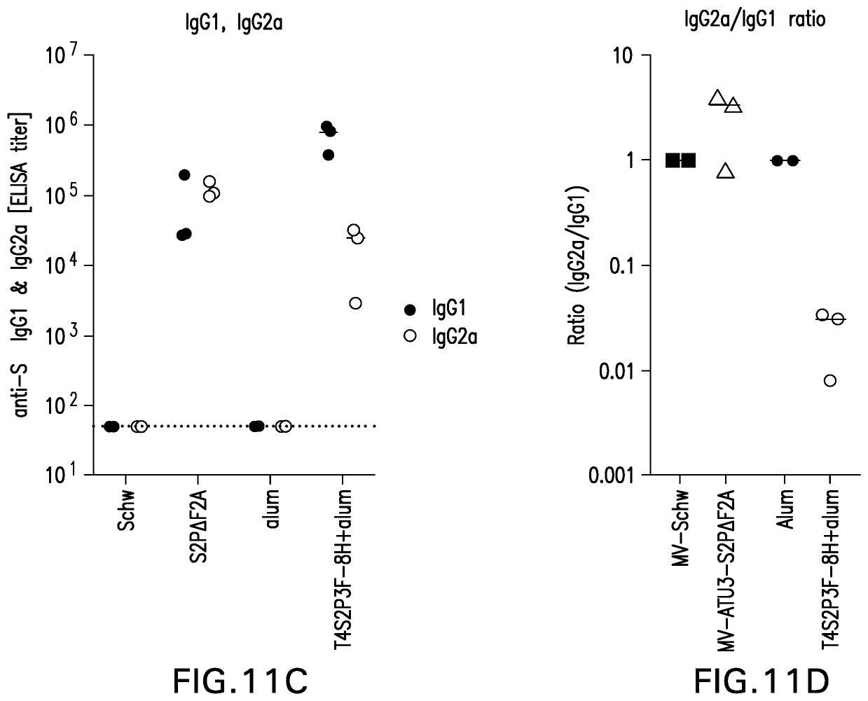

Based on the recommendation of the International Coalition of Medicinal Regulatory Agencies (ICMRA) on Mar. 18, 2020, it is important to ensure induction of a Th1 response to mitigate the risk of potential disease enhancement that was observed in animal models with SARS-CoV-1 vaccine candidates. As IgG isotype analysis allows obtaining a first indication of skewing of the ongoing CD4⁺ helper T cell response into a type 1 (Th1) or type 2 (Th2) response, the ratio of IgG2a to IgG1 antibody titers was measured by isotype-specific anti-S ELISA. As shown in FIGS. 11A and 11B, this analysis revealed that MV-ATU3-S, MV-ATU3-S2P, MV-ATU3-S2P3F, MV-ATU3-S2PΔF, and MV-ATU3-S2PΔF2A all induced higher IgG2a than IgG1 levels indicative of a Th1 response. Control experiments were performed by immunizing wt 129/Sv mice with alum-adjuvanted trimerized spike ectodomain. After prime and boost these mice had much higher IgG1 than IgG2a antibody titers (FIGS. 11C and 11D), indicating that the induced immune responses were predominantly of Th2-type as we previously observed after immunization with alum-adjuvanted SARS-CoV-1 spike ectodomain (Escriou, 2014).

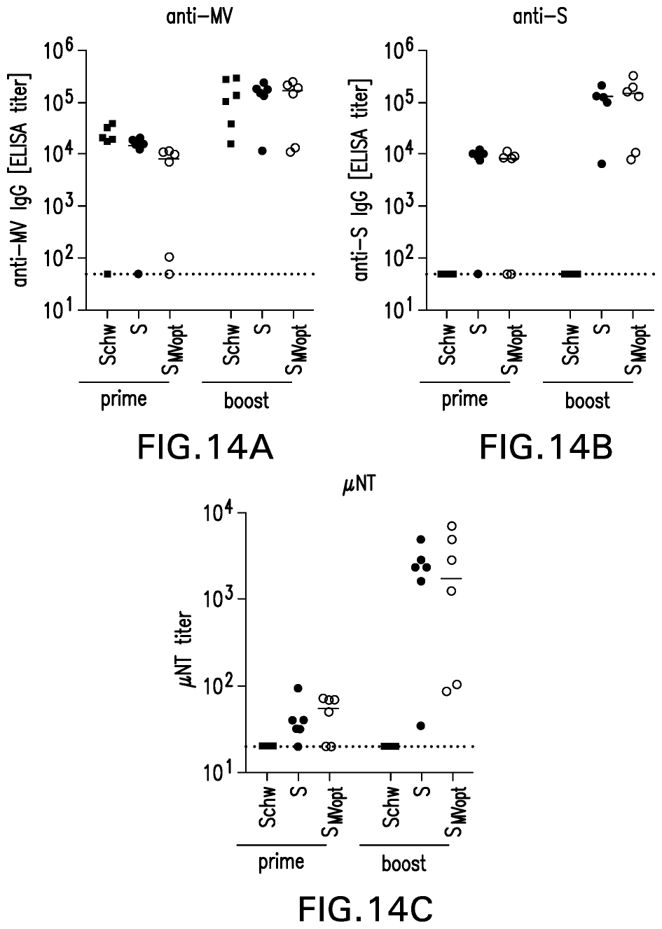

A comparison of the immunogenicity of MV-ATU3-S and MV-ATU2-S_{MVopt} as assessed by anti-MV ELISA, anti-S ELISA and SARS-CoV-2 microneutralization showed no differences in the elicited antibody responses by the two measles vector viruses after the first or after the second immunization (FIG. 14).

T Cell Responses

Splenic T cell responses were analyzed to directly assess the type of T helper cell responses as well as cytotoxic T cell responses. S protein-specific T cell responses were measured in splenocytes 19-25 days after boost immunization. T cells were stimulated with peptide pools spanning the full length of the S1 and S2 domains of the spike protein, respectively, containing 15-mer peptides with 10 amino acid overlaps. As control, measles-specific responses were assessed using a pool of 2 measles peptides specific for CD8⁺ T cells (Reuter et al, PLoS ONE, 2012, 7(3), e33989, I-8).

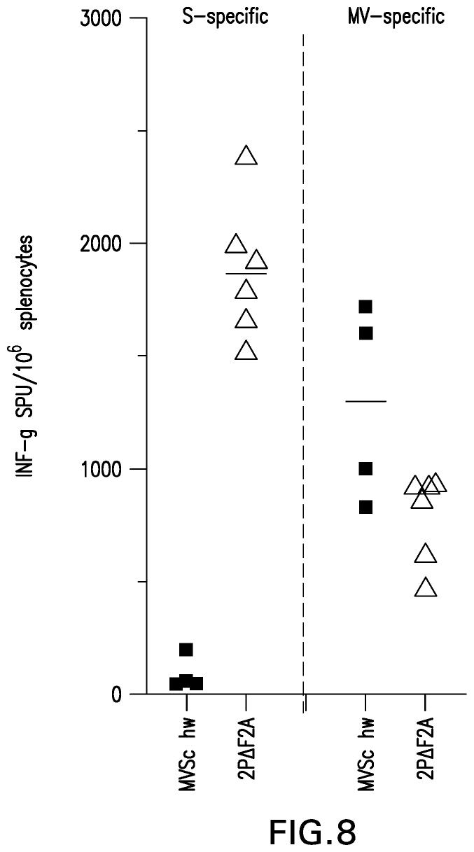

IFN-γ producing T cells were enumerated by ELISpot in mice immunized with MV-ATU3-S2PΔF2A and demonstrated cellular responses against peptides spanning the S protein (FIG. 8). Cellular responses to the measles vector backbone were also confirmed (FIG. 8).

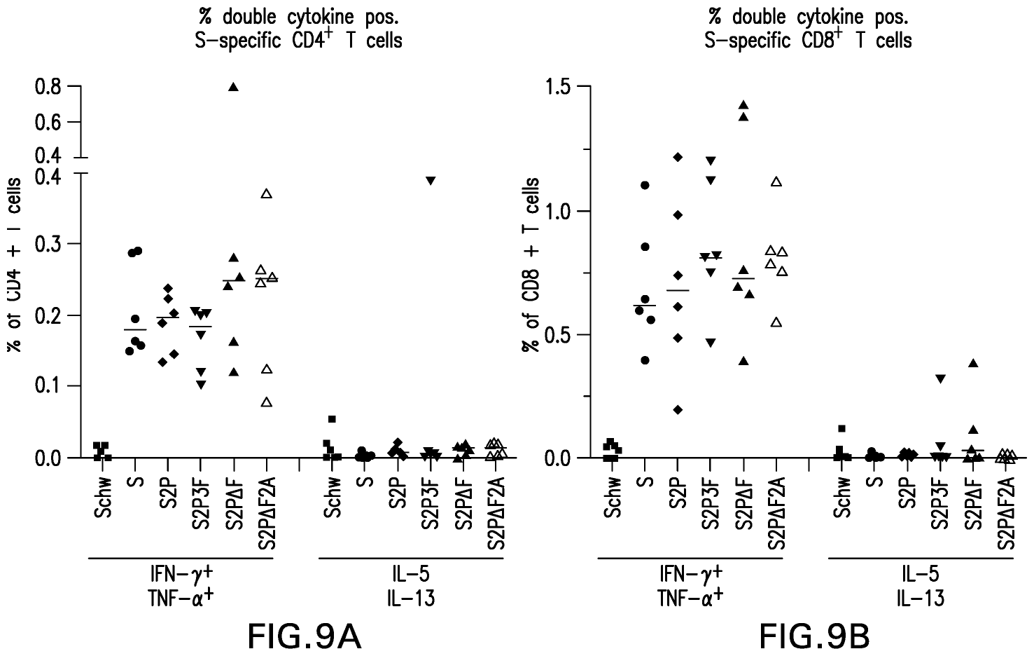

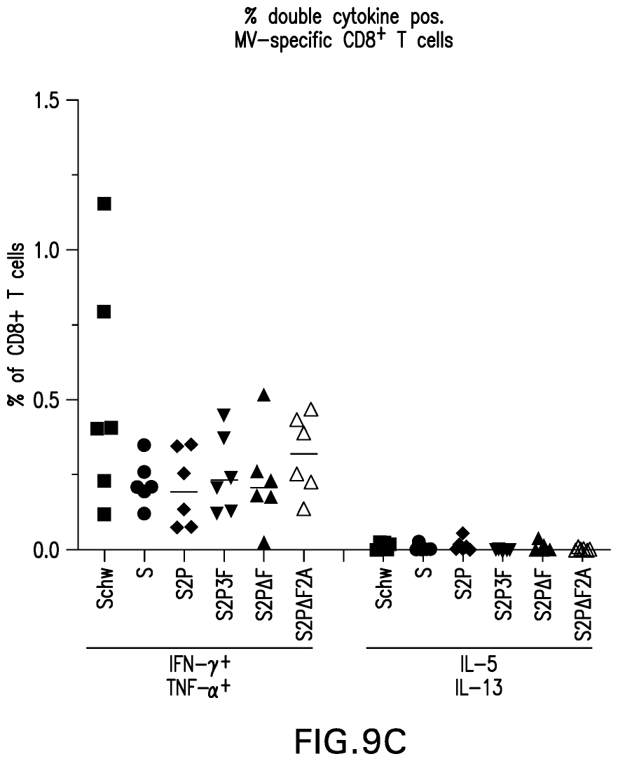

Cytokine production by CD4⁺ T cells and CD8⁺ T cells was characterized by intracellular cytokine staining analyzed by flow cytometry. Mice were immunized with MV-ATU3-S, MV-AUT3-S2P, MV-AUT3-S2P3F, MV-ATU3-S2PΔF, MV-ATU3-S2PΔF2A or with the parental MV Schwarz strain. In all mice immunized with recombinant MV expressing SARS-CoV-2 spike protein, intracellular cytokine staining of splenocytes detected IFN-γ and TNF-α double positive CD8⁺ and CD4⁺ T cells in response to S1 and S2 peptide pools spanning the whole S protein. This indicated a functional status of these T cells and confirmed that S-specific Th1-type responses were induced by the vaccine candidates (FIG. 9, Panels A and B). In addition, a CD8+ T cell response to the measles vector backbone was confirmed by demonstrating IFN-γ and TNF-α double positive CD8+ T cells in mice receiving the recombinant candidates as well as the parental MVSchw (FIG. 9, Panel A and C). In contrast, only a marginal fraction of S-specific T cells expressing IL-5 and IL-13, characteristic for Th2 responses, was detected in mice receiving any of the recombinant candidates (FIG. 9, Panels A and B).

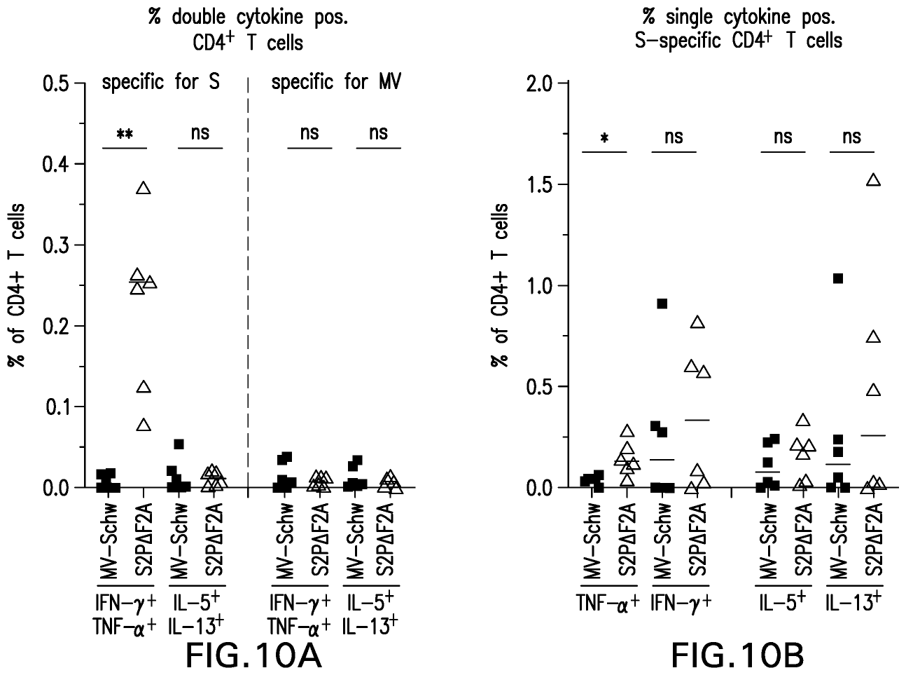

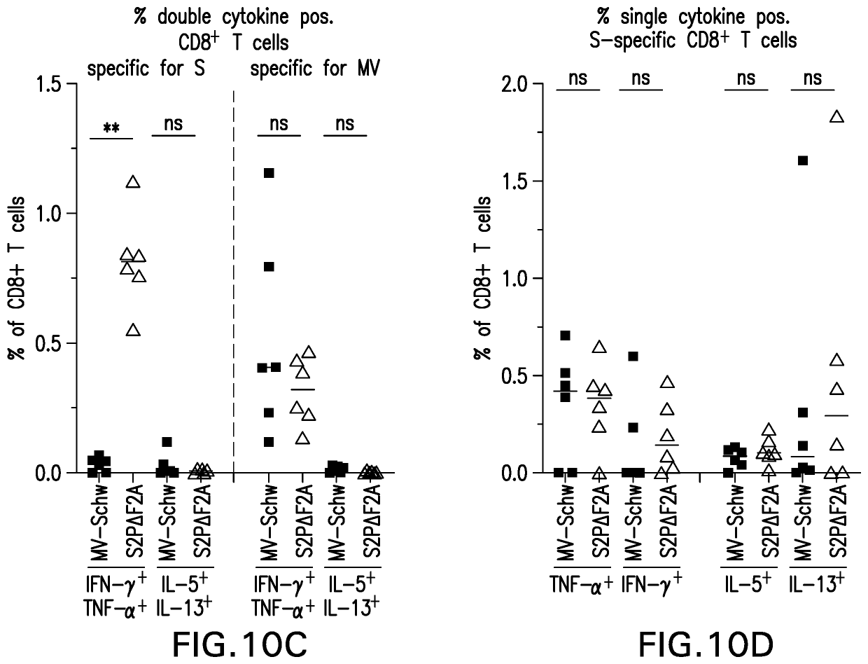

In order to characterize the T cell response in more detail, T cells producing a single cytokine in response to S peptide pools (FIG. 10, Panels B and D) were assessed in mice immunized with MV-ATU3-S2PΔF2A and the parental MV Schwarz in addition to double cytokine-positive T cells (FIG. 10, Panels A and C, same results as also included in FIG. 9). Only TNF-α producing CD4⁺ T cells were present in higher frequency in mice immunized with MV-ATU3-S2PΔF2A than in mice immunized with MVSchw control (FIG. 10, Panel B). All other CD4⁺ and CD8⁺ single cytokine producing T cells were found with similar frequency in MV-ATU3-S2PΔF2A and MWSchw immunized mice (FIG. 9, Panels B and D), indicating that these responses were not specific to the spike protein.

Taken together, these results indicated that a functional Th1-type T cell response targeting epitopes of the S protein is induced by immunization with recombinant MV expressing SARS-CoV-2 spike protein, including MV-ATU3-S, MV-AUT3-S2P, MV-AUT3-S2P3F, MV-ATU3-S2PΔF and MV-ATU3-S2PΔF2A. This response is characterized by the predominant induction of IFN-γ and TNF-α producing CD8⁺ T cells as well as IFN-γ and TNF-α producing CD4⁺ T cells.

Protection Against SARS-CoV-2 Challenge

To assess the potential of the measles vector constructs to induce protection against experimental challenge infection with SARS-CoV-2, the inventors used the model of transient expression of human ACE2 receptor in the lungs of IFNAR-KO mice to allow subsequent infection with SARS-CoV-2 (Ku et al, 2021).

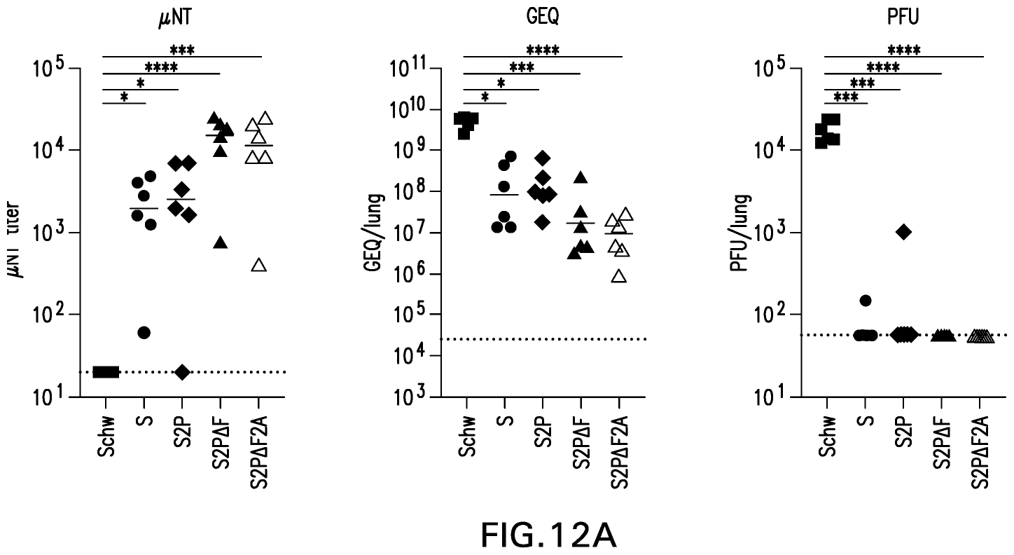

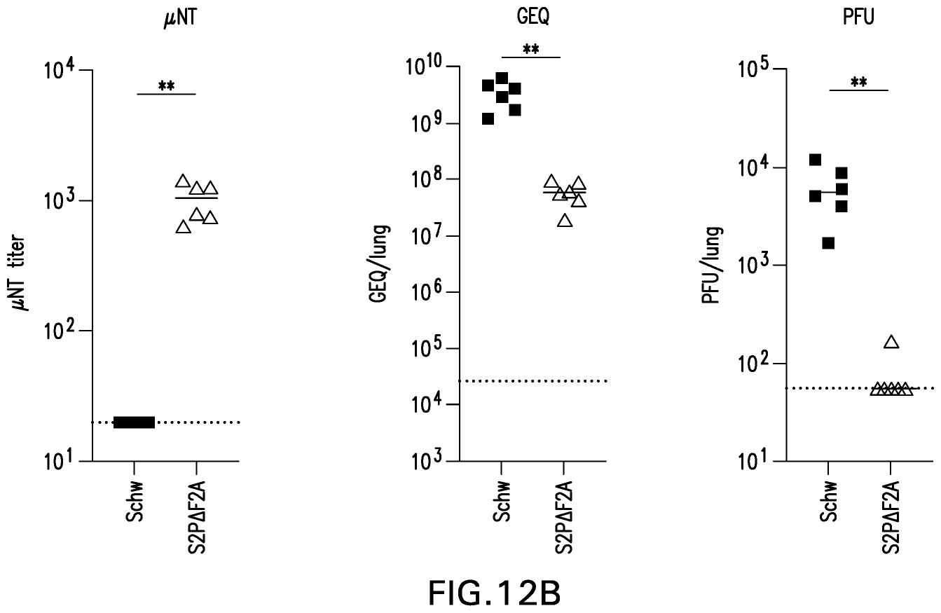

To assess protection after prime and boost immunization, mice were immunized with either MV-ATU3-S, MV-ATU3-S2P, MV-ATU3-S2PΔF, MV-ATU3-S2PΔF2A, or the parental MV Schwarz strain as control. Mice were instilled with AD5:hACE2 25 days after the second immunization and inoculated intranasally with 2×10⁴ pfu of SARS-CoV-2 four days later. The presence of SARS-CoV-2 virus in the lungs was assessed by RT-qPCR measuring genome equivalents (GEQ) RNA levels as well as by quantification of infectious virus in Vero cells. As shown in FIG. 12, Panel A, all measles vector constructs significantly reduced the viral load in the lungs compared to MVSchw parental virus. GEQ were reduced by approximately 1.5 logs by MV-ATU3-S, MV-ATU3-S2P and 2.5 logs by MV-ATU3-S2PΔF and MV-ATU3-S2PΔF2A. In addition, infectious virus was only detected at low residual titers in one mouse each in the groups immunized with MV-ATU3-S and MV-ATU3-S2P, specifically in the mice that responded poorly to the immunization and had very low neutralizing antibody titers, and not in any of the mice immunized with MV-ATU3-S2PΔF and MV-ATU3-S2PΔF2A. Altogether these results showed that vaccination with the recombinant MV significantly reduced the viral load in the lungs of the animals and particularly that the presence of infectious virus was prevented. Noteworthy, the efficiency of protection followed the hierarchy observed for the induction of neutralization antibody levels, MV-ATU3-S2PΔF and MV-ATU3-S2PΔF conferring better protection than MV-ATU3-S and MV-ATU3-S2P.

Noticeably, the level of neutralizing antibody titer in blood samples taken 9 days before the challenge inversely correlated with the efficiency of protection in individual mice (whatever immunogen) as assessed by levels of GEQ (p=0.0149) and PFU (p=0.0253). This suggests that neutralizing antibody levels in the blood contribute directly or indirectly to the reduction of SARS-CoV-2 replication after infection.

Based on the promising results after prime and boost immunization, protection after prime only was assessed in mice immunized with MV-ATU3-S2PΔF2A. This experiment was also designed to investigate the longevity of the antibody response up to 165 days after prime only immunization. Microneutralization titers measured at ˜6 months following prime (FIG. 12, Panel B, μNT) were high, in the range of 10³, yet about 10-fold below the levels reached after boost immunization with this vector (FIG. 12, Panel A, μNT). The challenge and viral load analysis were performed as described above. Significant reduction of GEQ/lung by 2 logs was observed (FIG. 12, Panel B). Infectious SARS-CoV-2 virus was only recovered at very low titer from the lungs of one out of six mice immunized with MV-ATU3-S2PΔF2A, demonstrating strong protection after prime immunization only with MV-ATU3-S2PΔF2A.

Protection after prime only was also assessed in mice immunized with MV-ATU3-S2PΔF2A in comparison to mice immunized with MV-ATU3-S2P and to mice immunized with empty vector MV-Schwarz, as a control. This experiment was designed to investigate short-term protection 4 weeks only after prime immunization.

As already shown in an experiment depicted in FIG. 6B, all mice immunized with MV-ATU3-S2P and MV-ATU3-S2PΔF2A responded with high titers of measles-specific and spike-specific antibodies, in the 10⁴ range, as measured by ELISA (FIG. 17, panel A). Significantly higher neutralizing antibody levels (p=0.0087 Mann-Whitney) were elicited by the MV-ATU3-S2PΔF2A construct with inactivated furin cleavage site [2.6±0.1 log 10(NT titers)] compared to those elicited by MV-ATU3-S2P with 2P-stabilized S [2.1±0.2 log 10(NT titers)].

Protection from challenge with SARS-CoV-2 was assessed as described above. As shown in FIG. 17, Panel B, immunization with both measles vector constructs significantly reduced the viral load in the lungs compared to MVSchw parental virus. GEQ RNA levels were reduced by approximately 0.8 log by MV-ATU3-S2P and 1.3 log by MV-ATU3-S2PΔF2A. In addition, infectious virus was only detected at low residual titers in half of 6 mice immunized with MV-ATU3-S2P, specifically in mice that had the lowest neutralizing antibody titers. No infectious virus was detected in mice immunized with MV-ATU3-S2PΔF2A.

Altogether, this experiment showed that vaccination with recombinant MVs significantly reduced the viral load in the lungs of animals as early as 4 weeks after prime immunization. It also confirms that the efficiency of protection follows the hierarchy observed for the induction of neutralization antibody levels, i.e. MV-ATU3-S2PΔF2A confers better protection after prime than MV-ATU3-S2P.

Enhanced Immunogenicity and Protective Potential of Recombinant MV Schwarz Viruses Expressing SARS-CoV-2 S6P, 6P3F and 6PΔF Protein.

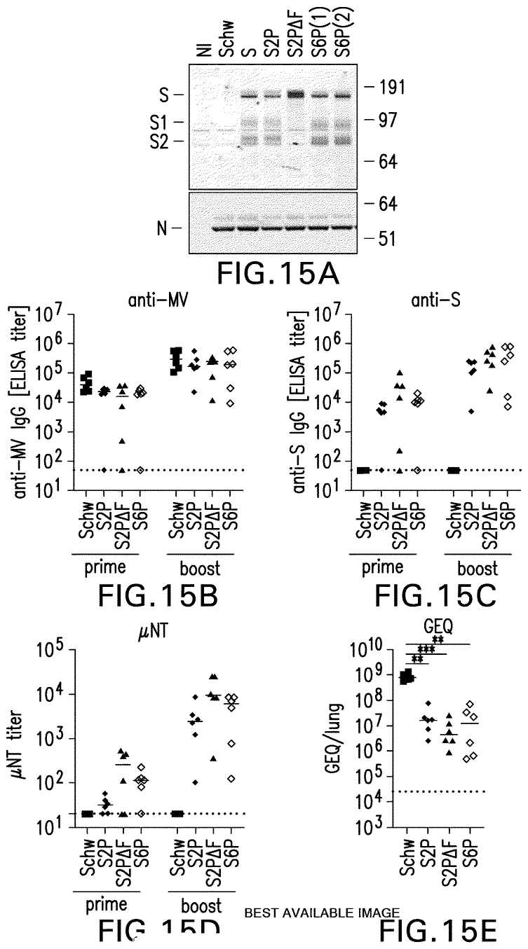

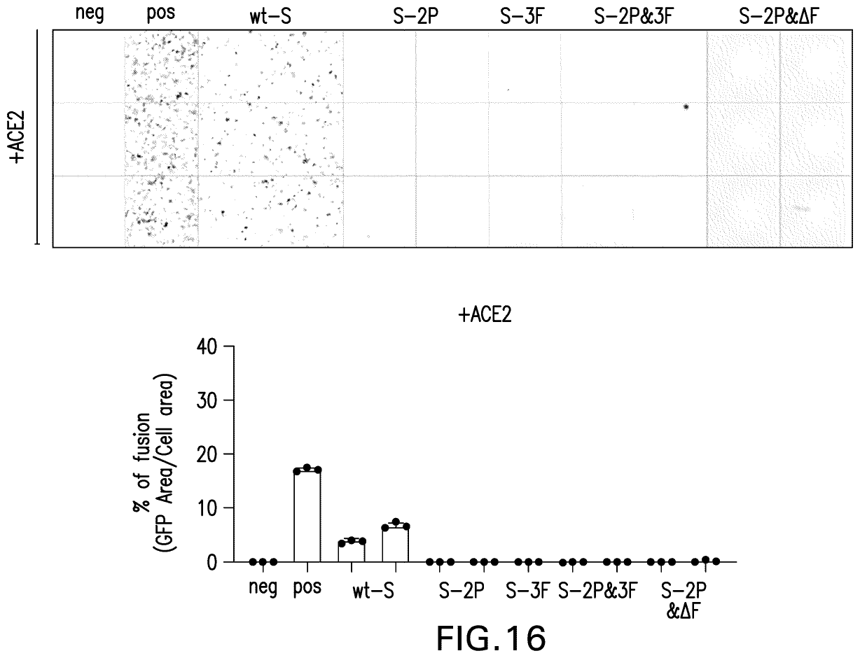

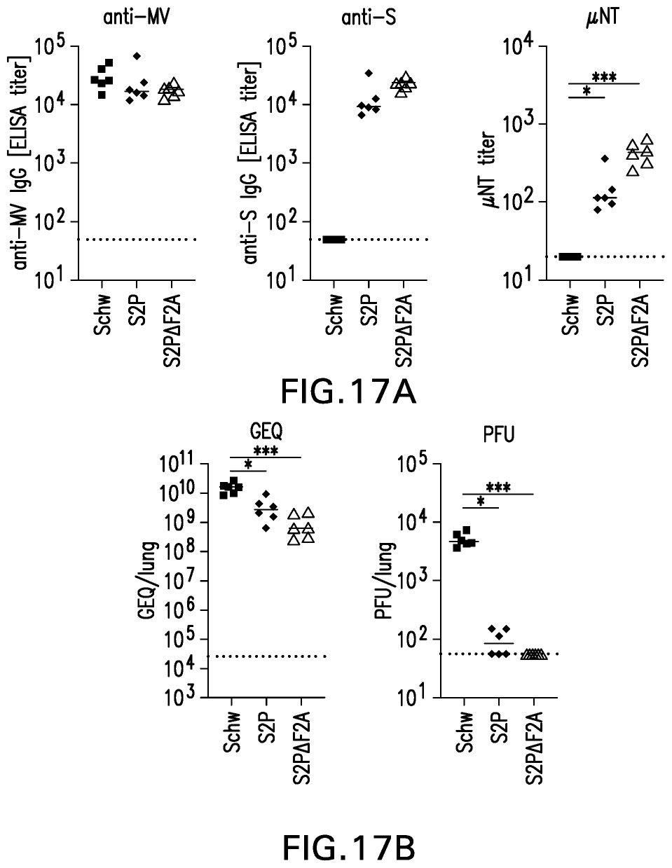

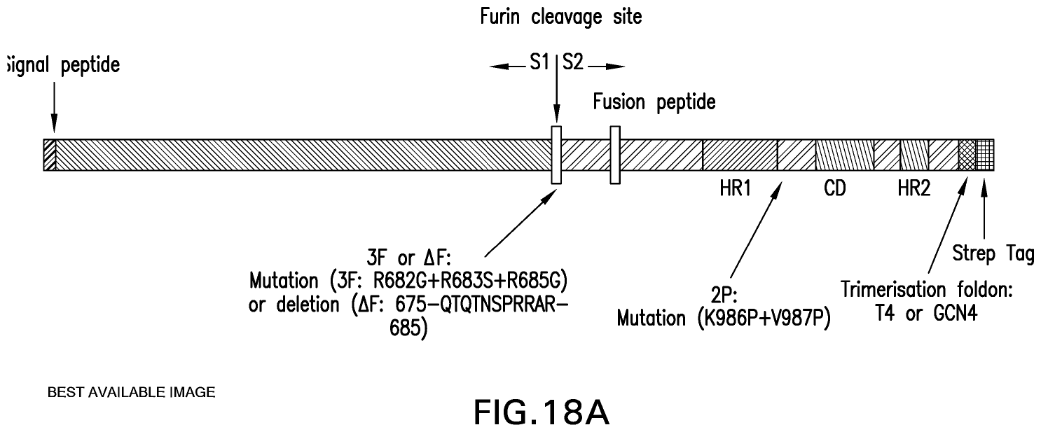

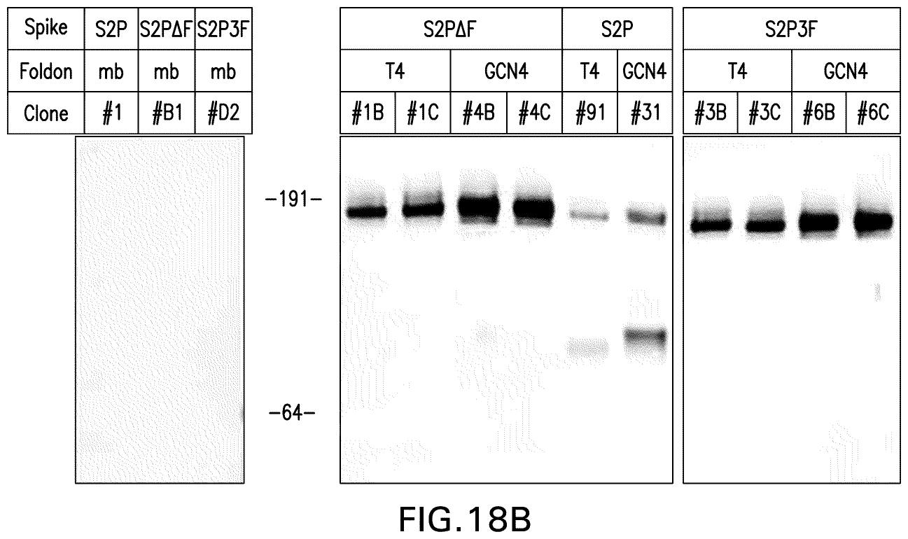

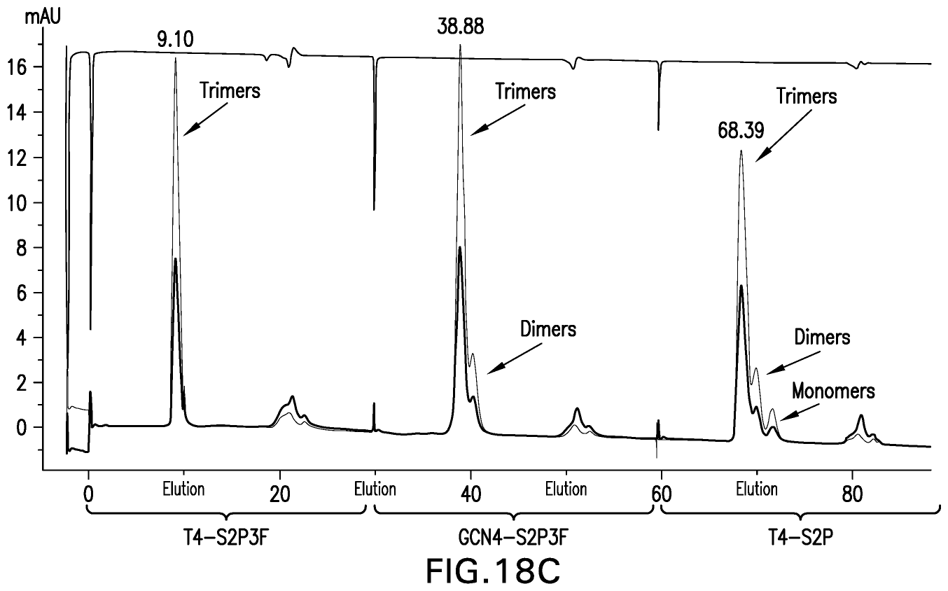

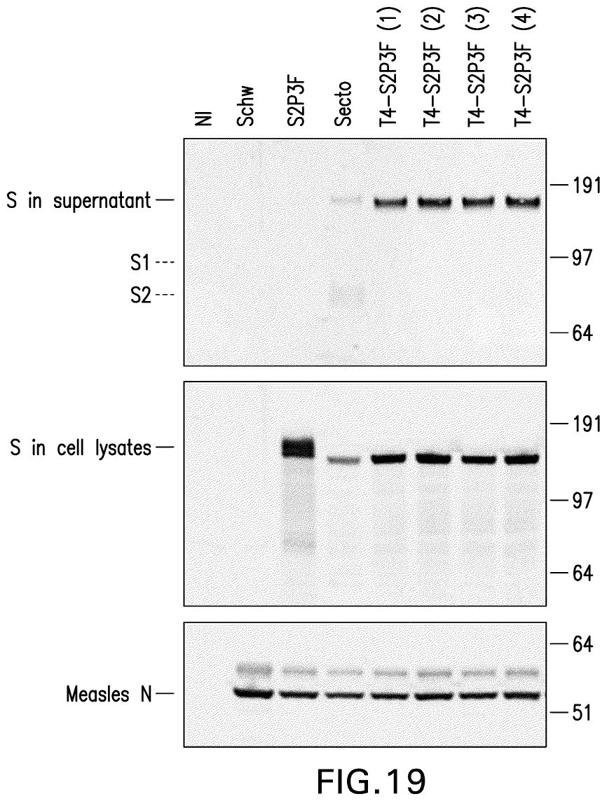

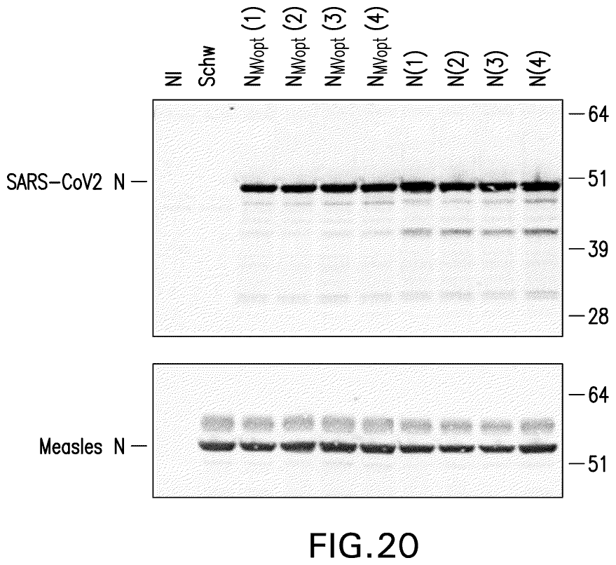

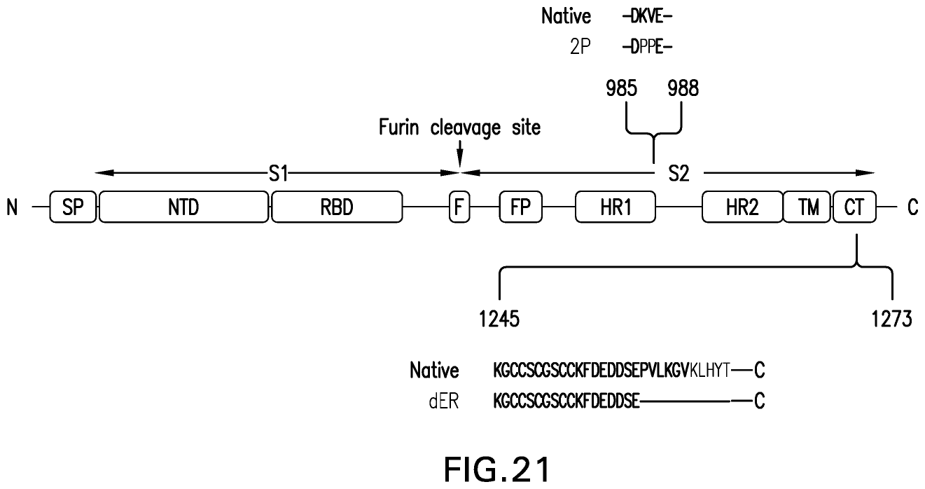

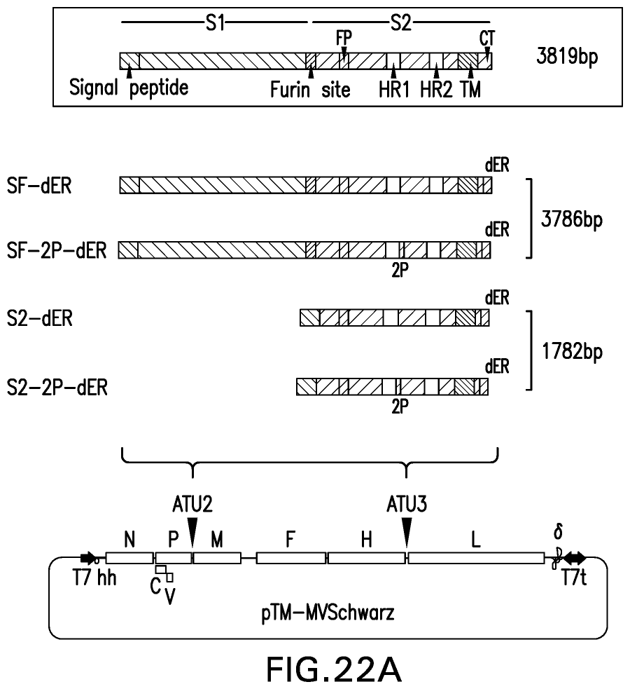

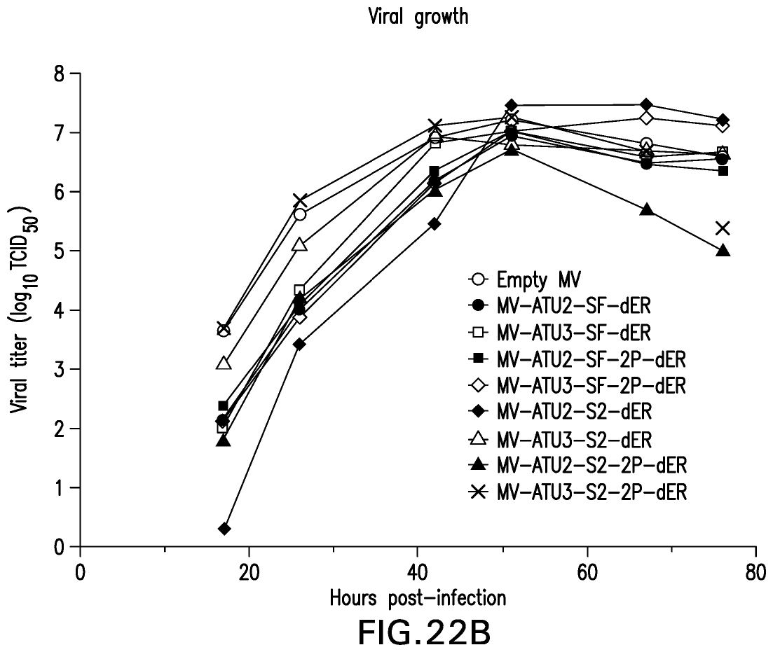

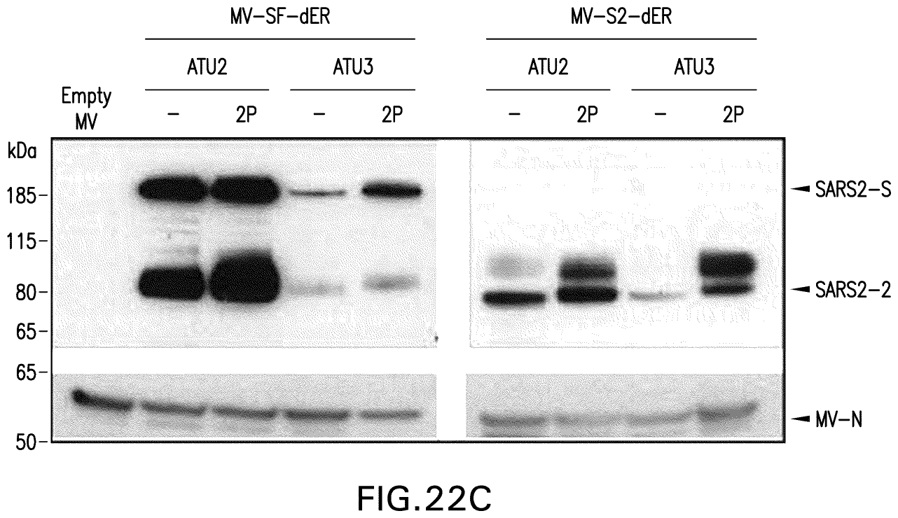

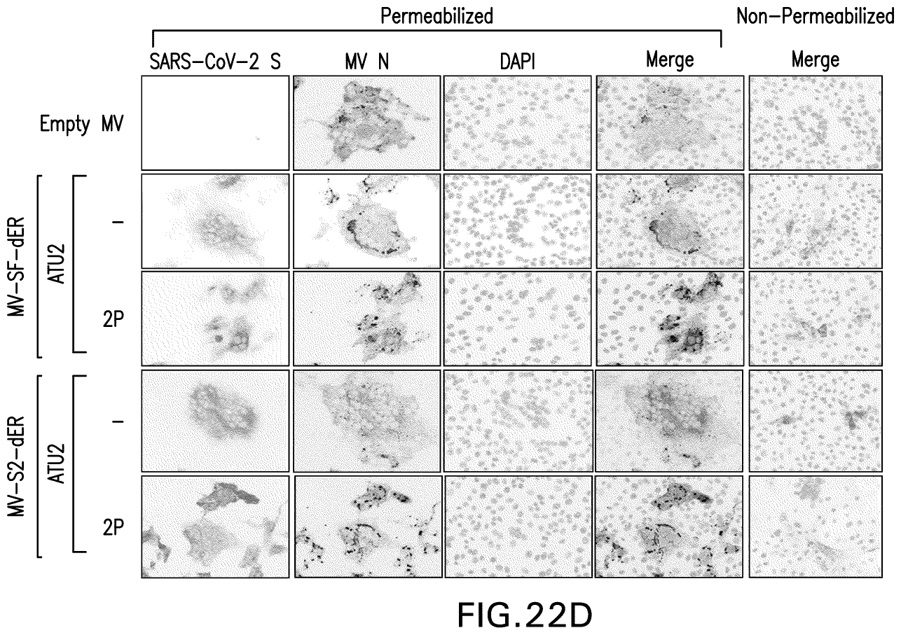

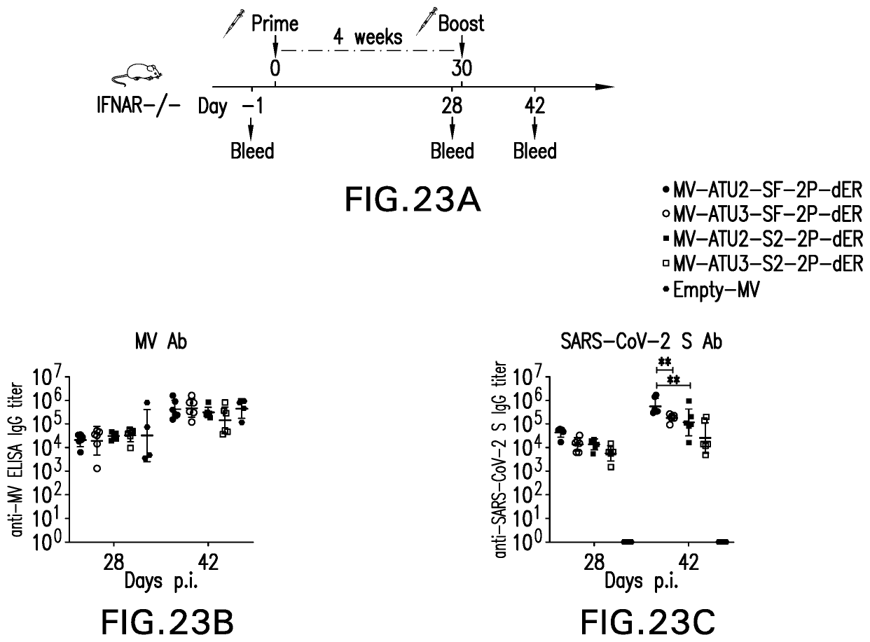

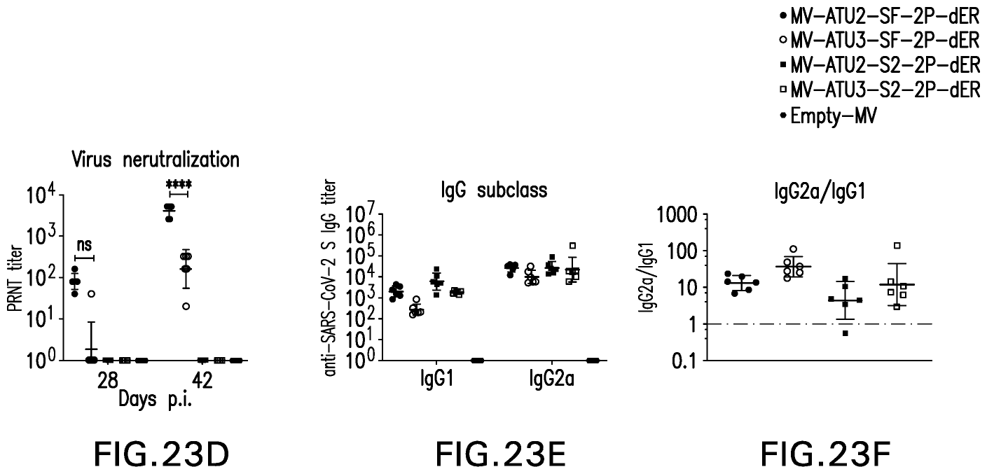

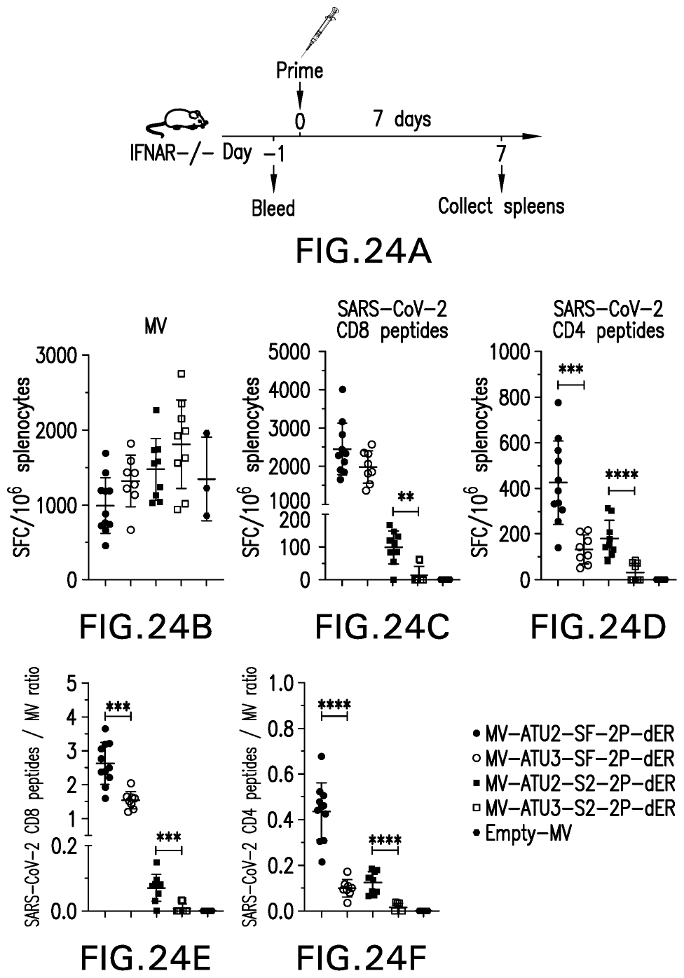

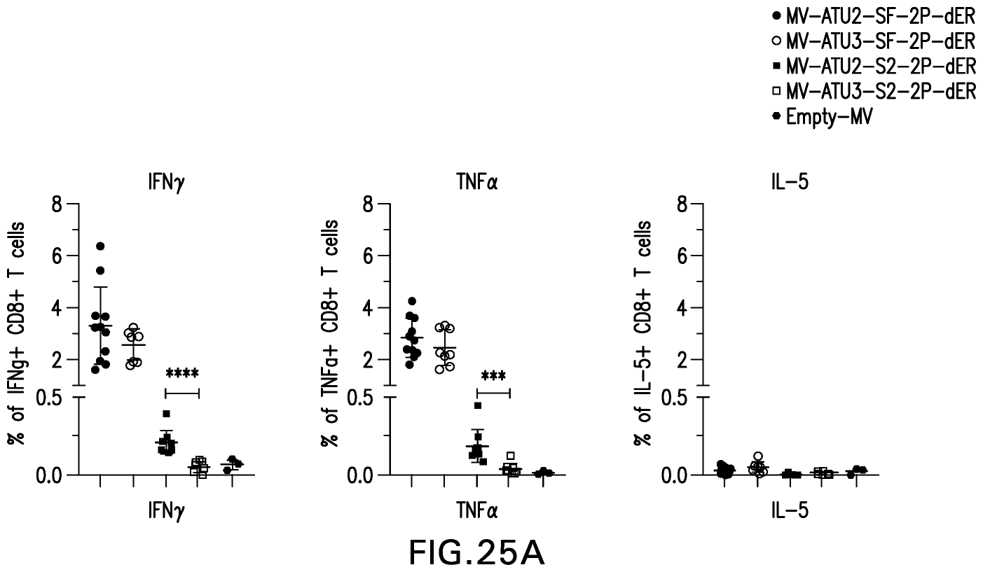

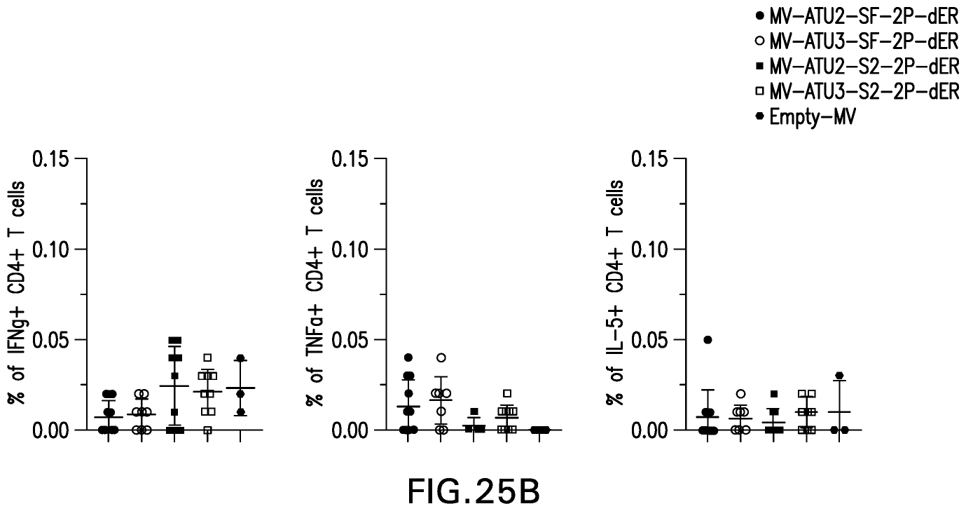

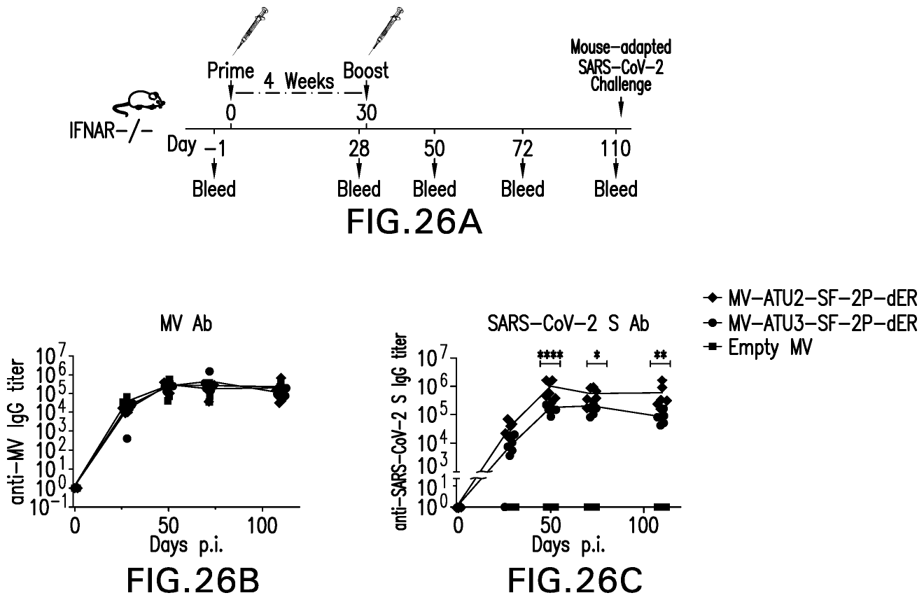

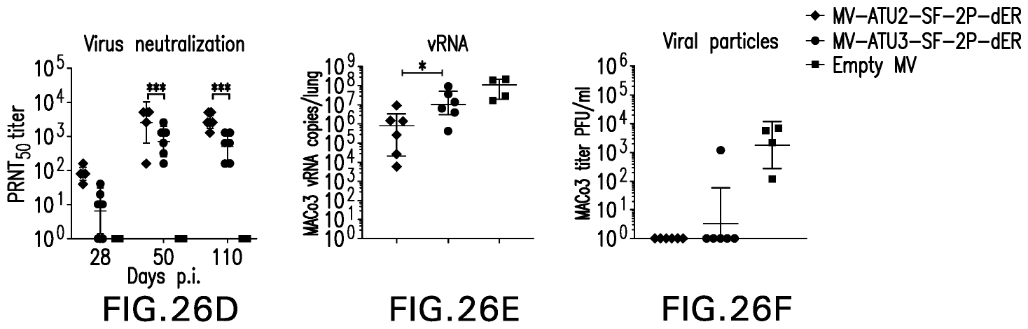

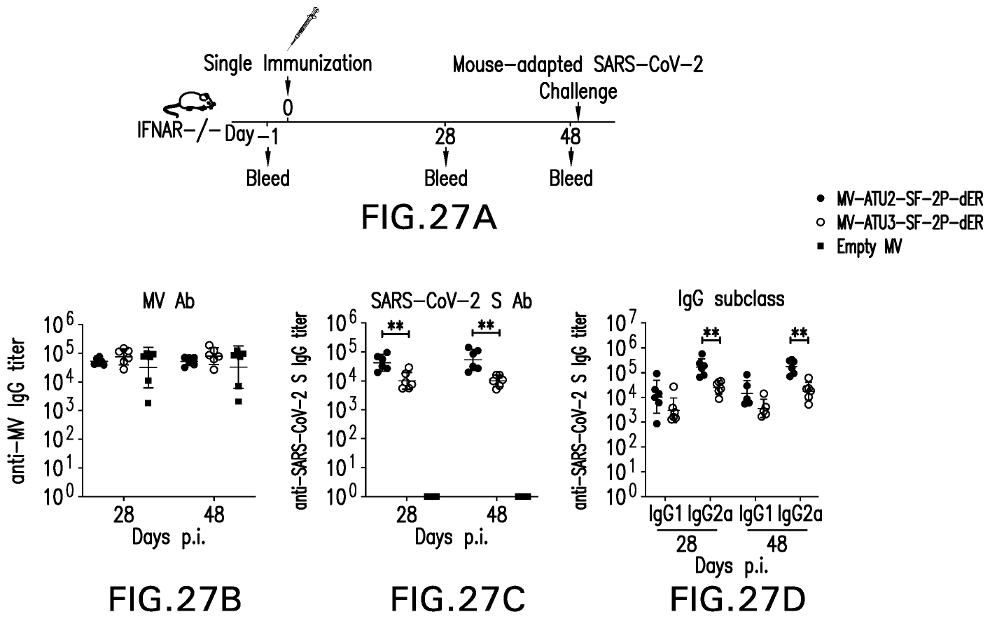

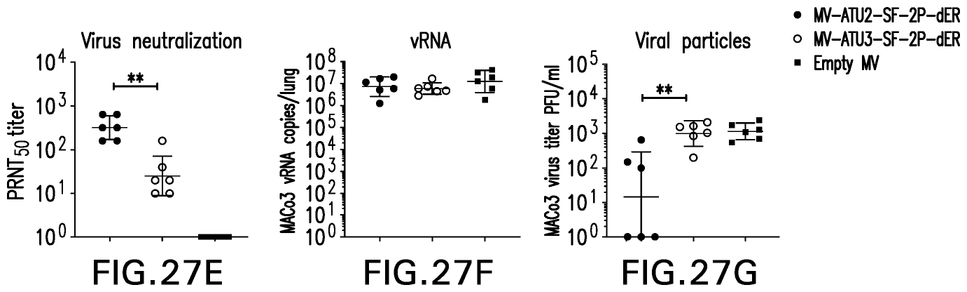

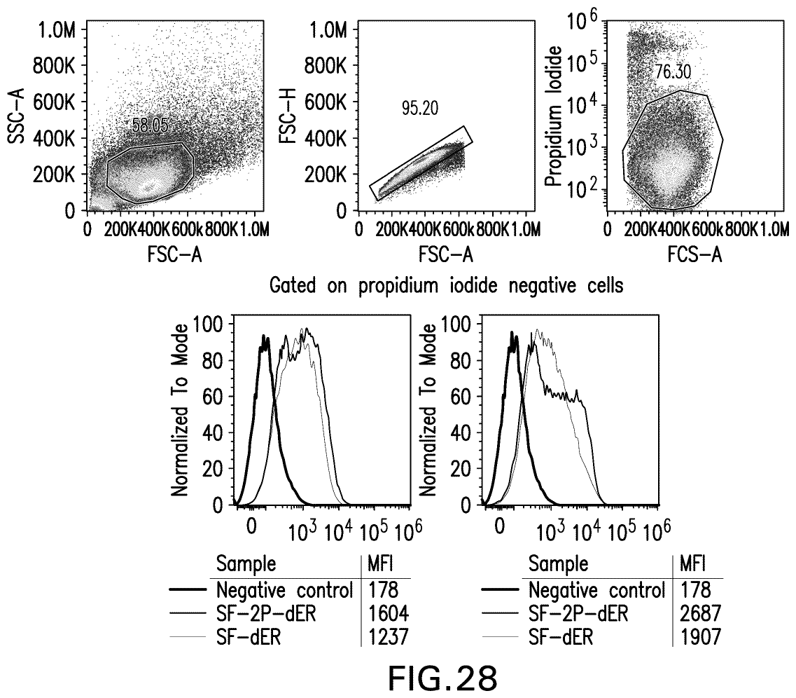

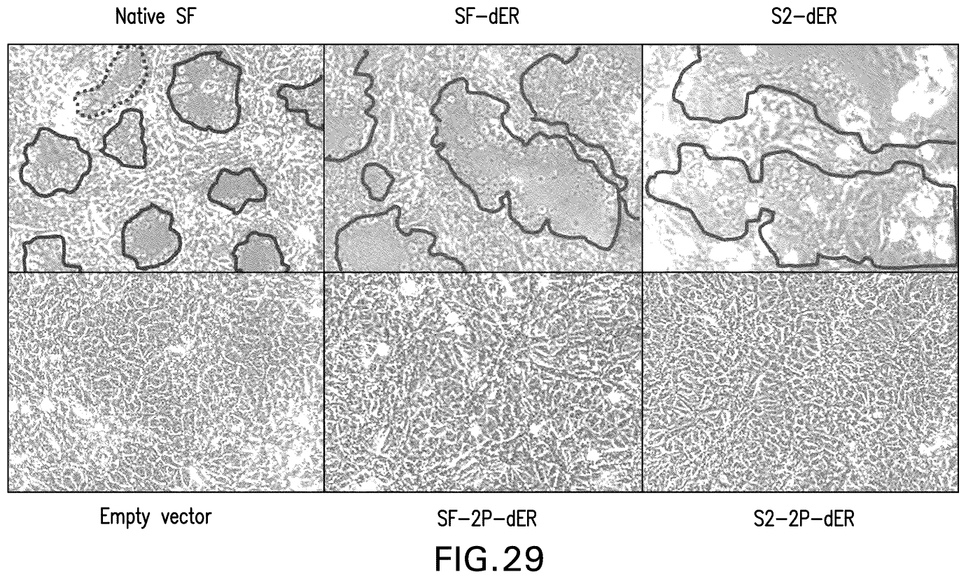

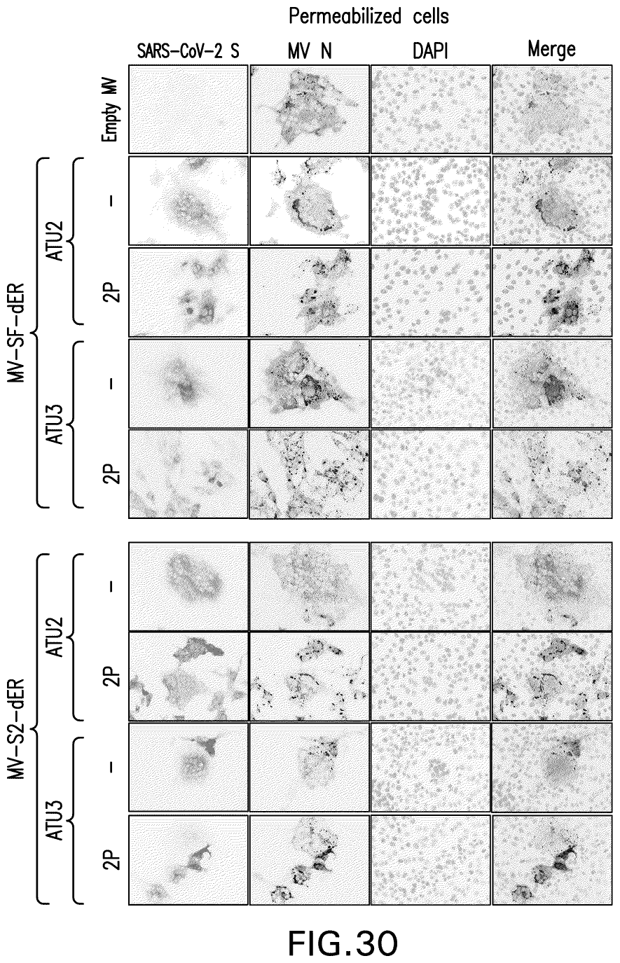

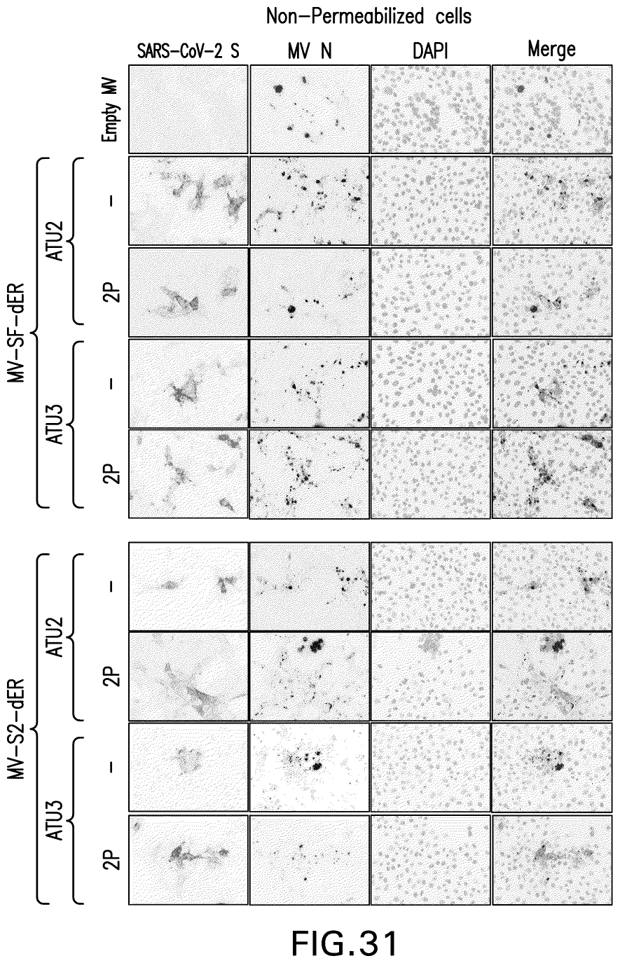

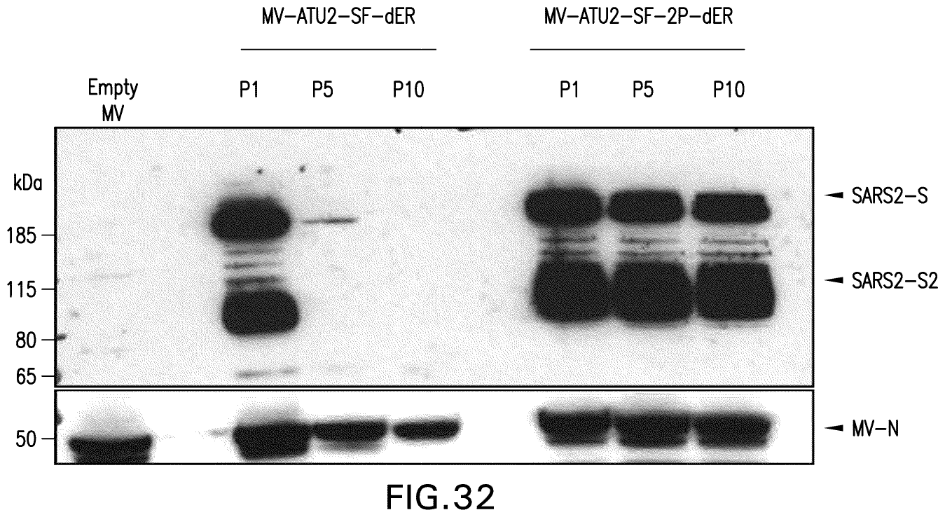

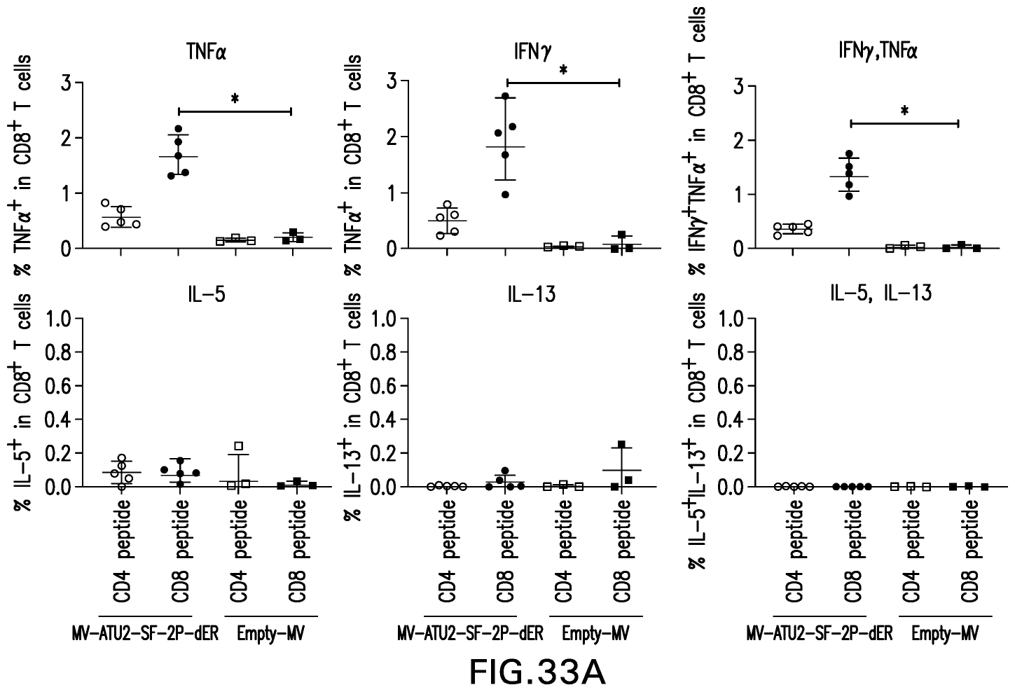

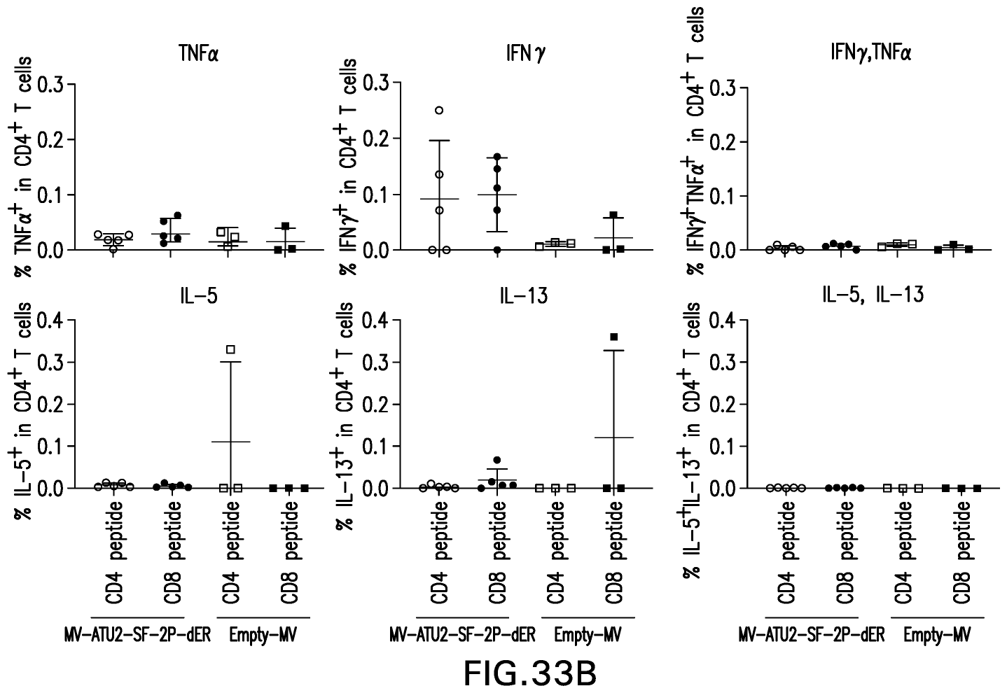

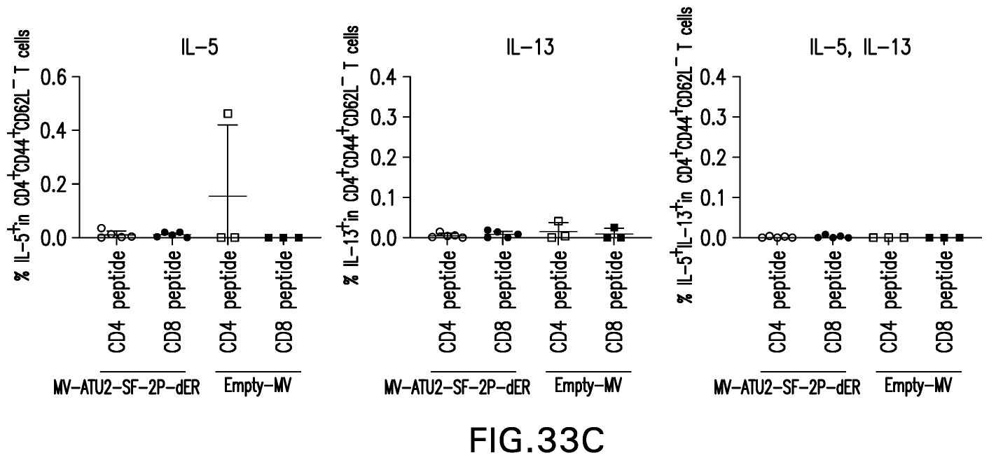

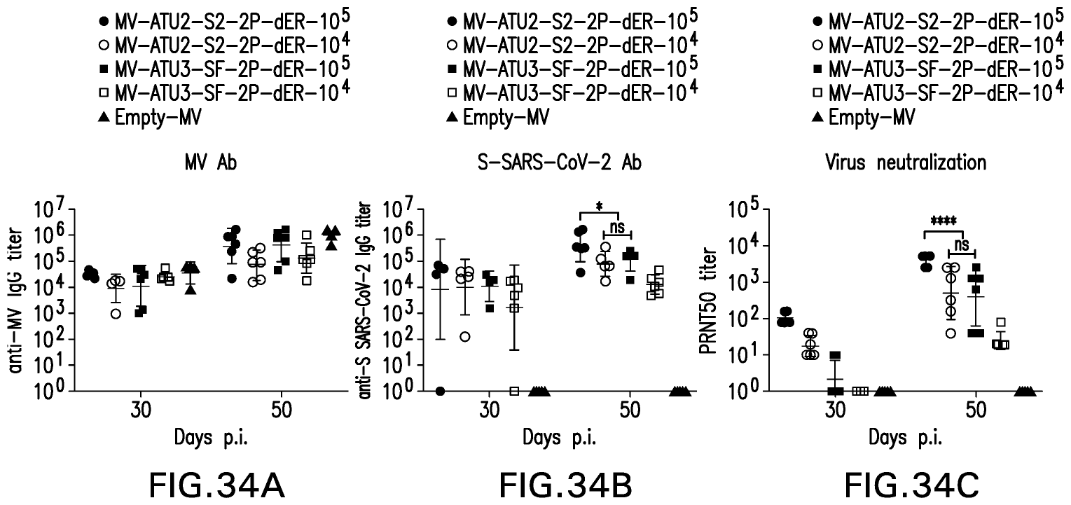

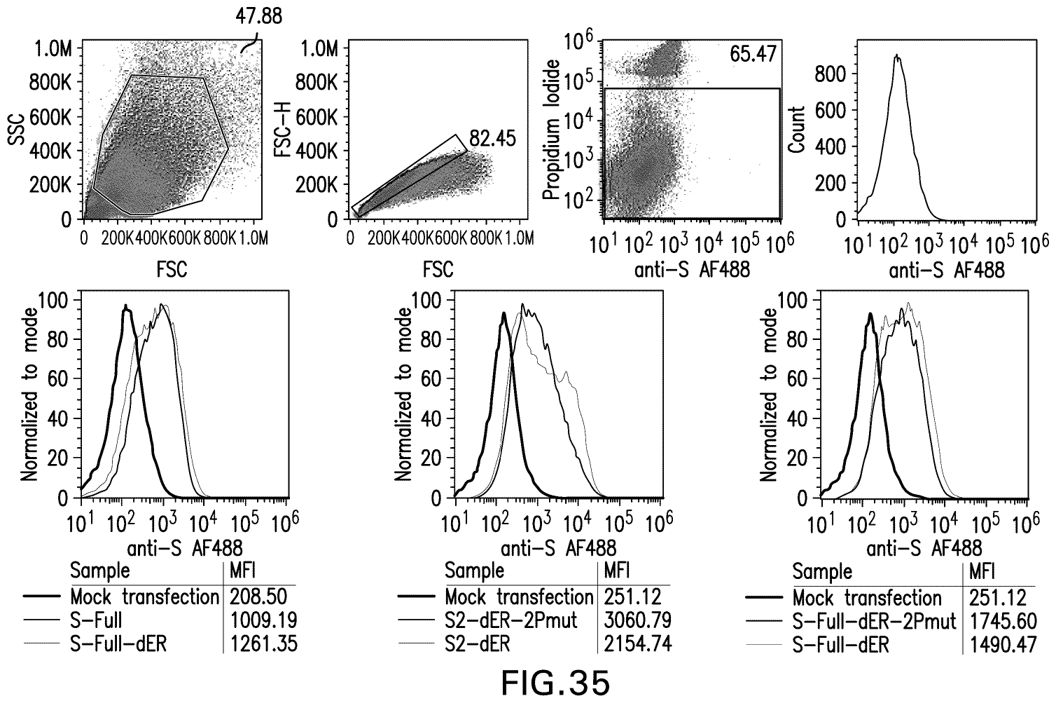

In an effort to further stabilize the spike protein, spike protein variants were designed by combining “6P” mutations (F817P, A892P, A899P, A942P, K986P, V987P) with the above-described “3F” mutations (R682G+R683S+R685G) or “ΔF” deletion (Q675-R685 loop of SEQ ID NO: 50) (FIG. 2). Modified spike genes encoding the variant spike proteins with the aforementioned modifications and combinations thereof were generated and inserted into the pKM-MVSchw-ATU3 vector. The resulting pKM3-S6P plasmid was used to successfully rescue the single recombinant MV-ATU3-S6P vaccine candidate using a helper-cell-based system as described above. MV-ATU3-S6P viral clones were propagated in Vero NK cells, grew to high titers of above 10⁷ TCID₅₀/mL and were confirmed to be genetically stable by Sanger sequencing of the ATU. Infection of Vero NK cells with MV-ATU3-S6P resulted in similar syncytia formation as infection with the parental MV Schwarz (not shown), indicating absence of enhanced fusion activity observed with MV-ATU3-S expressing native S and, thus, confirming efficient locking of S6P in the prefusion state. S expression was demonstrated in cells infected with each clone of MV-ATU3-S6P and showed partial cleavage into S1 and S2 domains (FIG. 15A). Noteworthy, similar expression levels were observed in cells infected with MV-ATU3-S, MV-ATU3-2P and MV-ATU3-S6P. This contrasts with the substantially higher expression levels observed by Hsieh et al. (Science, 2020) for the secreted and uncleaved ectodomains of S6P as compared to expression level of that of S2P upon transient expression in HEK293T cells. The fact that MV-ATU3-S6P expression level remains in the range of that of 2P will permit the comparative evaluation of the intrinsic immunogenicity properties of S2P and S6P. MV-ATU3-S6P3F and MV-ATU3-S6PΔF will be rescued and characterized essentially as described above to demonstrate expression of uncleaved and full-length forms (S6P3F, S6PΔF) of the S6P antigen in infected Vero NK cells.

Most mice immunized with MV-ATU3-S6P responded after prime with high titers of measles-specific and spike-specific antibodies, in the 10⁴ range, as measured by ELISA (FIG. 15, panel B and C). SARS-CoV-2 neutralizing titers were in the 10² range (FIG. 15, panel D). Among Measles responder mice after prime, there was a trend of increased SARS-CoV-2-specific ELISA and neutralizing titers in mice immunized with the following constructs: MV-ATU3-S2P [3.8±0.1 log 10(ELISA titers); 1.5±0.2 log 10(NT titers)]<MV-ATU3-S6P [4.1±0.1 log 10(ELISA titers); 2.1±0.2 log 10(NT titers)]<MV-ATU3-S2PΔF [4.6±0.3 log 10(ELISA titers); 2.5±0.3 log 10(NT titers)]. The difference between the neutralizing titers after prime of MV-ATU3-S2P and MV-ATU3-S6P is statistically significant (p=0.0079 Mann-Whitney test).

After boost, the anti-S antibody levels increased by about 10-fold, as observed for anti-MV antibodies and as already noted above for most spike variants vectorized by the Measles platform. Although not statically significant, MV-ATU3-S6P [3.9±0.1 log 10(NT titers)] elicited slightly higher neutralizing antibody levels after boost than MV-ATU3-S2P [3.5±0.3 log 10(NT titers)] and lower levels than MV-ATU3-S2PΔF (4.1±0.2 log 10(NT titers)).

Challenge and analysis of pulmonary viral loads were performed 4 weeks after boost immunization as described above. As shown in FIG. 15, Panel E, all measles vector constructs significantly reduced the viral load in the lungs compared to MVSchw parental virus. GEQ RNA levels were reduced by approximately 1.7 log by MV-ATU3-S2P and 2.2 log by MV-ATU3-S6P and MV-ATU3-S2PΔF2A. Noteworthy, the efficiency of protection followed the trend observed for the induction of neutralization antibody levels, MV-ATU3-S2PΔF conferring slightly better protection than MV-ATU3-S2P and MV-ATU3-S6P.

From these experiments, we conclude that the S6P performed better in terms of induction of neutralizing antibodies than the S2P antigen. Given the fact that both antigens are expressed at similar levels from the Measles vector, this suggests that S6P is more efficient locked in prefusion state than S2P and/or has higher stability. Given the fact that the “3F” mutations and the “ΔF” deletion synergize with the “2P” mutations for the induction of neutralizing antibodies, we anticipate that this may also be the case with the “6P” mutations and that MV-ATU3-S6P3F and MV-ATU3-S6PΔF will perform better for the induction of protective neutralizing antibodies than MV-ATU3-S6P and also than MV-ATU3-S2P3F and MV-ATU3-S2PΔF, respectively.

Immunogenicity and Protective Potential of Recombinant MV Schwarz Expressing SARS-CoV-2 Spike Variants Stabilized in the Closed Conformation.

As an alternative approach to induce protective antibody responses against SARS-CoV-2, the inventors designed full-length SARS-CoV-2 spike variants locked in their prefusion state by single/double Proline substitutions and covalently stabilized in the closed conformation by an additional disulfide bond. These include, but are not limited to combination of either the “2P” or “6P” mutations described above, and the double S383C/D985C “CC” mutation (SCCPP and SCC6P). This “CC” mutation was reported independently by McCallum et al, Henderson et al, and Xiong et al (2020) and shown to efficiently stabilize the spike ectodomain in its closed conformation with RBDs in down conformation. Alternatively, the inventors can combine any prefusion stabilization mutation with the double G413C/P987C “CC2” mutation, which was shown by Xiong et al to also stabilize the spike in its closed conformation. The inventors can also rely on SARS-CoV-2 spike variants covalently stabilized by the sole addition of disulfide bonds and hypothesize that such double cysteine mutations can stabilize by themselves the spike in the closed prefusion conformation.

Noteworthy, the furin cleavage site is not inactivated in the SCCPP and SCC6P constructs described above and the inventors anticipate that the RBD closed down state can limit access of the cleavage site in these constructs, thus making it resistant to furin-mediated proteolysis. Alternatively, in an effort to further stabilize the spike protein, spike protein variants can be designed by combining the “SCCPP” or “SCC6P” mutations with the above-characterized “3F” mutations or “ΔF” deletion.

Modified spike genes encoding the variant spike proteins with the aforementioned modifications and combinations thereof are generated and inserted into the pKM-MVSchw-ATU3 vector. The corresponding vaccine candidates can be rescued and characterized essentially as described above. These MV constructs may express uncleaved full-length spike in its closed conformation with RBDs in down conformation and largely occluded. The expressed closed spike can have much reduced binding affinity for the ACE-2 receptor of SARS-CoV-2, which is a major advantage for vectorization by the measles platform since it prevents the enhanced fusogenic phenotype the inventors observed for MV-ATU3-S and considered as a potential safety risk.

Immunogenicity and protective potential of these vaccine candidates can be evaluated in IFNAR-KO mice. MV constructs expressing “closed” spikes may induce different immune responses from those raised against native proteins (such as S2P) with lower levels of neutralizing, RBD-specific antibodies preventing ACE-2 binding and higher levels of antibodies binding to the RBD in down-position. The inventors expect these alternate antibodies to provide enhanced and/or broader protection against SARS-CoV-2 variants.

Immunogenicity and Protective Potential of Recombinant MV Schwarz Expressing a Secreted Form of SARS-CoV-2 Spike.

As an alternative approach to induce protective responses against SARS-CoV-2 and to avoid the enhanced fusogenic phenotype the inventors observed for MV candidates expressing full-length spike, the inventors evaluated MV candidates expressing spike ectodomain as a soluble and secreted form of S.

Several constructs were engineered from the fully codon-optimized spike gene and some of them were first cloned with a C-terminal Twin-Strep-Tag into the pCI plasmid (Promega) for transient expression analysis. These constructs are schematized in FIG. 18A and include:
A soluble and most likely monomeric form of the spike corresponding to its full-length ectodomain (Secto: M1-K1211), whose design is similar to the SARS Sol protective immunogen we successfully expressed with the MV platform (Escriou et al, 2014).
The soluble and monomeric S1 region (M1-P681) of S ectodomain from initiating methionine to Proline 681, which immediately precedes the predicted RRAR furin cleavage site.
A soluble and trimerized form of the spike (tri-Secto) corresponding to its full-length ectodomain fused to the GCN4 or T4 fibritin foldon through a Ser-Gly-Gly connecting linker.
Stabilized variants of tri-Secto, harboring the double K986P and V987P “2P” mutation (tristab-Secto), the “3F” mutations (R682G+R683S+R685G) and/or the “ΔF” deletion (Q675-R685 loop of SEQ ID NO: 50).

HEK 293T cells were transiently transfected with the set of pCI-Spike_ectomain plasmid DNAs, or, as control, with the pCI-S2P, pCI-S2PΔF and pCI-S3F plasmid DNAs, which encode full-length variants of spike. Supernatants were collected at 48 h post-transfection and spike ectodomain secretion was compared by Western Blot analysis using an anti-StrepTag monoclonal antibody. Only the soluble forms of the (trimerized) ectodomain were detected in the supernatants, indicating efficient secretion when the transmembrane and C-terminal cytosolic domains are truncated (FIG. 18B). Higher levels of spike in the supernatants were detected when the S1/S2 cleavage site was inactivated with the “3F” mutations or the “ΔF” deletion and when the GCN4 foldon was used rather than the T4 foldon. Since expression levels in total cell extracts were in the same range for all constructs (not shown), this suggests either more efficient secretion or increased stability in culture medium of uncleaved and GCN4-trimerized ectodomains.

The T4-S2P3F, GCN4-S2P3F and T4-S2P polypeptides were purified by affinity chromatography on StrepTactin columns from the supernatants of transiently transfected Expi293F cells and separated by size exclusion chromatography on a Superdex200 column (FIG. 18C). The elution profiles were recorded by absorbance at 280 nm (mAU) and showed that T4-S2P3F was exclusively composed of homotrimers, while GCN4-S2P3F and T4-S2P contained a significant proportion of dimers and dimers/monomers, respectively.

Altogether, these results indicate that the most efficient secretion and homotrimer folding are obtained when the T4 foldon is combined with the “2P” mutations and inactivation of the S1/S2 cleavage site.

As a proof of concept, fully codon-optimized cDNAs encoding the native spike ectodomain (Secto) and the best performing T4-S2P3F construct of the trimerized ectodomain variants (as assayed in the above described transient expression system) were inserted into BsiWI/BssHII-digested pKM-MVSchw-ATU3. The corresponding MV-ATU3-Secto and MV-ATU3-T4-S2P3F vaccine candidates were efficiently rescued using a helper-cell-based system as described above. Independent viral clones were propagated in Vero NK cells, grew to high titers of above TCID₅₀/mL. The correct sequence of the insert was confirmed by Sanger sequencing of the ATU, indicating that insertion of cDNA encoding secreted forms of S into the Measles vector results in genetically stable MV recombinants. Infection of Vero NK cells with MV-ATU3-Secto and MV-ATU3-T4-S2P3F resulted in similar syncytia formation as infection with the parental MV Schwarz (not shown), indicating good fitness and as expected, absence of enhanced fusogenic activity of these viruses.

Spike ectodomain expression and secretion in Vero cells infected with recombinant MVs was confirmed by Western Blot analysis using polyclonal rabbit antisera raised against recombinant S protein of SARS-CoV-2 (unpublished). As expected, the full-length S2P3F protein was only detected in cell lysates (FIG. 19, middle panel). In contrast, Secto and T4-S2P3F were clearly detected both in lysates and supernatants of infected Vero cells at 39 h post-infection (FIG. 19, upper and middle panels), indicating efficient secretion. Consistently, Secto and T4-S2P3F proteins secreted in the cell culture medium migrated with a higher apparent molecular weight than their counterparts observed within cell lysates, in agreement with these glycoproteins undergoing maturation upon transfer from the ER to the Golgi prior to secretion. Noteworthy, T4-S2P3F was present at markedly higher levels in lysates and supernatants of infected cells than Secto, which confirms more accurate folding and/or increased stability of S ectodomain when T4 foldon-mediated trimerization is combined with the “2P” mutations and inactivation of the S1/S2 cleavage site.

Immunogenicity of MV-ATU3-T4-S2P3F can be investigated in IFNAR-KO mice as described for the full-length constructs, with respect to induction of neutralizing antibody responses as well as CD4+ and CD8+ T cell responses. Induction of Th1 biased-responses and evaluation of fine tuning of the responses induced by secreted T4-S2P3F versus membrane-anchored S2P3F can be confirmed. The efficiency of protection can be assessed by quantifying pulmonary viral loads after intranasal transduction with Ad5:Ace2 and challenge with SARS-CoV-2.

Immunogenicity and Protective Potential of Recombinant MV Schwarz Expressing SARS-CoV-2 Nucleoprotein, Alone or in Combination with SARS-CoV-2 Spike.

The plasmid pKM-ATU2-N_2019-nCoV (2019-nCoV=SARS-CoV-2), abbreviated as pKM2-nCoV_NP or pKM-ATU2-N, has been described in section entitled “Plasmid vector constructs and vaccine candidate rescue” and was generated by inserting the fully codon-optimized SARS-CoV2 nucleoprotein (N) cDNA (SEQ ID NO: 21) into BsiWI/BssHII-digested pKM-MVSchw-ATU2 which contains an additional transcription unit between the phosphoprotein and matrix genes of the MV Schwarz genome. pKM-ATU2-N_{MVopt} was similarly generated using a measles-optimized sequence (SEQ ID NO: 37) in an effort to fine-tune nucleotide composition and expression levels of the transgene and promote enhanced fitness and stability of the recombinant measles viruses.

The pKM-ATU2-N and pKM-ATU2-N_{MVopt} plasmids have been used to successfully rescue the single recombinant MV-ATU2-N and MV-ATU2-N_{MVopt} vaccine candidates using a helper-cell-based system as described above. Independent viral clones were propagated in Vero NK cells, grew to high titers of above 10 TCID₅₀/mL. The correct sequence of the insert was confirmed by Sanger sequencing of the ATU, indicating that both the fully codon-optimized and the measles-optimized SARS-CoV-2 N genes inserted at position ATU2 remarkably allow the production of stable MV vectors. Infection of Vero NK cells with MV-ATU2-N and MV-ATU2-N_{MVopt} resulted in similar syncytia formation as infection with the parental MV Schwarz (not shown), indicating good fitness of these viruses.

SARS-CoV-2 nucleoprotein expression in Vero cells infected with recombinant MV was confirmed by Western Blot analysis using polyclonal rabbit antisera raised against SARS-CoV-2 N protein (unpublished). As shown in FIG. 20, a major band of very high intensity, close to saturating detection levels, was detected for all samples with the expected apparent molecular mass of 45 kDa, indicating expression of full length N protein. Intensity of the major band was stronger for MV-ATU2-N_{MVopt} than for MV-ATU2-N. Other bands of lower molecular mass were also observed for MV-ATU2-N_{MVopt} that probably correspond to minor degradation fragments. Altogether, this indicated that SARS-CoV-2 nucleoprotein was efficiently expressed from fully codon-optimized and measles-optimized genes inserted in ATU2 of the Measles vector and that the fully codon-optimized gene most likely drove higher expression of the nucleoprotein.

Immunogenicity of MV-ATU3-N and MV-ATU3-N_{MVopt} can be investigated in IFNAR-KO mice as described for the MV-S constructs, by monitoring induction of CD4+ and CD8+ T cell response. IFN-γ producing T cells can be enumerated by ELISpot after stimulation by a peptide pool spanning the N protein. Cytokine production by CD4⁺ T cells and CD8⁺ T cells can be characterized by intracellular cytokine staining analyzed by flow cytometry, allowing the inventors to confirm induction of Th1 biased-responses. The efficiency of protection can be assessed by quantifying pulmonary viral loads after intranasal transduction with Ad5:Ace2 and challenge with SARS-CoV-2.

The plasmid pKM-ATU2-N and any of the pKM3-Spike constructs, notably S6P and derived variants (S6P3F, S6PΔF and SCC6P), can be digested with SaII restriction enzyme and ligated to produce a series of double recombinant pKM-N&S plasmids harboring fully codon-optimized SARS-CoV N and S genes. Similar pKM-N_{MVopt}&S_{MVopt} plasmids can be constructed with measles-optimized SARS-CoV N and S genes. These plasmids can be used to rescue dual recombinant MV-N&S vaccine candidates, which can be comparatively characterized in vitro as described above. Immunogenicity can be investigated in IFNAR-KO mice as described for single recombinant constructs, with respect to induction of CD4+ and CD8+ T cell responses targeting N and S peptide pools and of neutralizing antibodies. Since clinical studies have suggested a protective role for both humoral and cell-mediated immunity in recovery from SARS-CoV-2 infection (Del Valle, 2020), it can be investigated whether the dual recombinant MV-N&S vaccine candidates provide better protection against intranasal challenge with SARS-CoV-2 than their single recombinant parental candidates.

### B. Example 2

1. Materials and Methods

### Cells and Viruses

Human embryonic kidney cells (HEK) 293T (ATCC CRL-3216), HEK293T7-NP helper cells (stably expressing MV-N and MV-P genes), African green monkey kidney cells (Vero) and Vero C1008 clone E6 (ATCC CRL-1586) were maintained at 37° C., 5% CO₂ in Dulbecco's modified Eagle medium (DMEM) (Thermo Fisher) supplemented with 5% (for Vero cells) or 10% (for HEK293T cells) heat-inactivated fetal bovine serum (FBS) (Corning), 100 units/ml of penicillin-streptomycin and 100 ug/ml of streptomycin. The SARS-CoV-2 BetaCoV/France/IDF0372/2020 strain was supplied by the National Reference Centre for Respiratory Viruses hosted by Institut Pasteur (Paris, France). The human sample from which strain BetaCoV/France/IDF0372/2020 was isolated has been provided by Dr. X. Lescure and Pr. Y. Yazdanpanah from the Bichat Hospital, Paris, France. The Mouse-adapated SARS-CoV-2 (MACo-3) has been described elsewhere.

Construction of pTM-MVSchwarz Expressing Modified SARS-CoV-2 S Protein Constructs

The SARS-CoV-2 spike (S) gene based on the sequence published by Zhou et al. (Zhou, 2020) was codon-optimized for expression in mammalian cells. Primers introducing restriction sites BsiWI and BssHII to the S 5′ and 3′ ends, respectively, were used to amplify nucleotides 1-3799 to generate full-length S (SF) with a deletion of its 11 C-terminal amino acids (SF-dER) (FIG. 21) for cloning into pCDNA 5.1. To generate S2 constructs, primers were designed for inverted PCR with BsmBI restriction sites and 4-nucleotide overlaps at the C-terminus of the native S signal peptide and S2 immediately adjacent to the furin cleavage site (Table 5A-5C). The amplification product, comprising the S2 region, the pCDNA backbone, and the S signal peptide, was digested with BsmBI (NEB) and self-ligated to generate S2-dER. To maintain the conformation of S in the prefusion state, two mutations were introduced at the hinge of HR1, K986P and V987P (2P mutation) (FIG. 21). Primers introducing the mutations were designed with mutated overlapping nucleotides and BsmBI sites (Tables 5A-5C). The SF-dER and S2-dER constructs were amplified, digested and self-ligated to create the prefusion-stabilized SF-2P-dER and S2-2P-dER constructs. The S constructs in the pCDNA background were transfected into Vero cells using FugeneHD. Transfected cells were observed at 24- and 48-hour post-transfection for fusogenic phenotypes.

All S genes were subsequently cloned into pTM-MVSchwarz encoding infectious MV cDNA corresponding to the anti-genome of the MV Schwarz vaccine strain. All the inserted genes were modified at the stop codon to ensure that the total number of nucleotides is a multiple of six (Calain, 1993).

### Virus Rescue, Propagation and Titration

Rescue of recombinant MV viruses was performed using a helper-cell-based system as described previously (Combredet, 2003). Briefly, helper HEK293T7-NP cells were individually transfected with 5 μg of pTM-MVSchwarz-based SARS-CoV-2 S plasmids and 0.02 μg of pEMC-La, plasmid expressing the MV polymerase (L) gene (Duprex, 2002). After overnight incubation at 37° C., the transfection medium was replaced with fresh DMEM medium. Heat shock was applied for 3 h at 42° C. before transfected cells were returned to the 37° C. incubator. After two days, transfected cells were transferred to 100-mm dishes seeded with monolayers of Vero cells. Syncytia that appeared after 2-3 days of co-culture were singly picked and transferred onto Vero cells seeded in 6-well plates. Infected cells were trypsinized and expanded in 75-cm² and then 150-cm² flasks, in DMEM with 5% FBS. When syncytia reached 80%-90% coverage (or when the maximum cytopathic effect (CPE) was observed, usually within 36-48 hours post infection), cells were scraped into a small volume of OptiMEM (Thermo Fisher). Cells were lysed by a single freeze-thaw cycle and cell lysates clarified by low-speed centrifugation. The infectious supernatant was then collected and stored at −80° C. Titers of the rMVs were determined on Vero cells seeded in 96-well plates at 7500 cells/well, and infected with serial ten-fold dilutions of virus in DMEM with 5% FBS. After incubation for 7 days, cells were stained with crystal violet, and TCID₅₀ values were calculated using the Karber method (Karber, 1931). Titers of SARS-CoV-2 and MACo3 were assessed on Vero-E6 in a similar plaque assay. The number of plaques were read 3 days post-infection.

Virus growth kinetics of rMVs was studied on monolayers of Vero cells in 6-well plates. Cells were infected with rMVs at an MOI of 0.1. One plate was used per rMV construct. At various time points post-infection, infected cells were scraped into 1 ml OptiMEM, lysed by freeze-thaw, clarified by centrifugation, and titered as described above.

To assess stability of S expression, cell lysates generated by a freeze-thaw cycle were used to infect Vero cells repeatedly for ten passages. Passage 1 (P1), P5 and P10 viruses were used to infect Vero in 6 well-plates in duplicate and assessed for S mRNA and protein levels using RT-PCR and western blotting, respectively.

### RT-PCR

To verify S expression from the rMV constructs, total RNA were extracted from infected Vero cells using the RNeasy Mini Kit (Qiagen). The cDNA synthesis and PCR steps were performed using the RNA LA PCR kit (Takara Bio) with primers (Tables 5A-5B) targeting ATU2 and ATU3, according to the manufacturer's instructions. RT-PCR products were verified by Sanger sequencing (Eurofins Genomics) using the primers indicated in Table 5C.

### Western Blot Analysis

Vero cells in 6-well plates were infected with various rMVs at an MOI of 0.1. At 36-48 h post-infection (80% syncytia), infected cells were lysed in RIPA lysis buffer (Thermo Fisher). Samples were briefly centrifuged and subjected to SDS-PAGE using the NuPAGE-pre-cast 4-12% gradient gel with NuPAGE-MOPs running buffer (Invitrogen). After transfer to a nitrocellulose membrane (GE Healthcare) and blocking with Tris-buffered saline (TBS) buffer with 0.1% Tween, 5% milk, the membrane was subsequently probed with a rabbit polyclonal anti-SARS-CoV S antibody recognizing the conserved 1124 aa-1140 aa epitope (ABIN199984, Antibody Online, 1:2000 dilution) followed by a horse-radish peroxidase (HRP)-conjugated swine anti-rabbit IgG antibody (P0399, Dako, 1:3000 dilution). Bands were visualized using SuperSignal West pico Plus chemiluminescent HRP substrate (Thermo Fisher). For loading control, membranes were stripped with 5% NaOH for 5 mins then blocked. Membranes were then re-probed with a mouse monoclonal anti-MV-N antibody (ab9397, Abcam, 1:20000 dilution) followed by an HRP-conjugated anti-mouse IgG (NA931V, GE Healthcare, 1:10000 dilution).

### Immunofluorescence Assay

Vero cells were infected with various rMVs at an MOI of 0.1. At 24-36 h post-infection, cells were fixed with 4% paraformaldehyde, blocked with 2% goat serum overnight and then treated with or without 0.1% saponin A (Sigma). Fixed cells were then probed with a mouse monoclonal anti-SARS-CoV S antibody (ab273433, Abcam, 1:300 dilution) as the primary antibody. An Alexa Fluor 488-conjugated goat anti-rabbit IgG (A-11008, Thermo Fisher) was used as the secondary antibody. Staining with anti-MV-N followed by Cy3-conjugated goat anti-rabbit (A10520, Jackson ImmunoResearch, 1:1000 dilution) was used to detect MV in the same infected cells. Nuclei were stained with DAPI. Images were collected using an inverted Leica DM IRB fluorescence microscope with a 20× objective.

### Flow Cytometry

pcDNA5.1 expression vectors encoding prefusion-stabilized or native conformation full-length S and S2 subunit antigens were used to transfect HEK293T cells using the JetPrime transfection kit (PolyPlus) according to the manufacturer's instructions. Forty-eight hours post-transfection, cells were stained for indirect immunofluorescence with 10 μg/ml of rabbit polyclonal anti-S antibody targeting S2 (ABIN199984) followed by Alexa Fluor 488-conjugated goat anti-rabbit IgG (A-11008). Propidium iodide was used to exclude dead cells by gating. Stained cells were acquired on the Attune NxT flow cytometer (Invitrogen) and data were analyzed using FlowJo v10.7 software (FlowJo LLC).

### Mice Immunizations and Challenge

All experiments were approved by the Office of Laboratory Animal Care at the Institut Pasteur and conducted in accordance with its guidelines. Groups of 6 to 8-week-old mice deficient for type-I IFN receptor (IFNAR^{-/-}) were intraperitoneally injected with 10⁵ TCID₅₀rMV, namely SF-2P-dER or S2-2P-dER in ATU2 or ATU3, or the control empty MV Schwarz. To study humoral responses, two immunizations were administered at a four-week interval. Sera were collected before the first immunization (day −1) and then before (day 28) and after (day 42) after the second immunization. All serum samples were heat-inactivated for 30 min at 56° C. To assess protection, mice that received either one or two immunizations were challenged with an intranasal inoculation of 1.5×10⁵ PFU mouse-adapted SARS-CoV-2 virus (MACo3). Three days after challenge, mice were sacrificed and lung samples collected. The presence of MACo3 virus in the lung was detected by determining viral growth, PFU of infectious viral particles, and measuring vRNA using Luna Universal Pr—be One-Step RT-qPCR kit following the manufacturer protocol (E3006). The primers and probes used correspond to the nCoV_IP4 panel (Table 5A-5C) as described on the WHO website (Protocol, Institut Pasteur, 2020).

TABLE 5A
Primers used for construction of SF-dER, S2-dER and
their 2P mutation counterparts.

| Primer name - | | |
|---|---|---|
| Construction | SEQ | |
| primers | ID NO | Sequence |
| BsiWI-Signal | 121 | TAACGTACGGCCACCATGTTCGTCTTTCTGGTATTG |
| BssHII-SF | 122 | TAAGCGCGCCTATTATTCGGAATCATCCTCATCGA |
| BsmBI-signal | 123 | TAACGTCTCCGCACTGGGAACTCACCAGAGGAAG |
| BsmBI-S2 - | 124 | TAACGTCTCAGTGCGTAGCAAGTCAGAGTATCATAG |
| BssHII-S2 | 125 | TAAGCGCGCTTATTCGGAATCATCCTCATCGAATTTAC |
| BsmBI-2P-fwd | 126 | AATCGTCTCACACCAGAGGCCGAAGTGCAGATTGATCGCCTG |
| BsmBI-2P-rev | 127 | ATACGTCTCAGGTGGGTCGAGCCGAGACAAGATGTCGTTC |

TABLE 5B
Primers used for sequencing of SF-dER, S2-dER and
their 2P mutation counterparts.

| Primer name - | | |
|---|---|---|
| Sequencing | SEQ | |
| primers | ID NO | Sequence |
| Signal-fwd1 | 128 | TCTGGTATTGCTTCCTCTGGTG |
| SF-fwd2 | 129 | TGCGCACTTGATCCATTGTC |
| S2-fwd3 | 130 | GTAAAGCACACTTCCCAAGAG |
| SF-fwd4 | 131 | GATCCTGGACATCACTCCATGC |
| SF-rev1 | 132 | TTCCACTTACATGGATAGCGTGG |
| S2-rev1 | 133 | TACTGTTATTACTATAAGCGACAG |
| S2-rev3 | 134 | GATCTCTAGCGGCGATATCTC |
| 3433 | 135 | GACCTTGGGAGGCAATCACT |
| oligo8a | 136 | GGAATCGCTGTCCTCAACAA |
| 9119 | 137 | ‘GATAGGG’TGCTAGTGAACCAAT |
| 9218 | 138 | TGGACCCTACGTTTTTCTTAATTCT |

TABLE 5C
Primers used for mouse-adapted SARS-CoV-2
vRNA detection.

| Primer name - | SEQ | |
|---|---|---|
| qRT-PCR primers | ID NO | Sequence |
| nCoV_IP4-14059Fw | 139 | GGTAACTGGTATGATTTCG |
| nCoV_IP4-14146Rv | 140 | CTGGTCAAGGTTAATATAGG |
| nCoV_IP4- | 141 | TCATACAAACCACGCCAGG |
| 14084Probe(+) | | [5']Hex [3']BHQ-119 |

### ELISA

Edmonston strain-derived MV antigens (Jena Bioscience) or recombinant S protein encompassing amino acid residues 16 to 1213 with R683A and R685A mutations (ABIN6952426, Antibodies Online) were coated on NUNC MAXISORP 96-well immuno-plates (Thermo Fisher) at 1 μg/ml in 1× phosphate-buffered saline (PBS). Coated plates were incubated overnight at 4° C., washed 3 times with washing buffer (PBS, 0.05% Tween), and further blocked for 1 h at 37° C. with blocking buffer (PBS, 0.05% Tween, 5% milk). Serum samples from immunized mice were serially diluted in the binding buffer (PBS, 0.05% Tween, 2.5% milk) and incubated on plates for 1 h at 37° C. After washing steps, an HRP-conjugated goat anti-mouse IgG (H+L) antibody (Jackson ImmunoResearch, 115-035-146, 1:5000 dilution) was added for 1 h at 37° C. Antibody binding was detected by addition of the TMB substrate (Eurobio) and the reaction was stopped with 100 μl of 30% H₂SO₄. The optical densities were recorded at 450 and 620 nm wavelengths using the EnSpire 2300 Multilabel Plate Reader (Perkin Elmer). Endpoint titers for each individual serum sample were calculated as the reciprocal of the last dilution giving twice the absorbance of the negative control sera. Isotype determination of the antibody responses was performed using HRP-conjugated isotype-specific (IgG1 or IgG2a) goat anti-mouse antibodies (AB97240 and AB97245, Abcam, 1:5000).

### Plaque Reduction Neutralization Test

Two-fold serial dilutions of heat-inactivated serum samples were incubated at 37 C for 1 h with 50 PFU of SARS-CoV-2 virus in DMEM medium without FBS and added to a monolayer of Vero E6 cells seeded in 24-well plates. Virus was allowed to adsorb for 2 h at 37° C. The supernatant was removed and the cells were overlaid with 1 ml of plaque assay overlay media (DMEM supplemented with 5% FBS and 1.5% carboxymethylcellulose). The plates were incubated at 37 C with 5% CO₂ for 3 days. Viruses were inactivated and cells were fixed and stained with a 30% crystal violet solution containing 20% ethanol and 10% formaldehyde (all from Sigma). Serum neutralization titer was counted on the dilution that reduced SARS-CoV-2 plaques by 50% (PRNT₅₀).

### ELISPOT

Splenocytes from immunized mice were isolated and red blood cells lysed using Hybri-Max Red Blood Cell Lysing Buffer (Sigma). The splenocytes were tested for their capacity to secrete IFN-γ upon specific stimulation. Multiscreen-HA 96-well plates (Millipore) were coated overnight at 4° C. with 100 μl per well of 10 μg/ml of anti-mouse IFN-γ (551216, BD Biosciences) in PBS before washing and blocking for 2 h at 37° C. with 200 μl complete MEM-α (MEM-α (Thermo Fisher) supplemented with 10% FBS, 1× non-essential amino acids, 1 mM sodium pyruvate, 2 mM L-glutamine, 10 mM HEPES, 1% penicillin-streptomycin, and 50 μM β-mercaptoethanol). The medium was replaced with 100 μl of cell suspension containing 1×10⁵ splenocytes in each well in triplicate and 100 μl of stimulating agent in complete MEM-α supplemented with 10 U/ml of mouse IL-2 (Roche). Stimulating agents used were 2.5 μg/ml concanavalin A (Sigma Aldrich) for positive controls, complete MEM-α for negative controls, MV Schwarz virus at an MOI of 1, or a SARS-CoV-2 S peptide pool (Tables 6A-6B) at 2 μg/ml per peptide. After incubation for 40 h at 37° C., 5% CO₂, plates were washed once with PBS, then three times with washing buffer (PBS, 0.05% Tween). A biotinylated anti-mouse IFN-γ antibody (554410, BD Biosciences) at 1 μg/ml in the washing buffer was added and plates were incubated for 120 min at room temperature. After extensive washing, 100 μl of streptavidin-alkaline phosphatase conjugate (Roche) was added at a dilution of 1:1000 and plates were further incubated for 1 h at room temperature. Wells were washed twice with the washing buffer and followed by a wash with PBS buffer without Tween. Spots were developed with BCIP/NBT (Sigma) and counted on a CTL ImmunoSpot® ELISPOT reader.

TABLE 6A
Peptide pools corresponding to the S1 and S2
subunits used to stimulate S-specific CD4⁺ T cells.

| CD4 peptide | SEQ | | Amino |
|---|---|---|---|
| sequence | ID NO | Subunit | acid position |
| QDLFLPFFSNVTWFH | 83 | S1 | 52-66 |
| STEIYQAGSTPCNGV | 84 | S1 | 469-483 |
| VLSFELLHAPATVCG | 85 | S1 | 512-526 |
| ENSVAYSNNSIAIPT | 86 | S2 | 702-716 |
| ITSGWTFGAGAALQI | 87 | S2 | 882-896 |
| QMAYRFNGIGVTQNV | 88 | S2 | 901-915 |
| GKIQDSLSSTASALG | 89 | S2 | 932-946 |
| IRAAEIRASANLAAT | 90 | S2 | 1013-1027 |
| GYHLMSFPQSAPHGV | 91 | S2 | 1046-1060 |
| PAQEKNFTTAPAICH | 92 | S2 | 1069-1083 |

TABLE 6B
Peptide pools corresponding to the
S1 and S2 subunits used to
stimulate S-specific CD8⁺ T cells.

| CD8 peptide | SEQ | | Amino acid |
|---|---|---|---|
| sequence | ID NO | Subunit | position |
| FVFLVLLPL | 93 | S1 | 2-10 |
| VNLTTRTOL | 94 | S1 | 16-24 |
| LFLPFFSNV | 95 | S1 | 54-62 |
| SNVTWFHAI | 96 | S1 | 60-68 |
| VTWFHAIHV | 97 | S1 | 62-70 |
| RGWIFGTTL | 98 | S1 | 102-110 |
| FQFCNDPFL | 99 | S1 | 133-141 |
| YSSANNCTF | 100 | S1 | 160-168 |
| VSQPFLMDL | 101 | S1 | 171-179 |
| KIYSKHTPI | 102 | S1 | 202-210 |
| INITRFQTL | 103 | S1 | 233-241 |
| AAAYYVGYL | 104 | S1 | 262-270 |
| VRFPNITNL | 105 | S1 | 327-335 |
| FNATRFASV | 106 | S1 | 342-350 |
| GNYNYLYRL | 107 | S1 | 447-455 |
| VGYQPYRVV | 108 | S1 | 503-511 |
| VVVLSFELL | 109 | S1 | 510-518 |
| VNFNFNGLT | 110 | S1 | 539-547 |
| YQDVNCTEV | 111 | S1 | 612-620 |
| SIIAYTMSL | 112 | S2 | 691-699 |
| VAYSNNSIA | 113 | S2 | 705-713 |
| FGGFNFSQI | 114 | S2 | 797-805 |
| AALQIPFAM | 115 | S2 | 892-900 |
| VVNQNAQAL | 116 | S2 | 951-959 |
| VVFLHVTYV | 117 | S2 | 1060-1068 |
| ISGINASVV | 118 | S2 | 1169-1177 |
| IWLGFIAGL | 119 | S2 | 1216-1224 |
| IAIVMVTIM | 120 | S2 | 1225-1233 |

### Intracellular Cytokine Staining

Splenocytes of vaccinated mice were extracted as previously described. Two million splenocytes per mouse per well were incubated in 200 μL of complete MEM-α medium (Thermo Fisher). ED Golgi Stop (554724, ED Biosciences) was added to the culture medium according to the manufacturer's instructions. Splenocytes were stimulated with a peptide pool covering the predicted CD4 and CD8 T-cell epitopes of the SARS-CoV-2 S protein (Table 6A-6B) at a final concentration of 2 μg/ml per peptide. PMA/lonomycin Cell Stimulation Cocktail (eBioscience) was used as a stimulation for positive controls, and medium alone was used for negative controls. Splenocytes were stimulated for 4 h at 37° C. Stimulated cells were incubated with Mouse BD Fc Block (553141, BD Biosciences), and stained with Live/Dead Fixable Aqua Viability Dye (ThermoFisher) to exclude dead cells by gating. Subsequently, cells were stained with CD3e PE (clone 145-2C11, 12-0031-83, eBioscience), CD4 PerCP-eFluor710 (clone RM4-5, 46-0042-82) and CD8 Alexa Fluor 488 (clone 53-6.7, 53-0081-82) antibodies from Invitrogen. Cells were fixed and permeabilized with BD Fixation/Permeabilization kit (BD Biosciences) and stained with IFN-γ APC/Fire750, (clone XMG1.2, 505860, BioLegend), TNF-α BV421 (clone MP6-XT22, 563387, BD Horizon) and IL-APC (clone TRFK5, 505860, BD Biosciences) antibodies. Samples were acquired using the Attune NxT flow cytometer (Invitrogen) and data were analyzed using FlowJo v10.7 software (FlowJo LLC).

### Statistical Information

Statistical analyses were performed using GraphPad Prism v.8.0.2. Results were considered significant if p<0.05. The lines in all graphs represent the geometric mean with error bars indicating geometric SD. Statistical analyses of antibody responses, ELISA and PRNT₅₀, were done using two-way ANOVA adjusted for multiple comparisons. The two-tailed nonparametric Mann-Whitney's U test was applied to compare differences between two groups.

2. Results

### Design of SARS-CoV-2 S Antigens

Based on previous work from the inventors with MV expressing SARS-CoV-1 S (Escriou, 2014) and since SARS-CoV and SARS-CoV-2 S proteins share a high degree of similarity (Chan, 2020), the full-length S protein of SARS-CoV-2 was chosen as the main antigen to be expressed by the MV vector. The inventors introduced a number of modifications in the native S sequence to improve its expression and immunogenicity (FIG. 21). First, the RNA sequence was codon-optimized to increase its expression in human cells. Second, the inventors substituted two amino acids with prolines, K986P and V987P, in the S2 region to generate a subset of 2P constructs, following a proven strategy to stabilize the S protein in its prefusion conformation, increasing its expression and immunogenicity (Kirchdoerfer, 2018; Pallesen, 2017; Wrapp, 2020). Third, to increase the surface expression of the S protein in MV-infected cells, the inventors deleted the 11 C-terminal amino acids (aa 1263-1273) from the S cytoplasmic tail to generate dER constructs. The cytoplasmic tail of coronaviruses S proteins contains one or two distinct retention signals: the endoplasmic reticulum retrieval signal (ERRS) comprising KxHxx of SEQ ID NO: 149 or KKxx motifs, and the tyrosine-dependent localization signal Yxxϕ (Ujiker, 2016). S proteins with ERRS are recruited into coatomer complex I (COPI) and recycled from the Golgi to the ER in retrograde. Thus, the repeated cycling of S proteins between the ER and the Golgi leads to S protein intracellular retention, while mutant S proteins lacking the ERRS are transported to the plasma membrane (McBride, 2007; Ujike, 2015). Similarly, the S proteins of Alphacoronaviruses with the Yxxϕ motif are retained in the ER with little or no S protein trafficking to the cell surface (Schwegmann-Wessels, 2004). The inventors therefore designed their SARS-CoV-2 S antigens with the deletion of all possible retention signals from the cytoplasmic tail.

To investigate the possibility of generating a broad-spectrum vaccine targeting both SARS-CoV-1 and SARS-CoV-2 clinical isolates, the inventors also designed S2 subunit antigens (FIG. 22a). The S2 subunit of SARS-CoV-2 is highly conserved among SARS-like CoVs and shares 99% identity with those of bat SARS-like CoVs (SL-CoV ZXC21 and ZC45) and of a human SARS-CoV-1 (Chan, 2020). The inventors therefore designed S2 subunit antigens, both in its native trimer and prefusion-stabilized form, with the signal peptide of the S protein inserted in the N-terminus to target the antigen to the cell surface.

Altogether, the inventors designed four different SARS-CoV-2 S constructs (FIG. 22a): 1) the native-conformation full-length S trimer (SF-dER); 2) the prefusion-stabilized full-length S (SF-2P-dER); 3) the native conformation trimer S2 subunit (S2-dER); and 4) the prefusion-stabilized S2 subunit (S2-2P-dER).

### Expression Profile of SARS-CoV-2 S Antigens

Full-length S and S2 sequences were firstly cloned into pCDNA and transfected into HEK293T cells to verify expression and assess surface protein localization by surface staining followed by flow cytometry. Prefusion-stabilized S constructs were observed to localize more strongly to the surface of transfected cells (FIG. 28). Functionality of the S proteins was analyzed by transfecting the same pCDNA vectors in Vero cells, which express ACE-2. Once S proteins bound to ACE-2 receptors, activation of the fusion protein can be observed through the formation of large syncytia among cells. Vero cells expressing the native S protein (full-length S with an intact CT) exhibited significant syncytium formation (FIG. 29), indicating that functional S proteins were expressed on the cell surface. Notably, the SdER mutants induced increased fusion compared to native SF, confirming the expected increased surface expression of the S protein when the ERRS is deleted. Interestingly, expression of the S2 subunit alone resulted in a hyper-fusion phenotype in Vero cells. This suggests the triggering of non-receptor-mediated membrane fusion by proteases cleaving at the S2′ site and freeing the fusion peptide. On the contrary, both the 2P-stabilized SF-2P-dER and S2-2P-dER did not induce syncytium formation, indicating that their fusion activity was abrogated by the 2P mutation.

Generation of rMVs Expressing SARS-CoV-2 S and S2 Proteins

The four antigenic constructs were individually cloned into the pTM-MVSchwarz plasmid at additional transcription units (ATU), with ATU2 located between the P and M genes of the MV genome and ATU3 between the H and L genes (Combredet, 2003) (FIG. 22a). Due to the decreasing expression gradient of MV genes cloning in ATU2 allows high-level expression of the antigen while cloning in ATU3 results in lower levels of expression (Plumet, 2005). The lower expression from ATU3 is a trade-off to facilitate rescue of rMV encoding antigens that are toxic or difficult to express.

All rMVs expressing the S proteins were successfully rescued by reverse genetics and propagated in Vero cells. Although the rMVs exhibited slightly delayed growth kinetics, final virus yields were high and identical to that of the parental MV Schwarz (˜10⁷ TCID₅₀/ml) (FIG. 22b). The expression of S antigens was detected in infected Vero cells by western blotting (WB) and immunofluorescence staining (IF) (FIGS. 22c, d and FIGS. 30 and 31). As expected, much higher antigen expression was observed from ATU2 vectors compared to ATU3 (FIG. 22c).

When using recombinant viral vectors as vaccines, the genetic stability of constructs is a major concern as it guarantees the effectiveness of the vaccine after multiple manufacturing steps. Such analysis of rMVs after serial passaging in Vero cells revealed that MV-ATU2-SF-dER, which expresses the native S from ATU2, was unstable, with loss of S expression by passage 5 (FIG. 32). In contrast, its 2P counterpart was stably and efficiently expressed up to passage 10. Therefore, the inventors discarded the vaccine candidates expressing native S and selected those expressing the prefusion-stabilized SF-2P and S2-2P constructs for further immunogenicity studies.

### Induction of SARS-CoV-2 Neutralizing Antibodies in Mice

The inventors investigated the immunogenicity of selected rMV vaccine candidates in IFNAR −/− mice susceptible to MV infection (Mura, 2018). Animals were immunized by one or two intraperitoneal administrations of the rMV candidates at 1×10⁵ TCID₅₀ on days 0 and 30 (FIG. 23a). Empty MV Schwarz was used for control vaccination. Mice sera were collected 4 weeks after the prime and 12 days after the boost. The presence of S- and MV-specific IgG antibodies was assessed by indirect ELISA using SARS-CoV-2 S recombinant protein and native MV antigens, respectively.

All animals raised high MV-specific IgG antibodies after prime at comparable titers in all groups (˜10⁴-10⁵ IgG titer), indicating efficient vaccine take in all the animals (FIG. 23b). Boost immunization increased MV-specific antibody titers in all groups, indicating that all animals received a successful prime-boost vaccination. Specific IgG antibodies to SARS-CoV-2 S were detected in 100% of immunized mice. Interestingly, rMVs expressing SF-2P-dER or S2-2P-dER antigens from ATU2 elicited higher levels of anti-S antibodies than the ATU3 vectors, particularly after boosting (FIG. 23c). Pre-immune sera and sera from control animals that received empty MV remained negative for anti-S antibodies (data not shown).

The inventors next assessed the presence of SARS-CoV-2 neutralizing antibodies (NAbs) using plaque reduction neutralization tests (PRNT) with SARS-CoV-2 virus infection of Vero E6 monolayers. After the prime, SARS-CoV-2 NAbs were found in all mice immunized with SF-2P-dER expressed from ATU2 but only one mouse immunized with the ATU3 construct (FIG. 23d). After the second immunization, NAb titers increased in both groups, with the ATU2 group exhibiting ten-fold higher NAb titers compared to the ATU3 group. No NAbs were detected in animals immunized with the S2 candidates despite the high levels of anti-S antibodies (FIG. 23c).

As IgG isotype switching can serve as indirect indicators of Th1 and Th2 responses (Finkelman, 1990), the inventors determined S-specific IgG1 and IgG2a isotype titers in the sera of immunized mice (FIGS. 23f, g). Similar to previous results from the inventors (Escriou, 2014), rMV candidates elicited significantly higher IgG2a antibody titers than IgG1, reflecting a predominant Th1-type immune response (FIGS. 23f, g). Since activated T cells play important roles in shaping Th1 and Th2 cytokine production, the inventors analyzed S-specific T-cell responses in MV-immunized mice in more detail.

### Induction of S-Specific T-Cell Responses

Cell-mediated immune responses elicited by immunization were first investigated using an IFN-γ ELISPOT assay. Groups of IFNAR^{-/-} mice were sacrificed one week after prime immunization (FIG. 24a). To evaluate S-specific responses, splenocytes were stimulated ex vivo with a pool of synthetic peptides covering the predicted CD8⁺ and CD4⁺ T-cell epitopes of the SARS-CoV-2 S protein, matching the MHC-I H-2K^{b}/H-2D^{b} and MHC-II I-A^{b} haplotype of 129sv IFNAR^{-/-} mice (Table 6A and 6B). Splenocytes were also stimulated with an empty MV virus to detect MV vector-specific T-cell responses.

High levels of T-cell responses to SARS-CoV-2 S and MV were elicited early after prime vaccination (FIG. 24). Splenocytes from mice vaccinated with MV-ATU2-SF-2P-dER yielded remarkably high IFN-γ secretion levels after stimulation with an MHC class I-restricted S peptide pool, yielding around 2,500 spot forming cells (SFC) per 10⁶ splenocytes. Lower IFN-γ responses were observed upon stimulation with MHC class II-restricted S peptides, at approximately 400 SFC/10⁶ splenocytes (FIGS. 24b, c). Splenocytes of these mice also exhibited relatively low vector-specific IFN-γ responses (˜990 SFC/10⁶ splenocytes), indicating a well-balanced S-to-MV vector response ratio (FIG. 24d). The ATU3 counterpart of the same vaccine tended to generate more vector-specific IFN-γ secreting cells (˜1,320 SFC/10⁶ splenocytes), while at the same time being less efficient in producing S-specific IFN-γ secreting cells after stimulation with MHC class II-restricted S peptides (˜130 SFC/10⁶ splenocytes). While IFN-γ responses after stimulation with MHC class I-restricted S peptides were not significantly different from those of its ATU2 counterpart (˜1,980 SFC/10⁶ splenocytes), the S-to-MV vector response ratio was significantly higher for MV-ATU2-SF-2P-dER (FIG. 24b).

In contrast, S-specific IFN-γ responses elicited by S2-only constructs were low after stimulation with either of the S peptide pools (FIGS. 24c, d). S-to-MV vector response ratios also remained very low in these animals, suggesting that immunization with the S2 protein subunit alone might be not sufficient to induce strong protective cellular immune responses (FIGS. 24e, f). MV-ATU3-S2-2P-dER and empty MV were unable to induce S-specific IFN-γ responses.

The inventors next studied S-specific CD4⁺ and CD8⁺ T cells by flow cytometric analysis after intracellular cytokine staining (ICS). S-specific IFN-γ⁺ and TNF-α⁺ responses were observed in for CD8⁺ T cells, while CD4⁺ T cells responded poorly to S peptide pool stimulation (FIGS. 25a, b). Similar to the ELISPOT results, SF-2P-dER expressed from ATU2 or ATU3 induced remarkably high and comparable percentages of S-specific IFN-γ⁺ and TNF-α⁺ CD8⁺ T cells, while the S2 protein expressed from ATU2 was ten times less immunogenic. MV-ATU3-S2-2P-dER and empty MV were unable to induce S-specific IFN-γ- or TNF-α-producing T-cells. IL-5-secreting cells (indicative of a Th2-biased response) were not detected in any of the immunization groups. An additional detailed analysis of T cell responses in mice immunized with MV-ATU2-SF-2P-dER confirmed the strong stimulation of CD8 compartment with high levels of S-specific IFN-γ⁺ and TNF-α⁺ producing CD8⁺ T cells, as well as double positive IFN-γ⁺/TNF-α⁺ producing CD8⁺ T cells. No IL-5 or IL-13 was detected in CD4⁺ or CD8⁺ T cells, as well as in CD4⁺/CD44⁺/CD62L⁻ memory T cells, confirming that S-specific memory T cells are also Th1-oriented (FIG. 33).

Taken together, these results demonstrate that MV-ATU2-SF-2P-dER induces a robust Th1-driven T-cell immune response to SARS-CoV-2 S antigens at significantly higher levels than MV-ATU3-SF-2P-dER. The S2 candidates elicited much lower cellular responses, as observed previously with NAb levels, indicating that S2 alone is not sufficient to induce an efficient immune response in these mice. The inventors therefore excluded the S2 candidates from further analysis.

Persistence of Neutralizing Antibodies and Protection from Intranasal Challenge

The inventors monitored the persistence of anti-S antibodies in mice immunized twice with either MV-ATU2-SF-2P-dER or MV-ATU3-SF-2P-dER (FIG. 26a). As usually observed for MV responses (FIG. 26b), S-specific IgG titers persisted and stabilized at high levels (10⁵-10⁶ limiting dilution titers) for both ATU2 and ATU3 candidates for up to three months after boosting (FIG. 26c). However, immunization with the ATU2 construct resulted in significantly higher levels of S-specific IgG and NAb titers (10³-10⁴ limiting dilution titers) over the duration of the experiment (FIG. 26d).

To determine whether these responses confer protection from SARS-CoV-2 infection, immunized mice were challenged intranasally with 1.5×10⁵ PFU of MACo3, a mouse-adapted SARS-CoV-2 virus. Three days after challenge, mice were sacrificed and the presence of virus was examined in lung homogenates. SARS-CoV-2 RNA was measured by RT-qPCR using RdRP gene-specific primers (Protocol, Institut Pasteur, 2020) (Table 5A-5C), and infectious virus levels were titered on Vero E6 cells. SARS-CoV-2 viral RNA was detected in the lungs of all immunized mice after challenge, with the ATU2 group showing an average 2 log₁₀ reduction and the ATU3 group a 1 log₁₀ reduction compared to the empty MV control group (FIG. 26e). However, no infectious virus was detected in the lungs of the ATU2 group and all but one of the ATU3 group (FIG. 26f). These results demonstrate that, although viral replication occurred at low levels, infectivity of the inoculated and progeny virus was efficiently neutralized.

Partial Protection from Intranasal Challenge after a Single Immunization

The inventors next determined whether a single immunization could protect IFNAR^{-/-} mice from challenge with the MACo3 virus (FIG. 27a). Immunized animals were examined for immune responses on days 28 and 48 post-immunization, prior to challenge. All animals exhibited MV- and S-specific antibodies (FIGS. 27b, c). Th1-associated IgG responses to the S antigen as well as SARS-CoV-2 NAbs were present before challenge, although at lower levels than after two immunizations (FIGS. 27d, e). Mice were then challenged intranasally and lung samples collected 3 days after challenge. Although no difference was observed in viral RNA levels between the test and control groups (FIG. 27f), half of the animals immunized with MV-ATU2-SF-2P-dER were negative for infectious virus in the lungs (FIG. 27g), indicating partial protection even after a single administration. In contrast, animals immunized with MV-ATU3-SF-2P-dER were not protected after a single administration. However after prime/boost, both constructs were found protective (FIG. 26).

3. Discussion

Here the inventors reported the development and testing of MV-based COVID-19 vaccine candidates targeting the SARS-CoV-2 S protein. Similar to other vaccine platforms, the full-length prefusion-stabilized S was the most immunogenic, eliciting the strongest humoral and cellular responses. Their lead candidate MV-ATU2-SF-2P-dER elicited high levels of neutralizing antibodies to SARS-CoV-2 and strong Th1-oriented Tcell reponses. Prime-boost immunization afforded protection from intranasal challenge with a mouse-adapted SARS-CoV-2 virus. Moreover, NAb titers persisted months after the immunization—such long-lasting immunity is a hallmark of replicating vector vaccines (Amanna, 2007). T-cell responses, essential to controlling and reducing viral load and viral spread (Rydyznski, 2020), were induced within seven days after a single immunization. Notably, dominance of Th1 responses suggested that these vaccine candidates were less likely to induce immunopathology due to vaccine-induced disease enhancement as previously reported for SARS-CoV-1 and MERS-CoV vaccine studies (Roberts, 2010; Luo, 2018; Qin, 2006; Niu, 2018; Zhang, 2016). In addition, after a single immunization, their lead candidate MV-ATU2-SF-2P-dER also provided sufficient immune protection according to WHO recommendations for a COVID-19 vaccine primary efficacy of at least 50% (Considerations for evaluation of COVID19, WHO, 2020). This suggests that their lead vaccine candidate could protect against both SARS-CoV-2 infection and disease.

To explore the possibility of generating a broad-spectrum vaccine, the inventors also tested a vaccine candidate expressing only the S2 subunit, which is highly conserved among SARS-CoV-1 and SARS-CoV-2 viruses. The S2 subunit has been shown to harbor immunodominant and neutralizing epitopes (Zhang, 2004; He, 2004; Wang, 2020). In this report, while S2 in the MV context induced high S-specific antibody titers, these antibodies could not neutralize the SARS-CoV-2 virus. In terms of cellular responses, S2 did not induce S-specific CD4⁺, CD8⁺ or IL-5⁺ T-cell responses. These observations suggested that the S2 subunit alone was insufficient for inducing immune protection. Given the high titers of non-neutralizing antibodies, it would be interesting to characterize their role in immune responses to SARS-CoV-2 infection and investigate whether they may contribute to immunopathology.

The results of the inventors also yielded interesting differences in the immunogenicity of rMV vaccines expressing the target antigen from ATU2 versus ATU3. S antigen was expressed at higher levels from ATU2, and this correlated with higher humoral and cellular responses. Reducing the immunization dose of the ATU2 candidate to 1×10⁴ TCID₅₀ still induced higher NAb titers than the ATU3 vaccine at 1×10⁵ TCID₅₀ (FIG. 34). Additionally, T-cell responses to the MV vector was also lower with ATU2 constructs. These observations suggested that higher antigen expression could be reducing virus replication in vivo, resulting in lower cellular responses to the vector itself. While MV vaccines have been shown to be effective despite pre-existing immunity to the vector, this more desirable balance in the immunogenicity of antigen and vector likely contributes to greater vaccine efficacy of the ATU2 construct. Nevertheless, as an rMV vehicle for future vaccines, the ATU3 concept is still useful for expressing antigens that are unstable, toxic, or otherwise difficult to express.

### BIBLIOGRAPHY

Combredet, C., V. Labrousse, L. Mollet, C. Lorin, F. Delebecque, B. Hurtrel, H. McClure, M. B. Feinberg, M. Brahic and F. Tangy (2003). “A molecularly cloned Schwarz strain of measles virus vaccine induces strong immune responses in macaques and transgenic mice.” J Virol 77(21): 11546-54.
Escriou, N., B. Callendret, V. Lorin, C. Combredet, P. Marianneau, M. Fevrier and F. Tangy (2014). “Protection from SARS coronavirus conferred by live measles vaccine expressing the spike glycoprotein.” Virology 452-453: 32-41.
Reed, L. J. and H. Muench (1938). “A simple method of estimating fifty percent endpoints.” Am. J. Hyg. 27: 493-497.
Reuter et al, PLoS ONE, 2012, 7(3), e33989, I-8.
Ku et al. (2021) Cell Host & Microbe—for challenge model with Ad5:hACE2.
Hsieh et al. (2020) Science 369, 1501-1505.
MCCallum et al. (2020) Nat. Struct. Mol. Biol., 27, 942-949.
Buchrieser et al. (2020) EMBO Journal, 39:e106267.
Plumet (2005) J. Virol (2005) 79(11):6900-8.
Del Valle (2020), Nat Med., Chen, J. Clin. Invest., 2020.
Xiong et al (2020), Nature Structural & Molecular Biology.
Combredet, C., V. Labrousse, L. Mollet, C. Lorin, F. Delebecque, B. Hurtrel, H. McClure, M. B. Feinberg, M. Brahic and F. Tangy (2003). “A molecularly cloned Schwarz strain of measles virus vaccine induces strong immune responses in macaques and transgenic mice.” J Virol 77(21): 11546-54.
Escriou, N., B. Callendret, V. Lorin, C. Combredet, P. Marianneau, M. Fevrier and F. Tangy (2014). “Protection from SARS coronavirus conferred by live measles vaccine expressing the spike glycoprotein.” Virology 452-453: 32-41.
Reed, L. J. and H. Muench (1938). “A simple method of estimating fifty percent endpoints.” Am. J. Hyg. 27: 493-497.
The GDP@Risk over five years from COVID-19 could range from $3.3 trillion to $82 trillion, says the Centre for Risk Studies www.jbs.cam.ac.uk/insight/2020/economic-impact/. In: Brain food/Economic impact) (2020).
Callaway E, Cyranoski D. China coronavirus: Six questions scientists are asking. Nature 577, 605-607 (2020).
Graham B S. Rapid COVID-19 vaccine development. Science (New York, N.Y.) 368, 945-946 (2020).
Zhou P, et al. A pneumonia outbreak associated with a new coronavirus of probable bat origin. Nature 579, 270-273 (2020).
Wu F, et al. A new coronavirus associated with human respiratory disease in China. Nature 579, 265-269 (2020).
Jaimes J, Millet J, Whittaker G. Proteolytic Cleavage of the SARS-CoV-2 Spike Protein and the Role of the Novel S1/S2 Site. Ssrn, 3581359 (2020).
Hoffmann M, et al. SARS-CoV-2 Cell Entry Depends on ACE2 and TMPRSS2 and Is Blocked by a Clinically Proven Protease Inhibitor. Cell 181, 271-280.e278 (2020).
Du L, He Y, Zhou Y, Liu S, Zheng B J, Jiang S. The spike protein of SARS-CoV—a target for vaccine and therapeutic development. Nature reviews Microbiology 7, 226-236 (2009).
Escriou N, et al. Protection from SARS coronavirus conferred by live measles vaccine expressing the spike glycoprotein. Virology 452-453, 32-41 (2014).
Liniger M, et al. Induction of neutralising antibodies and cellular immune responses against SARS coronavirus by recombinant measles viruses. Vaccine 26, 2164-2174 (2008).
Bodmer B S, Fiedler A H, Hanauer J R H, Prüfer S, Mühlebach M D. Live-attenuated bivalent measles virus-derived vaccines targeting Middle East respiratory syndrome coronavirus induce robust and multifunctional T cell responses against both viruses in an appropriate mouse model. Virology 521, 99-107 (2018).
Zhu F C, et al. Safety, tolerability, and immunogenicity of a recombinant adenovirus type-5 vectored COVID-19 vaccine: a dose-escalation, open-label, non-randomised, first-in-human trial. Lancet (London, England) 395, 1845-1854 (2020).
Bosch B J, et al. Severe acute respiratory syndrome coronavirus (SARS-CoV) infection inhibition using spike protein heptad repeat-derived peptides. Proceedings of the National Academy of Sciences of the United States of America 101, 8455-8460 (2004).
Keng C T, et al. Amino acids 1055 to 1192 in the S2 region of severe acute respiratory syndrome coronavirus S protein induce neutralizing antibodies: implications for the development of vaccines and antiviral agents. Journal of virology 79, 3289-3296 (2005).
Lip K M, et al. Monoclonal antibodies targeting the HR2 domain and the region immediately upstream of the HR2 of the S protein neutralize in vitro infection of severe acute respiratory syndrome coronavirus. Journal of virology 80, 941-950 (2006).
Zhang H, et al. Identification of an antigenic determinant on the S2 domain of the severe acute respiratory syndrome coronavirus spike glycoprotein capable of inducing neutralizing antibodies. Journal of virology 78, 6938-6945 (2004).
Zhong X, et al. B-cell responses in patients who have recovered from severe acute respiratory syndrome target a dominant site in the S2 domain of the surface spike glycoprotein. Journal of virology 79, 3401-3408 (2005).
Frantz P N, Teeravechyan S, Tangy F. Measles-derived vaccines to prevent emerging viral diseases. Microbes Infect 20, 493-500 (2018).
Ramsauer K, et al. Immunogenicity, safety, and tolerability of a recombinant measles-virus-based chikungunya vaccine: a randomised, double-blind, placebo-controlled, active-comparator, first-in-man trial. The Lancet Infectious diseases 15, 519-527 (2015).
Reisinger E C, et al. Immunogenicity, safety, and tolerability of the measles-vectored chikungunya virus vaccine MV-CHIK: a double-blind, randomised, placebo-controlled and active-controlled phase 2 trial. Lancet (London, England) 392, 2718-2727 (2019).
Mateo M, et al. Vaccines inducing immunity to Lassa virus glycoprotein and nucleoprotein protect macaques after a single shot. Science translational medicine 11, (2019).
Nümberger C, Bodmer B S, Fiedler A H, Gabriel G, Mühlebach M D. A Measles Virus-Based Vaccine Candidate Mediates Protection against Zika Virus in an Allogeneic Mouse Pregnancy Model. Journal of virology 93, (2019).
Malczyk A H, et al. A Highly Immunogenic and Protective Middle East Respiratory Syndrome Coronavirus Vaccine Based on a Recombinant Measles Virus Vaccine Platform. Journal of virology 89, 11654-11667 (2015).
Chan J F, et al. Genomic characterization of the 2019 novel human-pathogenic coronavirus isolated from a patient with atypical pneumonia after visiting Wuhan. Emerging microbes & infections 9, 221-236 (2020).
Kirchdoerfer R N, et al. Stabilized coronavirus spikes are resistant to conformational changes induced by receptor recognition or proteolysis. Scientific reports 8, 15701 (2018).
Pallesen J, et al. Immunogenicity and structures of a rationally designed prefusion MERS-CoV spike antigen. Proceedings of the National Academy of Sciences of the United States of America 114, E7348-e7357 (2017).
Wrapp D, et al. Cryo-EM structure of the 2019-nCoV spike in the prefusion conformation. Science (New York, N.Y.) 367, 1260-1263 (2020).
Ujike M, Huang C, Shirato K, Makino S, Taguchi F. The contribution of the cytoplasmic retrieval signal of severe acute respiratory syndrome coronavirus to intracellular accumulation of S proteins and incorporation of S protein into virus-like particles. The Journal of general virology 97, 1853-1864 (2016).
McBride C E, Li J, Machamer C E. The cytoplasmic tail of the severe acute respiratory syndrome coronavirus spike protein contains a novel endoplasmic reticulum retrieval signal that binds COPI and promotes interaction with membrane protein. Journal of virology 81, 2418-2428 (2007).
Ujike M, Taguchi F. Incorporation of spike and membrane glycoproteins into coronavirus virions. Viruses 7, 1700-1725 (2015).
Schwegmann-Wessels C, et al. A novel sorting signal for intracellular localization is present in the S protein of a porcine coronavirus but absent from severe acute respiratory syndrome-associated coronavirus. The Journal of biological chemistry 279, 43661-43666 (2004).
Combredet C, et al. A molecularly cloned Schwarz strain of measles virus vaccine induces strong immune responses in macaques and transgenic mice. Journal of virology 77, 11546-11554 (2003).
Plumet S, Duprex W P, Gerlier D. Dynamics of viral RNA synthesis during measles virus infection. Journal of virology 79, 6900-6908 (2005).
Mura M, Ruffie C, Billon-Denis E, Combredet C, Tournier J N, Tangy F. hCD46 receptor is not required for measles vaccine Schwarz strain replication in vivo: Type-I IFN is the species barrier in mice. Virology 524, 151-159 (2018).
Finkelman F D, et al. Lymphokine control of in vivo immunoglobulin isotype selection. Annual review of immunology 8, 303-333 (1990).
Protocol: Real-time RT-PCR assays for the detection of SARS-CoV-2 Institut Pasteur, Paris.). Protocol: Real-time RT-PCR assays for the detection of SARS-CoV-2 Institut Pasteur, Paris (2020).
Amanna I J, Carlson N E, Slifka M K. Duration of humoral immunity to common viral and vaccine antigens. The New England journal of medicine 357, 1903-1915 (2007).
Rydyznski Moderbacher C, et al. Antigen-Specific Adaptive Immunity to SARS-CoV-2 in Acute COVID-19 and Associations with Age and Disease Severity. Cell, (2020).
Roberts A, et al. Immunogenicity and protective efficacy in mice and hamsters of a beta-propiolactone inactivated whole virus SARS-CoV vaccine. Viral Immunol 23, 509-519 (2010).
Luo F, Liao F L, Wang H, Tang H B, Yang Z Q, Hou W. Evaluation of Antibody-Dependent Enhancement of SARS-CoV Infection in Rhesus Macaques Immunized with an Inactivated SARS-CoV Vaccine. Virol Sin 33, 201-204 (2018).
Qin E, et al. Immunogenicity and protective efficacy in monkeys of purified inactivated Vero-cell SARS vaccine. Vaccine 24, 1028-1034 (2006).
Niu P, et al. A novel human mAb (MERS-GD27) provides prophylactic and postexposure efficacy in MERS-CoV susceptible mice. Sci China Life Sci 61, 1280-1282 (2018).
Zhang N, et al. Identification of an ideal adjuvant for receptor-binding domain-based subunit vaccines against Middle East respiratory syndrome coronavirus. Cell Mol Immunol 13, 180-190 (2016).
CONSIDERATIONS FOR EVALUATION OF COVID19 VACCINESFORWHO EUL v24092020. (ed Vaccine and Immunization Devices Assessment Team (VAX) PUP, Regulation and Prequalification Department (RPQ), Access to Medicineand Health Products (MHP), World Health Organization, Geneva, Switzerland) (2020).
He Y, et al. Identification of immunodominant sites on the spike protein of severe acute respiratory syndrome (SARS) coronavirus: implication for developing SARS diagnostics and vaccines. J Immunol 173, 4050-4057 (2004).
Wang Q, Qiu Y, Li J Y, Zhou Z J, Liao C H, Ge X Y. A Unique Protease Cleavage Site Predicted in the Spike Protein of the Novel Pneumonia Coronavirus (2019-nCoV) Potentially Related to Viral Transmissibility. Virol Sin 35, 337-339 (2020).
Print R D B. Draft landscape of COVID-19 candidate vaccines.). World Health Organization (2020).
Ahi Y S, Bangari D S, Mittal S K. Adenoviral vector immunity: its implications and circumvention strategies. Curr Gene Ther 11, 307-320 (2011).
Folegatti P M, et al. Safety and immunogenicity of the ChAdOx1 nCoV-19 vaccine against SARS-CoV-2: a preliminary report of a phase 1/2, single-blind, randomised controlled trial. Lancet (London, England) 396, 467-478 (2020).
Zuniga A, et al. Attenuated measles virus as a vaccine vector. Vaccine 25, 2974-2983 (2007).
Domingo C, et al. Long-term immunity against yellow fever in children vaccinated during infancy: a longitudinal cohort study. The Lancet Infectious diseases 19, 1363-1370 (2019).
Pebody R G, et al. Immunogenicity of second dose measles-mumps-rubella (MMR) vaccine and implications for serosurveillance. Vaccine 20, 1134-1140 (2002).
Mura M, Combredet C, Najburg V, Sanchez David R Y, Tangy F, Komarova A V. Nonencapsidated 5′ Copy-Back Defective Interfering Genomes Produced by Recombinant Measles Viruses Are Recognized by RIG-1 and LGP2 but Not MDA5. Journal of virology 91, (2017).
Sankoh O, et al. The non-specific effects of vaccines and other childhood interventions: the contribution of INDEPTH Health and Demographic Surveillance Systems. Int J Epidemiol 43, 645-653 (2014).
Benn C S, FiskerAB, Whittle H C, Aaby P. Revaccination with Live Attenuated Vaccines Confer Additional Beneficial Nonspecific Effects on Overall Survival: A Review. EBioMedicine 10, 312-317 (2016).
Aaby P, et al. Non-specific effects of standard measles vaccine at 4.5 and 9 months of age on childhood mortality: randomised controlled trial. BMJ 341, c6495 (2010).
Buitrago-Garcia D, et al. Occurrence and transmission potential of asymptomatic and presymptomatic SARS-CoV-2 infections: A living systematic review and meta-analysis. PLoS Med 17, e1003346 (2020).
Calain P, Roux L. The rule of six, a basic feature for efficient replication of Sendai virus defective interfering RNA. Journal of virology 67, 4822-4830 (1993).
Duprex W P, Collins F M, Rima B K. Modulating the function of the measles virus RNA-dependent RNA polymerase by insertion of green fluorescent protein into the open reading frame. Journal of virology 76, 7322-7328 (2002).
Kärber G. Beitrag zur kollektiven Behandlung pharmakologischer Reihenversuche. Archiv f experiment Pathol u Pharmakol 162, 480-483 (1931)

1. A nucleic acid construct comprising:
(1) a cDNA molecule encoding a full length, antigenomic (+) RNA strand of an attenuated strain of measles virus (MV); and
(2) a first heterologous polynucleotide encoding:
(a) a spike (S) protein of SARS-CoV-2 of SEQ ID NO: 3, or
(b) an immunogenic fragment of the full-length S protein in (a) selected from the group consisting of the S1 polypeptide of SEQ ID NO: 11, the S2 polypeptide of SEQ ID NO: 13, the Secto polypeptide of SEQ ID NO: 7 and the tri-Secto polypeptide of SEQ ID NO: 16, or
(c) a variant of (a) or (b) in which from 1 to 10 amino acids are modified by insertion, substitution, or deletion.

2. The nucleic acid construct according to claim 1, wherein the variant in (c) encodes a polypeptide comprising:
(i) a mutation that maintains the expressed full length S protein in its prefusion conformation, and/or
(ii) a mutation that inactivates the furin cleavage site of the S protein, and/or
(iii) a mutation that inactivates the Endoplasmic Reticulum Retrieval Signal (EERS), and/or
(iv) a mutation that maintains the receptor-binding domain (RBD) localized in the S1 domain of the S protein in the closed conformation, and
wherein the first heterologous polynucleotide is positioned in an additional transcription unit (ATU) located between the P gene and the M gene of the MV (ATU2) or in an ATU located downstream of the H gene of the MV (ATU3).

3. The nucleic acid construct according to claim 2, wherein:
(i) the mutation that maintains the expressed full length S protein in its prefusion conformation is a mutation by substitution of two proline residues at positions 986 and 987 (K986P and V987P) of the amino acid sequence of the S protein of SARS-CoV-2 of SEQ ID NO: 3, or a mutation by substitution of six proline residues at positions 817, 892, 899, 942, 986 and 987 (F817P, A892P, A899P, A942P, K986P and V987P) of the amino acid sequence of the S protein of SARS-CoV-2 of SEQ ID NO: 3, and/or
(ii) the mutation that inactivates the furin cleavage site of the S protein is a mutation by substitution of three amino acid residues occurring in the S1/S2 furin cleavage site at positions 682, 683 and 685 (R682G, R683S and R685G) of the amino acid sequence of the S protein of SARS-CoV-2 of SEQ ID NO: 3, or a mutation by deletion of the loop encompassing the S1/S2 furin cleavage site between amino acid at position 675 and amino acid at position 685 of the S protein of SARS-CoV-2 of SEQ ID NO: 3, the loop consisting of the amino acid sequence QTQTNSPRRAR of SEQ ID NO: 50, and/or
(iii) the mutation that inactivates the EERS is a mutation by substitution of two alanine residues at positions 1269 and 1271 of the amino acid sequence of SEQ ID NO: 3, and/or
(iv) the mutation that maintains the RBD localized in the S1 domain of the S protein in the closed conformation is a mutation by substitution of two cysteine residues at positions 383 and 985 (S383C and D985C) of the amino acid sequence of the S protein of SARS-CoV-2 of SEQ ID NO: 3, or a mutation by substitution of two cysteine residues at positions 413 and 987 (G413C and P987C) of the amino acid sequence of the S protein of SARS-CoV-2 of SEQ ID NO: 3; and/or
(v) the variant in (c) encodes a polypeptide comprising a mutation selected from the group consisting of a deletion of the amino acid residues at positions 69 and 70 of the amino acid sequence of SEQ ID NO: 3, a deletion of the amino acid residues at positions 144 and 145 of the amino acid sequence of SEQ ID NO: 3, a mutation by substitution of the tyrosine residue at position 501 of the amino acid sequence of SEQ ID NO: 3 (N501Y), a mutation by substitution of the aspartic acid residue at position 570 of the amino acid sequence of SEQ ID NO: 3 (A570D), a mutation by substitution of the histidine residue at position 681 of the amino acid sequence of SEQ ID NO: 3 (P681H), a mutation by substitution of the isoleucine residue at position 716 of the amino acid sequence of SEQ ID NO: 3 (T7161), a mutation by substitution of the alanine residue at position 982 of the amino acid sequence of SEQ ID NO: 3 (S982A), a mutation by substitution of the histidine residue at position 1118 of the amino acid sequence of SEQ ID NO: 3 (D1118H), a mutation by substitution of the lysine residue at position 484 of the amino acid sequence of SEQ ID NO: 3 (E484K), a mutation by substitution of the asparagine residue at position 417 of the amino acid sequence of SEQ ID NO: 3 (K417N), a mutation by substitution of the threonine residue at position 417 of the amino acid sequence of SEQ ID NO: 3 (K417T) and a mutation by substitution of the glycine residue at position 614 of the amino acid sequence of SEQ ID NO: 3 (D614G).

4. The nucleic acid construct according to any one of claims 1 to 3, further comprising a second heterologous polynucleotide encoding at least one polypeptide of SARS-CoV-2 selected from the group consisting of: nucleocapsid (N) polypeptide or a variant thereof having at least 90% identity with the N polypeptide, matrix (M) polypeptide or a variant thereof having at least 90% identity with M polypeptide, E polypeptide or a variant thereof having at least 90% identity with E polypeptide, 8a polypeptide or a variant thereof having at least 90% identity with 8a polypeptide, 7a polypeptide or a variant thereof having at least 90% identity with 7a polypeptide, 3A polypeptide or a variant thereof having at least 90% identity with 3a polypeptide, and immunogenic fragments thereof; the second heterologous polynucleotide positioned within an additional transcription unit (ATU) at a location different from the ATU of the first heterologous polynucleotide.

5. The nucleic acid construct according to any one of claims 1 to 4, wherein the first heterologous polynucleotide encodes a polypeptide having an amino acid sequence selected from the group consisting of SEQ ID NOs: 5, 7, 9, 15, 17, 19, 43, 45, 47, 49, 52, 54, 56, 58, 60, 62 and 65.

6. The nucleic acid construct according to claim 4, wherein the second heterologous polynucleotide encodes at least one of the N polypeptide of SEQ ID NO: 22, the M polypeptide of sequence SEQ ID NO: 24 or its endodomain, the E polypeptide of sequence SEQ ID NO: 23, the ORF8 polypeptide of SEQ ID NO: 25, the ORF7a polypeptide of SEQ ID NO: 27, and the ORF3a polypeptide of SEQ ID NO: 26.

7. The nucleic acid construct according to any one of claims 1 to 6, wherein the first heterologous polynucleotide has the open reading frame selected from the group consisting of:
i. SEQ ID NO: 1 or 2 or 36 which encodes the S polypeptide,
ii. SEQ ID NO: 10 which encodes the S1 polypeptide,
iii. SEQ ID NO: 12 which encodes the S2 polypeptide,
iv. SEQ ID NO: 4 which encodes the stab-S polypeptide (S2P),
v. SEQ ID NO: 6 which encodes the Secto polypeptide,
vi. SEQ ID NO: 8 which encodes the stab-Secto polypeptide,
vii. SEQ ID NO:14 which encodes the stab-S2 polypeptide,
viii. SEQ ID NO: 16 which encodes the tri-Secto polypeptide,
ix. SEQ ID NO: 18 which encodes the tristab-Secto polypeptide,
x. SEQ ID NO: 42 which encodes the S3F polypeptide,
xi. SEQ ID NO: 44 which encodes the S2P3F polypeptide,
xii. SEQ ID NO: 46 which encodes the S2PΔF polypeptide,
xiii. SEQ ID NO: 48 which encodes the S2PΔF2A polypeptide,
xiv. SEQ ID NO: 51 which encodes the T4-S2P3F polypeptide (tristab-Secto-3F),
xv. SEQ ID NO: 53 which encodes the S6P polypeptide,
xvi. SEQ ID NO: 55 which encodes the S6P3F polypeptide,
xvii. SEQ ID NO: 57 which encodes the S6PΔF polypeptide,
xviii. SEQ ID NO: 59 which encodes the SCCPP polypeptide,
xix. SEQ ID NO: 61 which encodes the SCC6P polypeptide,
xx. SEQ ID NO: 63 which encodes the S_{MVopt}2P polypeptide,
xxi. SEQ ID NO: 64 which encodes the S_{MVopt}ΔF polypeptide, and
xxii. SEQ ID NO: 66 which encodes the S_{MVopt}2PΔF polypeptide.

8. The nucleic acid construct according to any one of claims 1 to 7, which is a cDNA construct comprising from 5′ to 3′ end the following polynucleotides coding for open reading frames:
(a) a polynucleotide encoding the N protein of the MV;
(b) a polynucleotide encoding the P protein of the MV;
(c) the first heterologous polynucleotide as defined in any one of claims 1-3, 4 and 6;
(d) a polynucleotide encoding the M protein of the MV;
(e) a polynucleotide encoding the F protein of the MV;
(f) a polynucleotide encoding the H protein of the MV;
(g) a polynucleotide encoding the L protein of the MV; and
wherein the polynucleotides are operatively linked within the nucleic acid construct and are under the control of a viral replication and transcriptional regulatory elements such as MV leader and trailer sequences and are framed by a T7 promoter and a T7 terminator and are framed by restriction sites suitable for cloning in a vector to provide a recombinant MV-CoV expression cassette.

9. The nucleic acid construct according to any one of claims 1 to 8, further comprising:
(a) a GGG motif followed by a hammerhead ribozyme sequence at the 5′-end of the nucleic acid construct, adjacent to a first nucleotide of the nucleotide sequence encoding a full-length antigenomic (+)RNA strand of an attenuated MV strain, in particular of a Schwarz strain or of a Moraten strain, and
(b) a nucleotide sequence of a ribozyme, in particular the sequence of the Hepatitis delta virus ribozyme (6), at the 3′-end of the recombinant MV-CoV nucleic acid molecule, adjacent to the last nucleotide of the nucleotide sequence encoding the full length anti-genomic (+)RNA strand.

10. The nucleic acid construct according to any one of claims 4 to 9, wherein the second heterologous polynucleotide encodes the N polypeptide of SARS-CoV-2, and the second heterologous polynucleotide being cloned in an ATU at a different location with respect to the ATU used for cloning the first heterologous polynucleotide.

11. The nucleic acid construct according to any one of claims 1 to 10, wherein:
(i) the first heterologous polynucleotide comprises a sequence selected from the group consisting of SEQ ID NO: 36, SEQ ID NO: 63, SEQ ID NO: 64 and SEQ ID NO: 66, and is positioned within ATU2, or
(ii) the first heterologous polynucleotide comprises a sequence selected from the group consisting of SEQ ID NO: 2, SEQ ID NO: 4, SEQ ID NO: 44, SEQ ID NO: 46, SEQ ID NO: 48, SEQ ID NO: 51, SEQ ID NO: 53, SEQ ID NO: 55, SEQ ID NO: 57, SEQ ID NO: 59 and SEQ ID NO: 61, and is positioned within ATU3.

12. The nucleic acid construct according to any one of claims 4 to 10, wherein:
(i) the first heterologous polynucleotide is positioned within ATU3 and the second heterologous polynucleotide is positioned within ATU2, or
(ii) the first heterologous polynucleotide is positioned within ATU2 and the second heterologous polynucleotide is positioned within ATU3.

13. The nucleic acid construct according to any one of claims 1 to 12, wherein the measles virus is an attenuated virus strain selected from the group consisting of the Schwarz strain, the Zagreb strain, the AIK-C strain, the Moraten strain, the Philips strain, the Beckenham 4A strain, the Beckenham 16 strain, the CAM-70 strain, the TD 97 strain, the Leningrad-16 strain, the Shanghai 191 strain and the Belgrade strain.

14. A transfer vector for the rescue of a recombinant Measles virus (MV), comprising the nucleic acid construct according to any one of claims 1 to 13.

15. A transfer vector comprising a sequence encoding a polypeptide of SARS-CoV-2 that is selected from the group consisting of:
i. SEQ ID NO: 1 or 2 or 36 (construct S),
ii. SEQ ID NO: 4 (construct stab-S),
iii. SEQ ID NO: 6 (construct Secto),
iv. SED ID NO: 8 (construct stab-Secto),
v. SEQ ID NO: 10 (construct S1),
vi. SEQ ID NO: 12 (construct S2),
vii. SEQ ID NO: 14 (construct stab-S2),
viii. SEQ ID NO: 16 (construct tri-Secto),
ix. SEQ ID NO: 18 (construct tristab-Secto),
x. SEQ ID NO: 42 (construct S3F),
xi. SEQ ID NO: 44 (construct S2P3F),
xii. SEQ ID NO: 46 (construct S2PΔF),
xiii. SEQ ID NO: 48 (construct S2PΔF2A),
xiv. SEQ ID NO: 21 or 37 (construct N),
xv. SEQ ID NO: 51 (construct T4-S2P3F (tristab-Secto-3F)),
xvi. SEQ ID NO: 53 (construct S6P),
xvii. SEQ ID NO: 55 (construct S6P3F),
xviii. SEQ ID NO: 57 (construct S6PΔF),
xix. SEQ ID NO: 59 (construct SCCPP),
xx. SEQ ID NO: 61 (construct SCC6P),
xxi. SEQ ID NO: 63 (construct S_{MVopt}2P),
xxii. SEQ ID NO: 64 (construct S_{MVopt}ΔF), and
xxiii. SEQ ID NO: 66 (construct S_{MVopt}2PΔF).

16. A recombinant measles virus of the Schwarz strain comprising in its genome an expression cassette operatively linked thereto, the expression cassette comprising the nucleic acid construct according to any one of claims 1 to 13.

17. The recombinant measles virus according to claim 16, further expressing at least one polypeptide selected from N, M, E, ORF7a, ORF8 and ORF3a of the SARS-CoV-2 strain, and immunogenic fragments thereof.

18. An immunogenic composition or a vaccine comprising (i) an effective dose of the recombinant measles virus according to claim 16 or 17, and (ii) a pharmaceutically acceptable vehicle, wherein the composition or the vaccine elicits a neutralizing humoral response and/or a cellular response against the polypeptide(s) of SARS-CoV-2 in an animal host after a single immunization.

19. The immunogenic composition or vaccine according to claim 18 for use in eliciting a protective humoral immune response and/or a cellular immune response against SARS-CoV-2 in a host in need thereof.

20. A process for rescuing recombinant measles virus expressing the polypeptide of SARS-CoV-2 encoded by the first heterologous polynucleotide as defined in any one of claims 1-3, 4 and 6 of SARS-CoV-2, comprising:
(a) co-transfecting helper cells stably expressing T7 RNA polymerase and measles virus N and P proteins with (i) the nucleic acid construct according to any one of claims 1 to 13 or with the plasmid vector according to claim 13 or 14, and with (ii) a vector encoding the MV L polymerase;
(b) maintaining the transfected cells in conditions suitable for the production of recombinant measles virus;
(c) infecting cells enabling propagation of the recombinant measles virus by co-cultivating them with the transfected cells of step (b); and
(d) harvesting the recombinant measles virus.

21. A nucleic acid molecule comprising a polynucleotide selected from the group consisting of:
i. SEQ ID NO: 1 or 2 or 36 (construct S);
ii. SEQ ID NO: 4 (construct stab-S);
iii. SEQ ID NO: 6 (construct Secto);
iv. SED ID NO: 8 (construct stab-Secto);
v. SEQ ID NO: 10 (construct S1),
vi. SEQ ID NO: 12 (construct S2),
vii. SEQ ID NO: 14 (construct stab-S2),
viii. SEQ ID NO: 16 (construct tri-Secto),
ix. SEQ ID NO: 18 (construct tristab-Secto),
x. SEQ ID NO: 42 (construct S3F),
xi. SEQ ID NO: 44 (construct S2P3F),
xii. SEQ ID NO: 46 (construct S2PΔF),
xiii. SEQ ID NO: 48 (construct S2PΔF2A),
xiv. SEQ ID NO: 21 or 37 (construct N),
xv. SEQ ID NO: 51 (construct T4-S2P3F (tristab-Secto-3F)),
xvi. SEQ ID NO: 53 (construct S6P),
xvii. SEQ ID NO: 55 (construct S6P3F),
xviii. SEQ ID NO: 57 (construct S6PΔF),
xix. SEQ ID NO: 59 (construct SCCPP),
xx. SEQ ID NO: 61 (construct SCC6P),
xxi. SEQ ID NO: 63 (construct S_{MVopt}2P),
xxii. SEQ ID NO: 64 (construct S_{MVopt}ΔF), and
xxiii. SEQ ID NO: 66 (construct S_{MVopt}2PΔF).

22. A polypeptide comprising an amino acid sequence selected from the group consisting of:
i. SEQ ID NO: 3 (construct S);
ii. SEQ ID NO: 5 (construct stab-S);
iii. SEQ ID NO: 7 (construct Secto);
iv. SED ID NO: 9 (construct stab-Secto);
v. SEQ ID NO: 11 (construct S1),
vi. SEQ ID NO: 13 (construct S2),
vii. SEQ ID NO: 15 (construct stab-S2),
viii. SEQ ID NO: 17 (construct tri-Secto),
ix. SEQ ID NO: 19 (construct tristab-Secto),
x. SEQ ID NO: 43 (construct S3F),
xi. SEQ ID NO: 45 (construct S2P3F),
xii. SEQ ID NO: 47 (construct S2PΔF),
xiii. SEQ ID NO: 49 (construct S2PΔF2A),
xiv. SEQ ID NO: 22 (construct N),
xv. SEQ ID NO: 52 (construct T4-S2P3F (tristab-Secto-3F)),
xvi. SEQ ID NO: 54 (construct S6P),
xvii. SEQ ID NO: 56 (construct S6P3F),
xviii. SEQ ID NO: 58 (construct S6PΔF),
xix. SEQ ID NO: 60 (construct SCCPP),
xx. SEQ ID NO: 62 (construct SCC6P), and
xxi. SEQ ID NO: 65 (construct S_{MVopt}ΔF).

23. A recombinant protein expressed by the transfer vector according to claim 14 or 15, further comprising an amino acid tag for purification.

24. A recombinant protein expressed in vitro or in vivo by the transfer vector according to claim 14 or 15.

25. In vitro use of an antigen having the sequence of any one of SEQ ID NOs: 3, 5, 7, 9, 11, 13, 15, 17, 19, 22, 23, 24, 25, 26, 27, 43, 45, 47, 49, 52, 54, 56, 58, 60, 62 and 65 for the detection of the presence of antibodies against the antigen in a biological sample previously obtained from an individual suspected of being infected by SARS-CoV-2, wherein the polypeptide is contacted with the biological sample to determine the presence of antibodies against the antigen.

26. A method for treating or preventing an infection by SARS-CoV-2 in a human host, comprising administering the immunogenic composition or vaccine according to claim 18 to the host.

27. A method for inducing a protective immune response against SARS-CoV-2 in a host, comprising administering the immunogenic composition or vaccine according to claim 18 to the host.

28. The method according to claim 26 or 27, comprising a first administration of the immunogenic composition and a second administration of the immunogenic composition.

29. The method according to claim 28, wherein the second administration is performed from one month to two months after the first administration.

30. A nucleic acid construct comprising:
(1) a cDNA molecule encoding a full length antigenomic (+) RNA strand of an attenuated strain of measles virus (MV); and
(2) a first heterologous polynucleotide encoding a S protein or immunogenic fragment thereof of SARS-CoV-2 comprising an insertion, substitution, or deletion in the 11 amino acid residue sequence of the S protein aligned with positions 1263 to 1273 of the amino acid sequence of SEQ ID NO: 3, and wherein the insertion, substitution, or deletion increases cell surface expression of the S protein or immunogenic fragment thereof,
wherein the first heterologous polynucleotide is positioned in an additional transcription unit (ATU) located between the P gene and the M gene of the MV (ATU2) or in an ATU located 3′ of the H gene of the MV (ATU3).

31. The nucleic acid construct of claim 30, wherein the S protein or immunogenic fragment thereof comprises a substitution in the 11 amino acid residue sequence of the S protein aligned with positions 1263 to 1273 of the amino acid sequence of SEQ ID NO: 3.

32. The nucleic acid construct of claim 30, wherein the S protein or immunogenic fragment thereof comprises a deletion of all or part of the 11 amino acid residue sequence of the S protein aligned with positions 1263 to 1273 of the amino acid sequence of SEQ ID NO: 3.

33. The nucleic acid construct of any one of claims 30 to 32, wherein the encoded S protein or immunogenic fragment thereof further comprises one or more additional substitutions that maintain the expressed S protein in its prefusion conformation.

34. The nucleic acid construct of claim 33, wherein the encoded S protein or immunogenic fragment thereof further comprises the amino acid substitutions K986P and V987P at the amino acid positions corresponding to positions K986 and V987 of the amino acid sequence of SEQ ID NO: 3.

35. The nucleic acid construct of any one of claims 30 to 34, wherein the encoded S protein or immunogenic fragment thereof is a dual domain S protein.

36. The nucleic acid construct of any one of claims 30 to 35, wherein the first heterologous polynucleotide is positioned in ATU2.

37. The nucleic acid construct of any one of claims 30 to 36, wherein the first heterologous polynucleotide encodes:
(a) a prefusion-stabilized SF-2P-dER polypeptide of SEQ ID NO: 76, or a variant thereof having at least 90% identity with SEQ ID NO: 76, wherein the variant does not vary at positions 986 and 987; or
(b) a prefusion-stabilized SF-2P-2a polypeptide of SEQ ID NO: 82, or a variant thereof having at least 90% identity with SEQ ID NO: 82, wherein the variant does not vary at positions 986, 987, 1269, and 1271.

38. The nucleic acid construct of claim 37, wherein the first heterologous polynucleotide encodes:
(a) a prefusion-stabilized SF-2P-dER polypeptide of SEQ ID NO: 76; or
(b) a prefusion-stabilized SF-2P-2a polypeptide of SEQ ID NO: 82.

39. The nucleic acid construct of claim 38, wherein the first heterologous polynucleotide comprises SEQ ID NO: 75 which encodes the SF-2P-dER polypeptide, or SEQ ID NO: 81 which encodes the SF-2P-2a polypeptide.

40. The nucleic acid construct of claim 39, wherein the first heterologous polynucleotide comprises SEQ ID NO: 75 which encodes the SF-2P-dER polypeptide.

41. The nucleic acid construct of any one of claims 30 to 40, further comprising a second heterologous polynucleotide encoding at least one polypeptide of SARS-CoV-2 selected from the group consisting of: nucleocapsid (N) polypeptide or a variant thereof having at least 90% identity with the N polypeptide; matrix (M) polypeptide or a variant thereof having at least 90% identity with M polypeptide; E polypeptide or a variant thereof having at least 90% identity with E polypeptide; 8a polypeptide or a variant thereof having at least 90% identity with 8a polypeptide; 7a polypeptide or a variant thereof having at least 90% identity with 7a polypeptide; 3A polypeptide or a variant thereof having at least 90% identity with 3a polypeptide; immunogenic fragments thereof, the second heterologous polynucleotide being positioned within an additional transcription unit (ATU) at a location different from the ATU of the first heterologous polynucleotide.

42. The nucleic acid construct of any one of claims 30 to 40, further comprising a second heterologous polynucleotide encoding at least one polypeptide of SARS-CoV-2 selected from the group consisting of: nucleocapsid (N) polypeptide; matrix (M) polypeptide; E polypeptide; 8a polypeptide; 7a polypeptide; 3A polypeptide; and immunogenic fragments thereof, the second heterologous polynucleotide being positioned within an additional transcription unit (ATU) at a location different from the ATU of the first heterologous polynucleotide.

43. The nucleic acid construct of claim 42, wherein the second heterologous polynucleotide encodes N polypeptide and the second heterologous polynucleotide is positioned within an additional transcription unit (ATU) at a location different from the ATU of the first heterologous polynucleotide.

44. The nucleic acid construct of any one of claims 30 to 40, wherein the second heterologous polynucleotide encodes at least one of the N polypeptide of SEQ ID NO: 22, the M polypeptide of sequence SEQ ID NO: 24 or its endodomain, the E polypeptide of sequence SEQ ID NO: 23, the ORF8 polypeptide of SEQ ID NO: 25, the ORF7a polypeptide of SEQ ID NO: 27 and/or the ORF3a polypeptide of SEQ ID NO: 26, the second heterologous polynucleotide being positioned within an additional transcription unit (ATU) at a location different from the ATU of the first heterologous polynucleotide.

45. The nucleic acid construct of any one of claims 41 to 44, wherein the second heterologous protein is within an ATU that is upstream of the N gene of the MV (ATU1), between the P and M genes of the MV (ATU2), or between the H and L genes of the MV (ATU3).

46. The nucleic acid construct of any one of claims 30 to 45, further comprising from 5′ to 3′ the following polynucleotides coding for open reading frames:
(a) a polynucleotide encoding the N protein of the MV;
(b) a polynucleotide encoding the P protein of the MV;
(c) the first heterologous polynucleotide;
(d) a polynucleotide encoding the M protein of the MV;
(e) a polynucleotide encoding the F protein of the MV;
(f) a polynucleotide encoding the H protein of the MV;
(g) a polynucleotide encoding the L protein of the MV; and
wherein the polynucleotides are operatively linked within the nucleic acid construct, are under the control of MV leader and trailer sequences, are framed by a T7 promoter and a T7 terminator, and are framed by restriction sites suitable for cloning in a vector to provide a recombinant MV-CoV expression cassette.

47. The nucleic acid construct of any one of claims 30 to 45, further comprising:
(a) a GGG motif followed by a hammerhead ribozyme sequence at the 5′-end of the nucleic acid construct, adjacent to the first nucleotide of a nucleotide sequence encoding a full-length antigenomic (+)RNA strand of an attenuated MV strain; and
(b) a nucleotide sequence of the Hepatitis delta virus ribozyme (6) at the 3′-end of the nucleic acid construct, adjacent to a last nucleotide of the nucleotide sequence encoding the full length anti-genomic (+)RNA strand of the attenuated MV strain.

48. The nucleic acid construct of any one of claims 30 to 47, wherein the measles virus is an attenuated virus strain selected from the group consisting of the Schwarz strain, the Zagreb strain, the AIK-C strain, the Moraten strain, the Philips strain, the Beckenham 4A strain, the Beckenham 16 strain, the CAM-70 strain, the TD 97 strain, the Leningrad-16 strain, the Shanghai 191 strain, and the Belgrade strain.

49. The nucleic acid construct of claim 48, wherein the measles virus is the Schwarz strain.

50. A plasmid vector comprising the nucleic acid construct of any one of claims 30 to 49, wherein the plasmid vector is SEQ ID NO: 29 or SEQ ID NO: 38.

51. A recombinant measles virus comprising in its genome the nucleic acid construct of any one of claims 30 to 49.

52. An immunogenic composition comprising the recombinant measles virus of claim 51 and a pharmaceutically acceptable vehicle.

53. The recombinant measles virus of claim 51 or the immunogenic composition of claim 52 for use in inducing an immune response against SARS-CoV-2 virus in a subject.

54. A method for preventing or treating an infection by SARS-CoV-2 in a subject, comprising administering the immunogenic composition of claim 52 to the subject.

55. A method for inducing an immune response against SARS-CoV-2 virus in a subject, comprising administering the immunogenic composition according to claim 52 to the subject.

56. The method of claim 54 or 55, comprising a first administration of the immunogenic composition and a second administration of the immunogenic composition.

57. The method according to claim 56, wherein the second administration is performed at from one to two months after the first administration.

58. A process for rescuing recombinant measles virus of claim 51, comprising:
(a) co-transfecting helper cells stably expressing T7 RNA polymerase and measles virus N and P proteins with (i) the nucleic acid construct according to any one of claims 30 to 49 or with the plasmid vector comprising the nucleic acid construct according to claim 50, and with (ii) a vector encoding the MV L polymerase;
(b) maintaining the transfected helper cells in conditions suitable for the production of recombinant measles virus;
(c) infecting cells enabling propagation of the recombinant measles virus by co-cultivating them with the transfected helper cells of step (b); and
(d) harvesting recombinant measles virus.

59. A nucleic acid molecule comprising a polynucleotide of SEQ ID NO: 75 (construct SF-2P-dER) or SEQ ID NO: 81 (construct SF-2P-2a).

60. A polypeptide which has an amino acid sequence of SEQ ID NO: 76 (construct SF-2P-dER) or SEQ ID NO: 82 (construct SF-2P-2a).

61. In vitro use of an antigen of the polypeptide of claim 60 for the detection of the presence of antibodies against the antigen in a biological sample previously obtained from an individual suspected of being infected by SARS-CoV-2, wherein the polypeptide is contacted with the biological sample to determine the presence of antibodies against the antigen.

62. A method comprising contacting a biological sample with the polypeptide of claim 60 and detecting the formation of antibody-antigen complexes between antibodies present in the biological sample and the polypeptide.

63. The method of claim 62, wherein the biological sample is obtained from an individual suspected of being infected by SARS-CoV-2.
